SEQUENCE LISTING (796 sequences)

SEQ ID NO: 1 - RNA, 30 nt, synthetic construct
  misc_feature 1..30: note = Description of Artificial Sequence: Synthetic Ctrl1 tiRNA oligonucleotide
  source 1..30: mol_type = other RNA
tgaagggttttttgtgtctctatttccttc

SEQ ID NO: 2 - RNA, 29 nt, synthetic construct
  misc_feature 1..29: note = Description of Artificial Sequence: Synthetic Ctrl2 tiRNA oligonucleotide
  source 1..29: mol_type = other RNA
tgtgagtcacgtgagggcagaatctgctc

SEQ ID NO: 3 - RNA, 31 nt, synthetic construct
  misc_feature 1..31: note = Description of Artificial Sequence: Synthetic Ctrl3 tiRNA oligonucleotide
  source 1..31: mol_type = other RNA
gcattcacttggatagtaaatccaagctgaa

SEQ ID NO: 4 - RNA, 30 nt, synthetic construct
  misc_feature 1..30: note = Description of Artificial Sequence: Synthetic 5' Ala tiRNA oligonucleotide
  source 1..30: mol_type = other RNA
gggggtgtagctcagtggtagagcgcgtgc

SEQ ID NO: 5 - RNA, 32 nt, synthetic construct
  misc_feature 1..32: note = Description of Artificial Sequence: Synthetic 5' Val tiRNA oligonucleotide
  source 1..32: mol_type = other RNA
gtttccgtagtgtagtggttatcacgttcgcc

SEQ ID NO: 6 - RNA, 30 nt, synthetic construct
  misc_feature 1..30: note = Description of Artificial Sequence: Synthetic 5'-Gly-GCC tiRNA oligonucleotide
  source 1..30: mol_type = other RNA
gcatgggtggttcagtggtagaattctcgc

SEQ ID NO: 7 - RNA, 31 nt, synthetic construct
  misc_feature 1..31: note = Description of Artificial Sequence: Synthetic 5'-GlyCCC tiRNA oligonucleotide
  source 1..31: mol_type = other RNA
gcgccgctggtgtagtggtatcatgcaagat

SEQ ID NO: 8 - RNA, 30 nt, synthetic construct
  misc_feature 1..30: note = Description of Artificial Sequence: Synthetic 5'-Pro tiRNA oligonucleotide
  source 1..30: mol_type = other RNA
ggctcgttggtctaggggtatgattctcgg

SEQ ID NO: 9 - RNA, 29 nt, synthetic construct
  misc_feature 1..29: note = Description of Artificial Sequence: Synthetic 5'-Gln tiRNA oligonucleotide
  source 1..29: mol_type = other RNA
ggttccatggtgtaatggttagcactctg

SEQ ID NO: 10 - RNA, 30 nt, synthetic construct
  misc_feature 1..30: note = Description of Artificial Sequence: Synthetic 5'-Cys tiRNA oligonucleotide
  source 1..30: mol_type = other RNA
gggggtatagctcagtggtagagcatttga

SEQ ID NO: 11 - RNA, 31 nt, synthetic construct
  misc_feature 1..31: note = Description of Artificial Sequence: Synthetic 5'-Met tiRNA oligonucleotide
  source 1..31: mol_type = other RNA
gcctcgttagcgcagtaggtagcgcgtcagt

SEQ ID NO: 12 - RNA, 29 nt, synthetic construct
  misc_feature 1..29: note = Description of Artificial Sequence: Synthetic 5'-Met-I tiRNA oligonucleotide
  source 1..29: mol_type = other RNA
agcagagtggcgcagcggaagcgtgctgg

SEQ ID NO: 13 - RNA, 43 nt, synthetic construct
  misc_feature 1..43: note = Description of Artificial Sequence: Synthetic 3'-Ala oligonucleotide
  source 1..43: mol_type = other RNA
cttagcatgcacgaggccccgggttcaatccccggcacctcca

SEQ ID NO: 14 - RNA, 38 nt, synthetic construct
  misc_feature 1..38: note = Description of Artificial Sequence: Synthetic 3'-Arg tiRNA oligonucleotide
  source 1..38: mol_type = other RNA
ggatcagaagattgagggttcgggtcccttcgtggtcg

SEQ ID NO: 15 - RNA, 38 nt, synthetic construct
  misc_feature 1..38: note = Description of Artificial Sequence: Synthetic 3'-Gly tiRNA oligonucleotide
  source 1..38: mol_type = other RNA
ccacgcgggaggcccgggttcgattcccggccaatgca

SEQ ID NO: 16 - RNA, 43 nt, synthetic construct
  misc_feature 1..43: note = Description of Artificial Sequence: Synthetic 3'-Gln tiRNA oligonucleotide
  source 1..43: mol_type = other RNA
gactctgaatccagcgatccgagttcaaatctcggtggaacct

SEQ ID NO: 17 - RNA, 42 nt, synthetic construct
  misc_feature 1..42: note = Description of Artificial Sequence: Synthetic 3'-Pro tiRNA oligonucleotide
  source 1..42: mol_type = other RNA
ttaggatgcgagaggtcccgggttcaaatcccggacgagccc

SEQ ID NO: 18 - RNA, 30 nt, synthetic construct
  misc_feature 1..30: note = Description of Artificial Sequence: Synthetic U4G tiRNA oligonucleotide
  source 1..30: mol_type = other RNA
tggggtgtagctcagtggtagagcgcgtgc

SEQ ID NO: 19 - RNA, 29 nt, synthetic construct
  misc_feature 1..29: note = Description of Artificial Sequence: Synthetic 4G tiRNA oligonucleotide
  source 1..29: mol_type = other RNA
ggggtgtagctcagtggtagagcgcgtgc

SEQ ID NO: 20 - RNA, 30 nt, synthetic construct
  misc_feature 1..30: note = Description of Artificial Sequence: Synthetic UU3G tiRNA oligonucleotide
  source 1..30: mol_type = other RNA
ttgggtgtagctcagtggtagagcgcgtgc

SEQ ID NO: 21 - RNA, 28 nt, synthetic construct
  misc_feature 1..28: note = Description of Artificial Sequence: Synthetic 3G tiRNA oligonucleotide
  source 1..28: mol_type = other RNA
gggtgtagctcagtggtagagcgcgtgc

SEQ ID NO: 22 - RNA, 30 nt, synthetic construct
  misc_feature 1..30: note = Description of Artificial Sequence: Synthetic GG-UU tiRNA oligonucleotide
  source 1..30: mol_type = other RNA
gggggtgtagctcagttttagagcgcgtgc

SEQ ID NO: 23 - RNA, 27 nt, synthetic construct
  misc_feature 1..27: note = Description of Artificial Sequence: Synthetic 27-mer tiRNA oligonucleotide
  source 1..27: mol_type = other RNA
gggggtgtagctcagtggtagagcgcg

SEQ ID NO: 24 - RNA, 24 nt, synthetic construct
  misc_feature 1..24: note = Description of Artificial Sequence: Synthetic 24-mer tiRNA oligonucleotide
  source 1..24: mol_type = other RNA
gggggtgtagctcagtggtagagc

SEQ ID NO: 25 - RNA, 21 nt, synthetic construct
  misc_feature 1..21: note = Description of Artificial Sequence: Synthetic 21-mer tiRNA oligonucleotide
  source 1..21: mol_type = other RNA
gggggtgtagctcagtggtag

SEQ ID NO: 26 - RNA, 31 nt, synthetic construct
  misc_feature 1..31: note = Description of Artificial Sequence: Synthetic Modified 5'-tiRNAMet oligonucleotide
  source 1..31: mol_type = other RNA
gggggtttagcgcagtaggtagcgcgtcagt

SEQ ID NO: 27 - RNA, 30 nt, synthetic construct; identical to SEQ ID NO: 1
  misc_feature 1..30: note = Description of Artificial Sequence: Synthetic Ctrl1-biotinylated tiRNA oligonucleotide
  misc_feature 1..30: note = 3' Biotin
  source 1..30: mol_type = other RNA

SEQ ID NO: 28 - RNA, 29 nt, synthetic construct
  misc_feature 1..29: note = Description of Artificial Sequence: Synthetic Ctrl2-biotinylated tiRNA oligonucleotide
  misc_feature 1..29: note = 3' Biotin
  source 1..29: mol_type = other RNA
tgtgagtcacgtgagggcagaatctgctc

SEQ ID NO: 29 - RNA, 31 nt, synthetic construct; identical to SEQ ID NO: 3
  misc_feature 1..31: note = Description of Artificial Sequence: Synthetic Ctrl3-biotinylated tiRNA oligonucleotide
  misc_feature 1..31: note = 3' Biotin
  source 1..31: mol_type = other RNA

SEQ ID NO: 30 - RNA, 30 nt, synthetic construct; identical to SEQ ID NO: 4
  misc_feature 1..30: note = Description of Artificial Sequence: Synthetic 5'-Ala-biotinylated tiRNA oligonucleotide
  misc_feature 1..30: note = 3' Biotin
  source 1..30: mol_type = other RNA

SEQ ID NO: 31 - RNA, 43 nt, synthetic construct; identical to SEQ ID NO: 13
  misc_feature 1..43: note = Description of Artificial Sequence: Synthetic 3'-Ala-biotinylated tiRNA oligonucleotide
  misc_feature 1..43: note = 3' Biotin
  source 1..43: mol_type = other RNA

SEQ ID NO: 32 - DNA, 30 bp, synthetic construct
  misc_feature 1..30: note = Description of Artificial Sequence: Synthetic 5'-Ala tiDNA oligonucleotide
  source 1..30: mol_type = other DNA
gggggtgtagctcagtggtagagcgcgtgc

SEQ ID NO: 33 - DNA, 24 bp, synthetic construct
  misc_feature 1..24: note = Description of Artificial Sequence: Synthetic Tel4 oligonucleotide
  source 1..24: mol_type = other DNA
ttagggttagggttagggttaggg

SEQ ID NO: 34 - DNA, 26 bp, synthetic construct
  misc_feature 1..26: note = Description of Artificial Sequence: Synthetic AS1411 oligonucleotide
  source 1..26: mol_type = other DNA
ggtggtggtggttgtggtggtggtgg

SEQ ID NO: 35 - DNA, 27 bp, synthetic construct
  misc_feature 1..27: note = Description of Artificial Sequence: Synthetic GT-oligonucleotide
  source 1..27: mol_type = other DNA
tgtttgtttgtttgtttgtttgtttgt

SEQ ID NO: 36 - DNA, 22 bp, synthetic construct
  misc_feature 1..22: note = Description of Artificial Sequence: Synthetic C-myc oligonucleotide
  source 1..22: mol_type = other DNA
ggggagggtggggagggtgggg

SEQ ID NO: 37 - RNA, 75 nt, synthetic construct
  misc_feature 1..75: note = Description of Artificial Sequence: Synthetic 5'-tiRNAAla oligonucleotide
  source 1..75: mol_type = other RNA
gggggtgtagctcagtggtagagcgcgtgcttagcatgcacgaggccccgggttcaatccccggcacctccaccc

SEQ ID NO: 38 - DNA, 25 bp, synthetic construct
  misc_feature 1..25: note = Description of Artificial Sequence: Synthetic Ctrl 25 oligonucleotide
  source 1..25: mol_type = other DNA
aaaaaactcgagatggcgcacgctg

SEQ ID NO: 39 - DNA, 26 bp, synthetic construct
  misc_feature 1..26: note = Description of Artificial Sequence: Synthetic CRO26 oligonucleotide
  source 1..26: mol_type = other DNA
cctcctcctccttctcctcctcctcc

SEQ ID NO: 40 - RNA, 32 nt, Homo sapiens
  source 1..32: mol_type = genomic RNA
gggggtgtagctcagtggtagagcgcgtgctt

SEQ ID NO: 41 - RNA, 31 nt, Homo sapiens
  source 1..31: mol_type = genomic RNA
gggggtgtagctcagtggtagagcgcgtgct

SEQ ID NO: 42 - RNA, 32 nt, Homo sapiens
  source 1..32: mol_type = genomic RNA
ggggggtgtagctcagtggtagagcgcgtgct

SEQ ID NO: 43 - RNA, 30 nt, Homo sapiens; identical to SEQ ID NO: 4
  source 1..30: mol_type = genomic RNA

SEQ ID NO: 44 - RNA, 31 nt, Homo sapiens
  source 1..31: mol_type = genomic RNA
ggggggtgtagctcagtggtagagcgcgtgc

SEQ ID NO: 45 - RNA, 32 nt, Homo sapiens
  source 1..32: mol_type = genomic RNA
gggggtntagctcagtggtagagcgcgtgctt

SEQ ID NO: 46 - RNA, 29 nt, Homo sapiens
  source 1..29: mol_type = genomic RNA
gggggtgtagctcagtggtagagcgcgtg

SEQ ID NO: 47 - RNA, 31 nt, Homo sapiens
  source 1..31: mol_type = genomic RNA
gtgtagctcagtggtagagcgcgtgcttcgc

SEQ ID NO: 48 - RNA, 31 nt, Homo sapiens
  source 1..31: mol_type = genomic RNA
ggggtntagctcagtggtagagcgcgtgctt

SEQ ID NO: 49 - RNA, 32 nt, Homo sapiens
  source 1..32: mol_type = genomic RNA
ggggggtntagctcagtggtagagcgcgtgct

SEQ ID NO: 50 - RNA, 31 nt, Homo sapiens
  source 1..31: mol_type = genomic RNA
ggggtgtagctcagtggtagagagcgtgctt

SEQ ID NO: 51 - RNA, 30 nt, Homo sapiens
  source 1..30: mol_type = genomic RNA
gggggtntagctcagtggtagagcgcgtgc

SEQ ID NO: 52 - RNA, 31 nt, Homo sapiens
  source 1..31: mol_type = genomic RNA
gggggtgtagctcagtggtagagagcgtgct

SEQ ID NO: 53 - RNA, 31 nt, Homo sapiens
  source 1..31: mol_type = genomic RNA
ggggatgtagctcagtggtagagcgcatgct

SEQ ID NO: 54 - RNA, 30 nt, Homo sapiens
  source 1..30: mol_type = genomic RNA
gggggattagctcaaatggtagagcgctcg

SEQ ID NO: 55 - RNA, 31 nt, Homo sapiens
  source 1..31: mol_type = genomic RNA
ggggatntagctcagtggtagagcgcatgct

SEQ ID NO: 56 - RNA, 27 nt, Homo sapiens
  source 1..27: mol_type = genomic RNA
ggctctgttgcgcaatggatagcgcat

SEQ ID NO: 57 - RNA, 28 nt, Homo sapiens
  source 1..28: mol_type = genomic RNA
tcctcattagtatagtggtgagtatccc

SEQ ID NO: 58 - RNA, 30 nt, Homo sapiens; identical to SEQ ID NO: 10
  source 1..30: mol_type = genomic RNA

SEQ ID NO: 59 - RNA, 29 nt, Homo sapiens
  source 1..29: mol_type = genomic RNA
gggggtatagctcagtggtagagcatttg

SEQ ID NO: 60 - RNA, 28 nt, Homo sapiens
  source 1..28: mol_type = genomic RNA
gggggtatagctcagtggtagagcattt

SEQ ID NO: 61 - RNA, 27 nt, Homo sapiens
  source 1..27: mol_type = genomic RNA
gggggtatagctcagtgggtagagcat

SEQ ID NO: 62 - RNA, 30 nt, Homo sapiens
  source 1..30: mol_type = genomic RNA
gggggtgtaactcagtggtagagcatttga

SEQ ID NO: 63 - RNA, 29 nt, Homo sapiens
  source 1..29: mol_type = genomic RNA
ggttccatggtgtaatggttagcactctg

SEQ ID NO: 64 - RNA, 32 nt, Homo sapiens
  source 1..32: mol_type = genomic RNA
ttggtggttcagtggtagaattctcgcctgcc

SEQ ID NO: 65 - RNA, 31 nt, Homo sapiens
  source 1..31: mol_type = genomic RNA
ttggtggttcagtggtagaattctcgcctgc

SEQ ID NO: 66 - RNA, 30 nt, Homo sapiens
  source 1..30: mol_type = genomic RNA
ttggtggttcagtggtagaattctcgcctg

SEQ ID NO: 67 - RNA, 29 nt, Homo sapiens
  source 1..29: mol_type = genomic RNA
tggtggttcagtggtagaattctcgcctg

SEQ ID NO: 68 - RNA, 31 nt, Homo sapiens
  source 1..31: mol_type = genomic RNA
attggtggttcagtggtagaattctcgcctg

SEQ ID NO: 69 - RNA, 29 nt, Homo sapiens
  source 1..29: mol_type = genomic RNA
ttggtggttcagtggtagaattctcgcct

SEQ ID NO: 70 - RNA, 31 nt, Homo sapiens
  source 1..31: mol_type = genomic RNA
ggcattggtggttcagtggtagaattctcgc

SEQ ID NO: 71 - RNA, 29 nt, Homo sapiens
  source 1..29: mol_type = genomic RNA
attggtggttcagtggtagaattctcgcc

SEQ ID NO: 72 - RNA, 31 nt, Homo sapiens
  source 1..31: mol_type = genomic RNA
agcattggtggttcagtggtagaattctcgc

SEQ ID NO: 73 - RNA, 29 nt, Homo sapiens
  source 1..29: mol_type = genomic RNA
cattggtggttcagtggtagaattctcgc

SEQ ID NO: 74 - RNA, 32 nt, Homo sapiens
  source 1..32: mol_type = genomic RNA
gggaggcccgggttcgtttcccggccaatgca

SEQ ID NO: 75 - RNA, 30 nt, Homo sapiens
  source 1..30: mol_type = genomic RNA
gcattggtggttcagtggtagaattctcac

SEQ ID NO: 76 - RNA, 32 nt, Homo sapiens
  source 1..32: mol_type = genomic RNA
cgggaggcccgggttcggttcccggccaatgc

SEQ ID NO: 77 - RNA, 27 nt, Homo sapiens
  source 1..27: mol_type = genomic RNA
ttggtggttcagtggtagaattctcgc

SEQ ID NO: 78 - RNA, 28 nt, Homo sapiens
  source 1..28: mol_type = genomic RNA
gacattggtggttcagtggtagaattct

SEQ ID NO: 79 - RNA, 27 nt, Homo sapiens
  source 1..27: mol_type = genomic RNA
tggttcagtggtagaattctcgcctcc

SEQ ID NO: 80 - RNA, 27 nt, Homo sapiens
  source 1..27: mol_type = genomic RNA
gcattggtatagtggtatcatgcaaga

SEQ ID NO: 81 - RNA, 32 nt, Homo sapiens
  source 1..32: mol_type = genomic RNA
agcgttggtggtatagtggtgagcatagctgc

SEQ ID NO: 82 - RNA, 30 nt, Homo sapiens
  source 1..30: mol_type = genomic RNA
ggccgtgatcgtatagtggttagtactctg

SEQ ID NO: 83 - RNA, 31 nt, Homo sapiens
  source 1..31: mol_type = genomic RNA
tcgccgtgatcgtatagtggttagtactctg

SEQ ID NO: 84 - RNA, 28 nt, Homo sapiens
  source 1..28: mol_type = genomic RNA
ggccgtgatcgtatagtggttagtactc

SEQ ID NO: 85 - RNA, 29 nt, Homo sapiens
  source 1..29: mol_type = genomic RNA
aggccgtgatcgtatagtggttagtactc

SEQ ID NO: 86 - RNA, 27 nt, Homo sapiens
  source 1..27: mol_type = genomic RNA
ggccgtgatcgtatagtggttagtact

SEQ ID NO: 87 - RNA, 26 nt, Homo sapiens
  source 1..26: mol_type = genomic RNA
ggccgtgatcgtatagtggttagtac

SEQ ID NO: 88 - RNA, 26 nt, Homo sapiens
  source 1..26: mol_type = genomic RNA
ggccggttagctcagttggttagagc

SEQ ID NO: 89 - RNA, 26 nt, Homo sapiens
  source 1..26: mol_type = genomic RNA
ggccggttagctcagttggtcagagc

SEQ ID NO: 90 - RNA, 31 nt, Homo sapiens
  source 1..31: mol_type = genomic RNA
ggccggttagctcagttggtaagagcttggt

SEQ ID NO: 91 - RNA, 31 nt, Homo sapiens
  source 1..31: mol_type = genomic RNA
ggggcggccggttagctcagttggtaagagc

SEQ ID NO: 92 - RNA, 26 nt, Homo sapiens
  source 1..26: mol_type = genomic RNA
ggccggttagctcagttggtaagagc

SEQ ID NO: 93 - RNA, 26 nt, Homo sapiens
  source 1..26: mol_type = genomic RNA
ggtagtgtggccgagcggtctaaggc

SEQ ID NO: 94 - RNA, 31 nt, Homo sapiens
  source 1..31: mol_type = genomic RNA
gtagtcgtggccgagtggttaaggctatgga

SEQ ID NO: 95 - RNA, 32 nt, Homo sapiens
  source 1..32: mol_type = genomic RNA
gacgaggtggccgagtggttaaggctatggat

SEQ ID NO: 96 - RNA, 32 nt, Homo sapiens
  source 1..32: mol_type = genomic RNA
gacgaggtggccgagtggttaaggctatggac

SEQ ID NO: 97 - RNA, 31 nt, Homo sapiens
  source 1..31: mol_type = genomic RNA
gacgaggtggccgagtggttaaggctatgga

SEQ ID NO: 98 - RNA, 30 nt, Homo sapiens
  source 1..30: mol_type = genomic RNA
gacgaggtggccgagtggttaaggctatgg

SEQ ID NO: 99 - RNA, 31 nt, Homo sapiens
  source 1..31: mol_type = genomic RNA
gacgaggtggccgagtggttaaggcaatgga

SEQ ID NO: 100 - RNA, 30 nt, Homo sapiens
  source 1..30: mol_type = genomic RNA
gacgaggtggccgagtggttaaggcaatgg

SEQ ID NO: 101 - RNA, 26 nt, Homo sapiens
  source 1..26: mol_type = genomic RNA
tgtagtcgtggccgagtggttaaggc

SEQ ID NO: 102 - RNA, 31 nt, Homo sapiens
  source 1..31: mol_type = genomic RNA
gcctggatagctcagttggtagagcatcaga

SEQ ID NO: 103 - RNA, 29 nt, Homo sapiens
  source 1..29: mol_type = genomic RNA
gcctggatagctcagttggtagagcatca

SEQ ID NO: 104 - RNA, 32 nt, Homo sapiens
  source 1..32: mol_type = genomic RNA
gcctgggtagctcagtcggtagagcatcagac

SEQ ID NO: 105 - RNA, 31 nt, Homo sapiens
  source 1..31: mol_type = genomic RNA
gcctgggtagctcagtcggtagagcatcaga

SEQ ID NO: 106 - RNA, 30 nt, Homo sapiens
  source 1..30: mol_type = genomic RNA
gcctgggtagctcagtcggtagagcatcag

SEQ ID NO: 107 - RNA, 32 nt, Homo sapiens
  source 1..32: mol_type = genomic RNA
gcagagtggcgcagcggaagcgtgctgggccc

SEQ ID NO: 108 - RNA, 32 nt, Homo sapiens
  source 1..32: mol_type = genomic RNA
ggcagagtggcgcagcggaagcgtgctgggcc

SEQ ID NO: 109 - RNA, 28 nt, Homo sapiens
  source 1..28: mol_type = genomic RNA
gcagagtggcgcagcggaagcgtgctgg

SEQ ID NO: 110 - RNA, 29 nt, Homo sapiens
  source 1..29: mol_type = genomic RNA
tgcagagtggcgcagcggaagcgtgctgg

SEQ ID NO: 111 - RNA, 29 nt, Homo sapiens
  source 1..29: mol_type = genomic RNA
gcagtggcgcagcggaagcgtgctgggcc

SEQ ID NO: 112 - RNA, 27 nt, Homo sapiens
  source 1..27: mol_type = genomic RNA
gcagagtggcgcagcggaagcgtgctg

SEQ ID NO: 113 - RNA, 32 nt, Homo sapiens
  source 1..32: mol_type = genomic RNA
cgcagagtcgcgcagcggaagcgtgctgggcc

SEQ ID NO: 114 - RNA, 31 nt, Homo sapiens
  source 1..31: mol_type = genomic RNA
cagagtcgcgcagcggaagcgtgctgggccc

SEQ ID NO: 115 - RNA, 31 nt, Homo sapiens
  source 1..31: mol_type = genomic RNA
agagttgcgcagcggaagcgtgctgggccca

SEQ ID NO: 116 - RNA, 27 nt, Homo sapiens
  source 1..27: mol_type = genomic RNA
gagatagcagagtggcgcagcggaagc

SEQ ID NO: 117 - RNA, 30 nt, Homo sapiens; identical to SEQ ID NO: 8
  source 1..30: mol_type = genomic RNA

SEQ ID NO: 118 - RNA, 30 nt, Homo sapiens
  source 1..30: mol_type = genomic RNA
aggctcgttggtctagtggtatgattctcg

SEQ ID NO: 119 - RNA, 30 nt, Homo sapiens
  source 1..30: mol_type = genomic RNA
gcccggatgatcctcagtggtctggggtgc

SEQ ID NO: 120 - RNA, 26 nt, Homo sapiens
  source 1..26: mol_type = genomic RNA
tgtagtcgtggccgagtggttaaggc

SEQ ID NO: 121 - RNA, 32 nt, Homo sapiens; identical to SEQ ID NO: 96
  source 1..32: mol_type = genomic RNA

SEQ ID NO: 122 - RNA, 32 nt, Homo sapiens; identical to SEQ ID NO: 95
  source 1..32: mol_type = genomic RNA

SEQ ID NO: 123 - RNA, 31 nt, Homo sapiens; identical to SEQ ID NO: 97
  source 1..31: mol_type = genomic RNA

SEQ ID NO: 124 - RNA, 31 nt, Homo sapiens; identical to SEQ ID NO: 99
  source 1..31: mol_type = genomic RNA

SEQ ID NO: 125 - RNA, 30 nt, Homo sapiens; identical to SEQ ID NO: 98
  source 1..30: mol_type = genomic RNA

SEQ ID NO: 126 - RNA, 30 nt, Homo sapiens; identical to SEQ ID NO: 100
  source 1..30: mol_type = genomic RNA

SEQ ID NO: 127 - RNA, 31 nt, Homo sapiens; identical to SEQ ID NO: 102
  source 1..31: mol_type = genomic RNA

SEQ ID NO: 128 - RNA, 29 nt, Homo sapiens
  source 1..29: mol_type = genomic RNA
gcctggatagctcagttggtagagcatca

SEQ ID NO: 129 - RNA, 32 nt, Homo sapiens; identical to SEQ ID NO: 108
  source 1..32: mol_type = genomic RNA

SEQ ID NO: 130 - RNA, 32 nt, Homo sapiens; identical to SEQ ID NO: 107
  source 1..32: mol_type = genomic RNA

SEQ ID NO: 131 - RNA, 28 nt, Homo sapiens
  source 1..28: mol_type = genomic RNA
gcagagtggcgcagcggaagcgtgctgg

SEQ ID NO: 132 - RNA, 28 nt, Homo sapiens
  source 1..28: mol_type = genomic RNA
cggaagcgtgctgggcccataacccaga

SEQ ID NO: 133 - RNA, 29 nt, Homo sapiens
  source 1..29: mol_type = genomic RNA
tgcagagtggcgcagcggaagcgtgctgg

SEQ ID NO: 134 - RNA, 29 nt, Homo sapiens
  source 1..29: mol_type = genomic RNA
gcagtggcgcagcggaagcgtgctgggcc

SEQ ID NO: 135 - RNA, 27 nt, Homo sapiens
  source 1..27: mol_type = genomic RNA
gcagagtggcgcagcggaagcgtgctg

SEQ ID NO: 136 - RNA, 32 nt, Homo sapiens; identical to SEQ ID NO: 113
  source 1..32: mol_type = genomic RNA

SEQ ID NO: 137 - RNA, 31 nt, Homo sapiens; identical to SEQ ID NO: 114
  source 1..31: mol_type = genomic RNA

SEQ ID NO: 138 - RNA, 31 nt, Homo sapiens; identical to SEQ ID NO: 115
  source 1..31: mol_type = genomic RNA

SEQ ID NO: 139 - RNA, 31 nt, Homo sapiens; identical to SEQ ID NO: 102
  source 1..31: mol_type = genomic RNA

SEQ ID NO: 140 - RNA, 29 nt, Homo sapiens
  source 1..29: mol_type = genomic RNA
gcctggatagctcagttggtagagcatca

SEQ ID NO: 141 - RNA, 32 nt, Homo sapiens
  source 1..32: mol_type = genomic RNA
ttccgtagtgtagtggttatcacgttcgcctc

SEQ ID NO: 142 - RNA, 30 nt, Homo sapiens
  source 1..30: mol_type = genomic RNA
ttccgtagtgtagtggttatcacgttcgcc

SEQ ID NO: 143 - RNA, 32 nt, Homo sapiens
  source 1..32: mol_type = genomic RNA
tccgtagtgtagtggttatcacgttcgcctga

SEQ ID NO: 144 - RNA, 31 nt, Homo sapiens
  source 1..31: mol_type = genomic RNA
tccgtagtgtagtggttatcacgttcgcctg

SEQ ID NO: 145 - RNA, 32 nt, Homo sapiens
  source 1..32: mol_type = genomic RNA
tccgtagtgtagtggttatcacgttcgcctca

SEQ ID NO: 146 - RNA, 30 nt, Homo sapiens
  source 1..30: mol_type = genomic RNA
tccgtagtgtagtggttatcacgttcgcct

SEQ ID NO: 147 - RNA, 32 nt, Homo sapiens
  source 1..32: mol_type = genomic RNA
gtttccgtagtgtagtggtcatcacgttcgcc

SEQ ID NO: 148 - RNA, 28 nt, Homo sapiens
  source 1..28: mol_type = genomic RNA
ccgtagtgtagtggttatcacgttcgcc

SEQ ID NO: 149 - RNA, 30 nt, Homo sapiens
  source 1..30: mol_type = genomic RNA
gtttccgtagtgtagtggtcatcacgttcg

SEQ ID NO: 150 - RNA, 27 nt, Homo sapiens
  source 1..27: mol_type = genomic RNA
cgtagtgtagtggttatcacgttcgcc

SEQ ID NO: 151 - RNA, 30 nt, Homo sapiens
  source 1..30: mol_type = genomic RNA
tccgtagtgtagtggttatcactttcgcct

SEQ ID NO: 152 - RNA, 31 nt, Homo sapiens
  source 1..31: mol_type = genomic RNA
tccgtagtgtactggttatcacgttcgcctg

SEQ ID NO: 153 - RNA, 28 nt, Homo sapiens
  source 1..28: mol_type = genomic RNA
cgtagtgtagtggtcatcacgttcgcct

SEQ ID NO: 154 - RNA, 32 nt, Homo sapiens; identical to SEQ ID NO: 40
  source 1..32: mol_type = genomic RNA

SEQ ID NO: 155 - RNA, 31 nt, Homo sapiens; identical to SEQ ID NO: 41
  source 1..31: mol_type = genomic RNA

SEQ ID NO: 156 - RNA, 30 nt, Homo sapiens; identical to SEQ ID NO: 4
  source 1..30: mol_type = genomic RNA

SEQ ID NO: 157 - RNA, 29 nt, Homo sapiens
  source 1..29: mol_type = genomic RNA
gggggtgtagctcagtggtagagcgcgtg

SEQ ID NO: 158 - RNA, 32 nt, Homo sapiens; identical to SEQ ID NO: 42
  source 1..32: mol_type = genomic RNA

SEQ ID NO: 159 - RNA, 32 nt, Homo sapiens; identical to SEQ ID NO: 64
  source 1..32: mol_type = genomic RNA

SEQ ID NO: 160 - RNA, 31 nt, Homo sapiens; identical to SEQ ID NO: 65
  source 1..31: mol_type = genomic RNA

SEQ ID NO: 161 - RNA, 30 nt, Homo sapiens; identical to SEQ ID NO: 66
  source 1..30: mol_type = genomic RNA

SEQ ID NO: 162 - RNA, 29 nt, Homo sapiens
  source 1..29: mol_type = genomic RNA
ttggtggttcagtggtagaattctcgcct

SEQ ID NO: 163 - RNA, 29 nt, Homo sapiens
  source 1..29: mol_type = genomic RNA
tggtggttcagtggtagaattctcgcctg

SEQ ID NO: 164 - RNA, 31 nt, Homo sapiens; identical to SEQ ID NO: 68
  source 1..31: mol_type = genomic RNA

SEQ ID NO: 165 - RNA, 29 nt, Homo sapiens
  source 1..29: mol_type = genomic RNA
attggtggttcagtggtagaattctcgcc

SEQ ID NO: 166 - RNA, 31 nt, Homo sapiens; identical to SEQ ID NO: 70
  source 1..31: mol_type = genomic RNA

SEQ ID NO: 167 - RNA, 27 nt, Homo sapiens
  source 1..27: mol_type = genomic RNA
ttggtggttcagtggtagaattctcgc

SEQ ID NO: 168 - RNA, 27 nt, Homo sapiens
  source 1..27: mol_type = genomic RNA
tggttcagtggtagaattctcgcctcc

SEQ ID NO: 169 - RNA, 29 nt, Homo sapiens
  source 1..29: mol_type = genomic RNA
cattggtggttcagtggtagaattctcgc

SEQ ID NO: 170 - RNA, 31 nt, Homo sapiens; identical to SEQ ID NO: 72
  source 1..31: mol_type = genomic RNA

SEQ ID NO: 171 - RNA, 30 nt, Homo sapiens; identical to SEQ ID NO: 75
  source 1..30: mol_type = genomic RNA

SEQ ID NO: 172 - DNA, 40 bp, Homo sapiens
  source 1..40: mol_type = genomic DNA
gtctctgtggcgcaatcggttagcgcgttcggctgttaac

SEQ ID NO: 173 - DNA, 40 bp, Homo sapiens
  source 1..40: mol_type = genomic DNA
gccgaaatagctcagttgggagagcgttagactgaagatc

SEQ ID NO: 174 - DNA, 21 bp, Homo sapiens
  source 1..21: mol_type = genomic DNA
gggagggtggggagggtgggg

SEQ ID NO: 175 - DNA, 32 bp, synthetic construct
  misc_feature 1..32: note = Description of Artificial Sequence: Synthetic ME8 probe
  source 1..32: mol_type = other DNA
ctttatgtttttggcgtcttccatctcgaggc

SEQ ID NO: 176 - DNA, 73 bp, Homo sapiens
  source 1..73: mol_type = genomic DNA
ggggaattagctcaagcggtagagcgctcccttagcatgcgagaggtagcgggatcgacgcccccattctcta

SEQ ID NO: 177 - DNA, 73 bp, Homo sapiens
  source 1..73: mol_type = genomic DNA
gggggattagctcaagcggtagggtgcctgcttagcatgcaagaggtagcaggatcgacgcctgcattctcca

SEQ ID NO: 178 - DNA, 71 bp, Homo sapiens
  source 1..71: mol_type = genomic DNA
gggggtgtagatcagtggtagggcgcacgcttagcatgcatgaggccctgggtcaatccccagcacctcca

SEQ ID NO: 179 - DNA, 73 bp, Homo sapiens
  source 1..73: mol_type = genomic DNA
gggggattagctcaagcggtagagcgcctgcttagcatgcaagaggtagcaggatcgatgcctgcattctcca

SEQ ID NO: 180 - DNA, 73 bp, Homo sapiens
  source 1..73: mol_type = genomic DNA
ggggaattggctcaagcggtagagcgcttgcttagcatgcaagaggtagcaggatcgacgcctgcactctcca

SEQ ID NO: 181 - DNA, 73 bp, Homo sapiens; identical to SEQ ID NO: 180
  source 1..73: mol_type = genomic DNA

SEQ ID NO: 182 - DNA, 73 bp, Homo sapiens
  source 1..73: mol_type = genomic DNA
ggggaattagcgcaagtggtagagtgcttgcttagcatgcaagaggtagtgggatcgatgcccacattctcca

SEQ ID NO: 183 - DNA, 73 bp, Homo sapiens
  source 1..73: mol_type = genomic DNA
ggggaattagcccaagtggtagagcgcttgcttagcatgcaagaggtagtgggatcgatgcccacattctcca

SEQ ID NO: 184 - DNA, 73 bp, Homo sapiens
  source 1..73: mol_type = genomic DNA
ggggaattagctcaagtggtagagcgctcgcttagcatgcgagaggtagtgggatcgatgcccgcattctcca

SEQ ID NO: 185 - DNA, 73 bp, Homo sapiens
  source 1..73: mol_type = genomic DNA
ggggaattagctcaagtggtagagcgcttgcttagcatgcaagaggtagtgggatcaatgcccacattctcca

SEQ ID NO: 186 - DNA, 73 bp, Homo sapiens
  source 1..73: mol_type = genomic DNA
ggggaattagctcaagcggtagagcgcttgcttagcatgcaagaggtagtgggatcgatgcccacattctcca

SEQ ID NO: 187 - DNA, 73 bp, Homo sapiens; identical to SEQ ID NO: 186
  source 1..73: mol_type = genomic DNA

SEQ ID NO: 188 - DNA, 73 bp, Homo sapiens
  source 1..73: mol_type = genomic DNA
ggggaattagctcaagtggtagagcgcttgcttagcatgcaagaggtagtgggatcgatgcccacattctcca

SEQ ID NO: 189 - DNA, 73 bp, Homo sapiens; identical to SEQ ID NO: 188
  source 1..73: mol_type = genomic DNA

SEQ ID NO: 190 - DNA, 73 bp, Homo sapiens; identical to SEQ ID NO: 188
  source 1..73: mol_type = genomic DNA

SEQ ID NO: 191 - DNA, 73 bp, Homo sapiens
  source 1..73: mol_type = genomic DNA
ggggaattagctcaaatggtagagcgctcgcttagcatgcgagaggtagcgggatcgatgcccgcattctcca

SEQ ID NO: 192 - DNA, 73 bp, Homo sapiens
  source 1..73: mol_type = genomic DNA
ggggaattagctcaagtggtagagcgcttgcttagcacgcaagaggtagtgggatcgatgcccacattctcca

SEQ ID NO: 193 - DNA, 73 bp, Homo sapiens
  source 1..73: mol_type = genomic DNA
ggggaattagctcaggcggtagagcgctcgcttagcatgcgagaggtagcgggatcgacgcccgcattctcca

SEQ ID NO: 194 - DNA, 73 bp, Homo sapiens; identical to SEQ ID NO: 193
  source 1..73: mol_type = genomic DNA

SEQ ID NO: 195 - DNA, 73 bp, Homo sapiens
  source 1..73: mol_type = genomic DNA
gggggattagctcaaatggtagagcgctcgcttagcatgcgagaggtagcgggatcgatgcccgcatcctcca

SEQ ID NO: 196 - DNA, 73 bp, Homo sapiens; identical to SEQ ID NO: 195
  source 1..73: mol_type = genomic DNA

SEQ ID NO: 197 - DNA, 72 bp, Homo sapiens
  source 1..72: mol_type = genomic DNA
gggggtgtagctcagtggtagagcgcgtgcttagcatgcacgaggccccgggttcaatccctggcacctcca

SEQ ID NO: 198 - DNA, 72 bp, Homo sapiens
  source 1..72: mol_type = genomic DNA
gggggtatagctcagcggtagagcgcgtgcttagcatgcacgaggtcctgggttcaatccccaatacctcca

SEQ ID NO: 199 - DNA, 72 bp, Homo sapiens
  source 1..72: mol_type = genomic DNA
gggggtgtagctcagtggtagagcgcgtgcttagcatgcacgaggccctgggttcaatccccagcacctcca

SEQ ID NO: 200 - DNA, 72 bp, Homo sapiens
  source 1..72: mol_type = genomic DNA
ggggatgtagctcagtggtagagcgcatgcttagcatgcatgaggtcccgggttcgatccccagcatctcca

SEQ ID NO: 201 - DNA, 72 bp, Homo sapiens
  source 1..72: mol_type = genomic DNA
gggggtgtagctcagtggtagagcgcgtgcttagcatgtacgaggtcccgggttcaatccccggcacctcca

SEQ ID NO: 202 - DNA, 72 bp, Homo sapiens
  source 1..72: mol_type = genomic DNA
gggggtgtagctcagtggtagagcgcgtgcttagcatgcacgaggccccgggttcaatccccggcacctcca

SEQ ID NO: 203 - DNA, 72 bp, Homo sapiens; identical to SEQ ID NO: 202
  source 1..72: mol_type = genomic DNA

SEQ ID NO: 204 - DNA, 72 bp, Homo sapiens
  source 1..72: mol_type = genomic DNA
gggggtatagctcagtggtagagcgcgtgcttagcatgcacgaggtcctgggttcgatccccagtacctcca

SEQ ID NO: 205 - DNA, 72 bp, Homo sapiens
  source 1..72: mol_type = genomic DNA
gggggtgtagatcagtggtagagcgcatgcttcgcatgtacgaggtccctggttcaatccctggtacctcca

SEQ ID NO: 206 - DNA, 72 bp, Homo sapiens
  source 1..72: mol_type = genomic DNA
gggggtgtagctcagtggtagagcgcgtgcttcgcatgtacgaggccccgggttcgacccccggctcctcca

SEQ ID NO: 207 - DNA, 72 bp, Homo sapiens
  source 1..72: mol_type = genomic DNA
ggggatgtagctcagtggtagagcgcgcgcttcgcatgtgtgaggtcccgggttcaatccccggcatctcca

SEQ ID NO: 208 - DNA, 72 bp, Homo sapiens
  source 1..72: mol_type = genomic DNA
ggggatgtagctcagtggtagagcgcatgcttcgcatgtatgaggccccgggttcgatccccggcatctcca

SEQ ID NO: 209 - DNA, 72 bp, Homo sapiens
  source 1..72: mol_type = genomic DNA
ggggatgtagctcagtggtagagcgcatgcttcgcatgtatgaggtcccgggttcgatccccggcatctcca

SEQ ID NO: 210 - DNA, 72 bp, Homo sapiens
  source 1..72: mol_type = genomic DNA
gggggtgtagctcagtggtagagcggatgctttgcatgtatgagactttgggttggatccccagcacctcca

SEQ ID NO: 211 - DNA, 71 bp, Homo sapiens
  source 1..71: mol_type = genomic DNA
gggggtgtagctcagtggtagagcgcatgctttgcatgtatgaggcctcggttcgatccccgacacctcca

SEQ ID NO: 212 - DNA, 72 bp, Homo sapiens
  source 1..72: mol_type = genomic DNA
gggggtgtagctcagtggtagagcacatgctttgcatgtgtgaggccccgggttcgatccccggcacctcca

SEQ ID NO: 213 - DNA, 72 bp, Homo sapiens
  source 1..72: mol_type = genomic DNA
gggggtgtagctcagtggtagagcgcatgctttgcatgtatgaggcctcgggttcgatccccgacacctcca

SEQ ID NO: 214 - DNA, 72 bp, Homo sapiens
  source 1..72: mol_type = genomic DNA
ggggatgtagctcagtggtagagcgcatgctttgcacgtatgaggccccgggttcaatccccggcatctcca

SEQ ID NO: 215 - DNA, 72 bp, Homo sapiens
  source 1..72: mol_type = genomic DNA
ggggatgtagctcagtggtagagcgcatgctttgcatgtatgaggccccgggttcgatccccggcatctcca

SEQ ID NO: 216 - DNA, 72 bp, Homo sapiens; identical to SEQ ID NO: 215
  source 1..72: mol_type = genomic DNA

SEQ ID NO: 217 - DNA, 72 bp, Homo sapiens
  source 1..72: mol_type = genomic DNA
ggggatgtagctcagtggtagagcgcatgctttgcatgtatgaggtcccgggttcgatccccggcatctcca

SEQ ID NO: 218 - DNA, 72 bp, Homo sapiens
  source 1..72: mol_type = genomic DNA
gggggtgtagctcagtggtagagcgcatgctttgcatgtatgaggtcccgggttcgatccccggcacctcca

SEQ ID NO: 219 - DNA, 73 bp, Homo sapiens
  source 1..73: mol_type = genomic DNA
gggccagtggcgcaatggataacgcgtctgactacggatcagaagattctaggttcgactcctggctggctcg

SEQ ID NO: 220 - DNA, 73 bp, Homo sapiens; identical to SEQ ID NO: 219
  source 1..73: mol_type = genomic DNA

SEQ ID NO: 221 - DNA, 73 bp, Homo sapiens; identical to SEQ ID NO: 219
  source 1..73: mol_type = genomic DNA

SEQ ID NO: 222 - DNA, 73 bp, Homo sapiens; identical to SEQ ID NO: 219
  source 1..73: mol_type = genomic DNA

SEQ ID NO: 223 - DNA, 73 bp, Homo sapiens
  source 1..73: mol_type = genomic DNA
gggccagtggcgcaatggataacgcgtctgactacggatcagaagattccaggttcgactcctggctggctcg

SEQ ID NO: 224 - DNA, 73 bp, Homo sapiens; identical to SEQ ID NO: 223
  source 1..73: mol_type = genomic DNA

SEQ ID NO: 225 - DNA, 73 bp, Homo sapiens; identical to SEQ ID NO: 223
  source 1..73: mol_type = genomic DNA

SEQ ID NO: 226 - DNA, 73 bp, Homo sapiens
  source 1..73: mol_type = genomic DNA
gacccagtggcctaatggataaggcatcagcctccggagctggggattgtgggttcgagtcccatctgggtcg

SEQ ID NO: 227 - DNA, 73 bp, Homo sapiens; identical to SEQ ID NO: 226
  source 1..73: mol_type = genomic DNA

SEQ ID NO: 228 - DNA, 73 bp, Homo sapiens
  source 1..73: mol_type = genomic DNA
ggccgcgtggcctaatggataaggcgtctgattccggatcagaagattgagggttcgagtcccttcgtggtcg

SEQ ID NO: 229 - DNA, 73 bp, Homo sapiens; identical to SEQ ID NO: 228
  source 1..73: mol_type = genomic DNA

SEQ ID NO: 230 - DNA, 73 bp, Homo sapiens; identical to SEQ ID NO: 228
  source 1..73: mol_type = genomic DNA

SEQ ID NO: 231 - DNA, 73 bp, Homo sapiens
  source 1..73: mol_type = genomic DNA
gccccagtggcctgatggataaggtactggcctcctaagccagggattgtgggttcgagttccacctggggta

SEQ ID NO: 232 - DNA, 73 bp, Homo sapiens
  source 1..73: mol_type = genomic DNA
gccccagtggcctaatggataaggcattggcctcctaagccagggattgtgggttcgagtcccatctggggtg

SEQ ID NO: 233 - DNA, 73 bp, Homo sapiens
  source 1..73: mol_type = genomic DNA
gccccggtggcctaatggataaggcattggcctcctaagccagggattgtgggttcgagtcccacccggggta

SEQ ID NO: 234 - DNA, 73 bp, Homo sapiens
  source 1..73: mol_type = genomic DNA
gccccagtggcctaatggataaggcactggcctcctaagccagggattgtgggttcgagtcccacctggggtg

SEQ ID NO: 235 - DNA, 73 bp, Homo sapiens
  source 1..73: mol_type = genomic DNA
gccccagtggcctaatggataaggcactggcctcctaagccagggattgtgggttcgagtcccacctggggta

SEQ ID NO: 236 - DNA, 73 bp, Homo sapiens
  source 1..73: mol_type = genomic DNA
ggccgtgtggcctaatggataaggcgtctgacttcggatcaaaagattgcaggtttgagttctgccacggtcg

SEQ ID NO: 237 - DNA, 73 bp, Homo sapiens
  source 1..73: mol_type = genomic DNA
gaccacgtggcctaatggataaggcgtctgacttcggatcagaagattgagggttcgaatcccttcgtggttg

SEQ ID NO: 238 - DNA, 73 bp, Homo sapiens
  source 1..73: mol_type = genomic DNA
gaccacgtggcctaatggataaggcgtctgacttcggatcagaagattgagggttcgaatcccttcgtggtta

SEQ ID NO: 239 - DNA, 73 bp, Homo sapiens
  source 1..73: mol_type = genomic DNA
gaccgcgtggcctaatggataaggcgtctgacttcggatcagaagattgagggttcgagtcccttcgtggtcg

SEQ ID NO: 240 - DNA, 73 bp, Homo sapiens
  source 1..73: mol_type = genomic DNA
gaccacgtggcctaatggataaggcgtctgacttcggatcagaagattgagggttcgaatccctccgtggtta

SEQ ID NO: 241 - DNA, 73 bp, Homo sapiens
  source 1..73: mol_type = genomic DNA
ggccgcgtggcctaatggataaggcgtctgacttcggatcagaagattgcaggttcgagtcctgccgcggtcg

SEQ ID NO: 242 - DNA, 74 bp, Homo sapiens
  source 1..74: mol_type = genomic DNA
gtctctgtggcgcaatggacgagcgcgctggacttctaatccagaggttccgggttcgagtcccggcagagatg

SEQ ID NO: 243 - DNA, 91 bp, Homo sapiens
  source 1..91: mol_type = genomic DNA
ggctctgtggcgcaatggatagcgcattggacttctagctgagcctagtgtggtcattcaaaggttgtgggttcgagtcccaccagagtcg

SEQ ID NO: 244 - DNA, 87 bp, Homo sapiens
  source 1..87: mol_type = genomic DNA
ggctctgtggcgcaatggatagcgcattggacttctagcctaaatcaagagattcaaaggttgcgggttcgagtccctccagagtcg

SEQ ID NO: 245 - DNA, 86 bp, Homo sapiens
  source 1..86: mol_type = genomic DNA
ggctctgtggcgcaatggatagcgcattggacttctagatagttagagaaattcaaaggttgtgggttcgagtcccaccagagtcg

SEQ ID NO: 246 - DNA, 88 bp, Homo sapiens
  source 1..88: mol_type = genomic DNA
ggctctgtggcgcaatggatagcgcattggacttctagtgacgaatagagcaattcaaaggttgtgggttcgaatcccaccagagtcg

SEQ ID NO: 247 - DNA, 85 bp, Homo sapiens
  source 1..85: mol_type = genomic DNA
ggctccgtggcgcaatggatagcgcattggacttctagaggctgaaggcattcaaaggttccgggttcgagtcccggcggagtcg

SEQ ID NO: 248 - DNA, 74 bp, Homo sapiens
  source 1..74: mol_type = genomic DNA
gtctctgtggcgcaatcggtcagagcgttcggctattaaccgaacggtgagtagttcaagaccacccagggacg

SEQ ID NO: 249 - DNA, 76 bp, Homo sapiens
  source 1..76: mol_type = genomic DNA
gtctctgtggcgccatcggttagtgccttcggctgtttgaaccgaaaggctggtggttcaagcccacccagagatg

SEQ ID NO: 250 - DNA, 74 bp, Homo sapiens
  source 1..74: mol_type = genomic DNA
atctccgtggagcaattggttagcgcgttcggccgttaaccggaaagttggtggttcgagcctacccagggacg

SEQ ID NO: 251 - DNA, 74 bp, Homo sapiens
  source 1..74: mol_type = genomic DNA
gtctctgtggtgcaatcggttagcgcgttcggctgttaaccataaggttggtggttacagaccacccagggacg

SEQ ID NO: 252 - DNA, 74 bp, Homo sapiens
  source 1..74: mol_type = genomic DNA
gtttctgtagcgcgatcggttagcgccttcggctgttaaacgaaaggttggtggttcgttcccaccccgggaca

SEQ ID NO: 253 - DNA, 74 bp, Homo sapiens
  source 1..74: mol_type = genomic DNA
gtctctgtggcgcaatcggttagcgcgtttgactgttaactgaaaggttggtggtgcaagcccatccagggatg

SEQ ID NO: 254 - DNA, 74 bp, Homo sapiens; identical to SEQ ID NO: 253
  source 1..74: mol_type = genomic DNA

SEQ ID NO: 255 - DNA, 74 bp, Homo sapiens; identical to SEQ ID NO: 253
  source 1..74: mol_type = genomic DNA

SEQ ID NO: 256 - DNA, 74 bp, Homo sapiens; identical to SEQ ID NO: 253
  source 1..74: mol_type = genomic DNA

SEQ ID NO: 257 - DNA, 74 bp, Homo sapiens; identical to SEQ ID NO: 253
  source 1..74: mol_type = genomic DNA

SEQ ID NO: 258 - DNA, 74 bp, Homo sapiens
  source 1..74: mol_type = genomic DNA
atctccgtggagcaattggttagcgcgttcggctgttaaccggaaagttggtggttcgagcctacccagggacg

SEQ ID NO: 259 - DNA, 74 bp, Homo sapiens
  source 1..74: mol_type = genomic DNA
gtctctgtggtgcaatcggttagcgcgttccgctgttaaccgaaagcttggtggtttgagcccacccagggatg

SEQ ID NO: 260 - DNA, 74 bp, Homo sapiens
  source 1..74: mol_type = genomic DNA
gtctctgtggtgcaatcggttagcgcgttcggctgttaaccataaggttggtggttagagaccacccagggacg

SEQ ID NO: 261 - DNA, 74 bp, Homo sapiens
  source 1..74: mol_type = genomic DNA
gtctctgtggcgcaatcggctagcgcgtttggctgttaactaaaaagttggtggttcgaacacacccagaggcg

SEQ ID NO: 262 - DNA, 74 bp, Homo sapiens
  source 1..74: mol_type = genomic DNA
gtctctgtggtgcaatcggttagcgcgttccgctgttaaccgaaagcttggtggttcgagcccacccagggatg

SEQ ID NO: 263 - DNA, 74 bp, Homo sapiens; identical to SEQ ID NO: 262
  source 1..74: mol_type = genomic DNA

SEQ ID NO: 264 - DNA, 74 bp, Homo sapiens
  source 1..74: mol_type = genomic DNA
gtctctgtggcgcaatcggctagcgcgtttggctgttaactaaaaggttggcggttcgaacccacccagaggcg

SEQ ID NO: 265 - DNA, 74 bp, Homo sapiens
  source 1..74: mol_type = genomic DNA
gtctctgtggcgcaatcggttagcgcgttcggctgttaactgaaaggttagtggttcgagcccacccggggacg

SEQ ID NO: 266 - DNA, 74 bp, Homo sapiens; identical to SEQ ID NO: 265
  source 1..74: mol_type = genomic DNA

SEQ ID NO: 267 - DNA, 74 bp, Homo sapiens
  source 1..74: mol_type = genomic DNA
gtctctgtggcgcaatcggctagcgcgtttggctgttaactaaaaggttggtggttcgaacccacccagaggcg

SEQ ID NO: 268 - DNA, 74 bp, Homo sapiens
  source 1..74: mol_type = genomic DNA
gtctctgtggcgtagtcggttagcgcgttcggctgttaaccgaaaagttggtggttcgagcccacccaggaacg

SEQ ID NO: 269 - DNA, 74 bp, Homo sapiens
  source 1..74: mol_type = genomic DNA
gtctctgtggcgcaatgggttagcgcgttcggctgttaaccgaaaggttggtggttcgagcccatccagggacg

SEQ ID NO: 270 - DNA, 74 bp, Homo sapiens
  source 1..74: mol_type = genomic DNA
gtctctgtggcgcaatcggttagcgcgttcggctgttaactgaaaggttggtggttcgagcccacccagggacg

SEQ ID NO: 271 - DNA, 74 bp, Homo sapiens
  source 1..74: mol_type = genomic DNA
gtctctgtggcgcaatcggttagcgcgttcggctgttaaccgaaagattggtggttcgagcccacccagggacg

SEQ ID NO: 272 - DNA, 74 bp, Homo sapiens
  source 1..74: mol_type = genomic DNA
gtctctgtggcgcaatcggttagcgcattcggctgttaaccgaaaggttggtggttcgagcccacccagggacg

SEQ ID NO: 273 - DNA, 74 bp, Homo sapiens; identical to SEQ ID NO: 272
  source 1..74: mol_type = genomic DNA

SEQ ID NO: 274 - DNA, 74 bp, Homo sapiens
  source 1..74: mol_type = genomic DNA
gtctctgtggcgcaatcggttagcgcgttcggctgttaaccgaaaggttggtggttcgagcccacccagggacg

SEQ ID NO: 275 - DNA, 74 bp, Homo sapiens; identical to SEQ ID NO: 274
  source 1..74: mol_type = genomic DNA

SEQ ID NO: 276 - DNA, 74 bp, Homo sapiens; identical to SEQ ID NO: 274
  source 1..74: mol_type = genomic DNA

SEQ ID NO: 277 - DNA, 74 bp, Homo sapiens; identical to SEQ ID NO: 274
  source 1..74: mol_type = genomic DNA

SEQ ID NO: 278 - DNA, 74 bp, Homo sapiens; identical to SEQ ID NO: 274
  source 1..74: mol_type = genomic DNA

SEQ ID NO: 279 - DNA, 74 bp, Homo sapiens; identical to SEQ ID NO: 274
  source 1..74: mol_type = genomic DNA

SEQ ID NO: 280 - DNA, 74 bp, Homo sapiens
  source 1..74: mol_type = genomic DNA
gtctctgtggcgcaatcggttagcgcgttcggctgttaaccgaaaggttggtggttcgatcccacccagggacg

SEQ ID NO: 281 - DNA, 72 bp, Homo sapiens
  source 1..72: mol_type = genomic DNA
tccttgttactatagtggtaagtatctctgcctgtcatgcatgagagagggggtcgattccctgacggggag

SEQ ID NO: 282 - DNA, 71 bp, Homo sapiens
  source 1..71: mol_type = genomic DNA
tccttgttagtatagtggtgagtgtttctgcctgtcatgtggagactggagtttgagtccccaacagggag

SEQ ID NO: 283 - DNA, 72 bp, Homo sapiens
  source 1..72: mol_type = genomic DNA
tactcgttagtatagtggtgcgtatccccgtctgtcacgcgggagagcggggttcgctctcccgacggggag

SEQ ID NO: 284 - DNA, 71 bp, Homo sapiens
  source 1..71: mol_type = genomic DNA
ttcttgttaatatagtggtgagtattcccacctgtcatgcgggagacggggttcaattccctgatggggag

SEQ ID NO: 285 - DNA, 72 bp, Homo sapiens
  source 1..72: mol_type = genomic DNA
tcctcatcagtatagtggtgagtatccccgcctgtcacgcgggagactggggttcgattccctgaggaggag

SEQ ID NO: 286 - DNA, 72 bp, Homo sapiens
  source 1..72: mol_type = genomic DNA
tcctcgttagtatggtggtgagtatccctgcctgtcacgcgggagaccggggttcgattccccaacggggag

SEQ ID NO: 287 - DNA, 72 bp, Homo sapiens
  source 1..72: mol_type = genomic DNA
tcctcgttagtatagtggtgagtgtccccgtctgtcacgcgggagaccggggttcgattccccgacggggag

SEQ ID NO: 288 - DNA, 72 bp, Homo sapiens
  source 1..72: mol_type = genomic DNA
tcctcgttagtatagtggtgagtatccccgcctgtcacgcgggagaccggggttcgattccccgacggggag

SEQ ID NO: 289 - DNA, 72 bp, Homo sapiens; identical to SEQ ID NO: 288
  source 1..72: mol_type = genomic DNA

SEQ ID NO: 290 - DNA, 72 bp, Homo sapiens; identical to SEQ ID NO: 288
  source 1..72: mol_type = genomic DNA

SEQ ID NO: 291 - DNA, 72 bp, Homo sapiens; identical to SEQ ID NO: 288
  source 1..72: mol_type = genomic DNA

SEQ ID NO: 292 - DNA, 72 bp, Homo sapiens; identical to SEQ ID NO: 288
  source 1..72: mol_type = genomic DNA

SEQ ID NO: 293 - DNA, 72 bp, Homo sapiens; identical to SEQ ID NO: 288
  source 1..72: mol_type = genomic DNA

SEQ ID NO: 294 - DNA, 72 bp, Homo sapiens; identical to SEQ ID NO: 288
  source 1..72: mol_type = genomic DNA

SEQ ID NO: 295 - DNA, 72 bp, Homo sapiens; identical to SEQ ID NO: 288
  source 1..72: mol_type = genomic DNA

SEQ ID NO: 296 - DNA, 72 bp, Homo sapiens; identical to SEQ ID NO: 288
  source 1..72: mol_type = genomic DNA

SEQ ID NO: 297 - DNA, 72 bp, Homo sapiens; identical to SEQ ID NO: 288
  source 1..72: mol_type = genomic DNA

SEQ ID NO: 298 - DNA, 72 bp, Homo sapiens; identical to SEQ ID NO: 288
  source 1..72: mol_type = genomic DNA

SEQ ID NO: 299 - DNA, 72 bp, Homo sapiens
  source 1..72: mol_type = genomic DNA
tcctcgttagtatagtggttagtatccccgcctgtcacgcgggagaccggggttcaattccccgacggggag

SEQ ID NO: 300 - DNA, 72 bp, Homo sapiens
  source 1..72: mol_type = genomic DNA
gggggtagggctcagggatagagcatttgactgcagatcaagaggtccccggttcgaatctaggtgccccct

SEQ ID NO: 301 - DNA, 72 bp, Homo sapiens
  source 1..72: mol_type = genomic DNA
gggggtatagctcacaggtagagcatttgactgcagatcaagaggtccccggttcaaatccggttactccct

SEQ ID NO: 302 - DNA, 72 bp, Homo sapiens
  source 1..72: mol_type = genomic DNA
gggcgtatagctcaggggtagagcatttgactgcagatcaagaggtccccagttcaaatctgggtgcccact

SEQ ID NO: 303 - DNA, 72 bp, Homo sapiens
  source 1..72: mol_type = genomic DNA
gggggtatagctcacaggtagagcatttgactgcagatcaagaggtccccggttcaaatctgggtgccccct

SEQ ID NO: 304 - DNA, 72 bp, Homo sapiens
  source 1..72: mol_type = genomic DNA
gggcgtatagctcaggggtagagcatttgactgcagatcaagaggtccccagttcaaatctgggtgccccct

SEQ ID NO: 305 - DNA, 72 bp, Homo sapiens
  source 1..72: mol_type = genomic DNA
gggggtatagctcaggggtagagcatttgactgcaaatcaagaggtccctgattcaaatccaggtgccccct

SEQ ID NO: 306 - DNA, 72 bp, Homo sapiens
  source 1..72: mol_type = genomic DNA
gggggtatagttcaggggtagagcatttgactgcagatcaagaggtccctggttcaaatccaggtgccccct

SEQ ID NO: 307 - DNA, 72 bp, Homo sapiens
  source 1..72: mol_type = genomic DNA
ggggatatagctcaggggtagagcatttgactgcagatcaagaggtccccggttcaaatccgggtgcccccc

SEQ ID NO: 308 - DNA, 72 bp, Homo sapiens
  source 1..72: mol_type = genomic DNA
gggggtatagctcaggggtagagcacttgactgcagatcaagaagtccttggttcaaatccaggtgccccct

SEQ ID NO: 309 - DNA, 72 bp, Homo sapiens
  source 1..72: mol_type = genomic DNA
gggggtatagctcaggggtagagcatttgactgcagatcaagaggtctctggttcaaatccaggtgccccct

SEQ ID NO: 310 - DNA, 72 bp, Homo sapiens
  source 1..72: mol_type = genomic DNA
gggggtatagctcaggggtagagcatttgactgcagatcaagaagtccccggttcaaatccgggtgccccct

SEQ ID NO: 311 - DNA, 72 bp, Homo sapiens
  source 1..72: mol_type = genomic DNA
gggggtatagctcaggggtagagcatttgactgcagatcaagaggtccccagttcaaatctgggtgccccct

SEQ ID NO: 312 - DNA, 72 bp, Homo sapiens
  source 1..72: mol_type = genomic DNA
gggggtatagcttaggggtagagcatttgactgcagatcaaaaggtccctggttcaaatccaggtgcccctt

SEQ ID NO: 313 - DNA, 72 bp, Homo sapiens
  source 1..72: mol_type = genomic DNA
gggggtatagcttagcggtagagcatttgactgcagatcaagaggtccccggttcaaatccgggtgccccct

SEQ ID NO: 314 - DNA, 72 bp, Homo sapiens
  source 1..72: mol_type = genomic DNA
gggggtatagctcaggggtagagcatttgactgcagatcaagaggtccctggttcaaatccaggtgcccccc

SEQ ID NO: 315 - DNA, 72 bp, Homo sapiens
  source 1..72: mol_type = genomic DNA
gggggtatagctcaggggtagagcatttgactgcagatcaagaggtccctggttcaaatccaggtgccccct

SEQ ID NO: 316 - DNA, 72 bp, Homo sapiens; identical to SEQ ID NO: 315
  source 1..72: mol_type = genomic DNA

SEQ ID NO: 317 - DNA, 72 bp, Homo sapiens; identical to SEQ ID NO: 315
  source 1..72: mol_type = genomic DNA

SEQ ID NO: 318 - DNA, 72 bp, Homo sapiens; identical to SEQ ID NO: 315
  source 1..72: mol_type = genomic DNA

SEQ ID NO: 319 - DNA, 72 bp, Homo sapiens
  source 1..72: mol_type = genomic DNA
gggggtatagctcaggggtagagcatttgactgcagatcaagaggtccccggttcaaatccgggtgccccct

SEQ ID NO: 320 - DNA, 73 bp, Homo sapiens
  source 1..73: mol_type = genomic DNA
gggggtatagctcaggtggtagagcatttgactgcagatcaagaggtccccggttcaaatccgggtgccccct

SEQ ID NO: 321 - DNA, 72 bp, Homo sapiens
  source 1..72: mol_type = genomic DNA
gggggtgtagctcagtggtagagcatttgactgcagatcaagaggtccctggttcaaatccaggtgccccct

SEQ ID NO: 322 - DNA, 73 bp, Homo sapiens
  source 1..73: mol_type = genomic DNA
gggggtatagctcagtgggtagagcatttgactgcagatcaagaggtccccggttcaaatccgggtgccccct

SEQ ID NO: 323 - DNA, 72 bp, Homo sapiens
  source 1..72: mol_type = genomic DNA
gggggtatagctcagtggtagagcatttgactgcagatcaagaggtccctggttcaaatccgggtgccccct

SEQ ID NO: 324 - DNA, 72 bp, Homo sapiens
  source 1..72: mol_type = genomic DNA
gggggtatagctcaggggtagagcacttgactgcagatcaagaggtccctggttcaaatccaggtgccccct

SEQ ID NO: 325 - DNA, 72 bp, Homo sapiens
  source 1..72: mol_type = genomic DNA
gggggtatagctcagtggtagagcatttgactgcagatcaagaggtccccggttcaaatccgggtgccccct

SEQ ID NO: 326 - DNA, 72 bp, Homo sapiens; identical to SEQ ID NO: 325
  source 1..72: mol_type = genomic DNA

SEQ ID NO: 327 - DNA, 72 bp, Homo sapiens; identical to SEQ ID NO: 325
  source 1..72: mol_type = genomic DNA

SEQ ID NO: 328 - DNA, 72 bp, Homo sapiens; identical to SEQ ID NO: 325
  source 1..72: mol_type = genomic DNA

SEQ ID NO: 329 - DNA, 72 bp, Homo sapiens
  source 1..72: mol_type = genomic DNA
gggggcatagctcagtggtagagcatttgactgcagatcaagaggtccctggttcaaatccaggtgccccct

SEQ ID NO: 330 - DNA, 74 bp, Homo sapiens
  source 1..74: mol_type = genomic DNA
ggcagtatggtagagtggttaagatcatgaactctgaagtcagagatacttgaatttgaatgctggttctgtca

SEQ ID NO: 331 - DNA, 78 bp, Homo sapiens
  source 1..78: mol_type = genomic DNA
ggcagtgtagcccagaggttcaagggcattcgctctggtatcagaagggtctgggttcaaatcccttgtgcactgctt

SEQ ID NO: 332 - DNA, 73 bp, Homo sapiens
  source 1..73: mol_type = genomic DNA
gagctgtagcatagtgattagggacatggactctggagccaaatctgcctgggttctagtcccagctgtctca

SEQ ID NO: 333 - DNA, 73 bp, Homo sapiens
  source 1..73: mol_type = genomic DNA
ggttccatggtgtaatggtaagcaccctggactctgaatccagcaaccagagttccagtctcagcgtggacct

SEQ ID NO: 334 - DNA, 72 bp, Homo sapiens
  source 1..72: mol_type = genomic DNA
ggttccatggtgtaatggtgaccactttggactctgaatacagtgatcagagttcaagtctcactggaacct

SEQ ID NO: 335 - DNA, 72 bp, Homo sapiens
  source 1..72: mol_type = genomic DNA
ggttccatggtgtaatggtgagcactttggactctgaatacagtgatcagagttcaagtctcactgggacct

SEQ ID NO: 336 - DNA, 72 bp, Homo sapiens; identical to SEQ ID NO: 335
  source 1..72: mol_type = genomic DNA

SEQ ID NO: 337 - DNA, 72 bp, Homo sapiens; identical to SEQ ID NO: 335
  source 1..72: mol_type = genomic DNA

SEQ ID NO: 338 - DNA, 72 bp, Homo sapiens
  source 1..72: mol_type = genomic DNA
ggttccatggtgtaatggtaagcactctggactctgaatccagccatctgagttcgagtctctgtggaacct

SEQ ID NO: 339 - DNA, 72 bp, Homo sapiens
  source 1..72: mol_type = genomic DNA
ggccccatggtgtaatggtcagcactctggactctgaatccagcgatccgagttcaaatctcggtgggaccc

SEQ ID NO: 340 - DNA, 72 bp, Homo sapiens
  source 1..72: mol_type = genomic DNA
ggttccatggtgtaatggttagcactctggactctgaatccggtaatccgagttcaaatctcggtggaacct

SEQ ID NO: 341 - DNA, 72 bp, Homo sapiens
  source 1..72: mol_type = genomic DNA
ggttccatggtgtaatggtaagcactctggactctgaatccagcgatccgagttcgagtctcggtggaacct

SEQ ID NO: 342 - DNA, 72 bp, Homo sapiens; identical to SEQ ID NO: 341
  source 1..72: mol_type = genomic DNA

SEQ ID NO: 343 - DNA, 72 bp, Homo sapiens
  source 1..72: mol_type = genomic DNA
ggttccatggtgtaatggtgagcactctggactctgaatccagcgatccgagttcgagtctcggtggaacct

SEQ ID NO: 344 - DNA, 72 bp, Homo sapiens; identical to SEQ ID NO: 343
  source 1..72: mol_type = genomic DNA

SEQ ID NO: 345 - DNA, 72 bp, Homo sapiens
  source 1..72: mol_type = genomic DNA
ggttccatggtgtaatggttagcactctggactctgaatccagcgatccgagttcaagtctcggtggaacct

SEQ ID NO: 346 - DNA, 72 bp, Homo sapiens
  source 1..72: mol_type = genomic DNA
ggttccatggtgtaatggttagcactctggactctgaatccagcgatccgagttcaaatctcggtggaacct

SEQ ID NO: 347 - DNA, 72 bp, Homo sapiens; identical to SEQ ID NO: 346
  source 1..72: mol_type = genomic DNA

SEQ ID NO: 348 - DNA, 72 bp, Homo sapiens; identical to SEQ ID NO: 346
  source 1..72: mol_type = genomic DNA

SEQ ID NO: 349 - DNA, 72 bp, Homo sapiens; identical to SEQ ID NO: 346
  source 1..72: mol_type = genomic DNA

SEQ ID NO: 350 - DNA, 72 bp, Homo sapiens; identical to SEQ ID NO: 346
  source 1..72: mol_type = genomic DNA

SEQ ID NO: 351 - DNA, 73 bp, Homo sapiens
  source 1..73: mol_type = genomic DNA
gtgcagagtagtacagtggttaaaccatggtctttggagccagactgcctggggtcggatcccagctctcaca

SEQ ID NO: 352 - DNA, 72 bp, Homo sapiens
  source 1..72: mol_type = genomic DNA
taggacttggtgtaatgggtagcacagagaattttggattctcaggggtgggttcaattcctttcgtcctag

SEQ ID NO: 353 - DNA, 73 bp, Homo sapiens
  source 1..73: mol_type = genomic DNA
gaccatgtggcctaagggaaaagacatctcactttgggtcagaagattgagggttcaagtcctttcatggtca

SEQ ID NO: 354 - DNA, 72 bp, Homo sapiens
  source 1..72: mol_type = genomic DNA
taggacatggtgtaataggtagaatggagaattttgaattctcaggggtaggttcaattcctatagttctag

SEQ ID NO: 355 - DNA, 72 bp, Homo sapiens
  source 1..72: mol_type = genomic DNA
ggtcccatggtgtaatggttagcactctgggctttgaatccagcaatccgagttcgaatcttggtgggacct

SEQ ID NO: 356 - DNA, 72 bp, Homo sapiens
  source 1..72: mol_type = genomic DNA
ggccccatggtgtaatggttagcactctggactttgaatccagcgatccgagttcaaatctcggtgggacct

SEQ ID NO: 357 - DNA, 72 bp, Homo sapiens; identical to SEQ ID NO: 356
  source 1..72: mol_type = genomic DNA

SEQ ID NO: 358 - DNA, 72 bp, Homo sapiens; identical to SEQ ID NO: 356
  source 1..72: mol_type = genomic DNA

SEQ ID NO: 359 - DNA, 72 bp, Homo sapiens
  source 1..72: mol_type = genomic DNA
ggtcccatggtgtaatggttagcactctggactttgaatccagcgatccgagttcaaatctcggtgggacct

SEQ ID NO: 360 - DNA, 69 bp, Homo sapiens
  source 1..69: mol_type = genomic DNA
tccctggtagtctagtggctaaagtttggcgctctcaccgccgggactggttgattccagatcagggga

SEQ ID NO: 361 - DNA, 72 bp, Homo sapiens
  source 1..72: mol_type = genomic DNA
cccctggtggtctatcggttaggattcagacctctcaccactgctacccatgctcgattcctggtcagggaa

SEQ ID NO: 362 - DNA, 72 bp, Homo sapiens
  source 1..72: mol_type = genomic DNA
ccccgggtggtgtagtggatgggatttggcgctctcaccaccatggcccggatttgattcccggtcagggaa

SEQ ID NO: 363 - DNA, 71 bp, Homo sapiens
  source 1..71: mol_type = genomic DNA
tccttgatgtctagtggttaggatttggtgctctcactgcagcagcctgggttcatttctcagtcagggaa

SEQ ID NO: 364 - DNA, 72 bp, Homo sapiens
  source 1..72: mol_type = genomic DNA
cccctggtggtctagtgcttaggattcggtgctctcaccgctgctgcctgcgttcgattcccggtcagggaa

SEQ ID NO: 365 - DNA, 72 bp, Homo sapiens
  source 1..72: mol_type = genomic DNA
tccctggtggtctagtggttaggattcggcgctctcaccgccgcggcccgggttcgattcccggtcaggaaa

SEQ ID NO: 366 - DNA, 72 bp, Homo sapiens
  source 1..72: mol_type = genomic DNA
tccctggtggtctagtggttaggattcggcgctctcaccgccgcggcccgggttcgattcccggtcagggaa

SEQ ID NO: 367 - DNA, 72 bp, Homo sapiens; identical to SEQ ID NO: 366
  source 1..72: mol_type = genomic DNA

SEQ ID NO: 368 - DNA, 72 bp, Homo sapiens; identical to SEQ ID NO: 366
  source 1..72: mol_type = genomic DNA

SEQ ID NO: 369 - DNA, 72 bp, Homo sapiens; identical to SEQ ID NO: 366
  source 1..72: mol_type = genomic DNA

SEQ ID NO: 370 - DNA, 72 bp, Homo sapiens; identical to SEQ ID NO: 366
  source 1..72: mol_type = genomic DNA

SEQ ID NO: 371 - DNA, 72 bp, Homo sapiens; identical to SEQ ID NO: 366
  source 1..72: mol_type = genomic DNA

SEQ ID NO: 372 - DNA, 72 bp, Homo sapiens; identical to SEQ ID NO: 366
  source 1..72: mol_type = genomic DNA

SEQ ID NO: 373 - DNA, 69 bp, Homo sapiens
  source 1..69: mol_type = genomic DNA
tccctggtagtctagtggctaaagtttggcgctttcaccgccgggactggttgattccagatcagggga

SEQ ID NO: 374 - DNA, 69 bp, Homo sapiens
  source 1..69: mol_type = genomic DNA
gcctgtggtctagtggttagaattcagtgttttcagtgctctagtccaggttcaattcctggtcaggga

SEQ ID NO: 375 - DNA, 69 bp, Homo sapiens
  source 1..69: mol_type = genomic DNA
ccctgtggtctagtggttaggagttggtgctttcgtcatgacagcccaggttcaattcctggttagaga

SEQ ID NO: 376 - DNA, 71 bp, Homo sapiens
  source 1..71: mol_type = genomic DNA
accctgtggtctagtggctaagactttgtgctttcattgctgcatcctaggttcaattcccagtcagggaa

SEQ ID NO: 377 - DNA, 73 bp, Homo sapiens
  source 1..73: mol_type = genomic DNA
tccctggtggtctagtggctaggattcggcgctttcaccgcctgcagctcgagttcgattcctggtcagggaa

SEQ ID NO: 378 - DNA, 72 bp, Homo sapiens
  source 1..72: mol_type = genomic DNA
gcgttggtggtgtagtggtgagcacagctgcctttcaagcagttaacgcgggttcgattcccgggtaacgaa

SEQ ID NO: 379 - DNA, 72 bp, Homo sapiens
  source 1..72: mol_type = genomic DNA
tccctggtggtctagtggctaggattcggcgctttcaccgccgcggcccgggttcgattcccggtcagggaa

SEQ ID NO: 380 - DNA, 72 bp, Homo sapiens; identical to SEQ ID NO: 379
  source 1..72: mol_type = genomic DNA

SEQ ID NO: 381 - DNA, 72 bp, Homo sapiens
  source 1..72: mol_type = genomic DNA
tccctggtggtctagtggctaggattcggcgctttcaccgccgcggcccgggttcgattcccggccagggaa

SEQ ID NO: 382 - DNA, 72 bp, Homo sapiens
  source 1..72: mol_type = genomic DNA
tcccacatggtctagcggttaggattcctggttttcacccaggcggcccgggttcgactcccggtgtgggaa

SEQ ID NO: 383 - DNA, 72 bp, Homo sapiens; identical to SEQ ID NO: 382
  source 1..72: mol_type = genomic DNA

SEQ ID NO: 384 - DNA, 72 bp, Homo sapiens
  source 1..72: mol_type = genomic DNA
tcccatatggtctagcggttaggattcctggttttcacccaggtggcccgggttcgactcccggtatgggaa

SEQ ID NO: 385 - DNA, 72 bp, Homo sapiens; identical to SEQ ID NO: 384
  source 1..72: mol_type = genomic DNA

SEQ ID NO: 386 - DNA, 71 bp, Homo sapiens
  source 1..71: mol_type = genomic DNA
gccttggtggtgcagtggtagaattctcgcctcccacgtgggagacccgggttcaattcccggccaatgca

SEQ ID NO: 387 - DNA, 71 bp, Homo sapiens
  source 1..71: mol_type = genomic DNA
gcgttggtggtttagtggtagaattctcgcctcccatgcgggagacccgggttcaattcccggccactgca

SEQ ID NO: 388 - DNA, 71 bp, Homo sapiens
  source 1..71: mol_type = genomic DNA
gcattggtggttcaatggtagaattctcgcctcccacgcaggagacccaggttcgattcctggccaatgca

SEQ ID NO: 389 - DNA, 71 bp, Homo sapiens
  source 1..71: mol_type = genomic DNA
gcgccgctggtgtagtggtatcatgcaagattcccattcttgcgacccgggttcgattcccgggcggcgca

SEQ ID NO: 390 - DNA, 71 bp, Homo sapiens; identical to SEQ ID NO: 389
  source 1..71: mol_type = genomic DNA

SEQ ID NO: 391 - DNA, 71 bp, Homo sapiens
  source 1..71: mol_type = genomic DNA
gcattggtggttcagtggtagaattctcgcctcccacgcgggagacccgggttcaattcccggccaatgca

SEQ ID NO: 392 - DNA, 71 bp, Homo sapiens; identical to SEQ ID NO: 391
  source 1..71: mol_type = genomic DNA

SEQ ID NO: 393 - DNA, 71 bp, Homo sapiens
  source 1..71: mol_type = genomic DNA
gcatgggtgattcagtggtagaattttcacctgccatgcaggaggtccaggttcatttcctggcctatgca

SEQ ID NO: 394 - DNA, 71 bp, Homo sapiens
  source 1..71: mol_type = genomic DNA
gcattggtggttcagtggtagaattctcgcctgccatgcgggcggccgggcttcgattcctggccaatgca

SEQ ID NO: 395 - DNA, 71 bp, Homo sapiens
  source 1..71: mol_type = genomic DNA
gcataggtggttcagtggtagaattcttgcctgccacgcaggaggcccaggtttgattcctggcccatgca

SEQ ID NO: 396 - DNA, 71 bp, Homo sapiens
  source 1..71: mol_type = genomic DNA
gcattggtggttcagtggtagaattctcgcctgccacgcgggaggcccgggtttgattcccggccagtgca

SEQ ID NO: 397 - DNA, 71 bp, Homo sapiens
  source 1..71: mol_type = genomic DNA
gcattggtggttcagtggtagaattctcgcctgccacgcgggaggcccgggttcgattcccggccaatgca

SEQ ID NO: 398 - DNA, 71 bp, Homo sapiens; identical to SEQ ID NO: 397
  source 1..71: mol_type = genomic DNA

SEQ ID NO: 399 - DNA, 71 bp, Homo sapiens; identical to SEQ ID NO: 397
  source 1..71: mol_type = genomic DNA

SEQ ID NO: 400 - DNA, 71 bp, Homo sapiens; identical to SEQ ID NO: 397
  source 1..71: mol_type = genomic DNA

SEQ ID NO: 401 - DNA, 71 bp, Homo sapiens; identical to SEQ ID NO: 397
  source 1..71: mol_type = genomic DNA

SEQ ID NO: 402 - DNA, 71 bp, Homo sapiens; identical to SEQ ID NO: 397
  source 1..71: mol_type = genomic DNA

SEQ ID NO: 403 - DNA, 71 bp, Homo sapiens
  source 1..71: mol_type = genomic DNA
gcatgggtggttcagtggtagaattctcgcctgccacgcgggaggcccgggttcgattcccggcccatgca

SEQ ID NO: 404 - DNA, 71 bp, Homo sapiens; identical to SEQ ID NO: 403
  source 1..71: mol_type = genomic DNA

SEQ ID NO: 405 - DNA, 71 bp, Homo sapiens; identical to SEQ ID NO: 403
  source 1..71: mol_type = genomic DNA

SEQ ID NO: 406 - DNA, 71 bp, Homo sapiens; identical to SEQ ID NO: 403
  source 1..71: mol_type = genomic DNA

SEQ ID NO: 407 - DNA, 71 bp, Homo sapiens; identical to SEQ ID NO: 403
  source 1..71: mol_type = genomic DNA

SEQ ID NO: 408 - DNA, 72 bp, Homo sapiens
  source 1..72: mol_type = genomic DNA
gcgttggtggtatagtggtgagcatagttgccttccaagcagttgacccgggctcgattcccgcccaacgca

SEQ ID NO: 409 - DNA, 72 bp, Homo sapiens
  source 1..72: mol_type = genomic DNA
gcgttggtggtatagtggtaagcatagctgccttccaagcagttgacccgggttcgattcccggccaacgca

SEQ ID NO: 410 - DNA, 72 bp, Homo sapiens
  source 1..72: mol_type = genomic DNA
gcgttggtggtatagtggtgagcatagctgccttccaagcagttgacccgggttcgattcccggccaacgca

SEQ ID NO: 411 - DNA, 72 bp, Homo sapiens; identical to SEQ ID NO: 410
  source 1..72: mol_type = genomic DNA

SEQ ID NO: 412 - DNA, 72 bp, Homo sapiens; identical to SEQ ID NO: 410
  source 1..72: mol_type = genomic DNA

SEQ ID NO: 413 - DNA, 72 bp, Homo sapiens; identical to SEQ ID NO: 410
  source 1..72: mol_type = genomic DNA

SEQ ID NO: 414 - DNA, 72 bp, Homo sapiens; identical to SEQ ID NO: 410
  source 1..72: mol_type = genomic DNA

SEQ ID NO: 415 - DNA, 72 bp, Homo sapiens; identical to SEQ ID NO: 410
  source 1..72: mol_type = genomic DNA

SEQ ID NO: 416 - DNA, 72 bp, Homo sapiens
  source 1..72: mol_type = genomic DNA
gcgttggtggtatagtggttagcatagctgccttccaagcagttgacccgggttcgattcccggccaacgca

SEQ ID NO: 417 - DNA, 72 bp, Homo sapiens
  source 1..72: mol_type = genomic DNA
gcagtgactgtatagtggttagcactctgtgttgtggccacagcaaccatggttcaaatctgagtcatgaca

SEQ ID NO: 418 - DNA, 72 bp, Homo sapiens
  source 1..72: mol_type = genomic DNA
gccatgatcgtatagtggttagtactctgcgctgtggccgcagcaacctcggttcgaatccgagtcacggca

SEQ ID NO: 419 - DNA, 72 bp, Homo sapiens
  source 1..72: mol_type = genomic DNA
gccgtgatcgtatagtggttagtactctgcgttgtggccgcagcaacctcggttcgaatccgagtcacggca

SEQ ID NO: 420 - DNA, 72 bp, Homo sapiens; identical to SEQ ID NO: 419
  source 1..72: mol_type = genomic DNA

SEQ ID NO: 421 - DNA, 72 bp, Homo sapiens; identical to SEQ ID NO: 419
  source 1..72: mol_type = genomic DNA

SEQ ID NO: 422 - DNA, 72 bp, Homo sapiens; identical to SEQ ID NO: 419
  source 1..72: mol_type = genomic DNA

SEQ ID NO: 423 - DNA, 72 bp, Homo sapiens; identical to SEQ ID NO: 419
  source 1..72: mol_type = genomic DNA

SEQ ID NO: 424 - DNA, 72 bp, Homo sapiens; identical to SEQ ID NO: 419
  source 1..72: mol_type = genomic DNA

SEQ ID NO: 425 - DNA, 72 bp, Homo sapiens; identical to SEQ ID NO: 419
  source 1..72: mol_type = genomic DNA

SEQ ID NO: 426 - DNA, 72 bp, Homo sapiens; identical to SEQ ID NO: 419
  source 1..72: mol_type = genomic DNA

SEQ ID NO: 427 - DNA, 72 bp, Homo sapiens; identical to SEQ ID NO: 419
  source 1..72: mol_type = genomic DNA

SEQ ID NO: 428 - DNA, 74 bp, Homo sapiens
  source 1..74: mol_type = genomic DNA
ggctggttagttcagttggttagagcgtggtgctaataacgccaaggtcgtgggttcgatccccatatcggcca

SEQ ID NO: 429 - DNA, 74 bp, Homo sapiens
  source 1..74: mol_type = genomic DNA
ggccggttagctcagtcggctagagcgtggtgctaataacgccaaggtcgcgggttcgatccccgtacgggcca

SEQ ID NO: 430 - DNA, 74 bp, Homo sapiens
  source 1..74: mol_type = genomic DNA
ggccggttagctcagttggtcagagcgtggtgctaataacgccaaggtcgcgggttcgatccccgtacgggcca

SEQ ID NO: 431 - DNA, 74 bp, Homo sapiens; identical to SEQ ID NO: 430
  source 1..74: mol_type = genomic DNA

SEQ ID NO: 432 - DNA, 74 bp, Homo sapiens
  source 1..74: mol_type = genomic DNA
ggccggttagctcagttggttagagcgtggtgctaataacgctaaggtcgcgggttcgatccccgtactggcca

SEQ ID NO: 433 - DNA, 74 bp, Homo sapiens
  source 1..74: mol_type = genomic DNA
ggccggttagctcagttggttagagcgtggtgctaataacgccaaggtcgcgggttcgatccccgtacgggcca

SEQ ID NO: 434 - DNA, 74 bp, Homo sapiens; identical to SEQ ID NO: 433
  source 1..74: mol_type = genomic DNA

SEQ ID NO: 435 - DNA, 74 bp, Homo sapiens; identical to SEQ ID NO: 433
  source 1..74: mol_type = genomic DNA

SEQ ID NO: 436 - DNA, 74 bp, Homo sapiens; identical to SEQ ID NO: 433
  source 1..74: mol_type = genomic DNA

SEQ ID NO: 437 - DNA, 74 bp, Homo sapiens; identical to SEQ ID NO: 433
  source 1..74: mol_type = genomic DNA

SEQ ID NO: 438 - DNA, 74 bp, Homo sapiens
  source 1..74: mol_type = genomic DNA
ggccggttagctcagttggttagagcgtggtgctaataacgccaaggtcgcgggttcgaaccccgtacgggcca

SEQ ID NO: 439 - DNA, 74 bp, Homo sapiens
  source 1..74: mol_type = genomic DNA
ggctggttagctcagttggttagagcgtggtgctaataacgccaaggtcgcgggttcgatccccgtactggcca

SEQ ID NO: 440 - DNA, 74 bp, Homo sapiens
  source 1..74: mol_type = genomic DNA
ggccggttagctcagttggttagagcgtggtgctaataacgccaaggtcgcgggttcgatccccgtactggcca

SEQ ID NO: 441 - DNA, 74 bp, Homo sapiens
  source 1..74: mol_type = genomic DNA
ggccggttagctcagttggttagagcgtggcgctaataacgccaaggtcgcgggttcgatccccgtacgggcca

SEQ ID NO: 442 - DNA, 74 bp, Homo sapiens
  source 1..74: mol_type = genomic DNA
ggccggttagctcagttggtaagagcgtggtgctgataacaccaaggtcgcgggctcgactcccgcaccggcca

SEQ ID NO: 443 - DNA, 74 bp, Homo sapiens; identical to SEQ ID NO: 442
  source 1..74: mol_type = genomic DNA

SEQ ID NO: 444 - DNA, 74 bp, Homo sapiens; identical to SEQ ID NO: 442
  source 1..74: mol_type = genomic DNA

SEQ ID NO: 445 - DNA, 74 bp, Homo sapiens; identical to SEQ ID NO: 442
  source 1..74: mol_type = genomic DNA

SEQ ID NO: 446 - DNA, 74 bp, Homo sapiens; identical to SEQ ID NO: 442
  source 1..74: mol_type = genomic DNA

SEQ ID NO: 447 - DNA, 74 bp, Homo sapiens; identical to SEQ ID NO: 442
  source 1..74: mol_type = genomic DNA

SEQ ID NO: 448 - DNA, 74 bp, Homo sapiens; identical to SEQ ID NO: 442
  source 1..74: mol_type = genomic DNA

SEQ ID NO: 449 - DNA, 74 bp, Homo sapiens; identical to SEQ ID NO: 442
  source 1..74: mol_type = genomic DNA

SEQ ID NO: 450 - DNA, 94 bp, Homo sapiens
  source 1..94: mol_type = genomic DNA
gctccagtggcgcaatcggttagcgcgcggtacttatatggcagtatgtgtgcgagtgatgccgaggttgtgagttcgagcctcacctggagca

SEQ ID NO: 451 - DNA, 94 bp, Homo sapiens
  source 1..94: mol_type = genomic DNA
gctccagtggcgcaatcggttagcgcgcggtacttatacaacagtatatgtgcgggtgatgccgaggttgtgagttcgagcctcacctggagca

SEQ ID NO: 452 - DNA, 94 bp, Homo sapiens
  source 1..94: mol_type = genomic DNA
gctccagtggcgcaatcggttagcgcgcggtacttataagacagtgcacctgtgagcaatgccgaggttgtgagttcaagcctcacctggagca

SEQ ID NO: 453 - DNA, 93 bp, Homo sapiens
  source 1..93: mol_type = genomic DNA
gctccagtggcgcaatcggttagcgcgcggtacttatacagcagtacatgcagagcaatgccgaggttgtgagttcgagcctcacctggagca

SEQ ID NO: 454 - DNA, 93 bp, Homo sapiens
  source 1..93: mol_type = genomic DNA
gctccagtggcgcaatcggttagcgcgcggtacttatatgacagtgcgagcggagcaatgccgaggttgtgagttcgatcctcacctggagca

SEQ ID NO: 455 - DNA, 82 bp, Homo sapiens
  source 1..82: mol_type = genomic DNA
ggtagcatggctgagtggtctaagattctgaattaagtctccagtctctttgggggcgtggttttcaatcccaccgctgcta

SEQ ID NO: 456 - DNA, 82 bp, Homo sapiens
  source 1..82: mol_type = genomic DNA
ggtagggtggccgagcggtctaaggcactgtattaagactccagtctcttcagaggcatgggtttgaatcccactgctgcca

SEQ ID NO: 457 - DNA, 73 bp, Homo sapiens
  source 1..73: mol_type = genomic DNA
gggccagtggctcaatggataatgcgtctgactaagaatcagaagattccagccttgactcctggctggctca

SEQ ID NO: 458 - DNA, 82 bp, Homo sapiens
  source 1..82: mol_type = genomic DNA
ggtagcgtggccgagtggtctaagacgctggattaaggctccagtctcttcgggggcgtgggtttgaatcccaccgctgcca

SEQ ID NO: 459 - DNA, 82 bp, Homo sapiens
  source 1..82: mol_type = genomic DNA
ggtagcgtggccgagcggtctaaggcgctggattaaggctccagtctcttcgggggcgtgggttcaaatcccaccgctgcca

SEQ ID NO: 460 - DNA, 82 bp, Homo sapiens
  source 1..82: mol_type = genomic DNA
ggtagcgtggccgagcggtctaaggcgctggattaaggctccagtctcttcgggggcgtgggttcgaatcccaccgctgcca

SEQ ID NO: 461 - DNA, 82 bp, Homo sapiens; identical to SEQ ID NO: 460
  source 1..82: mol_type = genomic DNA

SEQ ID NO: 462 - DNA, 82 bp, Homo sapiens; identical to SEQ ID NO: 460
  source 1..82: mol_type = genomic DNA

SEQ ID NO: 463 - DNA, 82 bp, Homo sapiens; identical to SEQ ID NO: 460
  source 1..82: mol_type = genomic DNA

SEQ ID NO: 464 - DNA, 82 bp, Homo sapiens
  source 1..82: mol_type = genomic DNA
ggtagcgtggccgagcggtctaaggcgctggattaaggctccagtctcttcggaggcgtgggttcgaatcccaccgctgcca

SEQ ID NO: 465 - DNA, 82 bp, Homo sapiens; identical to SEQ ID NO: 464
  source 1..82: mol_type = genomic DNA

SEQ ID NO: 466 - DNA, 82 bp, Homo sapiens; identical to SEQ ID NO: 464
  source 1..82: mol_type = genomic DNA

SEQ ID NO: 467 - DNA, 84 bp, Homo sapiens
  source 1..84: mol_type = genomic DNA
gtcaggatggccgagcagtcttaaggcgctgcgttcaaatcgcaccctccgctggaggcgtgggttcgaatcccacttttgaca

SEQ ID NO: 468 - DNA, 74 bp, Homo sapiens
  source 1..74: mol_type = genomic DNA
gcctccttagtgcagtaggtagcgcatcagtctcaaaatctgaatggtcctgagttcaagcctcagagggggca

SEQ ID NO: 469 - DNA, 106 bp, Homo sapiens
  source 1..106: mol_type = genomic DNA
gtcaggatggccgagtggtctaaggcgccagactcaaggtaagcaccttgcctgcgggctttctggtctccggatggaggcgtgggttcgaatcccacttctgaca

SEQ ID NO: 470 - DNA, 107 bp, Homo sapiens
  source 1..107: mol_type = genomic DNA
gtcaggatggccgagtggtctaaggcgccagactcaagttgctacttcccaggtttggggcttctggtctccgcatggaggcgtgggttcgaatcccacttctgaca

SEQ ID NO: 471 - DNA, 108 bp, Homo sapiens
  source 1..108: mol_type = genomic DNA
gtcaggatggccgagtggtctaaggcgccagactcaagcttactgcttcctgtgttcgggtcttctggtctccgtatggaggcgtgggttcgaatcccacttctgaca

SEQ ID NO: 472 - DNA, 106 bp, Homo sapiens
  source 1..106: mol_type = genomic DNA
gtcaggatggccgagtggtctaaggcgccagactcaagctaagcttcctccgcggtggggattctggtctccaatggaggcgtgggttcgaatcccacttctgaca

SEQ ID NO: 473 - DNA, 105 bp, Homo sapiens
  source 1..105: mol_type = genomic DNA
gtcaggatggccgagtggtctaaggcgccagactcaagcttggcttcctcgtgttgaggattctggtctccaatggaggcgtgggttcgaatcccacttctgaca

SEQ ID NO: 474 - DNA, 78 bp, Homo sapiens
  source 1..78: mol_type = genomic DNA
ggcagtggagtttagtggttaaggacctgctcagacatcacaggtaggtagatctgggttcaaaccctagccctggca

SEQ ID NO: 475 - DNA, 83 bp, Homo sapiens
  source 1..83: mol_type = genomic DNA
gtcaggatggccgagcggtctaaggcgctgcgttcaggtcgcagtctcccctggaggcgtgggttcgaatcccacttctgaca

SEQ ID NO: 476 - DNA, 83 bp, Homo sapiens; identical to SEQ ID NO: 475
  source 1..83: mol_type = genomic DNA

SEQ ID NO: 477 - DNA, 83 bp, Homo sapiens
  source 1..83: mol_type = genomic DNA
gtcaggatggccgagcggtctaaggcgctgcgttcaggtcgcagtctcccctggaggcgtgggttcgaatcccactcctgaca

SEQ ID NO: 478 - DNA, 83 bp, Homo sapiens; identical to SEQ ID NO: 477
  source 1..83: mol_type = genomic DNA

SEQ ID NO: 479 - DNA, 83 bp, Homo sapiens; identical to SEQ ID NO: 477
  source 1..83: mol_type = genomic DNA

SEQ ID NO: 480 - DNA, 83 bp, Homo sapiens; identical to SEQ ID NO: 477
  source 1..83: mol_type = genomic DNA

SEQ ID NO: 481 - DNA, 83 bp, Homo sapiens; identical to SEQ ID NO: 477
  source 1..83: mol_type = genomic DNA

SEQ ID NO: 482 - DNA, 83 bp, Homo sapiens; identical to SEQ ID NO: 477
  source 1..83: mol_type = genomic DNA

SEQ ID NO: 483 - DNA, 83 bp, Homo sapiens; identical to SEQ ID NO: 477
  source 1..83: mol_type = genomic DNA

SEQ ID NO: 484 - DNA, 75 bp, Homo sapiens
  source 1..75: mol_type = genomic DNA
gttaagatggcagagcccggcaattgcataagacttaaaactttataatcagaggttcaactcctctcattaaca

SEQ ID NO: 485 - DNA, 83 bp, Homo sapiens
  source 1..83: mol_type = genomic DNA
actcatttggctgagtggttaaggcattggacttaagatccaatggagtagtggctgtgtgggtttaaaccccactactggta

SEQ ID NO: 486 - DNA, 75 bp, Homo sapiens
  source 1..75: mol_type = genomic DNA
gttaagatggcagagcctggtaattgcataaaacttaaaattttataatcagaggttcaactcctcttcttaaca

SEQ ID NO: 487 - DNA, 83 bp, Homo sapiens
  source 1..83: mol_type = genomic DNA
accgggatggctgagtggttaaggcgttggacttaagatccaatggacaggtgtccgcgtgggttcgagccccactcccggta

SEQ ID NO: 488 - DNA, 83 bp, Homo sapiens
  source 1..83: mol_type = genomic DNA
accagaatggccgagtggttaaggcgttggacttaagatccaatggattcatatccgcgtgggttcgaaccccacttctggta

SEQ ID NO: 489 - DNA, 83 bp, Homo sapiens
  source 1..83: mol_type = genomic DNA
accgggatggccgagtggttaaggcgttggacttaagatccaatgggctggtgcccgcgtgggttcgaaccccactctcggta

SEQ ID NO: 490 - DNA, 83 bp, Homo sapiens
  source 1..83: mol_type = genomic DNA
accaggatggccgagtggttaaggcgttggacttaagatccaatggacatatgtccgcgtgggttcgaaccccactcctggta

SEQ ID NO: 491 - DNA, 82 bp, Homo sapiens
  source 1..82: mol_type = genomic DNA
ggtagcgtggccgagtggtctaaggcgctggatttaggctccagtcatttcgatggcgtgggttcgaatcccaccgctgcca

SEQ ID NO: 492 - DNA, 82 bp, Homo sapiens
  source 1..82: mol_type = genomic DNA
ggtagtgtggccgagcggtctaaggcgctggatttaggctccagtctcttcgggggcgtgggttcgaatcccaccactgcca

SEQ ID NO: 493 - DNA, 82 bp, Homo sapiens
  source 1..82: mol_type = genomic DNA
ggtagcgtggccgagcggtctaaggcgctggatttaggctccagtctcttcggaggcgtgggttcgaatcccaccgctgcca

SEQ ID NO: 494 - DNA, 73 bp, Homo sapiens
  source 1..73: mol_type = genomic DNA
aaccgaatagcttagttgatgaagcgtgagactcttaatctcagggtagtgggttcaagccccacattggaca

SEQ ID NO: 495 - DNA, 73 bp, Homo sapiens
  source 1..73: mol_type = genomic DNA
ctgcagctagctcagtcggtagagcatgagactcttaatctcagggtcatgggttcgtgccccatgttgggtg

SEQ ID NO: 496 - DNA, 73 bp, Homo sapiens
  source 1..73: mol_type = genomic DNA
gcccagctagctcagtcggtagagcataagactcttaatctcagggttgtggattcgtgccccatgctgggtg

SEQ ID NO: 497 - DNA, 73 bp, Homo sapiens
  source 1..73: mol_type = genomic DNA
gcccgactacctcagtcggtggagcatgggactcttcatcccagggttgtgggttcgagccccacattgggca

SEQ ID NO: 498 - DNA, 73 bp, Homo sapiens
  source 1..73: mol_type = genomic DNA
gcctggctagctcagtcggcaaagcatgagactcttaatctcagggtcgtgggctcgagctccatgttgggcg

SEQ ID NO: 499 - DNA, 73 bp, Homo sapiens
  source 1..73: mol_type = genomic DNA
gcccagctagctcagtcggtagagcatgagactcttaatctcagggtcatgggtttgagccccacgtttggtg

SEQ ID NO: 500 - DNA, 73 bp, Homo sapiens
  source 1..73: mol_type = genomic DNA
gacgagctagctcagtcggtagagcatgggactcttaatcccagggtcgtgggtttgagccccatgttgggca

SEQ ID NO: 501 - DNA, 73 bp, Homo sapiens
  source 1..73: mol_type = genomic DNA
gcccggctagctcagtcgatagagcatgagactcttaatctcagggtcgtgggttcgagccgcacgttgggcg

SEQ ID NO: 502 - DNA, 73 bp, Homo sapiens
  source 1..73: mol_type = genomic DNA
gcccggctagctcagtcggtagagcatgggactcttaatctcagggtcgtgggttcgagccccacgttgggcg

SEQ ID NO: 503 - DNA, 73 bp, Homo sapiens
  source 1..73: mol_type = genomic DNA
gcccggctagctcagtcggtagagcatgagacccttaatctcagggtcgtgggttcgagccccacgttgggcg

SEQ ID NO: 504 - DNA, 73 bp, Homo sapiens
  source 1..73: mol_type = genomic DNA
gcccggctagctcagtcggtagagcatgagactcttaatctcagggtcgtgggttcgagccccacgttgggcg

SEQ ID NO: 505 - DNA, 73 bp, Homo sapiens; identical to SEQ ID NO: 504
  source 1..73: mol_type = genomic DNA

SEQ ID NO: 506 - DNA, 73 bp, Homo sapiens; identical to SEQ ID NO: 504
  source 1..73: mol_type = genomic DNA

SEQ ID NO: 507 - DNA, 73 bp, Homo sapiens; identical to SEQ ID NO: 504
  source 1..73: mol_type = genomic DNA

SEQ ID NO: 508 - DNA, 73 bp, Homo sapiens; identical to SEQ ID NO: 504
  source 1..73: mol_type = genomic DNA

SEQ ID NO: 509 - DNA, 73 bp, Homo sapiens
  source 1..73: mol_type = genomic DNA
gcccggctagctcagtcggtagagcatgggactcttaatcccagggtcgtgggttcgagccccacgttgggcg

SEQ ID NO: 510 - DNA, 73 bp, Homo sapiens; identical to SEQ ID NO: 509
  source 1..73: mol_type = genomic DNA

SEQ ID NO: 511 - DNA, 71 bp, Homo sapiens
  source 1..71: mol_type = genomic DNA
accctgtggtacaggggctaatatgctgggcctttaccacttcagcccaggttcgattcctggtcagggaa

SEQ ID NO: 512 - DNA, 73 bp, Homo sapiens
  source 1..73: mol_type = genomic DNA
acctgggtagcttagttggtagagcattggacttttaatttgagggcccaggtttcaagtccctgtttgggtg

SEQ ID NO: 513 - DNA, 73 bp, Homo sapiens
  source 1..73: mol_type = genomic DNA
acccagatagctcagtcagtagagcatcagacttttaatctgagggtccaaggttcatgtccctttttgggtg

SEQ ID NO: 514 - DNA, 73 bp, Homo sapiens
  source 1..73: mol_type = genomic DNA
acctgggtagctcagtaggtagaacatcagacttttaatctgagggtctagggttcaagtccctgtccaggcg

SEQ ID NO: 515 - DNA, 73 bp, Homo sapiens
  source 1..73: mol_type = genomic DNA
gcccggagagctcagtgggtagagcatcagacttttaatctgagggtccagggttcaagtcctcgttcgggca

SEQ ID NO: 516 - DNA, 77 bp, Homo sapiens
  source 1..77: mol_type = genomic DNA
gcccacgtagctcaatggtcagagcgtgcggcttttaaccgcaaggaaggctgcgagttcgaccctcgccgtgggct

SEQ ID NO: 517 - DNA, 73 bp, Homo sapiens
  source 1..73: mol_type = genomic DNA
gcctggatagctcagttggtagaacatcagacttttaatctgacggtgcagggttcaagtccctgttcaggcg

SEQ ID NO: 518 - DNA, 73 bp, Homo sapiens
  source 1..73: mol_type = genomic DNA
gcctgggtagctcagtcggtagagcatcagacttttaatctgagggtccagggttcaagtccctgtccaggcg

SEQ ID NO: 519 - DNA, 73 bp, Homo sapiens
  source 1..73: mol_type = genomic DNA
gcccggatagctcagtcggtagagcatcagacttttaatctgagggtccggggttcaagtccctgttcgggcg

SEQ ID NO: 520 - DNA, 73 bp, Homo sapiens
  source 1..73: mol_type = genomic DNA
gcctggatagctcagtcggtagagcatcagacttttaatctgagggtccagggttcaagtccctgttcaggcg

SEQ ID NO: 521 - DNA, 73 bp, Homo sapiens
  source 1..73: mol_type = genomic DNA
gcccggatagctcagtcggtagagcatcagacttttaatctgagggtccagggttcaagtccctgttcgggcg

SEQ ID NO: 522 - DNA, 73 bp, Homo sapiens; identical to SEQ ID NO: 521
  source 1..73: mol_type = genomic DNA

SEQ ID NO: 523 - DNA, 73 bp, Homo sapiens; identical to SEQ ID NO: 521
  source 1..73: mol_type = genomic DNA

SEQ ID NO: 524 - DNA, 73 bp, Homo sapiens; identical to SEQ ID NO: 521
  source 1..73: mol_type = genomic DNA

SEQ ID NO: 525 - DNA, 73 bp, Homo sapiens; identical to SEQ ID NO: 521
  source 1..73: mol_type = genomic DNA

SEQ ID NO: 526 - DNA, 73 bp, Homo sapiens
  source 1..73: mol_type = genomic DNA
gcctggatagctcagttggtagagcatcagacttttaatctgagggtccagggttcaagtccctgttcaggcg

SEQ ID NO: 527 - DNA, 73 bp, Homo sapiens
  source 1..73: mol_type = genomic DNA
gcctggatagctcagttggtagagcatcagacttttaatctgagggtccagggttcaagtccctgttcaggca

SEQ ID NO: 528 - DNA, 75 bp, Homo sapiens
  source 1..75: mol_type = genomic DNA
agcagagtggtgcagtggaagcatacctatgggcccataacccagaggttgatggatggaaaccatcctctgcta

SEQ ID NO: 529 - DNA, 72 bp, Homo sapiens
  source 1..72: mol_type = genomic DNA
agcagagtggcgcagcggaagcgtgctgggcccataacccagaggtcgatggatctaaaccatcctctgcta

SEQ ID NO: 530 - DNA, 73 bp, Homo sapiens
  source 1..73: mol_type = genomic DNA
gccctcttagtgcagctggcagcgcgtcagtttcataatctgaaagtcctgagttcaagcctcagagagggca

SEQ ID NO: 531 - DNA, 72 bp, Homo sapiens
  source 1..72: mol_type = genomic DNA
agcagagtggcgcagcggaagcgtgctgggcccataacccagaggtcgatggatcgaaaccatcctctgcta

SEQ ID NO: 532 - DNA, 72 bp, Homo sapiens; identical to SEQ ID NO: 531
  source 1..72: mol_type = genomic DNA

SEQ ID NO: 533 - DNA, 72 bp, Homo sapiens; identical to SEQ ID NO: 531
  source 1..72: mol_type = genomic DNA

SEQ ID NO: 534 - DNA, 72 bp, Homo sapiens; identical to SEQ ID NO: 531
  source 1..72: mol_type = genomic DNA

SEQ ID NO: 535 - DNA, 72 bp, Homo sapiens; identical to SEQ ID NO: 531
  source 1..72: mol_type = genomic DNA

SEQ ID NO: 536 - DNA, 72 bp, Homo sapiens; identical to SEQ ID NO: 531
  source 1..72: mol_type = genomic DNA

SEQ ID NO: 537 - DNA, 72 bp, Homo sapiens; identical to SEQ ID NO: 531
  source 1..72: mol_type = genomic DNA

SEQ ID NO: 538 - DNA, 72 bp, Homo sapiens; identical to SEQ ID NO: 531
  source 1..72: mol_type = genomic DNA

SEQ ID NO: 539 - DNA, 73 bp, Homo sapiens
  source 1..73: mol_type = genomic DNA
gcctcgttagcgcagtaggcagcgcgtcagtctcataatctgaaggtcgtgagttcgagcctcacacggggca

SEQ ID NO: 540 - DNA, 73 bp, Homo sapiens
  source 1..73: mol_type = genomic DNA
gccctcttagcgcagctggcagcgcgtcagtctcataatctgaaggtcctgagttcaagcctcagagagggca

SEQ ID NO: 541 - DNA, 73 bp, Homo sapiens
  source 1..73: mol_type = genomic DNA
gccctcttagcgcagcgggcagcgcgtcagtctcataatctgaaggtcctgagttcgagcctcagagagggca

SEQ ID NO: 542 - DNA, 73 bp, Homo sapiens; identical to SEQ ID NO: 541
  source 1..73: mol_type = genomic DNA

SEQ ID NO: 543 - DNA, 73 bp, Homo sapiens; identical to SEQ ID NO: 541
  source 1..73: mol_type = genomic DNA

SEQ ID NO: 544 - DNA, 73 bp, Homo sapiens
  source 1..73: mol_type = genomic DNA
gcctccttagcgcagtaggcagcgcgtcagtctcataatctgaaggtcctgagttcgaacctcagagggggca

SEQ ID NO: 545 - DNA, 73 bp, Homo sapiens; identical to SEQ ID NO: 544
  source 1..73: mol_type = genomic DNA

SEQ ID NO: 546 - DNA, 73 bp, Homo sapiens
  source 1..73: mol_type = genomic DNA
gccctcttagcgcagtgggcagcgcgtcagtctcataatctgaaggtcctgagttcgagcctcagagagggca

SEQ ID NO: 547 - DNA, 73 bp, Homo sapiens
  source 1..73: mol_type = genomic DNA
gcctcgttagcgcagtaggtagcgcgtcagtctcataatctgaaggtcgtgagttcgatcctcacacggggca

SEQ ID NO: 548 - DNA, 76 bp, Homo sapiens
  source 1..76: mol_type = genomic DNA
gccgaaatagctcaattgggagagtgttagactgaagatcttctgcaggtctctggttcaattccgggtttcgaca

SEQ ID NO: 549 - DNA, 74 bp, Homo sapiens
  source 1..74: mol_type = genomic DNA
gctgaaatagctcagttgggagagcgttagactgaagatcttaaagttccctggttcaaccctgggtttcagcc

SEQ ID NO: 550 - DNA, 73 bp, Homo sapiens
  source 1..73: mol_type = genomic DNA
gccaaaattgctcagttgggagagcgttagactgaagatctaaaggtccctggttcgatcccgggtttcacca

SEQ ID NO: 551 - DNA, 74 bp, Homo sapiens
  source 1..74: mol_type = genomic DNA
gccgaaatagctcagttgggagagcgttagaccgaagatcttaaaggtccctggttcaatcccgggtttcggca

SEQ ID NO: 552 - DNA, 73 bp, Homo sapiens
  source 1..73: mol_type = genomic DNA
gccgagatagctcagttgggagagcgttagactgaagatctaaaggtccctggttcaatcccgggtttcggca

SEQ ID NO: 553 - DNA, 73 bp, Homo sapiens
  source 1..73: mol_type = genomic DNA
gccgaaatagctcagttgggagagcgttagactgaagatctaaaggtccctggttcaatcccgggtttcggca

SEQ ID NO: 554 - DNA, 73 bp, Homo sapiens
  source 1..73: mol_type = genomic DNA
gccgaaatagctcagttgggagagcgttagactgaagatctaaaggtccctggttcgatcccgggtttcggca

SEQ ID NO: 555 - DNA, 73 bp, Homo sapiens; identical to SEQ ID NO: 554
  source 1..73: mol_type = genomic DNA

SEQ ID NO: 556 - DNA, 73 bp, Homo sapiens; identical to SEQ ID NO: 554
  source 1..73: mol_type = genomic DNA

SEQ ID NO: 557 - DNA, 73 bp, Homo sapiens; identical to SEQ ID NO: 554
  source 1..73: mol_type = genomic DNA

SEQ ID NO: 558 - DNA, 73 bp, Homo sapiens; identical to SEQ ID NO: 554
  source 1..73: mol_type = genomic DNA

SEQ ID NO: 559 - DNA, 73 bp, Homo sapiens; identical to SEQ ID NO: 554
  source 1..73: mol_type = genomic DNA

SEQ ID NO: 560 - DNA, 72 bp, Homo sapiens
  source 1..72: mol_type = genomic DNA
ggctcgttggtctaggggtatgattctcgcttagggtgcgagaggtcccgggttcaaatcccggacgagccc

SEQ ID NO: 561 - DNA, 72 bp, Homo sapiens; identical to SEQ ID NO: 560
  source 1..72: mol_type = genomic DNA

SEQ ID NO: 562 - DNA, 72 bp, Homo sapiens; identical to SEQ ID NO: 560
  source 1..72: mol_type = genomic DNA

SEQ ID NO: 563 - DNA, 72 bp, Homo sapiens; identical to SEQ ID NO: 560
  source 1..72: mol_type = genomic DNA

SEQ ID NO: 564 - DNA, 72 bp, Homo sapiens; identical to SEQ ID NO: 560
  source 1..72: mol_type = genomic DNA

SEQ ID NO: 565 - DNA, 72 bp, Homo sapiens; identical to SEQ ID NO: 560
  source 1..72: mol_type = genomic DNA

SEQ ID NO: 566 - DNA, 72 bp, Homo sapiens; identical to SEQ ID NO: 560
  source 1..72: mol_type = genomic DNA

SEQ ID NO: 567 - DNA, 72 bp, Homo sapiens; identical to SEQ ID NO: 560
  source 1..72: mol_type = genomic DNA

SEQ ID NO: 568 - DNA, 72 bp, Homo sapiens
  source 1..72: mol_type = genomic DNA
ggctcgttggtctaggggtatgattctcgcttaggatgcgagaggtcccgggttcaaatcccggacgagccc

SEQ ID NO: 569 - DNA, 95 bp, Homo sapiens
  source 1..95: mol_type = genomic DNA
ggctcgttggtctaggggtgtggttctcgcttagggaccacagggacaagcccgggagacccaagaggtcccgggttcaaatcccggacgagccc

SEQ ID NO: 570 - DNA, 72 bp, Homo sapiens
  source 1..72: mol_type = genomic DNA
ggctcgttggtctaggggtatgattctcgcttcgggtgtgagaggtcccgggttcaaatcccggacgagccc

SEQ ID NO: 571 - DNA, 72 bp, Homo sapiens
  source 1..72: mol_type = genomic DNA
ggctcgttggtctaggggtatgattctcgcttcgggtgcgagaggtcccgggttcaaatcccggacgagccc

SEQ ID NO: 572 - DNA, 72 bp, Homo sapiens; identical to SEQ ID NO: 571
  source 1..72: mol_type = genomic DNA

SEQ ID NO: 573 - DNA, 72 bp, Homo sapiens; identical to SEQ ID NO: 571
  source 1..72: mol_type = genomic DNA

SEQ ID NO: 574 - DNA, 72 bp, Homo sapiens
  source 1..72: mol_type = genomic DNA
ggctcgttggtctaggggtatgattctcgctttgggtgcgagaggtcccgggttcaaatcccggacgagccc

SEQ ID NO: 575 - DNA, 72 bp, Homo sapiens; identical to SEQ ID NO: 574
  source 1..72: mol_type = genomic DNA

SEQ ID NO: 576 - DNA, 72 bp, Homo sapiens; identical to SEQ ID NO: 574
  source 1..72: mol_type = genomic DNA

SEQ ID NO: 577 - DNA, 72 bp, Homo sapiens; identical to SEQ ID NO: 574
  source 1..72: mol_type = genomic DNA

SEQ ID NO: 578 - DNA, 72 bp, Homo sapiens; identical to SEQ ID NO: 574
  source 1..72: mol_type = genomic DNA

SEQ ID NO: 579 - DNA, 72 bp, Homo sapiens
  source 1..72: mol_type = genomic DNA
ggctcgttggtctaggggtatgattctcggtttgggtccgagaggtcccgggttcaaatcccggacgagccc

SEQ ID NO: 580 - DNA, 72 bp, Homo sapiens
  source 1..72: mol_type = genomic DNA
ggctcgttggtctagtggtatgattctcgctttgggtgcgagaggtcccgggttcaaatcccggacgagccc

SEQ ID NO: 581 - DNA, 82 bp, Homo sapiens
  source 1..82: mol_type = genomic DNA
gatagcaaggccgagcggtctaaggctccggattaaggcgccggtgtcttcggaggcatgggttcgaattccacctctgcca

SEQ ID NO: 582 - DNA, 78 bp, Homo sapiens
  source 1..78: mol_type = genomic DNA
gaccaattagcaagcacagttggctagaacatggtgctaataaggccacggtcaggggttcaattcccttatgggctg

SEQ ID NO: 583 - DNA, 75 bp, Homo sapiens
  source 1..75: mol_type = genomic DNA
tggctgatgagctcagctggtgggagcatggtgttaatgaggctgaggtcgtgggttcaatccccaccgggctat

SEQ ID NO: 584 - DNA, 74 bp, Homo sapiens
  source 1..74: mol_type = genomic DNA
ggccggttaacttaattggttagagcgtggtgctaataatgtcaaggttgcgggttggatccccgaacgggcca

SEQ ID NO: 585 - DNA, 176 bp, Homo sapiens
  source 1..176: mol_type = genomic DNA
ggctgtatagctcagtggtagagcatttgactacagaatcctatactcaggggaaggagaactgggggtttctcagtgggtcaaaggacttgtagtggtaaatcaaaagcaactctataagctatgtaacaaactttaaagtcatatgtagctgggttcaaatcctgtttctgcca

SEQ ID NO: 586 - DNA, 74 bp, Homo sapiens
  source 1..74: mol_type = genomic DNA
ggccggttagctcagttggttagagcgtgctgctactaatgccagggtcgaggtttcgatccccgtacgggcct

SEQ ID NO: 587 - DNA, 72 bp, Homo sapiens
  source 1..72: mol_type = genomic DNA
ggtaatgtagcctcgtggttaggggctgcattctagagctatgctgcccaggttcaaattctggtgccactc

SEQ ID NO: 588 - DNA, 72 bp, Homo sapiens
  source 1..72: mol_type = genomic DNA
gggggtatagctcagggacagagcacgtggttagcatgcgtgaggtcctgcgttcaacttccagtatttcca

SEQ ID NO: 589 - DNA, 72 bp, Homo sapiens
  source 1..72: mol_type = genomic DNA
cgctctttggtctaggggtatgatcttcgcttagggtgcgagaggtgcctggatcaactccttcacaagccg

SEQ ID NO: 590 - DNA, 71 bp, Homo sapiens
  source 1..71: mol_type = genomic DNA
ggctggttggtctagggctatgattctcacttagggtgcaagaggtcctggttcaaatcccagaggagccc

SEQ ID NO: 591 - DNA, 72 bp, Homo sapiens
  source 1..72: mol_type = genomic DNA
ggctggttggtctagggctatgattctcacttagggtgcaaaaggtcctgggttcaaatcccagaggagccc

SEQ ID NO: 592 - DNA, 68 bp, Homo sapiens
  source 1..68: mol_type = genomic DNA
catagggtagtggctaagaacctaaactctaaatttagatgtcctgagttcaaatcccagctgtatgc

SEQ ID NO: 593 - DNA, 73 bp, Homo sapiens
  source 1..73: mol_type = genomic DNA
gcttctgtaatgtagtggttatcacattcgcctcacacatgaaaggtcaccagtttgagaccgggccaaaaca

SEQ ID NO: 594 - DNA, 73 bp, Homo sapiens
  source 1..73: mol_type = genomic DNA
gtttctgtagtatagtggttatcatgtttgcctcacatgtgaaagacccttggctcgagactggagggaaaca

SEQ ID NO: 595 - DNA, 73 bp, Homo sapiens
  source 1..73: mol_type = genomic DNA
gtttctgtggtgtagtggttattatgttcgcttcacatatgaaaggtctctggttcgagactgcgtgggaaca

SEQ ID NO: 596 - DNA, 73 bp, Homo sapiens; identical to SEQ ID NO: 595
  source 1..73: mol_type = genomic DNA

SEQ ID NO: 597 - DNA, 72 bp, Homo sapiens
  source 1..72: mol_type = genomic DNA
tactcagtggtctagtggttaggattcagcgctcccaccgccgcagcccgggttcgattcccggtcatggaa

SEQ ID NO: 598 - DNA, 71 bp, Homo sapiens
  source 1..71: mol_type = genomic DNA
gcactggtggttcagtggtagaattctcgcctcccacgcgggagacccgggtttaattcccggtcaagata

SEQ ID NO: 599 - DNA, 69 bp, Homo sapiens
  source 1..69: mol_type = genomic DNA
gcattcgtagttcagcggcagaaatttcgtctcctacgcgggagactcgggttcgacttcggccatgca

SEQ ID NO: 600 - DNA, 71 bp, Homo sapiens
  source 1..71: mol_type = genomic DNA
ggatttgtggtccagtggtagaattctcaccgcctgcataggagaccctggtttaattcctggccaatgca

SEQ ID NO: 601 - DNA, 71 bp, Homo sapiens
  source 1..71: mol_type = genomic DNA
ggattggtggtccagtggtagaattctcaccgcctgcataggagaccctggtttaattcctggccaatgca

SEQ ID NO: 602 - DNA, 71 bp, Homo sapiens
  source 1..71: mol_type = genomic DNA
gcattggtagttcagcggtggcattctccccacctacgcgggagacctgggttcaactcccggccaaagca

SEQ ID NO: 603 - DNA, 71 bp, Homo sapiens; identical to SEQ ID NO: 602
  source 1..71: mol_type = genomic DNA

SEQ ID NO: 604 - DNA, 73 bp, Homo sapiens
  source 1..73: mol_type = genomic DNA
ggcgggataatgtagtggttaaaggcatgggctctagagccagacttcctgggttcaaatctcagacctgcta

SEQ ID NO: 605 - DNA, 68 bp, Homo sapiens
  source 1..68: mol_type = genomic DNA
ggcagggtagggtagaggttaaaaccatggattctagagccagatgggttcaaatcccggctctgccg

SEQ ID NO: 606 - DNA, 72 bp, Homo sapiens
  source 1..72: mol_type = genomic DNA
tccctggtgttccggtggttaggatttggcattctcactgttgtggtgcggattcaatcctggcttagggta

SEQ ID NO: 607 - DNA, 73 bp, Homo sapiens
  source 1..73: mol_type = genomic DNA
ccgtggatagcccagcggctatgggagccgggctctcactctgacgtcctgggttcaagtcccagtgtgcaca

SEQ ID NO: 608 - DNA, 73 bp, Homo sapiens
  source 1..73: mol_type = genomic DNA
cccctggcggtctagtggttaggattcggcgctctcatccaccgcggcctgggttcgactcgtggtcagagtg

SEQ ID NO: 609 - DNA, 75 bp, Homo sapiens
  source 1..75: mol_type = genomic DNA
ctctggtggtttagtggctaggattcacctctctcactgctgcagcccagggttccattccctgggagtcagatg

SEQ ID NO: 610 - DNA, 72 bp, Homo sapiens
  source 1..72: mol_type = genomic DNA
ccccttgtagtctagtggttagaattctgcggtctcacagccgcggcccgggttcgattcccattccgggaa

SEQ ID NO: 611 - DNA, 72 bp, Homo sapiens
  source 1..72: mol_type = genomic DNA
ttattattatacctgtggttaggattcggcgctctcaccgccacgacccgggttcaattcccggtcagggaa

SEQ ID NO: 612 - DNA, 72 bp, Homo sapiens
  source 1..72: mol_type = genomic DNA
cccctggtagtctagtggttaggctttgccgctctcagtgccgctgcctgggttggattcccagtcatgtga

SEQ ID NO: 613 - DNA, 72 bp, Homo sapiens
  source 1..72: mol_type = genomic DNA
tccctgcttgtctagtggttagaattcagcactctcactgccacagcccaggttcaattccctgtcagagaa

SEQ ID NO: 614 - DNA, 73 bp, Homo sapiens
  source 1..73: mol_type = genomic DNA
ttccccttggtctagtggttaggattcaacactctcaccgccgcagcccgggtttgattcccaggcagggaag

SEQ ID NO: 615 - DNA, 72 bp, Homo sapiens
  source 1..72: mol_type = genomic DNA
cccctggtggtctagtgcttaggatttggcactctcgccaccgcagcctgcgttcaattcccggtcagggaa

SEQ ID NO: 616 - DNA, 72 bp, Homo sapiens
  source 1..72: mol_type = genomic DNA
tccctggtggtctaatggttaggagtcggcactctcaccgccgcggctggggtttgattcccagtcatgtaa

SEQ ID NO: 617 - DNA, 76 bp, Homo sapiens
  source 1..76: mol_type = genomic DNA
gtgagactgcacagcccagtggtgcagggcatggctctgacacctggcggcctgggttcaaatcccagcttctaca

SEQ ID NO: 618 - DNA, 72 bp, Homo sapiens
  source 1..72: mol_type = genomic DNA
ggttccatgatgtaatggtgagcgctttggactctgagtacggtgatcagcgttcaagtctcagtgggacct

SEQ ID NO: 619 - DNA, 72 bp, Homo sapiens
  source 1..72: mol_type = genomic DNA
ggtagtgtagtctactggttaaacgcttgggctctgacattaacgtcctgggttcaaatcccagctttgtca

SEQ ID NO: 620 - DNA, 75 bp, Homo sapiens
  source 1..75: mol_type = genomic DNA
ctaggacgtggtgtaataggtagcacagagaattctggattctcaggggtaggttcaattcctatagaacctagg

SEQ ID NO: 621 - DNA, 72 bp, Homo sapiens
  source 1..72: mol_type = genomic DNA
ggttccatggtgtaatggtgagggctttggactctgactacagtgatcagagttcaagtctcagtgggacct

SEQ ID NO: 622 - DNA, 72 bp, Homo sapiens
  source 1..72: mol_type = genomic DNA
ggttccatgggttaatggtgagcaccctggactctgaatcaagcgatccgagttcaaatctcggtggtacct

SEQ ID NO: 623 - DNA, 74 bp, Homo sapiens
  source 1..74: mol_type = genomic DNA
gcctggctacctcagttggtagagcatgggactcttaatcccagagtcagtgggttcaagcctcacattgagtg

SEQ ID NO: 624 - DNA, 73 bp, Homo sapiens
  source 1..73: mol_type = genomic DNA
gcccagctagctcagccggtagagcacaagactcttaatctcagggtcgtgggtttgagccctgtgttgagca

SEQ ID NO: 625 - DNA, 73 bp, Homo sapiens
  source 1..73: mol_type = genomic DNA
gtctagctagatcagttggtagagcataagactcttaatctcagggtcatgggtttgagccctacgttgggcg

SEQ ID NO: 626 - DNA, 73 bp, Homo sapiens
  source 1..73: mol_type = genomic DNA
accagcatgtctcagtcggtatagtgtgagactcttaatctcagggtcgtgggttcaagccccacattgggcg

SEQ ID NO: 627 - DNA, 73 bp, Homo sapiens
  source 1..73: mol_type = genomic DNA
gccaaaatagctcagctgggagagtattaggttgaagatacaaagttccttggctcaatccagagtttggggg

SEQ ID NO: 628 - DNA, 73 bp, Homo sapiens
  source 1..73: mol_type = genomic DNA
gctgagatagctcggttgggagggcatcagcctgaagatctaaaggagactggttcaattctgggttttggca

SEQ ID NO: 629 - DNA, 72 bp, Homo sapiens
  source 1..72: mol_type = genomic DNA
tgcatggttgtctagtggctaggattcggtgctgaaagcgtcacggcccgggttcgattcccggtcagggaa

SEQ ID NO: 630 - DNA, 72 bp, Homo sapiens
  source 1..72: mol_type = genomic DNA
tgcatggttgtctagtggctaggattcggtgctgaaagagccacggcccgggttcgattcccggtcagggaa

SEQ ID NO: 631 - DNA, 73 bp, Homo sapiens
  source 1..73: mol_type = genomic DNA
gccaaaatagctcagctgggagagcattagactgaagatctaaaggtctctggtttgatcctgggtttcagaa

SEQ ID NO: 632 - DNA, 73 bp, Homo sapiens
  source 1..73: mol_type = genomic DNA
gggggtatatctcagggggcagagcatttgactgcagatcaagaggtccccggttgaaatccgggtgctggat

SEQ ID NO: 633 - DNA, 73 bp, Homo sapiens
  source 1..73: mol_type = genomic DNA
ggcagtgtggcatagtggttagaaatgtgcgctctggggctgctgatcccaggctcaaaccctggcgctgtca

SEQ ID NO: 634 - DNA, 73 bp, Homo sapiens
  source 1..73: mol_type = genomic DNA
gtcagtgttgcacaacggttaagtgaagaggctgtaaacccagactggatgggttcaattcccatctctgccg

SEQ ID NO: 635 - DNA, 76 bp, Homo sapiens
  source 1..76: mol_type = genomic DNA
tcaattatagctcagtggtagagcatttaactgtagatcaagaggtccctggatcaactctgggtgccccctttaa

SEQ ID NO: 636 - DNA, 72 bp, Homo sapiens
  source 1..72: mol_type = genomic DNA
tccttgttactatagtggtgagtatctctgcctgtcatgcgtgagagagggggtcgattccccgacggggag

SEQ ID NO: 637 - DNA, 74 bp, Homo sapiens
  source 1..74: mol_type = genomic DNA
gtctctgtggcacaatcggttagcttgttcggctgttaatctagaggttggtggttagagcccactgagggatg

SEQ ID NO: 638 - DNA, 74 bp, Homo sapiens
  source 1..74: mol_type = genomic DNA
gtctctgtggcacaatcggttagagcgttcggctgttaatctaaaggttggtggctagaacccactgagggacg

SEQ ID NO: 639 - DNA, 74 bp, Homo sapiens
  source 1..74: mol_type = genomic DNA
gtctctgtggcacaatcggttagcgcgttcggctgttaatctagaggttggtggttagagcccactgagggatg

SEQ ID NO: 640 - DNA, 75 bp, Homo sapiens
  source 1..75: mol_type = genomic DNA
gttaagatggcatagcccagcaattgcataaaacttaagactttataattagaagttcaacacctcttcttaaca

SEQ ID NO: 641 - DNA, 75 bp, Homo sapiens
  source 1..75: mol_type = genomic DNA
gttaagatggcagagcccagcgattgcataaaacttaacactttataatcagaggttcaactcctcttcttaaca

SEQ ID NO: 642 - DNA, 75 bp, Homo sapiens
  source 1..75: mol_type = genomic DNA
gttaagatggcagagcccagcaattgcataaaacttaaaactttacaatcagaggttcaactcctcttcttaaca

SEQ ID NO: 643 - DNA, 75 bp, Homo sapiens
  source 1..75: mol_type = genomic DNA
gttaagatggcagagcccagcaattgcataaatcttaaaactttataatcagaggttcgactcctcttcttaaca

SEQ ID NO: 644 - DNA, 75 bp, Homo sapiens
  source 1..75: mol_type = genomic DNA
gttaagacggcagagcccggcaattgcgtaaaatttacaactttatgggcagaggttcaattcctcttcttaaca

SEQ ID NO: 645 - DNA, 82 bp, Homo sapiens
  source 1..82: mol_type = genomic DNA
ggtagtgtggttgaatggtctaaggcactgaatttaggctccagtctctttggggacgtgggtttaaatcccactgctgcaa

SEQ ID NO: 646 - DNA, 72 bp, Homo sapiens
  source 1..72: mol_type = genomic DNA
gtgttgatggtatagtggtgagcatagctgccttccaagcaattgacccgacttcaattcccagccaacgca

SEQ ID NO: 647 - DNA, 72 bp, Homo sapiens
  source 1..72: mol_type = genomic DNA
gtgttgatggtatagtggtgagcatagctgccttccaagcaattgacccgacttcgattcccagccaatgca

SEQ ID NO: 648 - DNA, 69 bp, Homo sapiens
  source 1..69: mol_type = genomic DNA
gaaaaagtcataggggttatgaggctggcttgaaaccagccttaggaggttcaattccttccttttttg

SEQ ID NO: 649 - DNA, 70 bp, Homo sapiens
  source 1..70: mol_type = genomic DNA
gagaaggtcatagaggttatgggattggcttgaaaccagtctctggggggttcgattccctcctttttca

SEQ ID NO: 650 - DNA, 70 bp, Homo sapiens
  source 1..70: mol_type = genomic DNA
gggggagtggtgtggttacgaatgtggcctctgcaagcagacagcctgggttcaattcccagcttggcca

SEQ ID NO: 651 - DNA, 72 bp, Homo sapiens
  source 1..72: mol_type = genomic DNA
gtggatgtagtttagtggtagaacgcgcgctttgcatgtatgaggtcccggtttcgatccctggcgtttcca

SEQ ID NO: 652 - DNA, 77 bp, Homo sapiens
  source 1..77: mol_type = genomic DNA
gccaggagagctcagtggtgatgggatgagatctggactcacacctctaggcctgggttcaaatcccaggtctagcg

SEQ ID NO: 653 - DNA, 72 bp, Homo sapiens
  source 1..72: mol_type = genomic DNA
taggacttggtgtaataggtagcacgaagagatttggattctcaggggtaggttcaattcctatagttctgg

SEQ ID NO: 654 - DNA, 71 bp, Homo sapiens
  source 1..71: mol_type = genomic DNA
ggcctgttggtctagaggtatgattctcgctttgggtgcgagaggccccggtgcgagtcccagaggagccc

SEQ ID NO: 655 - DNA, 72 bp, Homo sapiens
  source 1..72: mol_type = genomic DNA
ggcagcctggcttagtggaaagggaataggctttagagccagactgcctgggtttgaatcccagccccgcca

SEQ ID NO: 656 - DNA, 69 bp, Homo sapiens
  source 1..69: mol_type = genomic DNA
tccctggtctagtggttaggatttattattttcatggctgtggcctgagttcaatttccaatcagggaa

SEQ ID NO: 657 - DNA, 72 bp, Homo sapiens
  source 1..72: mol_type = genomic DNA
tccctggtggtctggtggctagaatttagcgctttcaccgccgcagctcgggttggattaccagtcagggaa

SEQ ID NO: 658 - DNA, 73 bp, Homo sapiens
  source 1..73: mol_type = genomic DNA
tccgtggtggtctagtggctaggattcggcgctttcaccgcctgcagctcgagttcgattcctggtcagggaa

SEQ ID NO: 659 - DNA, 71 bp, Homo sapiens
  source 1..71: mol_type = genomic DNA
gcaatggtggttcagtggtagaattctcgcctttcacacaggagacccgggttcaattcctgacccatgta

SEQ ID NO: 660 - DNA, 72 bp, Homo sapiens
  source 1..72: mol_type = genomic DNA
tgtctggtggtcaagtggctaggatttggcgctttcactgccgcggcccgcgttcgattcccggtcagggaa

SEQ ID NO: 661 - DNA, 72 bp, Homo sapiens
  source 1..72: mol_type = genomic DNA
tccttggtggtctagtggctaggattcggtgctttcacctgtgcggcccgggttcaattcccgatgaaggaa

SEQ ID NO: 662 - DNA, 72 bp, Homo sapiens
  source 1..72: mol_type = genomic DNA
taggacgtggtgtgataggtagcacagagaattttggattctcaggggtaggttaaattcctatagtactag

SEQ ID NO: 663 - DNA, 72 bp, Homo sapiens
  source 1..72: mol_type = genomic DNA
taggacgtggtgtgataggtagcatggggaattttggattctcaggggtgggttcaattcctatagttctag

SEQ ID NO: 664 - DNA, 74 bp, Homo sapiens
  source 1..74: mol_type = genomic DNA
ggcaatgtagcattgtggctaagtgcacaggctttggaaactggcaggcctgggttcaaatcccagcttattca

SEQ ID NO: 665 - DNA, 72 bp, Homo sapiens
  source 1..72: mol_type = genomic DNA
taggatgtggtgtaataggtggcatggagaattttggattatcaggggtaggttcaattcctatagttctag

SEQ ID NO: 666 - DNA, 72 bp, Homo sapiens
  source 1..72: mol_type = genomic DNA
tagtacatagtgtaataggtagcacagataattttggattctcaggggtaggttcaattcttatagttctag

SEQ ID NO: 667 - DNA, 73 bp, Homo sapiens
  source 1..73: mol_type = genomic DNA
agcagtgtagcctagtggctaggtcctctgactttgaaaccacgtggtctgggtttaagtctcagctgtgcta

SEQ ID NO: 668 - DNA, 72 bp, Homo sapiens
  source 1..72: mol_type = genomic DNA
taggatgtggtgtgacaggtagcatggagaattttggattctcagggttaggttcaattcctatagttctag

SEQ ID NO: 669 - DNA, 73 bp, Homo sapiens
  source 1..73: mol_type = genomic DNA
agctgtatattatagtggaataaatgtggactttgaagttagatacacctgggttcaaatcccagtgctgtca

SEQ ID NO: 670 - DNA, 72 bp, Homo sapiens
  source 1..72: mol_type = genomic DNA
taggacgtggtgtgataggtagcacggagaattttggattctcagggatgggttcaattcctgtagttctag

SEQ ID NO: 671 - DNA, 75 bp, Homo sapiens
  source 1..75: mol_type = genomic DNA
tttaggacgtggtgtaataggtagcacagagaattttggattctcaggtgcaggttcaattcctatattctagag

SEQ ID NO: 672 - DNA, 72 bp, Homo sapiens
  source 1..72: mol_type = genomic DNA
taggacgtggtgtgataggtagcatggagaattttggattctcagggatgggttcaattcctatagtcctag

SEQ ID NO: 673 - DNA, 75 bp, Homo sapiens
  source 1..75: mol_type = genomic DNA
tctaggatgtggtgtgataggtagcatggagaattttggattctcaggggtaggttcaattcctatattctagaa

SEQ ID NO: 674 - DNA, 72 bp, Homo sapiens
  source 1..72: mol_type = genomic DNA
taggatgtggtgtattaggtagcacagagaattttggattctcaggggtaggttcgattcctataattctac

SEQ ID NO: 675 - DNA, 72 bp, Homo sapiens
  source 1..72: mol_type = genomic DNA
taggacgtggtgtagtaggtagcatggagaatgttgaattctcaggggtaggttcaattcctatagttctag

SEQ ID NO: 676 - DNA, 72 bp, Homo sapiens
  source 1..72: mol_type = genomic DNA
taggacatggtgtgataggtagcatggagaattttggattctcaggggtaggttcaattcctacagttctag

SEQ ID NO: 677 - DNA, 73 bp, Homo sapiens
  source 1..73: mol_type = genomic DNA
ggctgtgtacctcagtgggcaagggtatggactttgaagccagactatttgggttcaaatcccagcttggcct

SEQ ID NO: 678 - DNA, 70 bp, Homo sapiens
  source 1..70: mol_type = genomic DNA
tcctatagcccagtgattaggattctttgcttttactaccatgacctgggttcaatacccagtcagggaa

SEQ ID NO: 679 - DNA, 73 bp, Homo sapiens
  source 1..73: mol_type = genomic DNA
gttggggtaactcagttggtagagtagcagacttttcatctgagggtccagggtttaagtccatgtccaggca

SEQ ID NO: 680 - DNA, 73 bp, Homo sapiens
  source 1..73: mol_type = genomic DNA
acccagatagctcagttgatagagcatcagacttttaatctgagggtccagggttcatgtccctgttccttaa

SEQ ID NO: 681 - DNA, 74 bp, Homo sapiens
  source 1..74: mol_type = genomic DNA
gcctgggtagctcagtcggtagagctatcagacttttagcctgaggattcagggttcaatcccttgctggggcg

SEQ ID NO: 682 - DNA, 73 bp, Homo sapiens
  source 1..73: mol_type = genomic DNA
gccaggatagttcaggtggtagagcatcagacttttaacctgagggttcagggttcaagtctctgtttgggcg

SEQ ID NO: 683 - DNA, 74 bp, Homo sapiens
  source 1..74: mol_type = genomic DNA
gaccgtgtggccttaatggataaggtgtctgacttcagatcagaagattgagggtttgagtccctttgtggtca

SEQ ID NO: 684 - DNA, 86 bp, Homo sapiens
  source 1..86: mol_type = genomic DNA
gctcggatgatcctcagtggtctggggtgcaggcttcaaacctgtagctgtctagtgacagagtggttcaattccacctttgtagg

SEQ ID NO: 685 - DNA, 86 bp, Homo sapiens
  source 1..86: mol_type = genomic DNA
gcccggatgatcctcagtggtctggggtgcaggcttcaaacctgtagctgtctagcgacagagtggttcaattccacctttcgggc

SEQ ID NO: 686 - DNA, 82 bp, Homo sapiens
  source 1..82: mol_type = genomic DNA
tgagttgtagctgagtggttaaggcaacgagctagaaattcgttggtttctctctgtgcaggtttgaatcctgctaattatg

SEQ ID NO: 687 - DNA, 72 bp, Homo sapiens
  source 1..72: mol_type = genomic DNA
gggtgtatggctcaggggtagagaatttgactagagatcaagaggtccctggttcaaatccaggtgccccct

SEQ ID NO: 688 - DNA, 82 bp, Homo sapiens
  source 1..82: mol_type = genomic DNA
gtagtcgtggccgagtggttaaggtgatggactagaaacccattggggtctccccgcgcaggttcgaatcctgccgactacg

SEQ ID NO: 689 - DNA, 82 bp, Homo sapiens
  source 1..82: mol_type = genomic DNA
gtagtcgtggccgagtggttaaggcgatggactagaaatccattggggtttccccacgcaggttcgaatcctgccgactacg

SEQ ID NO: 690 - DNA, 82 bp, Homo sapiens
  source 1..82: mol_type = genomic DNA
gtagtcgtggccgagtggttaaggcgatggactagaaatccattggggtctccccgcgcaggttcgaatcctgccgactacg

SEQ ID NO: 691 - DNA, 82 bp, Homo sapiens; identical to SEQ ID NO: 690
  source 1..82: mol_type = genomic DNA

SEQ ID NO: 692 - DNA, 82 bp, Homo sapiens; identical to SEQ ID NO: 690
  source 1..82: mol_type = genomic DNA

SEQ ID NO: 693 - DNA, 82 bp, Homo sapiens; identical to SEQ ID NO: 690
  source 1..82: mol_type = genomic DNA

SEQ ID NO: 694 - DNA, 82 bp, Homo sapiens; identical to SEQ ID NO: 690
  source 1..82: mol_type = genomic DNA

SEQ ID NO: 695 - DNA, 82 bp, Homo sapiens; identical to SEQ ID NO: 690
  source 1..82: mol_type = genomic DNA

SEQ ID NO: 696 - DNA, 82 bp, Homo sapiens
  source 1..82: mol_type = genomic DNA
gtagtcgtggccgagtggttaaggcgatggactagaaatccattggggtttccccgcgcaggttcgaatcctgccgactacg

SEQ ID NO: 697 - DNA, 82 bp, Homo sapiens
  source 1..82: mol_type = genomic DNA
gtcacggtggccgagtggttaaggcgttggactcgaaatccaatggggtttccccgcacaggttcgaatcctgttcgtgacg

SEQ ID NO: 698 - DNA, 82 bp, Homo sapiens
  source 1..82: mol_type = genomic DNA
gctgtgatggccgagtggttaaggtgttggactcgaaatccaatgggggttccccgcgcaggttcaaatcctgctcacagcg

SEQ ID NO: 699 - DNA, 82 bp, Homo sapiens
  source 1..82: mol_type = genomic DNA
gctgtgatggccgagtggttaaggcgttggactcgaaatccaatggggtctccccgcgcaggttcaaatcctgctcacagcg

SEQ ID NO: 700 - DNA, 82 bp, Homo sapiens
  source 1..82: mol_type = genomic DNA
gctgtgatggccgagtggttaaggcgttggactcgaaatccaatggggtctccccgcgcaggttcgaatcctgctcacagcg

SEQ ID NO: 701 - DNA, 84 bp, Homo sapiens
  source 1..84: mol_type = genomic DNA
ggagaggcctggccgagtggttaaggcgatggactgctaatccattgtgctctgcacgcgtgggttcgaatcccatcctcgtcg

SEQ ID NO: 702 - DNA, 82 bp, Homo sapiens
  source 1..82: mol_type = genomic DNA
gacgaggtggccgagtggttaaggcgatggactgctaatccattgtgctctgcacacgtgggttcgaatcccatcctcgtcg

SEQ ID NO: 703 - DNA, 82 bp, Homo sapiens
  source 1..82: mol_type = genomic DNA
gacgaggtggccgagtggttaaggcgatggactgctaatccattgtgctctgcacgcgtgggttcgaatcccatcctcgtcg

SEQ ID NO: 704 - DNA, 82 bp, Homo sapiens; identical to SEQ ID NO: 703
  source 1..82: mol_type = genomic DNA

SEQ ID NO: 705 - DNA, 82 bp, Homo sapiens; identical to SEQ ID NO: 703
  source 1..82: mol_type = genomic DNA

SEQ ID NO: 706 - DNA, 82 bp, Homo sapiens
  source 1..82: mol_type = genomic DNA
gacgaggtggccgagtggttaaggcgatggactgctaatccattgtgctttgcacgcgtgggttcgaatcccatcctcgtcg

SEQ ID NO: 707 - DNA, 82 bp, Homo sapiens
  source 1..82: mol_type = genomic DNA
gacgaggtggccgagtggttaaggcgatggactgctaatccattgtgctctgcacgcgtgggttcgaatcccaccttcgtcg

SEQ ID NO: 708 - DNA, 82 bp, Homo sapiens
  source 1..82: mol_type = genomic DNA
gacgaggtggccgagtggttaaggcgatggactgctaatccattgtgctctgcacgcgtgggttcgaatcccaccctcgtcg

SEQ ID NO: 709 - DNA, 70 bp, Homo sapiens
  source 1..70: mol_type = genomic DNA
gagaaggtcacagaggttatgggattggcttgaaaccagtctgtggggggttcgattccctcctttttca

SEQ ID NO: 710 - DNA, 82 bp, Homo sapiens
  source 1..82: mol_type = genomic DNA
gtagtcgtggccgagtggttaaggcgatggacttgaaatccattggggtttccccgcgcaggttcgaatcctgtcggctacg

SEQ ID NO: 711 - DNA, 82 bp, Homo sapiens
  source 1..82: mol_type = genomic DNA
gtagtcgtggccgagtggttaaggcgatggacttgaaatccattggggtctccccgcgcaggttcgaatcctgccgactacg

SEQ ID NO: 712 - DNA, 82 bp, Homo sapiens
  source 1..82: mol_type = genomic DNA
gtagtcgtggccgagtggttaaggcgatggacttgaaatccattggggtttccccgcgcaggttcgaatcctgccgactacg

SEQ ID NO: 713 - DNA, 82 bp, Homo sapiens
  source 1..82: mol_type = genomic DNA
gcagcgatggccgagtggttaaggcgttggacttgaaatccaatggggtctccccgcgcaggttcgaaccctgctcgctgcg

SEQ ID NO: 714 - DNA, 74 bp, Homo sapiens
  source 1..74: mol_type = genomic DNA
ggcagtgacgcctactggtttagagcacagattctagatcgagacattcctgggttcaaatcccagcactgttg

SEQ ID NO: 715 - DNA, 71 bp, Homo sapiens
  source 1..71: mol_type = genomic DNA
ggcagtgacatgtaatggttatgagggtggactttaaccacactgcctaggttcaaatcctgactctgtca

SEQ ID NO: 716 - DNA, 71 bp, Homo sapiens
  source 1..71: mol_type = genomic DNA
gcccggatagttcagttggtagagcatcagacttaatcagagggtccagggttcaagtccctgtttgggtg

SEQ ID NO: 717 - DNA, 74 bp, Homo sapiens
  source 1..74: mol_type = genomic DNA
agcaccatggcttagctggttaaagcacctgtctagtaaacaggagatcctgagtttcaattccaatggtgcct

SEQ ID NO: 718 - DNA, 74 bp, Homo sapiens
  source 1..74: mol_type = genomic DNA
ggccctgtggcttagctggtcaaagcgcctgtctagtaaacaggagatcctgggttcgaatcccagcggggcct

SEQ ID NO: 719 - DNA, 74 bp, Homo sapiens
  source 1..74: mol_type = genomic DNA
ggcttcgtggcttagctggttaaagcgcctgtctagtaaacaggagatcctgggttcgaatcccagcgaggcct

SEQ ID NO: 720 - DNA, 74 bp, Homo sapiens
  source 1..74: mol_type = genomic DNA
ggcgccgtggcttagctggttaaagcgcctgtctagtaaacaggagatcctgggttcgaatcccagcggtgcct

SEQ ID NO: 721 - DNA, 74 bp, Homo sapiens
  source 1..74: mol_type = genomic DNA
ggctccgtagcttagttggttaaagcgcctgtctagtaaacaggagatcctgggttcgactcccagcggggcct

SEQ ID NO: 722 - DNA, 74 bp, Homo sapiens
  source 1..74: mol_type = genomic DNA
ggctccgtggcttagctggttaaagcgcctgtctagtaaacaggagatcctgggttcgaatcccagcggggcct

SEQ ID NO: 723 - DNA, 74 bp, Homo sapiens; identical to SEQ ID NO: 722
  source 1..74: mol_type = genomic DNA

SEQ ID NO: 724 - DNA, 74 bp, Homo sapiens
  source 1..74: mol_type = genomic DNA
ggcgccgtggcttagttggttaaagcgcctgtctagtaaacaggagatcctgggttcgaatcccagcggtgcct

SEQ ID NO: 725 - DNA, 74 bp, Homo sapiens; identical to SEQ ID NO: 724
  source 1..74: mol_type = genomic DNA

SEQ ID NO: 726 - DNA, 74 bp, Homo sapiens; identical to SEQ ID NO: 724
  source 1..74: mol_type = genomic DNA

SEQ ID NO: 727 - DNA, 72 bp, Homo sapiens
  source 1..72: mol_type = genomic DNA
ggcagagtggtgcagcggaagcgtgctgggcccgtaacccagaggtcaatggatcgaagccatccttggcta

SEQ ID NO: 728 - DNA, 74 bp, Homo sapiens
  source 1..74: mol_type = genomic DNA
ggccctgtagctcagcggttggagcgctggtctcgtaaacctaggggtcgtgagttcaaatctcaccagggcct

SEQ ID NO: 729 - DNA, 72 bp, Homo sapiens
  source 1..72: mol_type = genomic DNA
ggcgcggtggccaagtggtaaggcgtcggtctcgtaaaccgaagatcgcgggttcgaaccccgtccgtgcct

SEQ ID NO: 730 - DNA, 74 bp, Homo sapiens
  source 1..74: mol_type = genomic DNA
ggctctgtggcttagttggctaaagcgcctgtctcgtaaacaggagatcctgggttcgaatcccagcggggcct

SEQ ID NO: 731 - DNA, 72 bp, Homo sapiens
  source 1..72: mol_type = genomic DNA
ggcgcggtggccaagtggtaaggcgtcggtctcgtaaaccgaagatcacgggttcgaaccccgtccgtgcct

SEQ ID NO: 732 - DNA, 74 bp, Homo sapiens
  source 1..74: mol_type = genomic DNA
ggctctatggcttagttggttaaagcgcctgtctcgtaaacaggagatcctgggttcgactcccagtggggcct

SEQ ID NO: 733 - DNA, 72 bp, Homo sapiens
  source 1..72: mol_type = genomic DNA
ggctccatagctcaggggttagagcactggtcttgtaaaccagggtcgcgagttcaaatctcgctggggcct

SEQ ID NO: 734 - DNA, 73 bp, Homo sapiens
  source 1..73: mol_type = genomic DNA
ggccctatagctcaggggttagagcactggtcttgtaaaccaggggtcgcgagttcaaatctcgctggggcct

SEQ ID NO: 735 - DNA, 73 bp, Homo sapiens
  source 1..73: mol_type = genomic DNA
ggctccatagctcaggggttagagcactggtcttgtaaaccaggggtcgcgagttcaaatctcgctggggcct

SEQ ID NO: 736 - DNA, 73 bp, Homo sapiens
  source 1..73: mol_type = genomic DNA
ggctccatagctcaggggttagagcgctggtcttgtaaaccaggggtcgcgagttcaattctcgctggggcct

SEQ ID NO: 737 - DNA, 73 bp, Homo sapiens
  source 1..73: mol_type = genomic DNA
ggctccatagctcagtggttagagcactggtcttgtaaaccaggggtcgcgagttcgatcctcgctggggcct

SEQ ID NO: 738 - DNA, 74 bp, Homo sapiens
  source 1..74: mol_type = genomic DNA
ggctctatggcttagttggttaaagcgcctgtcttgtaaacaggagatcctgggttcgaatcccagtagagcct

SEQ ID NO: 739 - DNA, 74 bp, Homo sapiens
  source 1..74: mol_type = genomic DNA
ggaaggatggggccaagctggaaagcctgtgggctccacagtcatgtgcctgggttcaattcccagttctgcat

SEQ ID NO: 740 - DNA, 72 bp, Homo sapiens
  source 1..72: mol_type = genomic DNA
gacctcgtggcgcaacggcagcgcgtctgactccagatcagaaggttgcgtgttcaaatcacgtcggggtca

SEQ ID NO: 741 - DNA, 72 bp, Homo sapiens
  source 1..72: mol_type = genomic DNA
gacctcgtggcgcaacggtagcgcgtctgactccagatcagaaggctgcgtgttcgaatcacgtcggggtca

SEQ ID NO: 742 - DNA, 72 bp, Homo sapiens
  source 1..72: mol_type = genomic DNA
gacctcgtggcgcaacggtagcgcgtctgactccagatcagaaggttgcgtgttcaaatcacgtcggggtca

SEQ ID NO: 743 - DNA, 72 bp, Homo sapiens; identical to SEQ ID NO: 742
  source 1..72: mol_type = genomic DNA

SEQ ID NO: 744 - DNA, 72 bp, Homo sapiens; identical to SEQ ID NO: 742
  source 1..72: mol_type = genomic DNA

SEQ ID NO: 745 - DNA, 72 bp, Homo sapiens
  source 1..72: mol_type = genomic DNA
gacctcgtggcgcaatggtagcgcgtctgactccagatcagaaggttgcgtgttcaagtcacgtcggggtca

SEQ ID NO: 746 - DNA, 72 bp, Homo sapiens
  source 1..72: mol_type = genomic DNA
ggcctcgtggcgcaacggtagcgcgtctgactccagatcagaaggttgcgtgttcaaatcacgtcggggtca

SEQ ID NO: 747 - DNA, 99 bp, Homo sapiens
  source 1..99: mol_type = genomic DNA
ggcagaggagggtgcagttggcagcctgtccaagtccagcacggttggagcacaggatttagaatgggatggtcctgggttcaaaccccagctgcgccc

SEQ ID NO: 748 - DNA, 93 bp, Homo sapiens
  source 1..93: mol_type = genomic DNA
ccttcaatagttcagctggtagagcagaggactatagctacttcctcagtaggagacgtccttaggttgctggttcgattccagcttgaagga

SEQ ID NO: 749 - DNA, 73 bp, Homo sapiens
  source 1..73: mol_type = genomic DNA
gggggtatagctcagggctagagctttttgactgtagagcaagaggtccctggttcaaatccaggttctccct

SEQ ID NO: 750 - DNA, 94 bp, Homo sapiens
  source 1..94: mol_type = genomic DNA
ccttcgatagctcagctggtagagcggaggactgtagcctgtagaaacatttgtggacatccttaggtcgctggttcgattccggctcgaagga

SEQ ID NO: 751 - DNA, 94 bp, Homo sapiens
  source 1..94: mol_type = genomic DNA
ccttcgatagctcagctggtagagcggaggactgtagattgtatagacatttgcggacatccttaggtcgctggttcgattccagctcgaagga

SEQ ID NO: 752 - DNA, 94 bp, Homo sapiens
  source 1..94: mol_type = genomic DNA
ccttcgatagctcagctggtagagcggaggactgtagactgcggaaacgtttgtggacatccttaggtcgctggttcaattccggctcgaagga

SEQ ID NO: 753 - DNA, 94 bp, Homo sapiens
  source 1..94: mol_type = genomic DNA
ccttcgatagctcagctggtagagcggaggactgtagattgtacagacatttgcggacatccttaggtcgctggttcgattccggctcgaagga

SEQ ID NO: 754 - DNA, 89 bp, Homo sapiens
  source 1..89: mol_type = genomic DNA
ccttcgatagctcagctggtagagcggaggactgtagtacttaatgtgtggtcatccttaggtcgctggttcgattccggctcgaagga

SEQ ID NO: 755 - DNA, 91 bp, Homo sapiens
  source 1..91: mol_type = genomic DNA
ccttcgatagctcagttggtagagcggaggactgtagttggctgtgtccttagacatccttaggtcgctggttcgaatccggctcgaagga

SEQ ID NO: 756 - DNA, 90 bp, Homo sapiens
  source 1..90: mol_type = genomic DNA
ctttcgatagctcagttggtagagcggaggactgtaggttcattaaactaaggcatccttaggtcgctggttcgaatccggctcgaagga

SEQ ID NO: 757 - DNA, 89 bp, Homo sapiens
  source 1..89: mol_type = genomic DNA
ccttcgatagctcagttggtagagcggaggactgtaggctcattaagcaaggtatccttaggtcgctggttcgaatccggctcggagga

SEQ ID NO: 758 - DNA, 89 bp, Homo sapiens
  source 1..89: mol_type = genomic DNA
ccttcgatagctcagctggtagagcggaggactgtaggggtttgaatgtggtcatccttaggtcgctggttcgaatccggctcggagga

SEQ ID NO: 759 - DNA, 89 bp, Homo sapiens
  source 1..89: mol_type = genomic DNA
ccttcgatagctcagttggtagagcggaggactgtagtggatagggcgtggcaatccttaggtcgctggttcgattccggctcgaagga

SEQ ID NO: 760 - DNA, 88 bp, Homo sapiens
  source 1..88: mol_type = genomic DNA
tcttcaatagctcagctggtagagcggaggactgtaggtgcacgcccgtggccattcttaggtgctggtttgattccgacttggagag

SEQ ID NO: 761 - DNA, 93 bp, Homo sapiens
  source 1..93: mol_type = genomic DNA
ccttcgatagctcagctggtagagcggaggactgtagctacttcctcagcaggagacatccttaggtcgctggttcgattccggctcgaagga

SEQ ID NO: 762 - DNA, 89 bp, Homo sapiens
  source 1..89: mol_type = genomic DNA
ccttcgatagctcagctggtagagcggaggactgtaggcgcgcgcccgtggccatccttaggtcgctggttcgattccggctcgaagga

SEQ ID NO: 763 - DNA, 73 bp, Homo sapiens
  source 1..73: mol_type = genomic DNA
gtttccatagtgtactggttatcacattcacctaacacgcgaaaggtccttggtttgaaaccaggcagaaaca

SEQ ID NO: 764 - DNA, 72 bp, Homo sapiens
  source 1..72: mol_type = genomic DNA
gggggtgtagctcagtggtagagcgtatgcttaacattcatgaggctctgggttcgatccccagcacttcca

SEQ ID NO: 765 - DNA, 73 bp, Homo sapiens
  source 1..73: mol_type = genomic DNA
gtttccgtagtgtagtggttatcacgtttgcctaacacgcgaaaggtccccggttcgaaaccgggcagaaaca

SEQ ID NO: 766 - DNA, 73 bp, Homo sapiens
  source 1..73: mol_type = genomic DNA
gtttccgtagtgtagtggttatcacgttcgcctaacacgcgaaaggtccgcggttcgaaaccgggcggaaaca

SEQ ID NO: 767 - DNA, 73 bp, Homo sapiens
  source 1..73: mol_type = genomic DNA
gtttccgtagtgtagtggttatcacgttcgcctaacacgcgaaaggtccctggatcaaaaccaggcggaaaca

SEQ ID NO: 768 - DNA, 73 bp, Homo sapiens
  source 1..73: mol_type = genomic DNA
gtttccgtagtgtagtggtcatcacgttcgcctaacacgcgaaaggtccccggttcgaaaccgggcggaaaca

SEQ ID NO: 769 - DNA, 73 bp, Homo sapiens
  source 1..73: mol_type = genomic DNA
gtttccgtagtgtagtggttatcacgttcgcctaacacgcgaaaggtccccggttcgaaaccgggcggaaaca

SEQ ID NO: 770 - DNA, 73 bp, Homo sapiens; identical to SEQ ID NO: 769
  source 1..73: mol_type = genomic DNA

SEQ ID NO: 771 - DNA, 73 bp, Homo sapiens; identical to SEQ ID NO: 769
  source 1..73: mol_type = genomic DNA

SEQ ID NO: 772 - DNA, 73 bp, Homo sapiens; identical to SEQ ID NO: 769
  source 1..73: mol_type = genomic DNA

SEQ ID NO: 773 - DNA, 73 bp, Homo sapiens; identical to SEQ ID NO: 769
  source 1..73: mol_type = genomic DNA

SEQ ID NO: 774 - DNA, 74 bp, Homo sapiens
  source 1..74: mol_type = genomic DNA
ttttctgtagtgtagttgttaacacgttcgcctcacacgcttaaagttctctggttggataccagatggaaatg

SEQ ID NO: 775 - DNA, 73 bp, Homo sapiens
  source 1..73: mol_type = genomic DNA
gtttctgtggtgtagtggttatcatgttcgcctcacacgagaaaagtccctgattcgagactgggtgggaacg

SEQ ID NO: 776 - DNA, 73 bp, Homo sapiens
  source 1..73: mol_type = genomic DNA
gtttctgtagtatggtggttatcacgttagtctcacacgtgaaaggtccctggttcgaaaccaggtggaaaca

SEQ ID NO: 777 - DNA, 71 bp, Homo sapiens
  source 1..71: mol_type = genomic DNA
gcactggtggttcagtggtagaattctcgcctcacacgcgggacacccgggttcaattcccggtcaaggca

SEQ ID NO: 778 - DNA, 73 bp, Homo sapiens
  source 1..73: mol_type = genomic DNA
gtttccgtagtgtagtggttattatgttcgcctcacacgcgaaaagtccccggttcgaaatcaggcgggaaca

SEQ ID NO: 779 - DNA, 73 bp, Homo sapiens
  source 1..73: mol_type = genomic DNA
gtttccgtagtggagtggttatcacgttcgcctcacacgcgaaaggtccccggtttgaaaccaggcggaaaca

SEQ ID NO: 780 - DNA, 74 bp, Homo sapiens
  source 1..74: mol_type = genomic DNA
gtttccgtagtgtagtggttatcacgttcgcctcacacgcgtaaaggtccccggttcgaaaccgggcggaaaca

SEQ ID NO: 781 - DNA, 73 bp, Homo sapiens
  source 1..73: mol_type = genomic DNA
gtttccgtagtgtagtggttatcacgttcgcctcacacgcgaaaggtccccggttcgaaactgggcggaaaca

SEQ ID NO: 782 - DNA, 73 bp, Homo sapiens
  source 1..73: mol_type = genomic DNA
gtttccgtagtgtagcggttatcacattcgcctcacacgcgaaaggtccccggttcgatcccgggcggaaaca

SEQ ID NO: 783 - DNA, 73 bp, Homo sapiens
  source 1..73: mol_type = genomic DNA
gcttctgtagtgtagtggttatcacgttcgcctcacacgcgaaaggtccccggttcgaaaccgggcagaagca

SEQ ID NO: 784 - DNA, 73 bp, Homo sapiens
  source 1..73: mol_type = genomic DNA
gtttccgtagtgtagtggttatcacgttcgcctcacacgcgaaaggtccccggttcgaaaccgggcggaaaca

SEQ ID NO: 785 - DNA, 73 bp, Homo sapiens; identical to SEQ ID NO: 784
  source 1..73: mol_type = genomic DNA

SEQ ID NO: 786 - DNA, 73 bp, Homo sapiens; identical to SEQ ID NO: 784
  source 1..73: mol_type = genomic DNA

SEQ ID NO: 787 - DNA, 73 bp, Homo sapiens; identical to SEQ ID NO: 784
  source 1..73: mol_type = genomic DNA

SEQ ID NO: 788 - DNA, 73 bp, Homo sapiens; identical to SEQ ID NO: 784
  source 1..73: mol_type = genomic DNA

SEQ ID NO: 789 - DNA, 73 bp, Homo sapiens; identical to SEQ ID NO: 784
  source 1..73: mol_type = genomic DNA

SEQ ID NO: 790 - DNA, 73 bp, Homo sapiens
  source 1..73: mol_type = genomic DNA
gtttccgtggtgtagtggttatcacattcgccttacacgcgaaaggtcctcgggtcgaaaccgagcggaaaca

SEQ ID NO: 791 - DNA, 73 bp, Homo sapiens
  source 1..73: mol_type = genomic DNA
ggttccatagtgtagtggttatcacatctgctttacacgcagaaggtcctgggttcaagccccagtggaacca

SEQ ID NO: 792 - DNA, 73 bp, Homo sapiens
  source 1..73: mol_type = genomic DNA
ggttccatagtgtagcggttatcacgtctgctttacacgcagaaggtcctgggttcgagccccagtggaacca

SEQ ID NO: 793 - DNA, 73 bp, Homo sapiens
  source 1..73: mol_type = genomic DNA
ggttccatagtgtagtggttatcacgtctgctttacacgcagaaggtcctgggttcgagccccagtggaacca

SEQ ID NO: 794 - DNA, 73 bp, Homo sapiens; identical to SEQ ID NO: 793
  source 1..73: mol_type = genomic DNA

SEQ ID NO: 795 - DNA, 72 bp, Homo sapiens
  source 1..72: mol_type = genomic DNA
taggatatggtttaataggtagcatggagaattttggagtctcagggataggttcaattcctatagttccag

SEQ ID NO: 796 - DNA, 72 bp, Homo sapiens
  source 1..72: mol_type = genomic DNA
ggtcccatggtgtaatggttagcactctggactttgaatccagcaatccgagttcgaatctcggtgggacct

(19) United States
(12) Patent Application Publication
(10) Pub. No.: US 2024/0240181 A1
(43) Pub. Date: Jul. 18, 2024

(54) NEUROPROTECTIVE MOLECULES AND METHODS OF TREATING NEUROLOGICAL DISORDERS AND INDUCING STRESS GRANULES

(71) Applicant: The Brigham and Women's Hospital, Inc., Boston, MA (US)

(72) Inventors: Paul Anderson, Belmont, MA (US); Pavel Ivanov, Brighton, MA (US); Mohammed Emara, Newton, MA (US)

(21) Appl. No.: 18/507,432

(22) Filed: Nov. 13, 2023

Related U.S. Application Data
(63) Continuation of application No. 16/577,857, filed on Sep. 20, 2019.
Application No. 16/577,857 is a continuation of application No. 15/590,344, filed on May 9, 2017, now Pat. No. 10,457,939.
Application No. 15/590,344 is a continuation of application No. 13/808,863, filed on Mar. 29, 2013, now Pat. No. 9,676,810.
(60) Provisional application No. 61/362,526, filed on Jul. 8, 2010.

(51) Int. Cl. C12N 15/113; C07H 21/02; C12N 15/11

(52) U.S. Cl. CPC C12N 15/113; C07H 21/02; C12N 15/11; C12N 2310/18; C12N 2310/531

(57) ABSTRACT

Provided herein are neuroprotective molecules containing a sequence of 25-35 contiguous nucleotides between nucleotide 1 and nucleotide 50 of a mature human tRNA and at least four contiguous guanosine-containing nucleotides, where the sequence of 25-35 contiguous nucleotides contains a D-loop stem structure, the at least four contiguous guanosine-containing nucleotides are located at the 5′ end of the neuroprotective molecule, and the neuroprotective molecule contains at least one deoxyribonucleotide. Also provided are neuroprotective molecules containing a sequence of 25-35 contiguous nucleotides between nucleotide 1 and nucleotide 50 of a mature human tRNA^{CYS}; and at least four contiguous guanosine-containing nucleotides, where the sequence of 25-35 contiguous nucleotides contains a D-loop stem structure and the at least four contiguous guanosine-containing nucleotides are located at the 5′ end of the neuroprotective molecule.

### CLAIM OF PRIORITY

This application is a continuation of U.S. patent application Ser. No. 16/577,857, filed Sep. 20, 2019, which is a continuation of U.S. patent application Ser. No. 15/590,344, filed May 9, 2017, now U.S. Pat. No. 10,457,939, which is a continuation of U.S. patent application Ser. No. 13/808,863, with a 371 filing date of Mar. 29, 2013, now U.S. Pat. No. 9,676,810, which is a 35 U.S.C. § 371 national phase application of International Application No. PCT/US2011/043400, filed Jul. 8, 2011, which claims the benefit of U.S. Provisional Application Ser. No. 61/362,526, filed Jul. 8, 2010, the entire contents of each of which are incorporated by reference herein.

### STATEMENT AS TO FEDERALLY-SPONSORED RESEARCH

This invention was made with government support under grant No. AI0658568 awarded by the National Institutes of Health. The government has certain rights in the invention.

### SEQUENCE LISTING

This application contains a Sequence Listing that has been submitted electronically as an XML file named “29618-0020004_SL_ST26.XML.” The XML file, created on Nov. 9, 2023, is 754,445 bytes in size. The material in the XML file is hereby incorporated by reference in its entirety.

### TECHNICAL FIELD

This invention relates to neuroprotective molecules and methods of treating neurological diseases associated with neuron death, inducing stress granule formation in a cell, inhibiting translation in a cell, and decreasing stress-induced cell death.

### BACKGROUND

Amyotrophic lateral sclerosis (ALS) is a progressive neurodegenerative disease distinguished by a specific loss of motor neurons in the brain, brain stem, and spinal cord. Initial symptoms of loss of motor neuron activity, including distal muscle weakness and wasting, increased muscle tone with hyperreflexia, and diaphragmatic and/or bulbar weakness are first noticed at an average age of 55. Death occurs from respiratory failure at an average of 4 years after disease onset. The only recognized treatment for ALS is riluzole which extends survival by only about 3 months with no improvement in motor muscular function.

Although ALS is a rare disease (˜1-2 persons per 100,000 population), an understanding of its pathogenesis is likely to impact other neurological diseases that share some pathological features (e.g., frontotemporal lobal degeneration, Alzheimer's disease, Huntington's disease, spinal motor atrophy and fragile X mental retardation). Approximately 10% of ALS cases are familial and the rest are sporadic. Mutations in the Cu,Zn superoxide dismutase 1 gene (ALS1: SOD1) have been identified in ˜20% of familial and in ˜3% of sporadic ALS patients, making SOD1 mutations the most common cause of this disease. The RNA-binding proteins TDP-43 and FUS/TLS are the next most commonly mutated genes, each of which is responsible for ˜4% of familial cases. A number of dominantly-inherited genes associated with atypical ALS phenotypes are each responsible for 1-3% of ALS cases (e.g., ALS with dementia/parkinsonism: microtubule-associated protein tau (MAPT): progressive lower motor neuron disease: dynactin subunit 1 (DCTN1); ALS8: vesicle-associated membrane protein-associated protein B/C (VAPB): juvenile onset autosomal dominant ALS: senataxin (SETX)).

ALS genes involved in RNA metabolism include TDP-43 and FUS/TLS: two RNA-binding proteins implicated in transcription, splicing, and translation. Both of these proteins are components of stress granules: cytoplasmic foci that are characterized as signaling centers assembled from proteins and untranslated mRNAs in cells exposed to adverse conditions. ALS-linked genes associated with RNA/stress response pathways include: mutant SOD1 which inactivates a retrograde transport pathway that causes proteins to accumulate in the endoplasmic reticulum and trigger the unfolded protein response (UPR), a pro-survival program; vesicle-associated membrane protein (VAPB), an endoplasmic reticulum protein that interacts with ATF6, a UPR-activated transcription factor; senataxin, a DNA/RNA helicase associated with tRNA metabolism and the oxidative stress response; and angiogenin (ANG), a stress-activated ribonuclease that promotes motor neuron survival and extends the survival of SOD1^{G93A} mice in a murine model of ALS. These genes indicate an association between mutations in proteins involved in stress granule formation and ALS, a neurological disorder associated with neuron death.

### SUMMARY

Provided herein are neuroprotective molecules that contain a sequence of 25-35 contiguous nucleotides that is at least 80% identical to a contiguous sequence between nucleotide 1 and nucleotide 50 of a mature human tRNA that are useful for inducing or increasing stress granule formation in a cell, decreasing protein translation in a cell, reducing stress-induced cell death, and treating a neurological disorder associated with neuron death in a subject. The invention is based on the discovery that these neuroprotective molecules are taken up by motor neurons in the absence of transfection agents, induce stress granule formation in motor neurons, and confer protection against stress-induced motor neuron death.

Provided herein are neuroprotective molecules containing a sequence of 25-35 contiguous nucleotides that is at least 80% identical to a contiguous sequence between nucleotide 1 and nucleotide 50 of a mature human RNA, and at least four (e.g., at least five or six) contiguous guanosine-containing nucleotides, where the sequence of 25-35 contiguous nucleotides contains a D-loop stem structure, the at least four contiguous guanosine-containing nucleotides are located at the 5′ end of the neuroprotective molecule, and the neuroprotective molecule contains at least one (e.g., at least two, three, or four) deoxyribonucleotide. In some embodiments, the sequence of 25-35 contiguous nucleotides is at least 80% identical to a contiguous sequence between nucleotide 1 and nucleotide 50 of a mature human tRNA having a sequence selected from the group consisting of: SEQ ID NOS: 4, 5, 8-11, 13-17, 32, 37, and 40-173.

Also provided are neuroprotective molecules containing a sequence of 25-35 contiguous nucleotides that is at least 80% identical to a contiguous sequence between nucleotide 1 and nucleotide 50 of a mature human tRNA selected from the group of: tRNA^{Arg}, tRNA^{Asp}, tRNA^{Glu}, tRNA^{Gln}, tRNA^{Gly}, tRNA^{His}, tRNA^{Ile}, tRNA^{Leu}, tRNA^{Lys}, tRNA^{Met}, tRNA^{Pro}, tRNA^{SeC}, tRNA^{Ser}, tRNA^{Sup}, tRNA^{Thr}, tRNA^{Trp}, tRNA^{Tyr}, tRNA^{Val}, tRNA^{Asn}, and tRNA^{Phe}; and at least four (e.g., at least five or six) contiguous guanosine-containing nucleotides, where the sequence of 25-35 contiguous nucleotides contains a D-loop stem structure and the at least four contiguous guanosine-containing nucleotides are located at the 5′ end of the neuroprotective molecule. In some embodiments, the sequence of 25-35 contiguous nucleotides is at least 80% identical to a contiguous sequence between nucleotide 1 and nucleotide 50 of a mature human tRNA having a sequence selected from the group of SEQ ID NOS: 5, 8, 9, 11, 14-17, 32, 37, 56, 57, and 63-173. In some embodiments, the sequence of 25-35 contiguous nucleotides is at least 80% identical to a contiguous sequence between nucleotide 1 and nucleotide 50 of a mature human tRNA having a sequence selected from the group of SEQ ID NOS: 11 and 107-116.

In some embodiments, the neuroprotective molecule further comprises a 5′-monophosphate. In some embodiments, the neuroprotective molecule contains at least one modified nucleotide (e.g, a nucleotide containing a modified base and/or a modified sugar). In some embodiments, the neuroprotective molecule contains at least one modification in the phosphate backbone. In some embodiments, the neuroprotective molecule contains a 5′ and/or a 3′ protective group. In some embodiments, the neuroprotective molecule has a total length of between 39 to 60 nucleotides (e.g., 39 to 50 nucleotides or 50 to 60 nucleotides).

Also provided are pharmaceutical compositions that contain at least one neuroprotective molecule described herein (e.g., any of the neuroprotective molecules described herein).

Also provided are methods of inducing or increasing stress granule formation in a cell that include administering to a cell a neuroprotective molecule (e.g., any of the neuroprotective molecules described herein) or an isolated C-myc oligonucleotide containing the sequence of SEQ ID NO: 174, where the neuroprotective molecule or the isolated C-myc oligonucleotide is administered in an amount sufficient to induce or increase stress granule formation in the cell.

Also provided are methods of decreasing protein translation in a cell that include administering to a cell a neuroprotective molecule (e.g., any of the neuroprotective molecules described herein) or an isolated C-myc oligonucleotide containing the sequence of SEQ ID NO: 174, where the neuroprotective molecule or the isolated C-myc oligonucleotide is administered in an amount sufficient to decrease protein translation in the cell.

Also provided are methods of decreasing stress-induced cell death that include administering to the cell a neuroprotective molecule (e.g., any of the neuroprotective molecules described herein) or an isolated C-myc oligonucleotide containing the sequence of SEQ ID NO: 174, where the neuroprotective molecule or the isolated C-myc oligonucleotide is administered in an amount sufficient to decrease stress-induced cell death.

In some embodiments, the cell is in vitro. In some embodiments, the cell is in vivo. In some embodiments, the cell is a neuron (e.g., a motor neuron).

Also provided are methods of treating a neurological disorder associated with neuron death (necrosis and/or apoptosis, or stress-induced cell death) in a subject that include administering a neuroprotective molecule (e.g., any of the neuroprotective molecules described herein) or an isolated C-myc oligonucleotide containing the sequence of SEQ ID NO: 174, where the neuroprotective molecule or the isolated C-myc oligonucleotide is administered in an amount sufficient to treat the neurological disorder associated with neuron death in the subject.

In some embodiments, the neurological disorder associated with neuron death is selected from the group of: amyotrophic lateral sclerosis, Alzheimer's disease, Parkinson's disease, Huntington's disease, muscular dystrophy, multiple sclerosis, and stroke. In some embodiments, the neuroprotective molecule or the isolated C-myc oligonucleotide is administered intravenously, intraarterially, intracranially, ocularly, intraperitoneally, subcutaneously, or intramuscularly. In some embodiments, the subject is administered at least one additional therapeutic agent.

Also provided are methods of identifying a candidate translation inhibitor nucleic acid that include (a) attaching to the 5′ end of a nucleic acid sequence of between 20-50 nucleotides at least four (e.g., at least five or six) guanosine-containing nucleotides, and (b) determining the level of protein translation in the presence of the molecule produced in (a), where a decrease in the level of protein translation in the presence of the molecule produced in (a) relative to the level of protein translation in the absence of the molecule produced in (a) identifies the molecule produced in (a) as a candidate translation inhibitory nucleic acid.

In some embodiments, the nucleic acid sequence of between 20-50 nucleotides contains at least 50% (e.g., at least 55%, 60%, 65%, or 70%) guanosine-containing/cytosine-containing nucleotides. In some embodiments, the nucleic acid sequence of between 20-50 nucleotides is at least 80% identical to a contiguous sequence in a mature human tRNA sequence. In some embodiments, the level of protein translation is determined in a cell. In some embodiments, the cell is a fibroblast or a motor neuron. In some embodiments, the cell is human. In some embodiments, the level of protein translation is determined in a cell lysate (e.g., a rabbit reticulocyte lysate).

By the phrase “mature human tRNA” is meant a human transfer RNA molecule in which the intron sequences have been removed.

By the phrase “D-loop stem structure” is meant a secondary loop structure present in mature human tRNAs that is formed by approximately nucleotides 5 to 28 of a mature human tRNA or a significantly similar stem-loop structure. Several exemplary three-dimensional structures of mature human tRNAs have been described and modeled by those skilled in the art.

By the phrase “deoxyribonucleotide” is meant a nucleotide that contains a 2′-deoxyribose. This term also includes nucleotides that contain modifications at other positions within the deoxyribose sugar or in the attached base.

By the phrase “guanosine-containing nucleotide” is meant a nucleotide that contains a guanosine or a modified guanosine. In some embodiments, the guanosine-containing nucleotide has the ability to form a G-quadruplex.

By the phrase “cytosine-containing nucleotide” is meant a nucleotide that contains a cytosine or a modified cytosine.

By the term “G-quadraplex” is meant a square structural arrangement of four guanines that is stabilized by Hoogsteen hydrogen bonds. A G-quadraplex can also be stabilized by the presence of a monovalent cation (e.g., potassium) in the center of the tetrad. A G-quadraplex can be formed by a DNA, a RNA, an LNA, or a PNA molecule (or any combination thereof). In some embodiments, the G-quadraplex is formed by four guanines that are present in the same molecule (e.g., any of the neuroprotective molecules or C-myc oligonucleotides described herein).

By the term “modified nucleotide” is meant a nucleotide that has a modification in its sugar and/or in its base. Non-limiting examples of modified nucleotides are described herein. Additional examples of modified nucleotides are known in the art.

By the phrase “5′ or 3′ protective group” is meant a moiety that is attached to the 5′- or the 3′-end of an oligonucleotide that prevents nuclease degradation or decreases (e.g., significantly decreases) the rate of nuclease degradation of the oligonucleotide. In some embodiments, the neuroprotective molecules or the C-myc oligonucleotides described herein contain a 5′ and/or 3′ protective group. Non-limiting examples of 5′ and 3′ protective groups are described herein. Additional examples of 5′ and 3′ protective groups are known in the art.

By the term “stress granule formation” is meant the formation or detection of at least one stress granule in a cell. Stress granule formation in a cell can be detected, for example, by microscopy (e.g., immunofluorescence microscopy) or the detection of phosphorylated eIF2α. Additional methods for detecting stress granule formation in a cell are described herein and are known in the art.

By the term “stress granule” is meant an aggregate of proteins and mRNAs that form in a cell under stress conditions. The poly(A)-mRNAs in a stress granule are present in stalled pre-initiation complexes. A stress granule can contain one or more (e.g., two, three, four, or five) of the following proteins/complexes, including but not limited to: 40S ribosomal subunits, eIF4E, eIF4G, eIF4A, eIF4B, poly(A) binding protein (Pabp), eIF3, and eIF2. Additional examples of proteins that can be found in stress granules are described herein. Additional examples of proteins that can be found in stress granules are known in the art (see, for example, Buchan et al., Mol. Cell 36:932, 2009).

By the term “protein translation” is meant the synthesis of a polypeptide from a messenger ribonucleic acid (RNA) molecule (e.g., a capped or uncapped mRNA molecule). The mRNA molecule typically contains a polyA 3′-tail. Protein translation can be performed in a cell or in a cellular lysate. Methods for detecting protein translation or the rate of protein translation are described herein. Additional methods for detecting protein translation or the rate of protein translation are known in the art.

By the term “neurological disorder associated with neuron death” is meant a neurological disease that is characterized by neuron death (e.g., motor neuron death) or has an etiology that involves neuron death (e.g., motor neuron death). Neuron death (apoptosis or necrosis) in these disorders can occur by a variety of different means (e.g., oxidative stress, excitotoxicity, and neuroinflammation). Methods for detecting neuron death in vivo or in vitro are described herein and are known in the art. Non-limiting examples of neurological disorders associated with neuron death are described herein. Additional examples of neurological disorders associated with neuron death are known in the art. In general, the methods do not require that cell death be detected in a subject prior to treatment, if the subject has a disorder known in the art to be associated with neuron death.

By the term “treating” is meant decreasing the number of symptoms or decreasing (e.g., a significant or detectable decrease) the severity, frequency, or duration of one or more symptoms of a disease in a subject. The term “treating” can also include decreasing (e.g., a significant or detectable decrease) the rate of neuron death in a subject having a neurological disorder associated with neuron death (e.g., compared to the rate of neuron death in the same subject prior to treatment or compared to the rate of neuron death in a control subject having the same disease but not receiving treatment or receiving a different treatment). A decrease in neuron death or the rate of neuron death in a subject can be assessed in a subject by a decrease in the rate of the development of one or more symptoms of a neurological disease associated with neuron death or a decrease in the worsening or exacerbation of one of more symptoms of a neurological disease associated with neuron death in a subject.

By the term “stress-induced cell death” is meant cell death that is triggered by or occurs in response to a cellular stress condition (e.g., oxidative stress, neuroexicitotoxicity, and neuroinflammation). Methods for detecting stress-induced cell death are described herein. Additional methods of detecting stress-induced cell death are known in the art.

Unless otherwise defined, all technical and scientific terms used herein have the same meaning as commonly understood by one of ordinary skill in the art to which this invention belongs. Methods and materials are described herein for use in the present invention: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a graph showing the relative amount of luciferase expression in rabbit reticulocyte lysate (RRL) containing uncapped luciferase mRNA following treatment with a control RNA mix (Ctrl 1-2-3), natural 5′-tiRNAs (Nat 5′ end), or natural 3′-tiRNAs (Nat 3′ end) compared to luciferase expression in untreated lysates (bottom), and a Northern blot showing the levels of luciferase mRNA in these experiments (top). The means and standard deviations shown are from three independent experiments (*p=0.01-0.02, Student's t-test, n=3).

FIG. 2 is a graph showing the relative amount of luciferase expression in RRL containing uncapped luciferase mRNA (50 ng/10 μL) following treatment with synthetic 5′-tiRNAs or 3′-tiRNAs (100 picomoles/10 μL) compared to luciferase expression in untreated lysates (*p<0.05, Student's t-test, n=4) (bottom), and a Northern blot showing the levels of luciferase mRNA in these experiments (top).

FIG. 3 is a graph showing the dose-dependent inhibition of uncapped luciferase mRNA translation in RRL following treatment with control RNAs (ctrl-1, ctrl-2, and ctrl-3), synthetic 5′-tiRNA^{Ala} (5′-Ala), or synthetic 3′-tiRNA^{Ala} (3″-Ala). Translation of the reporter in the absence of any RNA (No RNA) was assigned a relative value of 100%. The error bars reflect the standard deviations of the mean (*p<0.05, relative to no RNA and all three control RNAs (ctrl-1, ctrl-2, and ctrl-3), Student's t-test, n=3).

FIG. 4 is a graph showing the relative amount of luciferase expression in RRL containing capped luciferase mRNA (10 ng/10 μL) in the presence or absence of the indicated synthetic RNAs (100 picomoles/10 μL) in the absence (dark bars) or presence (light bars) of cap analogue (m⁷GpppG, 0.1 mM) (*p<0.05, comparing 5′-tiRNA^{Ala} in the presence or absence of the cap analogue, Student's t-test, n=3) (bottom), and a Northern blot showing the levels of luciferase mRNA in these experiments (top).

FIG. 5 is a graph showing the relative ratio of Firefly to Renilla luciferase counts produced in a RRL assay using uncapped (dark bars) or capped (light bars) pF/R bicistronic transcripts in the presence of 5′-tiRNA^{Ala} (*p<0.05, compared to no RNA and three control RNAs (ctrl-1, -2, and -3), Student's t-test, n=3-4) (right panel), and a graph showing the luciferase counts from EMCV IRES-driven translation of the Renilla ORF relative to the no RNA control (100%) in the RRL using uncapped (dark bars) or capped (light bars) pF/R bicistronic transcripts in the presence of 5′-tiRNA^{Ala} (left panel).

FIG. 6 is two graphs showing the luciferase expression in RRL assays using monocistronic transcripts encoding Renilla luciferase under the control of different EMCV IRES variants (WT EMCV-UA₆ (upper graph) and EMVC-UA₇ (lower graph)) in the presence of control RNAs (ctrl-1, -2, and -3), 5′-tiRNA^{Ala}, or 3′-tiRNA^{Ala} (*p<0.05 compared to no RNA and three control RNAs (ctrl-1, -2, and -3), Student's t-test, n=3).

FIG. 7 is a graph showing the luciferase expression in RRL assays using uncapped Firefly luciferase RNA in the presence of control RNA or synthetic 5′-tiRNA^{Ala} (5″-Ala) supplemented with a two-molar excess of recombinant eIF4E protein (+4E). Translation of the reporter in the absence of any RNA (no RNA) is assigned a relative value of 100%. The error bars reflect standard deviations of the mean.

FIG. 8 is a graph showing luciferase expression in translation assays using uncapped polyA-biotinylated Renilla luciferase transcript in RRL supplemented with U2OS extract in the presence of the indicated control RNAs or tiRNAs (*p<0.05, compared to no RNA and three control RNAs (ctrl-1, -2, and -3), Student's t-test, n=3) (top), and an immunoblot of the streptavidin pull-down of uncapped polyA-biotinylated transcripts (bottom). In vitro translation was performed in the presence of control RNA (Ctrl), 5′-tiRNA^{Ala}, or cap analogue (Cap, m⁷GpppG, 0.1 mM). Streptavidin beads were used to pull down reporter RNA-protein complexes. The bound proteins were identified by Western blotting using the indicated antibodies.

FIG. 9 is a graph showing luciferase expression in translation assays using capped polyA-biotinylated Renilla luciferase transcript in RRL supplemented with U2OS extract in the presence of the indicated control RNAs or tiRNAs (*p<0.05, compared to no RNA and three control RNAs (ctrl-1, -2, and -3), Student's t-test, n=3), and an immunoblot of the streptavidin pull-down of capped polyA-biotinylated transcripts (bottom). In vitro translation was performed in the presence of control RNA (Ctrl), 5′-tiRNA^{Ala}, or without any RNA (No RNA). Streptavidin beads were used to pull down reporter RNA-protein complexes. The bound proteins were identified by Western blotting using the indicated antibodies.

FIG. 10 is two Western blots showing the displaced components following incubation of eIF4E complexes (assembled on m⁷GTP-Sepharose using U2OS lysates) with various components. The right blot shows the displaced components following incubation of the complexes with the indicated 3′-end biotinylated RNAs or cap analogue (Cap, m⁷GpppG, 0.1 mM). Streptavidin beads were used to pull down displaced RNA-bound proteins and were identified by Western blotting (left blot).

FIG. 11 is an immunoblot showing the displaced components following incubation of eIF4E complexes (assembled on m⁷GTP-Sepharose using RRL supplemented U2OS extract) with no RNA, control RNA (ctrl), 5″-tiRNA^{Ala}, or 5′-tiRNA^{Cys}. The integrity of the eIF4F complex was monitored by Western blotting using anti-eIF4E, anti-eIF4G, and anti-eIF4A antibodies.

FIG. 12 is the secondary structure of 5′-tiRNA^{Ala}.

FIG. 13 is the predicted secondary structures of the control RNAs and 5′-tiRNA^{Ala}, 5′-tiRNA^{Cys}, 5′-tiRNA^{Met}, the mutants of 5′-tiRNA^{Ala}, and the modified 5′-tiRNA^{Met}.

FIG. 14 is a graph of the relative luciferase expression in RRL in the presence of synthetic control RNAs (ctrl-1, -2, and -3), 5′-tiRNA^{Ala}, or the indicated mutants relative to the luciferase expression that is observed in the absence of any RNA (No RNA=100%) (*p<0.05, Student's t-test, n=4).

FIG. 15 is a Western blot showing the displacement of eIF4F complexes from m⁷GTP-Sepharose in U2OS lysates following the addition of the indicated RNAs to the beads.

FIG. 16 is a graph showing the percentage of cells with stress granules following transfection with the control and synthetic RNAs shown (200 cells/experiment: * p<0.05, compared to a control without any RNA, Student's t-test, n=3).

FIG. 17 is a Western blot showing the level of phospho-eIF2a in U2OS cells treated with pateamine A (PA: 15 nM) or sodium arsenite (SA: 70 μM), or transfected with no RNA, control RNA (ctrl-1, -2, or -3), 5′-tiRNA^{Ala}, and its mutants. Phospho-eIF2a was detected with polyclonal antibodies specific for eIF2a phosphorylated at S52 (Assay Designs) (upper panels). Detection of TIA-1 (lower panels) was used as a loading control.

FIG. 18 is a graph of the relative luciferase expression in RRL in the presence of synthetic control RNAs (ctrl-1, -2, and -3) or the indicated 5′-tiRNAs relative to the luciferase expression that is produced in the absence of any RNA (No RNA=100%) (*p<0.05, Student's t-test, n=3).

FIG. 19 is a Western blot showing the displacement of eIF4F complexes from m⁷GTP-Sepharose in U2OS lysates following addition of the indicated RNAs to the beads. The Western blot was developed to detect eIF4F complexes and eIF4E-BP1.

FIG. 20 is a graph showing the percentage of cells with stress granules following transfection with the indicated RNAs (200 cells/experiment: * p<0.05, Student's t-test, n=3).

FIG. 21 is an immunoblot showing proteins bound to biotinylated control RNA and 5′-tiRNA^{Ala} that were purified using streptavidin beads, subjected to the indicated salt washes (0.1 M, 0.3 M, or 0.5 M NaCl), and eluted.

FIG. 22 is an immunoblot showing the expression of specific binding proteins following transfection with a control (Ctrl) or the different siRNAs shown (Target) (left panel) (tubulin was used as a specificity and loading control), and a graph showing the percentage of cells having stress granules following treatment with the indicated siRNAs, followed by mock transfection or transfection with control RNA (Ctrl) or 5′-tiRNA^{Ala} (200 cells/experiment; AGO2 siRNA, n=7; PABP1 siRNA, n=3; YB-1 siRNA, n=9; Vigilin siRNA, n=3; FXR1 siRNA, n=3) (*p<0.0001 when comparing the percentage of cells with stress granules in control and YB-1 siRNA treated cells; no other treatment reached statistical significance).

FIG. 23 shows the nucleotide sequences of the G-rich oligodeoxynucleotides used in some experiments described herein. The 5′-terminal guanines are indicated in bold.

FIGS. 24A-B are two graphs showing the relative luciferase count from translation assays performed using uncapped (A) or capped (B) Firefly luciferase mRNA and RRL lysate in the presence of control G-rich oligonucleotides (Ctrl 25, Ctrl 30, and phCtrl) or the indicated G-rich oligodeoxynucleotides. The percentage luciferase expression shown is relative to the luciferase expression observed in the absence of any DNA oligonucleotide (No DNA=100%). The means and standard deviations from at least three independent experiments are shown (*p<0.05).

FIG. 25 shows the percentage of U2OS cells having stress granules following transfection with the indicated control or G-rich oligodeoxynucleotides (0.75 μM) using Lipofectamine. After 24 hours, the cells were processed for immunofluorescence microscopy using antibodies reactive with the stress granule components eIF3, G3BP, and TIA-1. The percentage of cells with stress granules is depicted as the mean #standard error (n=3) (*p<0.05, compared with control oligodeoxynucleotides).

FIGS. 26A-B show two immunoblots showing the displacement of eIF4F from eIF4F:m⁷GTP-Sepharose complexes formed in U2OS (A) or NSC34 (B) lysates upon co-incubation with the indicated 3′-biotinylated control or G-rich oligodeoxynucleotides. Synthetic 5′-tiRNA^{Ala} served as a positive control for displacement of eIF4F complex from m⁷GTP beads (A: left panel). Streptavidin beads were used to pull down displaced DNA-bound proteins that were identified by immunoblotting (A: right panel).

FIGS. 27A-D are four photomicrographs of NSC34 cells treated with no DNA (A: upper left panel) or 1.5 μM of biotin-labeled control DNA (p-ctrl) (B: upper right panel), AS1411 (p-PS1411) (C: lower left panel), or C-myc oligodeoxynucleotide (D: lower right panel), and then stained with Cy3-streptavidin 72 hours after oligodeoxynucleotide treatment. White arrows indicate the presence of the biotin-labeled oligodeoxynucleotides in the cell.

FIG. 28 is a graph showing the percent dead NSC34 cells (percent Trypan Blue-positive NSC34 cells) following treatment with the indicated G-rich deoxyoligonucleotides for 72 hours. The means and standard deviations are from four independent experiments (*p<0.05 compared to controls). The increased shading of the bars indicates an increased concentration of the G-rich deoxyoligonucleotide.

FIG. 29 is a graph showing the percent dead NSC34 cells following pretreatment for 72 hours with the indicated G-rich oligodeoxynucleotides and subsequent exposure to serum starvation or rotenone treatment (10 μM) for 24 hours. No treatment (No treat) served as a control. The percentage of dead cells was determined by Trypan Blue staining. The means and standard deviations are from four independent experiments (*p<0.05 compared to controls).

### DETAILED DESCRIPTION

Neuroprotective molecules that are capable of inhibiting protein translation, inducing or increasing stress granule formation in a cell (e.g., a motor neuron), and decreasing stress-induced cell death (e.g., motor neuron death) have been discovered. These neuroprotective molecules are based, in part, on sequences present within mature human tRNAs. Some of these neuroprotective molecules have been shown to be taken up by motor neurons in the absence of transfection agents. The structural features of the neuroprotective molecules are described below, as well as their use in methods of inhibiting protein translation in a cell, inducing or increasing stress granule formation in a cell, reducing stress-induced cell death, and treating a neurological disorder associated with neuron death. Also provided herein are methods of identifying a candidate translation inhibitory nucleic acid.

### Compositions

Provided herein are neuroprotective molecules containing a sequence of 25-35 contiguous nucleotides (e.g., 25-30 nucleotides or 30-35 nucleotides) that is at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or 100% identical) to a contiguous sequence between nucleotide 1 to nucleotide 50 (e.g., between nucleotide 1 to nucleotide 30) of a mature human tRNA (e.g., any of the mature human tRNA sequences described herein or known in the art), and at least four (e.g., five, six, seven, or eight) guanosine-containing nucleotides, where the sequence of 25-35 contiguous nucleotides contains a D-loop stem structure, the at least four contiguous guanosine-containing nucleotides are located at the 5′-end of the neuroprotective molecule, and the neuroprotective molecule contains at least one (e.g., two, three, four, five, six, seven, eight, nine, or ten) deoxyribonucleotide. In some embodiments, the sequence of 25-35 contiguous nucleotides is at least 80% identical to a continguous sequence of between nucleotide 1 and nucleotide 50 of a mature human tRNA having a sequence selected from: SEQ ID NOS: 4, 5, 8-11, 13-17, 32, 37, and 40-173.

Also provided are neuroprotective molecules containing a sequence of 25-35 contiguous nucleotides (e.g., 25-30 nucleotides or 30-35 nucleotides) that is at least 80% identical (e.g., at least 81%, 82%, 83%, 84%, 85%, 86%, 87%, 88%, 89%, 90%, 91%, 92%, 93%, 94%, 95%, 96%, 97%, 98%, 99%, or 100% identical) to a contiguous sequence between nucleotide 1 and nucleotide 50 (e.g., between nucleotide 1 to nucleotide 30) of a mature human tRNA selected from the group of: tRNA^{Arg}, tRNA^{Asp}, tGlu^{Glu}, tRNA^{Gln}, tRNA^{Gly}, tRNA^{His}, tRNA^{Ile}, tRNA^{Leu} tRNA^{Lys}, tRNA^{Met}, tRNA^{Pro}, tRNA^{SeC}, tRNA^{Ser}, tRNA^{Sup}, tRNA^{Thr}, tRNA^{Trp}, tRNA^{Tyr}, tRNA^{Val}, tRNA^{Asn}, and tRNA^{Phe} (e.g., any of the sequences described herein or known in the art), and at least four (e.g., five, six, seven, or eight) contiguous guanosine-containing nucleotides, where the sequence of 25-35 contiguous nucleotides contains a D-loop stem structure and the at least four contiguous guanosine-containing nucleotides are located at the 5′ end of the neuroprotective molecule. In some embodiments, the sequence of 25-35 contiguous nucleotides is at least 80% identical to a contiguous sequence between nucleotide 1 and nucleotide 50 of a mature human RNA having a sequence selected from the group of SEQ ID NOS: 5, 8, 9, 11, 14-17, 32, 37, 56, 57, and 63-173. In some embodiments, the sequence of 25-35 contiguous nucleotides is at least 80% identical to a contiguous sequence between nucleotide 1 and nucleotide 50 of a mature human tRNA having a sequence selected from the group of SEQ ID NOS: 11 and 107-116.

The comparison of sequences and determination of percent identity between two sequences can be accomplished using a mathematical algorithm. The percent identity between two amino acid sequences is determined using the Needleman and Wunsch J. Mol. Biol. 48:444-453, 1970) algorithm, which has been incorporated into the GAP program in the GCG software package (available the at gcg.com), using either a Blossum 62 matrix or a PAM250 matrix, and a gap weight of 16 and a length weight of 1. The percent identity between two nucleotide sequences is determined using the GAP program in the GCG software package (available the Accelrys Inc. website), using a NWSgapdna.CMP matrix and a gap weight of 40 and a length weight of 1. The calculation of percent identity as described herein, recognizes a uracil-containing nucleotide (deoxyuracil and ribouracil) as being the same as a “T” (deoxythymine or ribothymine).

In some embodiments, the neuroprotective molecules can contain a sequence of 25, 26, 27, 28, 29, 30, 31, 32, 33, 34, or 35 contiguous nucleotides that is at least 80% identical to a contiguous sequence present between nucleotide 1 and nucleotide 50 of a mature human tRNA. In some embodiments, the contiguous sequence between nucleotide 1 and nucleotide 50 of a mature human tRNA starts at nucleotide number 1, 2, 3, 4, 5, 6, or 7 of the mature human tRNA sequence.

Non-limiting examples of mature human tRNAs are listed below. Any of the human tRNA molecules described herein or known in the art can be used to design the neuroprotective molecules described herein.

Non-limiting exemplary Ala tRNA sequences are listed below.

Ala tRNA
(SEQ ID NO: 40)
ggggguguag cucaguggua gagcgcgugc uu
(SEQ ID NO: 41)
ggggguguag cucaguggua gagcgcgugc u
(SEQ ID NO: 42)
ggggggugua gcucaguggu agagcgcgug cu
(SEQ ID NO: 43)
ggggguguag cucaguggua gagcgcgugc
(SEQ ID NO: 44)
ggggggugua gcucaguggu agagcgcgug c
SEQ ID NO: 45)
gggggunuag cucaguggua gagcgcgugc uu
(n is a, c, g, or u;
(SEQ ID NO: 46)
ggggguguag cucaguggua gagcgcgug
(SEQ ID NO: 47)
guguagcuca gugguagagc gcgugcuucg c
SEQ ID NO: 48)
ggggunuagc ucagugguag agcgcgugcu u
(n is a, c, g, or u;
SEQ ID NO: 49)
ggggggunua gcucaguggu agagcgcgug cu
(n is a, c, g, or u;
(SEQ ID NO: 50)
gggguguagc ucagugguag agagcgugcu u
(SEQ ID NO: 51)
gggggunuag cucaguggua gagcgcgugc
(SEQ ID NO: 52)
ggggguguag cucaguggua gagagcgugc u
(SEQ ID NO: 53)
ggggauguag cucaguggua gagcgcaugc u
(SEQ ID NO: 54)
gggggauuag cucaaauggu agagcgcucg
(SEQ ID NO: 55)
ggggaunuag cucaguggua gagcgcaugc u

A non-limiting exemplary Arg tRNA sequence is ggcucuguug cgcaauggau agcgcau (SEQ ID NO: 56) and a non-limiting exemplary Asp tRNA sequence is uccucauuag uauaguggug aguauccc (SEQ ID NO: 57).

Non-limiting exemplary Cys tRNA sequences are listed below.

Cys tRNA
(SEQ ID NO: 58)
ggggguauag cucaguggua gagcauuuga
(SEQ ID NO: 59)
ggggguauag cucaguggua gagcauuug
(SEQ ID NO: 60)
ggggguauag cucaguggua gagcauuu
(SEQ ID NO: 61)
ggggguuuag cucagugggu agagcau
(SEQ ID NO: 62)
ggggguguaa cucaguggua gagcauuuga

A non-limiting exemplary Gln tRNA sequence is gguuccaugg uguaaugguu agcacucug (SEQ ID NO: 63).

Non-limiting exemplary Gly tRNA sequences are listed below.

| | Gly tRNA |
|---|---|
| (SEQ ID NO: 64) | |
| | uuggugguuc agugguagaa uucucgccug cc |
| (SEQ ID NO: 65) | |
| | uuggugguuc agugguagaa uucucgccug c |
| (SEQ ID NO: 66) | |
| | uuggugguuc agugguagaa uucucgccug |
| (SEQ ID NO: 67) | |
| | uggugguuca gugguagaau ucucgccug |
| (SEQ ID NO: 68) | |
| | auuggugguu cagugguaga auucucgccu g |
| (SEQ ID NO: 69) | |
| | uuggugguuc agugguagaa uucucgccu |
| (SEQ ID NO: 70) | |
| | ggcauuggug guucaguggu agaauucucg c |
| (SEQ ID NO: 71) | |
| | auuggugguu cagugguaga auucucgcc |
| (SEQ ID NO: 72) | |
| | agcauuggug guucaguggu agaauucucg c |
| (SEQ ID NO: 73) | |
| | cauugguggu ucagugguag aauucucgc |
| (SEQ ID NO: 74) | |
| | gggaggcccg gguucguuuc ccggccaaug ca |
| (SEQ ID NO: 75) | |
| | gcauuggugg uucaguggua gaauucucac |
| (SEQ ID NO: 76) | |
| | cgggaggccc ggguucgguu cccggccaau gc |
| (SEQ ID NO: 77) | |
| | uuggugguuc agugguagaa uucucgc |
| (SEQ ID NO: 78) | |
| | gacauuggug guucaguggu agaauucu |
| (SEQ ID NO: 79) | |
| | ugguucagug guagaauucu cgccucc |
| (SEQ ID NO: 80) | |
| | gcauugguau agugguauca ugcaaga |
| (SEQ ID NO: 81) | |
| | agcguuggug guauaguggu gagcauagcu gc |

Non-limiting examples of His tRNA sequences are listed below.

| | His tRNA |
|---|---|
| (SEQ ID NO: 82) | |
| | ggccgugauc guauaguggu uaguacucug |
| (SEQ ID NO: 83) | |
| | ucgccgugau cguauagugg uuaguacucu g |
| (SEQ ID NO: 84) | |
| | ggccgugauc guauaguggu uaguacuc |
| (SEQ ID NO: 85) | |
| | aggccgugau cguauagugg uuaguacuc |
| (SEQ ID NO: 86) | |
| | ggccgugauc guauaguggu uaguacu |
| (SEQ ID NO: 87) | |
| | ggccgugauc guauaguggu uaguac |

Non-limiting examples of Ile tRNA sequences are listed below.

| | Ile tRNA |
|---|---|
| (SEQ ID NO: 88) | |
| | ggccgguuag cucaguuggu uagagc |
| (SEQ ID NO: 89) | |
| | ggccgguuag cucaguuggu cagagc |
| (SEQ ID NO: 90) | |
| | ggccgguuag cucaguuggu aagagcuugg u |
| (SEQ ID NO: 91) | |
| | ggggcggccg guuagcucag uugguaagag c |
| (SEQ ID NO: 92) | |
| | ggccgguuag cucaguuggu aagugc |

Non-limiting examples of Leu tRNA sequences are listed below.

| | Leu tRNA |
|---|---|
| (SEQ ID NO: 93) | |
| | gguagugugg ccgagcgguc uuaggc |
| (SEQ ID NO: 94) | |
| | guagucgugg ccgagugguu aaggcuaugg a |
| (SEQ ID NO: 95) | |
| | gacgaggugg ccgagugguu aaggcuaugg au |
| (SEQ ID NO: 96) | |
| | gacgaggugg ccgagugguu aaggcuaugg ac |
| (SEQ ID NO: 97) | |
| | gacgaggugg ccgagugguu aaggcuaugg a |
| (SEQ ID NO: 98) | |
| | gacgaggugg ccgagugguu aaggcuaugg |
| (SEQ ID NO: 99) | |
| | gacgaggugg ccgagugguu aaggcaaugg a |
| (SEQ ID NO: 100) | |
| | gacgaggugg ccgagugguu aaggcaaugg |
| (SEQ ID NO: 101) | |
| | uguagucgug gccgaguggu uaaggc |

Non-limiting examples of Lys tRNA sequences are listed below.

| | Lys tRNA |
|---|---|
| (SEQ ID NO: 102) | |
| | gccuggauag cucaguuggu agagcaucag a |
| (SEQ ID NO: 103) | |
| | gccuggauag cucaguuggu agagcauca |
| (SEQ ID NO: 104) | |
| | gccuggguag cucagucggu agagcaucag ac |
| (SEQ ID NO: 105) | |
| | gccuggguag cucagucggu agagcaucag a |
| (SEQ ID NO: 106) | |
| | gccuggguag cucagucggu agagcaucag |

Non-limiting examples of Met tRNA sequences are listed below.

Met tRNA
(SEQ ID NO: 107)
gcagaguggc gcagcggaag cgugcugggc cc
(SEQ ID NO: 108)
ggcagagugg cgcagcggaa gcgugcuggg cc
(SEQ ID NO: 109)
gcagaguggc gcagcggaag cgugcugg
(SEQ ID NO: 110)
ugcagagugg cgcagcggaa gcgugcugg
(SEQ ID NO: 111)
gcaguggcgc agcggaagcg ugcugggcc
(SEQ ID NO: 112)
gcagaguggc gcagcggaag cgugcug
(SEQ ID NO: 113)
cgcagagucg cgcagcggaa gcgugcuggg cc
(SEQ ID NO: 114)
cagagucgcg cagcggaagc gugcugggcc c
(SEQ ID NO: 115)
agaguugcgc agcggaagcg ugcugggccc a
(SEQ ID NO: 116)
gagauagcag aguggcgcag cggaagc

Non-limiting examples of Pro tRNA sequences are ggcucguugg ucuaggggua ugauucucgg (SEQ ID NO: 117) and aggcucguug gucuaguggu augauucucg (SEQ ID NO: 118).

A non-limiting example of a SeC (selenocysteine) tRNA sequence is gcccggauga uccucagugg ucuggggugc (SEQ ID NO: 119).

Non-limiting examples of Ser tRNA sequences are listed below.

Ser tRXA
(SEQ ID NO: 120)
uguagucgug gccgaguggu uaaggc
(SEQ ID NO: 121)
gacgaggugg ccgagugguu aaggcuaugg ac
(SEQ ID NO: 122)
gacgaggugg ccgagugguu aaggcuaugg au
(SEQ ID NO: 123)
gacgaggugg ccgagugguu aaggcuaugg a
(SEQ ID NO: 124)
gacgaggugg ccgagugguu aaggcaaugg a
(SEQ ID NO: 125)
gacgaggugg ccgagugguu aaggcuaugg
(SEQ ID NO: 126)
gacgaggugg ccgagugguu aaggcaaugg

Non-limiting examples of Sup (suppressor) tRNA sequences are gccuggauag cucaguuggu agagcaucaga (SEQ ID NO: 127) and gccuggauag cucaguuggu agagcauca (SEQ ID NO: 128).

Non-limiting examples of Thr tRNA sequences are listed below.

Thr tRNA
(SEQ ID NO: 129)
ggcagagugg cgcagcggaa gcgugcuggg cc
(SEQ ID NO: 130)
gcagaguggc gcagcggaag cgugcugggc cc
(SEQ ID NO: 131)
gcagaguggc gcagcggaag cgugcugg
(SEQ ID NO: 132)
cggaagcgug cugggcccau aacccaga
(SEQ ID NO: 133)
ugcagagugg cgcagcggau gcgugcugg
(SEQ ID NO: 134)
gcaguggcgc agcggaagcg ugcugggcc
(SEQ ID NO: 135)
gcagaguggc gcagcggaag cgugcug
(SEQ ID NO: 136)
cgcagagucg cgcagcggaa gcgugcuggg cc
(SEQ ID NO: 137)
cagagucgcg cagcggaagc gugcugggcc c
(SEQ ID NO: 138)
agaguugcgc agcggaagcg ugcugggccc a

Non-limiting examples of Tyr tRNA sequences include gccuggauag cucaguuggu agagcaucaga (SEQ ID NO: 139) and gccuggauag cucaguuggu agagcauca (SEQ ID NO: 140).

Non-limiting examples of Val tRNA sequences are listed below.

Val tRNA
(SEQ ID NO: 141)
uuccguagug uagugguuau cacguucgcc uc
(SEQ ID NO: 142)
uuccguagug uagugguuau cacguucgcc
(SEQ ID NO: 143)
uccguagugu agugguuauc acguucgccu ga
(SEQ ID NO: 144)
uccguagugu agugguuauc acguucgccu g
(SEQ ID NO: 145)
uccguagugu agugguuauc acguucgccu ca
(SEQ ID NO: 146)
uccguagugu agugguuauc acguucgccu
(SEQ ID NO: 147)
guuuccguag uguagugguc aucacguucg cc
(SEQ ID NO: 148)
ccguagugua gugguuauca cguucgcc
(SEQ ID NO: 149)
guuuccguag uguagugguc aucacguucg
(SEQ ID NO: 150)
cguaguguag ugguuaucac guucgcc
(SEQ ID NO: 151)
uccguagugu agugguuauc acuuucgccu
(SEQ ID NO: 152)
uccguagugu acugguuauc acguucgccu g
(SEQ ID NO: 153)
cguaguguag uggucaucac guucgccu
(SEQ ID NO: 154)
ggggguguag cucaguggua gagcgcgugc uu
(SEQ ID NO: 155)
ggggguguag cucaguggua gagcgcgugc u
(SEQ ID NO: 156)
ggggguguag cucaguggua gagcgcgugc
(SEQ ID NO: 157)
ggggguguag cucaguggua gagcgcgug
(SEQ ID NO: 158)
ggggggugua gcucaguggu agagcgcgug cu
(SEQ ID NO: 159)
uuggugguuc agugguagaa uucucgccug cc
(SEQ ID NO: 160)
uuggugguuc agugguagaa uucucgccug c
(SEQ ID NO: 161)
uuggugguuc agugguagaa uucucgccug
(SEQ ID NO: 162)
uuggugguuc agugguagaa uucucgccu
(SEQ ID NO: 163)
uggugguuca gugguagaau ucucgccug
(SEQ ID NO: 164)
auuggugguu cagugguaga auucucgccu g
(SEQ ID NO: 165)
auuggugguu cagugguaga auucucgcc
(SEQ ID NO: 166)
ggcauuggug guucaguggu agaauucucg c
(SEQ ID NO: 167)
uuggugguuc agugguagaa uucucgc
(SEQ ID NO: 168)
ugguucagug guagaauucu cgccucc
(SEQ ID NO: 169)
cauugguggu ucagugguag aauucucgc
(SEQ ID NO: 170)
agcauuggug guucaguggu agaauucucg c
(SEQ ID NO: 171)
gcauuggugg uucaguggua gaauucucac

A non-limiting example of a Asn tRNA sequence is gtctctgtgg cgcaateggt tagegcgttc ggctgttaac (SEQ ID NO: 172). A non-limiting example of a Phe tRNA sequence is gccgaaatag ctcagttggg agagcgttag actgaagatc (SEQ ID NO: 173). Additional non-limiting examples of human mature tRNA sequences that can be used to generate any of the neuroprotective molecules described herein are listed in the Sequence Appendix and are described in U.S. Patent Application Publication No. 20110046209 (herein incorporated by reference). Additional examples of human mature tRNA sequences that can be used to generate any of the neuroprotective molecules described herein are known in the art (see, NCBI website, the UCSC genomic tRNA database website (address: gtrnadb.ucsc.edu), and the Unversitat Leipzig tRNAdb website (address:trandb.bioinf.uni-leipzig.edu).

The sequence of 25-35 contiguous nucleotides that is at least 80% identical to a contiguous sequence present between nucleotide 1 and nucleotide 50 of a mature human tRNA incorporated in the neuroprotective molecules described herein should be sufficient to allow the formation of a D-stem loop structure in the neuroprotective molecule. The D-stem loop structure of mature human tRNAs is typically comprised of a region extending from about nucleotide 6 to about nucleotide 27. The formation of a D-loop stem structure within the neuroprotective molecules described herein can be assessed by its gel migration (e.g., in the presence or absence of denaturing agents) or by circular dichroism. Additional methods for the detection of the D-loop stem structure within a nucleic acid are known in the art.

In some embodiments, the guanosine-containing nucleotide can contain a ribose. In some embodiments, the guanosine-containing nucleotide can contain a deoxyribose. In some embodiments, the guanosine-containing nucleotide can contain a modified sugar (e.g., any of the modified sugars described herein). In some embodiments, the guanosine-containing nucleotide can contain modified guanosine (e.g., 7-methyl guanosine or 6-thioguanosine). In preferred embodiments, the at least four contiguous guanosine-containing nucleotides form a G-quadraplex (e.g., a G-quadraplex with guanosines within the neuroprotective molecule).

The neuroprotective molecules described herein can contain one or more (e.g., two, three, four, of five) modified nucleotides. The modified nucleotides can contain a modified base or a modified sugar. Non-limiting examples of modified bases include: xanthine, diaminopurine, 8-oxo-N⁶-methyladenine, 7-deazaxanthine, 7-deazaguanine, N⁴,N⁴-ethanocytosin, N⁶, N⁶-ethano-2,6-diaminopurine, 5-methylcytosine, 5-(C³-C₆)-alkynyl-cytosine, 5-fluorouracil, 5-bromouracil, pseudoisocytosine, 2-hydroxy-5-methyl-4-triazolopyridin, isocytosine, isoguanine, and inosine. Additional non-limiting examples of modified bases include those nucleobases described in U.S. Pat. Nos. 5,432,272 and 3,687,808 (herein incorporated by reference), Freier et al., Nucleic Acid Res. 25:4429-4443, 1997: Sanghvi, Antisense Research and Application, Chapter 15, Ed. S. T. Crooke and B. Lebleu, CRC Press, 1993: Englisch, et al., Angewandte Chemie 30:613-722, 1991, Kroschwitz, Concise Encyclopedia of Polymer Science and Engineering, John Wiley & Sons, pp. 858-859, 1990: and Cook, Anti-Cancer Drug Design 6:585-607, 1991. Additional non-limiting examples of modified bases include universal bases (e.g., 3-nitropyrole and 5-nitroindole). Other modified bases include pyrene and pyridyloxazole derivatives, pyrenyl, pyrenylmethylglycerol derivatives, and the like. Other preferred universal bases include pyrrole, diazole, or triazole derivatives, including those universal bases known in the art.

In some embodiments, the modified nucleotide can contain a modification in its sugar moiety. A non-limiting examples of modified nucleotides that contains a modified sugar are locked nucleic acids (LNAs). LNA monomers are described in WO 99/14226 and U.S. Patent Application Publications Nos. 20110076675, 20100286044, 20100279895, 20100267018, 20100261175, 20100035968, 20090286753, 20090023594, 20080096191, 20030092905, 20020128381, and 20020115080 (herein incorporated by reference). Additional non-limiting examples of LNAs are disclosed in U.S. Pat. Nos. 6,043,060, 6,268,490, WO 01/07455, WO 01/00641, WO 98/39352, WO 00/56746, WO 00/56748, and WO 00/66604 (herein incorporated by reference), as well as in Morita et al., Bioorg. Med. Chem. Lett. 12(1):73-76, 2002; Hakansson et al., Bioorg. Med. Chem. Lett. 11(7):935-938, 2001; Koshkin et al., J. Org. Chem. 66(25): 8504-8512, 2001; Kvaerno et al., J. Org. Chem. 66(16):5498-5503, 2001; Hakansson et al., J. Org. Chem. 65(17):5161-5166, 2000; Kvaerno et al., J. Org. Chem. 65(17):5167-5176, 2000; Pfundheller et al., Nucleosides Nucleotides 18(9): 2017-2030, 1999; and Kumar et al., Bioorg. Med. Chem. Lett. 8(16):2219-2222, 1998. In some embodiments, the modified nucleotide is an oxy-LNA monomer, such as those described in WO 03/020739.

The neuroprotective molecules described herein can also contain a modification in the phosphodiester backbone. For example, at least one linkage between any two contiguous (adjoining) nucleotides in the neuroprotective molecule can be connected by a moiety containing 2 to 4 groups/atoms selected from the group of: —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —OCH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —OCH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —OCH₂—CO—NR^{H}—, —OCH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}—, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—. —O—S(O)₂—O—, —O—S(O)₂—CH₂—, —O—S(O)₂—NR^{H}—, —NR^{H}—S(O)₂—CH₂—, —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O,S)—S—, —O—P(S)₂—S—, —S—, —P(O)₂—S—, —S—P(O,S)—S—, —S—P(S)₂—S—, —O—PO(R″)—O—, —O—PO(OCH₃)—O—, —O—PO—(OCH₂CH₃)—O—, —O—PO(OCH₂S—R)—O—, —O—PO(BH₃)—O—, —O—PO(NHRN)—O—, —O—P(O)₂—NR^{H}—, —NRHP(O)₂—O—, —O—P(O,NR^{H})₂—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—, —O—P(O,S)—O—, —O—P(S)₂—O—, —NR^{H}—P(O)₂—O—, —O—P(O,NR^{H})—O—, —O—PO(R″)—O—, —O—PO(CH₃)—O—, and —O—PO(NHRN)—O—, where R^{H} is selected form hydrogen and C₁₋₄-alkyl, and R″ is selected from C₁₋₆-alkyl and phenyl. Further illustrative examples are given in Mesmacker et. al., Curr. Opin. Struct. Biol. 5:343-355, 1995; and Freier et al., Nucleic Acids Research 25:4429-43, 1997. The left-hand side of the inter-nucleoside linkage is bound to the 5-membered ring as substituent P* at the 3′-position, whereas the right-hand side is bound to the 5′-position of a preceding monomer.

In some embodiments, any of the neuroprotective molecules described herein can be modified at either the 3′ and/or 5′ end by any type of modification known in the art. For example, either or both ends may be capped with a protecting group, attached to a flexible linking group, attached to a reactive group to aid in attachment to the substrate surface. Non-limiting examples of 3′ and/or 5′ blocking groups include: 2-amino-2-oxyethyl, 2-aminobenzoyl, 4-aminobenzoyl, acetyl, acetyloxy, (acetylamino)methyl, 3-(9-acridinyl), tricyclo[3.3.1.1(3,7)]dec-1-yloxy, 2-aminoethyl, propenyl, (9-anthracenylmethoxy)carbonyl, (1, 1-dmimethylpropoxy)carbonyl, (1,1-dimethylpropoxy)carbonyl, [1-methyl-1-[4-(phenylazo)phenyl]ethoxy]carbonyl, bromoacetyl, (benzoylamino)methyl, (2-bromoethoxy)carbonyl, (diphenylmethoxy)carbonyl, 1-methyl-3-oxo-3-phenyl-1-propenyl, (3-bromo-2-nitrophenyl)thio, (1, 1-dimethylethoxy)carbonyl, [[(1, 1-dimethylethoxy)carbonyl]amino]ethyl, 2-(phenylmethoxy)phenoxy. (1=[1,1′-biphenyl]-4-yl-1-methylethoxy) carbonyl, bromo, (4-bromophenyl)sulfonyl, 1H-benzotriazol-1-yl, [(phenylmethyl)thio]carbonyl, [(phenylmetyl)thio]methyl, 2-methylpropyl, 1,1-dimethylethyl, benzoyl, diphenylmethyl, phenylmethyl, carboxyacetyl, aminocarbonyl, chlorodifluoroacetyl, trifluoromethyl, cyclohexylcarbonyl, cycloheptyl, cyclohexyl, cyclohexylacetyl, chloro, carboxymethyl, cyclopentylcarbonyl, cyclopentyl, cyclopropylmethyl, ethoxycarbonyl, ethyl, fluoro, formyl, 1-oxohexyl, iodo, methyl, 2-methoxy-2-oxoethyl, nitro, azido, phenyl, 2-carboxy benzoyl, 4-pyridinylmethyl, 2-piperidinyl, propyl, 1-methylethyl, sulfo, and ethenyl. Additional examples of 5′ and 3′ blocking groups are known in the art. In some embodiments, the 5′ and/or 3′ blocking groups prevent nuclease degradation of the neuroprotective molecule.

The neuroprotective molecules described herein can be synthesized using any methods known in the art for synthesizing nucleic acids (see, e.g., Usman et al., J. Am. Chem. Soc. 109:7845, 1987; Scaringe et al., Nucleic Acid Res. 18:5433, 1990; Wincott et al., Methods Mol. Biol. 74:59, 1997; and Milligan, Nucleic Acid Res. 21:8783, 1987). These typically make use of common nucleic acid protecting and coupling groups. Synthesis can be performed on commercial equipment designed for this purpose, e.g., a 394 Applied Biosystems, Inc synthesizer, using protocols supplied by the manufacturer. Additional methods for synthesizing the neuroprotective molecules described herein are known in the art. Alternatively, neuroprotective molecules of the invention can be specially ordered from commercial vendors that synthesize oligonucleotides.

Any of the neuroprotective molecules described herein can be tested for activity (e.g., ability to inhibit or decrease protein translation, the ability to induce or increase stress granule formation in a cell, the ability to translocate into a cell in the absence of cell transfection reagents, the ability to treat a neurological disorder associated with neuron death, or the ability to confer upon cells (e.g., neurons) resistance to stress-induced cell death). Methods for detecting or assessing protein translation (e.g., the amount of translation over a specific period of time or rate of protein translation), methods for detecting or assessing the formation of at least one stress granule in a cell, methods of detecting or assessing the cellular uptake, methods of determining treatment of a neurological disorder associated with neuron death in a subject, and methods of determining whether a neuroprotective molecule confers protection against stress-induced cell death are described herein. Additional methods for assessing these activities are known in the art.

### Pharmaceutical Compositions

Also provided herein are pharmaceutical compositions that contain at least one (e.g., one, two, three, or four) of the neuroprotective molecules (e.g., any of the neuroprotective molecules described herein) or C-myc oligonucleotides described herein. Two or more (e.g., two, three, or four) of any of the neuroprotective molecules or C-myc oligonucleotides described herein can be present in a pharmaceutical composition in any combination. The pharmaceutical compositions may be formulated in any manner known in the art.

Pharmaceutical compositions are formulated to be compatible with their intended route of administration (e.g., intravenous, intracranial, ocular, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents such as benzyl alcohol or methyl parabens, chlorobutanol, phenol, ascorbic acid, thimerosal, and the like, antioxidants such as ascorbic acid or sodium bisulfite, chelating agents such as ethylenediaminetetraacetic acid, buffers such as acetates, citrates, or phosphates, and isotonic agents such as sugars (e.g., dextrose), polyalcohols (e.g., manitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating such as lecithin, or a surfactant. Absorption of the neuroprotective molecule or C-myc oligonucleotide can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid: Alza Corporation and Nova Pharmaceutical, Inc.).

Compositions containing one or more of any of the neuroprotective molecules or C-myc oligonucleotides described herein can be formulated for parenteral (e.g., intravenous, intraarterial, intramuscular, intradermal, subcutaneous, or intraperitoneal), intracranial, or ocular administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) neuroprotective molecules or C-myc oligonucleotides (e.g., any of the neuroprotective molecules or C-myc oligonucleotides described herein) will be an amount that treats a neurological disorder associated with neuron death (e.g., amyotrophic lateral sclerosis) in a subject (e.g., a human), decreases the severity, frequency, and/or duration of one or more symptoms of a neurological disorder associated with neuron death (e.g., amyotrophic lateral sclerosis) in a subject (e.g., a human), decreases the rate of neuron death (apoptosis and/or necrosis) in a subject (e.g., a human) having a neurological disorder associated with neuron death, induces or increases stress granule formation in a neuron (e.g., a motor neuron) in a subject having a neurological disorder associated with neuron death (e.g., amyotrophic lateral sclerosis), and/or decreases or inhibits protein translation in a neuron (e.g. a motor neuron) in a subject (e.g., a human) having a neurological disorder associated with neuron death (e.g., as compared to a control subject having the same disease but not receiving treatment or a different treatment, or the same subject prior to treatment). The effectiveness and dosing of any of the neuroprotective molecules or C-myc oligonucleotides described herein can be determined by a health care professional using methods known in the art, as well as by the observation of one or more symptoms of a neurological disorder associated with neuron death in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the neuroprotective molecules or C-myc oligonucleotides described herein per kilogram of the subject's weight (e.g., about 100 ng/kg to about 500 mg/kg: 500 ng/kg to about 1 μg/kg: 1 μg/kg to about 500 mg/kg: about 100 μg/kg to about 500 mg/kg: about 100 μg/kg to about 50 mg/kg: about 10 μg/kg to about 5 mg/kg: about 10 μg/kg to about 0.5 mg/kg: or about 1 μg/kg to about 50 μg/kg). While these doses cover a broad range, one of ordinary skill in the art will understand that therapeutic agents, including the neuroprotective molecules and the C-myc oligonucleotides, vary in their potency, and effective amounts can be determined by methods known in the art. Typically, relatively low doses are administered at first, and the attending health care professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the neuroprotective molecule or C-myc oligonucleotide in vivo.

The pharmaceutical compositions can be included in a container, pack, or dispenser together with instructions for administration.

### Methods of Inducing/Increasing Stress Granule Formation in a Cell

Provided herein are methods of inducing or increasing stress granule formation in a cell that include administering to a cell a neuroprotective molecule (e.g., any of the neuroprotective molecules described herein) or an isolated C-myc oligonucleotide containing the sequence of GGGAGGGTGG GGAGGGTGGGG (SEQ ID NO: 174) (e.g., any of the C-myc oligonucleotides described herein), wherein the neuroprotective molecule or isolated C-myc oligonucleotide is administered in an amount sufficient to induce or increase (e.g., a significant or detectable increase) stress granule formation in the cell. In some embodiments, the increase in stress granule formation is the formation of at least one stress granule in a cell that does not contain stress granules prior to treatment.

Stress granules are formed in the cytosol and nucleus of mammalian cells (e.g., neurons) in response to stress stimuli (e.g., oxidative stress). Stress granules contain both proteins and messenger RNAs (e.g., polyA mRNAs) that are stalled in translation pre-initiation complexes. Stress granules range between 100 nm to 200 nm in size and are not surrounded by a cellular membrane. A variety of proteins have been identified as being present within stress granules and the presence of stress granules can be identified by the localization of one or more (e.g., two, three, or four) of the following proteins/complexes in distinct foci within a cell (e.g., within the cytoplasm or nucleus of a cell): 40S ribosomal subunit, Ago2, AKAP350, APOBEC3G, Ataxin-2/Pbp1, BRF1, Calreticulin, Caprin1, CCAR1, Ccr4, CIRP, CPEB, CUG-BP1, Dcp1/Dcp1a, Dcp2: DDX1, DDX3/Ded1, DICI/DHC1, DIS1, Eap1, Ebs1, Edc1-2, Edc3, eIF2, eIF2B, eIF3, CIF4A, eIF4E, eIF4E-T, eIF4G, cRF1, cRF3, FAK, FAST, FBP/KSRP, FMRP, FXR1P, FXR2P, G3BP, Gbp2, Ge-1/Hedls, Grb7, GW 182, hMex3A, hMex3B, hnRNP A1, hnRNP A3, hnRNP K, hnRNP Q, Hrp1, Htt, Hsp27, HuD, HuR, IP5K, Importin-8, KHC/KLC, Lin28, LINE1 ORFIp, Lsm1. MBNL1, MEX67, MLN51, Musahi, Nrp1, NXF7, p97/NAT1, PABP/Pab1, Pan2/3, Patl. PCBP2, Plakophilin 1/3, PMR1, Pop2/Caf1, Prohibitin 2, PRTB, Pum1, Pum2, RACK1, RBM42, Rap55/Sed6, RCK/Dhh1, RHAU, Roquin, Rpm2, RSK2, Sam68, SERBP1, SGNP, Smaug 1, Staufen, SMN, TDP-43, TDRD3, TIA-1/Pub1, TIA-R/Ngrl, TNRC6B, TRAF2, TTP, Upf1, Upf2, Upf3, Vts1, Xml, YB-1, Ygr250c, and ZBP1. Typically, the presence of stress granules in a cell are detected using microscopy (e.g., immunofluorescence microscopy) using one or more antibodies that recognize the localization one or more of the following proteins in discrete foci in the cell: 40S ribosomal subunit, eIF4E, eIF4G, eIF4A, eIF4B, poly(A) binding protein (Pabp), eIF3, and eIF2. The presence of a stress granule can also be detected by assessment of the level of phosphorylated eIF2a present in the cell (e.g., an increased level of phospho-eIF2a indicates the presence of stress granules in the cell). Additional methods for detecting the formation or presence of stress granules in the cell are known in the art.

The formation of stress granule formation in a cell can be compared to the same cell prior to treatment or can be compared to at least one second cell or a population of cells not receiving treatment or receiving a different treatment. In some embodiments, the cell is treated with the at least one neuroprotective molecule or the C-myc oligonucleotide for at least 2 hours (e.g., at least 6 hours, 12 hours, 16 hours, 20 hours, or 24 hours) before the formation of stress granules is determined in the cell.

An increase in stress granule formation in a cell (e.g., a motor neuron) can be indirectly observed in a subject having a neurological disorder associated with neuron death (e.g., stress-induced motor neuron death) by a decrease (e.g., a detectable decrease) in the rate of the development of at least one symptom of a neurological disorder associated with neuron death or a decrease in the rate of the worsening or exacerbation of at least one symptom (e.g., an increase in the intensity, duration, or frequency of at least one symptom over time) of a neurological disorder associated with neuron death in a subject.

In some embodiments, a C-myc oligonucleotide containing the sequence of SEQ ID NO: 174 is administered to the cell. The C-myc oligonucleotides that can be used in these methods can include at least one modified nucleotide (e.g., any of the modified oligonucleotides described herein). In addition, the C-myc oligonucleotides can contain at least one modification in the phosphate backbone (the phosphodiester linkage between two adjoining nucleotides) (e.g., any of linkages described herein). The C-myc oligonucleotides described herein can also include a 5′ and/or a 3′ blocking group (e.g., a 5′ and/or 3′ blocking group that decreases or inhibits nuclease degradation) (e.g., any of the 5′ and/or 3′ blocking groups described herein or known in the art). In some embodiments, the C-myc oligonucleotides have a total length of between 21 to 50 nucleotides (e.g., 21-30 nucleotides, 30-40 nucleotides, or 40-50 nucleotides).

In various embodiments of these methods, the cell can be a neuron (e.g., a motor neuron), a fibroblast, an epithelial cell, an endothelial cell, or a muscle cell. In any of the methods described herein, the cell is a human cell. In some embodiments of these methods, the cell is in vitro (in tissue culture). In some embodiments of these methods, the cell is in vivo (in a human). In some embodiments, the cell is ex vivo (e.g., a primary human neuron or a primary rat neuron).

The neuroprotective molecule or C-myc oligonucleotide can be administered to the cell by a laboratory worker (a research scientist), a health care professional (e.g., a physician, a physician's assistant, or a nurse), or a subject (e.g., self-administration). In instances where the neuroprotective molecule or C-myc oligonucleotide are administered to a to a cell in vivo (in a subject), the dosing and administration of the neuroprotective molecule or C-myc oligonucleotide can be performed as described below.

### Methods of Decreasing Protein Translation in a Cell

The data in the Examples show that the neuroprotective molecules and the C-myc oligonucleotides described herein inhibit protein translation of mRNA (capped and uncapped mRNAs) by displacing eIF4G/eIF4A from uncapped RNAs and by displacing eIF4F from the m⁷G cap of mRNAs. Accordingly, provided herein are methods of decreasing protein translation in a cell that include administering to a cell a neuroprotective molecule (e.g., any of the neuroprotective molecules described herein) or an isolated C-myc oligonucleotide (e.g., any of the C-myc oligonucleotides described herein), where the neuroprotective molecule or the isolated C-myc oligonucleotide is administered in an amount sufficient to decrease (e.g., a significant or detectable decrease) protein translation in the cell.

In some embodiments, the decrease in protein translation is detected by the amount of protein translation (new proteins translated) over a specific period of time (e.g., at least 1 hour, 2 hours, 4 hours, or 6 hours). In some embodiments, the decrease is protein translation is a decrease in the rate of protein translation in the cell (e.g., as measured by radioisotope labeling (³⁵S) of newly translated proteins or by detection of the biological activity of a newly expressed protein). The decrease in protein translation in a cell can be compared to a control cell or population of cells not receiving the treatment or receiving a different treatment. Additional methods for detecting protein translation are described herein and are known in the art.

An decrease in protein translation in a cell (e.g., a motor neuron) can be indirectly observed in a subject having a neurological disorder associated with neuron death (e.g., stress-induced motor neuron death) by a decrease (e.g., a detectable decrease) in the rate of the development of at least one symptom of a neurological disorder associated with neuron death or a decrease in the rate of the worsening or exacerbation of at least one symptom (e.g., an increase in the intensity, duration, or frequency of at least one symptom over time) of a neurological disorder associated with neuron death in a subject.

In various embodiments of these methods, the cell can be a neuron (e.g., a motor neuron), a fibroblast, an epithelial cell, an endothelial cell, or a muscle cell. In any of the methods described herein, the cell is a human cell. In some embodiments of these methods, the cell is in vitro (in tissue culture). In some embodiments of these methods, the cell is in vivo (in a human). In some embodiments the cell is ex vivo (e.g., a primary human neuron or a primary rat neuron).

The neuroprotective molecule or C-myc oligonucleotide can be administered to the cell by a laboratory worker (a research scientist), a health care professional (e.g., a physician, a physician's assistant, or a nurse), or a subject (e.g., self-administration). In instances where the neuroprotective molecule or C-myc oligonucleotide are administered to a cell in vivo (administered to a subject), the dosing and administration of the neuroprotective molecule or C-myc oligonucleotide can be performed as described below.

Methods of Treating Neurological Disorders Associated with Neuron Death

Also provided herein are methods of treating neurological disorders associated with neuron death (apoptosis and/or necrosis, or stress-induced neuron death). These methods include administering a neuroprotective molecule (e.g., any of the neuroprotective molecules described herein) or an isolated C-myc oligonucleotide (e.g., any of the C-myc oligonucleotides described herein), where the neuroprotective molecule or C-myc oligonucleotide are administered in an amount sufficient to treat the neurological disorder associated with neuron death in the subject.

Neurological disorders associated with neuron death are a group of diseases that are characterized by neuron death (e.g., motor neuron death or stress-induced motor neuron death) and/or have an etiology that involves neuron death (e.g., motor neuron death or stress-induced motor neuron death). Non-limiting examples of neurological disorders associated with neuron death include amyotrophic lateral sclerosis, Alzheimer's disease, Parkinson's disease, Huntington's disease, muscular dystrophy, multiple sclerosis, and stroke. Neuronal death in this group of disorders can be induced by a variety of stress stimuli, including, for example, oxidative stress, excitotoxicity, and neuroinflammation. The data in the Examples herein show that induction of stress granules (e.g., by administration of any of the neuroprotective molecules or C-myc oligonucleotides described herein) mediates an increase in resistance to stress-induced neuronal death. Thus, by virtue of their ability to induce an increase in stress granule formation in neurons (e.g., motor neurons) in a subject, administration of the neuroprotective molecules and the C-myc oligonucleotides described herein can treat a neurological disorder associated with neuron death in a subject. Some of the neuroprotective molecules and C-myc oligonucleotides described herein have the further advantage that they are taken up by neurons (e.g., motor neurons) without the addition of a transfection agent.

Non-limiting symptoms of neurological disorders associated with neuron death include: hyperflexia, weak muscles, twitching, speech problems, breathing problems, swallowing difficulties, loss of memory, confusion, disorientation, difficulty writing, depression, anxiety, social withdrawal, mood swings, aggressiveness, changes in sleeping habits, tremors, bradykinesia, rigid muscles, impaired balance, involuntary facial movements, numbness or weakness in limbs, partial or complete loss of vision, fatigue, dizziness, paralysis on one side of body or face, and headache. A neurological disorder associated with neuronal death can be diagnosed by a health care professional by detecting or observing one or more (e.g., two, three, or four) symptoms (any of the above listed symptoms) in a subject.

In some embodiments, the administering results in a decrease in the number of symptoms of a neurological disorder associated with neuron death (e.g., as compared to the number of symptoms in a subject prior to treatment or to a subject having the same neurological disorder and not receiving treatment or receiving a different treatment). In some embodiments, the administering results in a decrease (e.g., a detectable or observable decrease) in the severity, frequency, or duration of one or more symptoms of a neurological disorder associated with neuron death (e.g., those symptoms listed herein) (e.g., as compared to a subject having the same neurological disorder and not receiving treatment or receiving a different treatment).

In some embodiments, the administering results in a decrease (e.g., a detectable or observable decrease) in the rate of the development of one or more new symptoms of a neurological disorder associated with neuron death in a subject having a neurological disorder associated with neuron death (e.g., as compared to the rate of the development of one or more new symptoms of a neurological disorder associated with neuron death in a subject having the same neurological disorder and not receiving treatment or receiving a different treatment). In some embodiments, the administering results in a decrease (e.g., a detectable or observable decrease) in the rate of worsening or exacerbation of one or more symptoms of a neurological disorder associated with neuron death (e.g., an increase in the severity, frequency, or duration of one or more symptoms of a neurological disorder associated with neuron death over time) (e.g., any of those symptoms described herein) (e.g., as compared to a subject having the same neurological disorder and not receiving treatment or receiving a different treatment).

In some embodiments, the administering results in an increase in stress granule formation in a neuron (e.g., a motor neuron) in a subject that has a neurological disorder associated with neuron death (e.g., as compared to a subject having the same neurological disorder and not receiving treatment or receiving a different treatment). In some embodiments, the administering results in a decrease or inhibition in protein translation in a cell (e.g., a motor neuron) in a subject that has a neurological disorder associated with neuron death (e.g., as compared to a subject having the same neurological disorder and not receiving treatment or receiving a different treatment). In some embodiments, the administering results in a decrease in neuron death or a decrease in the rate of neuron death (e.g., stress-induced motor neuron death) over a period of time (e.g., at least one month, at least one year, or at least five years) (e.g., as compared to a subject having the same neurological disorder and not receiving treatment or receiving a different treatment). A decrease in protein translation in a neuron (e.g., a motor neuron), an increase in stress granule in a neuron (e.g., a motor neuron), or a decrease in the amount or rate of neuron death over time can be indirectly observed by a physician by a decrease (e.g., a detectable or observable decrease) in the rate of the development of at least one new symptom of a neurological disorder associated with neuron death in a subject (e.g., a decrease in further development of at least one additional symptom of the disorder) or a decrease in the rate of exacerbation of at least one symptom (e.g., a decreased rate of increasing frequency, severity, and/or duration of at least one symptom of the neurological disorder associated with neuron death) (e.g., as compared to a subject having the same neurological disorder and not receiving treatment or receiving a different treatment).

Any of the neuroprotective molecules or C-myc oligonucleotides described herein can be used in these methods. In preferred embodiments, the neuroprotective molecule or C-myc oligonucleotide is taken up by a neuron in the subject in the absence of a transfection agent. In some embodiments, the neuroprotective molecule or the C-myc oligonucleotide contains a modified nucleotide (e.g., any of the modified bases and/or sugars described herein). In some embodiments, the neuroprotective molecule or the C-myc oligonucleotide contains at least one modification in its phosphate (phosphodiester) backbone (e.g., any of the moieties described herein that can be used to link two adjoining nucleotides).

The neuroprotective molecule or C-myc oligonucleotide can be administered by a health care professional (e.g., a physician, a physician's assistant, a nurse, or a laboratory or clinic worker), the subject (i.e., self-administration), or a friend or family member of the subject. The administering can be performed in a clinical setting (e.g., at a clinic or a hospital), in an assisted living facility, or at a pharmacy.

The neuroprotective molecule or C-myc oligonucleotide can be administered to a subject that has been diagnosed as having a neurological disorder associated with neuron death. In some non-limiting embodiments, the subject is a man or a woman, an adult, or an adolescent. The subject can have experienced one or more symptoms of the neurological disorder associated with neuron death for at least one year, two years, three years, four years, or five years. The subject can also be diagnosed as having a later or severe form (an advanced stage) of the neurological disorder associated with neuron death.

In some embodiments of any of the methods described herein, the subject is administered at least one (e.g., at least 1, 2, 3, 4, 5, 6, 7, 8, 9, 10, 15, 20, 25, or 30) dose of a composition containing at least one (e.g., one, two, three, or four) of any of the neuroprotective molecules, C-myc oligonucleotides, or pharmaceutical compositions described herein. In any of the methods described herein, the neuroprotective molecule, C-myc oligonucleotide, or pharmaceutical composition (e.g., any of the neuroprotective molecules, C-myc oligonucleotides, or pharmaceutical compositions described herein) can be administered intravenously, intaarterially, subcutaneously, intraperitoneally, intracranially, ocularly, or intramuscularly to the subject. In some embodiments, the neuroprotective molecule, C-myc oligonucleotide, or pharmaceutical composition is administered intracranially, ocularly, or to the spinal fluid.

In some embodiments, the subject is administered the neuroprotective molecule, C-myc oligonucleotide, or pharmaceutical composition (e.g., any of the neuroprotective molecules, C-myc oligonucleotides, or pharmaceutical compositions described herein) and at least one additional therapeutic agent. The at least one additional therapeutic agent can be selected from the group consisting of: mexiletine, phenytoin, baclofen, dantrolene, carbamazepine, corticosteroids, ß-interferons, glatiramer, fingolimod, natalizumab, mitoxantrone, aspirin, tissue plasminogen activator, anti-cholinergics (e.g., benzotropine and trihexyphenidyl), glutamate (NMDA) blocking drugs (e.g., amantadine), riluzole, cholesterase inhibitors (e.g., donepezil, galactamine, and rivastigmine), memantine, levodopa, carbidopa, dopamine agonists (e.g., pramipexole, ropinicole, and apomorphine), monoamine oxidase B inhibitors (e.g., selegiline and vasagiline), catechol O-methyl transferase inhibitors (e.g., tolcapone and entracapone), tetrabenazine, and anti-psychotic drugs (e.g., haloperidol and clozapine). In some embodiments, at least one additional therapeutic agent and at least one neuroprotective molecule or C-myc oligonucleotide (e.g., any of the neuroprotective molecules or C-myc oligonucleotides described herein) are administered in the same composition (e.g., the same pharmaceutical composition). In some embodiments, the at least one additional therapeutic agent and the neuroprotective molecule or C-myc oligonucleotide are administered to the subject using different routes of administration (e.g., at least one additional therapeutic agent delivered by oral administration and the neuroprotective molecule or C-myc oligonucleotide delivered by intravenous administration).

In any of the methods described herein, the neuroprotective molecule, C-myc oligonucleotide, or pharmaceutical composition (e.g., any of the neuroprotective molecules, C-myc oligonucleotides, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different neuroprotective molecules and/or C-myc oligonucleotides are administered in the same composition (e.g., a liquid composition). In some embodiments, the neuroprotective molecule or C-myc oligonucleotide and at least one additional therapeutic agent are administered in the same composition (e.g., a liquid composition). In some embodiments, the neuroprotective molecule or C-myc oligonucleotide and the at least one additional therapeutic agent are administered in two different compositions (e.g., a liquid composition containing the neuroprotective molecule or C-myc oligonucleotide and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to administering the neuroprotective molecule, C-myc oligonucleotide, or pharmaceutical composition (e.g., any of the neuroprotective molecules, C-myc oligonucleotides, or pharmaceutical compositions described herein). In some embodiments, the one or more additional therapeutic agents can be administered to the subject after administering the neuroprotective molecule, C-myc oligonucleotide, or pharmaceutical composition (e.g., any of the neuroprotective molecules, C-myc oligonucleotides, or pharmaceutical compositions described herein). In some embodiments, the one or more additional therapeutic agents and the neuroprotective molecule, C-myc oligonucleotide, or pharmaceutical composition (e.g., any of the neuroprotective molecules, C-myc oligonucleotides, or pharmaceutical compositions described herein) are administered to the subject such that there is an overlap in the bioactive period of the one or more additional therapeutic agents and the neuroprotective molecule or C-myc oligonucleotide (e.g., any of the neuroprotective molecules or C-myc oligonucleotides described herein) in the subject.

In some embodiments, the subject can be administered the neuroprotective molecule, C-myc oligonucleotide, or pharmaceutical composition (e.g., any of the neuroprotective molecules, C-myc oligonucleotides, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, or 10 years). A skilled medical professional may determine the length of the treatment period using any of the methods described herein for diagnosing or following the effectiveness of treatment (e.g., using the methods above and those known in the art). As described herein, a skilled medical professional can also change the identity and number (e.g., increase or decrease) of neuroprotective molecules or C-myc oligonucleotides (and/or one or more additional therapeutic agents) administered to the subject and can also adjust (e.g., increase or decrease) the dosage or frequency of administration of the neuroprotective molecule or C-myc oligonucleotide (and/or one or more additional therapeutic agents) to the subject based on an assessment of the effectiveness of the treatment (e.g., using any of the methods described herein and known in the art). A skilled medical professional can further determine when to discontinue treatment (e.g., for example, when the subject's symptoms are significantly decreased).

### Methods of Identifying a Candidate Translation Inhibitory Nucleic Acid

Also provided herein are methods of identifying a candidate translation inhibitory nucleic acid. These methods include (a) attaching to the 5′ end of a nucleic acid sequence of between 20-50 nucleotides (e.g., 20-30 nucleotides, 30-40 nucleotides, or 40-50 nucleotides) at least four (e.g., at least five, six, or seven) guanosine-containing nucleotides, and (b) determining the level of protein translation in the presence of the molecule produced in (a). A decrease (e.g., a significant or detectable decrease) in the level of protein translation in the presence of the molecule produced in (a) relative to the level of protein synthesis in the absence of the molecule produced in (a) identifies the molecule produced in (a) as a candidate translation inhibitory nucleic acid.

In some embodiments, the nucleic acid sequence of between 20-50 nucleotides contains at least 50% (e.g., at least 55%, 60%, 65%, or 70%) guanosine-containing/cytosine-containing nucleotides. In some embodiments, the sequence of between 20-50 nucleotides is at least 80% identical to a contiguous sequence between nucleotide 1 and nucleotide 50 of a mature human tRNA sequence (e.g., any of the tRNA sequences described or referenced herein, or any mature human tRNA sequence known in the art). The nucleic acid sequence of between 20-50 nucleotides can contain at least one modified nucleotide (e.g., can contain any of the base modifications or sugar modifications described herein). The nucleic acid sequence of between 20-50 nucleotides can also contain at least one modification in the phosphate (phosphodiester) backbone (e.g., any of the linking moieties between two adjoining nucleotides described herein). The molecule produced in (a) can also contain a 5′ and/or 3′ protective group (e.g., a protective group that decreases nuclease degradation of the molecule produced in (a)) (e.g., any of the 5′ or 3′ protective groups described herein or known in the art).

In some embodiments of these methods, the level of protein translation is determined in a cell (e.g., a fibroblast, a neuron (e.g., a motor neuron), an endothelial cell, an epithelial cell, or a muscle cell) (e.g., a cell in vitro). In some embodiments, the level of protein translation is determined in a cell lysate (e.g., a reticulocyte lysate). In some embodiments of all of the above methods, the cell is a human cell.

The level of protein synthesis in these methods can be the total amount of protein translation that occurs over a specific period of time (e.g., at least 2 hours, 6 hours, 12 hours, 16 hours, 20 hours, or 24 hours) or the rate of protein synthesis. Methods for measuring or detecting protein translation are described herein. Additional methods for measuring or detecting protein synthesis are known in the art.

Candidate translation inhibitory nucleic acids identified in these methods can be further modified by the incorporation of modified nucleotides (e.g., modified bases and/or sugars), by introducing a modification in the phosphate (phosphodiester) backbone (e.g., introduction of one of the linking moieties described herein), and/or by adding a 5′ and/or 3′ blocking group to the molecule produced in (a), and further tested to determine the molecule's ability to decrease protein translation.

### EXAMPLE

The invention is further described in the following examples, which do not limit the scope of the invention described in the claims.

### Example 1. Endogenous 5′-tiRNAs Inhibit Translation

Experiments were performed in order to determine whether natural 5′- and 3′-tRNA fragments (tiRNAs) purified from angiogenin-treated U2OS cells would inhibit translation of uncapped luciferase transcripts in rabbit reticulocyte lysate (RRL). The following methods were used to perform these experiments.

### Tissue Culture and Cell Treatment

U2OS cells were maintained at 37ºC in a CO₂ incubator in Minimal Essential Medium (DMEM) supplemented with 10% fetal bovine serum (FBS) (Sigma) and 1% of penicillin/streptomycin (Sigma). The cells were treated with angiogenin (0.5 μg/mL) as described in Kedersha et al., Methods Enzymol. 448:521-552, 2008.

Isolation of tiRNAs

Extraction of tiRNAs from angiogenin-treated U2OS cells was done as previously described (Yamasaki et al., J. Cell Biol. 185:35-42, 2009).

In Vitro Translation of mRNA Reporters in Rabbit Reticulocyte Lysates (RRLs)

The Flexi Rabbit Reticulocyte Lysate (RRL) System (Promega) was used for the in vitro analysis of mRNA translation according to the manufacture's recommendations with some modifications. In all cases, translation reactions (10 μl final volume) contained 70% of reticulocyte lysates or mixtures of RRL supplemented with 20% of microccocal nuclease-treated U2OS extract (RRL+20% U2OS lysate) and 8 units of RNasin Ribonuclease Inhibitor (Promega). Fifty ng of uncapped Firefly RNA (Promega) were used per translation reaction. One hundred picomoles of control RNAs or tiRNAs were added to translation reactions, mixed, and incubated for 30 minutes at 30° C. The reactions were stopped at 4° C. and the activity of Firefly luciferase was measured using ⅕ of the translation reactions with the Luciferase Assay System (Promega) according to the manufacture's recommendations (2 second measurement delay followed by 10 second measurement read).

### Northern Blot Analysis

RNA was separated on either a 10% or 15% TBE-UREA Gel (Invitrogen) and transferred to Supercharge nylon transfer membranes (Nytran SPC; 0.45 μm pore size; Whatman). The RNA on the membranes was UV cross-linked using a FB-UVXL-1000 cross-linker (Fisher Scientific). The membranes were then pre-hybridized in hybridization solution (5×SSC, 20 mM Na₂HPO₄ pH 7.4, 7% SDS, 1×Denhardt's) at 47ºC for 30 minutes and then hybridized with the 32P 5′-end labeled oligonucleotide probe (ME8: 5′CTTTATGTTTTTGGCGTCTTCCATCTCGAGGC3″: SEQ ID NO: 175) overnight at 47° C. The membranes were washed twice for 15 minutes at 47° C. with 3×SSC/5% SDS and once with 1×SSC/1% SDS for 15 minutes at room temperature. After washing, RNA signals were detected by autoradiography on X-Omat film (Kodak) after overnight exposure at −80° C.

### Results

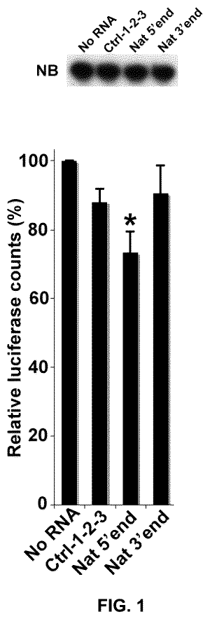

Uncapped Firefly luciferase mRNA (Promega) was translated in RRL in the presence of a control RNA mix (Ctrl 1-2-3; derived from Piwi-interacting RNAs (piRNAs) or randon sequences), natural 5′-tiRNAs (Nat 5′ end), or natural 3′-tiRNAs (Nat 3′end) using the methods described above. Luciferase expression was compared to the level of expression in the absence of RNA (No RNA=100%). The data from these experiments show that natural 5′-tiRNAs (but not 3′-tiRNAs) gel-purified from angiogenin-treated U2OS cells significantly inhibit translation of uncapped luciferase transcripts in RRLs (FIG. 1, bottom). Northern blotting analysis confirmed that the luciferase transcripts were not degraded during the experiment (FIG. 1, top).

### Example 2. Selected Synthetic 5′-tiRNAs are Potent Inhibitors of Translation

Since natural tiRNA preparations are contaminated with ribosomal and mRNA fragments, experiments were performed to compare the activity of synthetic 5′-end phosphorylated 5′-tiRNAs (5′-tiRNAs) and unphosphorylated 3′-tiRNAs (3′-tiRNAs) (Emara et al., J. Biol. Chem. 285:10959-10968, 2010) in the above-described in vitro RRL translation assay. The synthetic tiRNAs used in this study were synthesized by Integrated DNA Technology and were at least 95% homogenous. The synthetic sequences used in these experiments were:

Ctrl1:
(SEQ ID NO: 1)
5′-UGA AGG GUU UUU UGU GUC UCU AUU UCC UUC-3′
(piR006650);
Ctrl2:
(SEQ ID NO: 2)
5′-phospho-UGU GAG UCA CGU GAG GGC AGA AUC
UGC UC-3′
(piR58620);
Ctrl3:
(SEQ ID NO: 3)
5′-phospho-GCA UUC ACU UGG AUA GUA AAU CCA
AGC UGA A-3′
(random);
5′Ala:
(SEQ ID NO: 4)
5′-Phospho-GGG GGU GUA GCU CAG UGG UAG AGC
GCG UGC-3′;
5′-Val:
(SEQ ID NO: 5)
5′-phospho-GUU UCC GUA GUG UAG UGG UUA UCA
CGU UCG CC-3′;
5′-Gly-GCC:
(SEQ ID NO: 6)
5′-Phospho-GCA UGG GUG GUU CAG UGG UAG AAU
UCU CGC-3′;
5′-GlyCCC:
(SEQ ID NO: 7)
5′-phospho-GCG CCG CUG GUG UAG UGG UAU CAU
GCA AGA U-3′;
5′-Pro:
(SEQ ID NO: 8)
5′-Phospho-GGC UCG UUG GUC UAG GGG UAU GAU
UCU CGG-3′;
5′-Gln:
(SEQ ID NO: 9)
5′-Phospho-GGU UCC AUG GUG UAA UGG UUA GCA
CUC UG-3′;
5′-Cys:
(SEQ ID NO: 10)
5′-Phospho-GGG GGU AUA GCU CAG UGG UAG AGC
AUU UGA-3′;
5′-Met:
(SEQ ID NO: 11)
5′-Phospho-GCC UCG UUA GCG CAG UAG GUA GCG
CGU CAG U-3′;
5′-Met-I:
(SEQ ID NO: 12)
5′-Phospho-AGC AGA GUG GCG CAG CGG AAG CGU
GCU GG-3′;
3′-Ala:
(SEQ ID NO: 13)
5′-CUU AGC AUG CAC GAG GCC CCG GGU UCA AUC
CCC GGC ACC UCCA-3′;
3′-Arg:
(SEQ ID NO: 14)
5′-GGA UCA GAA GAU UGA GGG UUC GGG UCC CUU
CGU GGU CG-3′;
3′-Gly:
(SEQ ID NO: 15)
5′-CCA CGC GGG AGG CCC GGG UUC GAU UCC CGG
CCA AUG CA-3′;
3′-Gln:
(SEQ ID NO: 16)
5′-GAC UCU GAA UCC AGC GAU CCG AGU UCA AAU
CUC GGU GGA ACC U-3′;
and
3′-Pro:
(SEQ ID NO: 17)
5′-UUA GGA UGC GAG AGG UCC CGG GUU CAA AUC
CCG GAC GAG CCC-3′.

The levels of luciferase mRNA were also determined in these experiments using Northern blot using the methods described above.

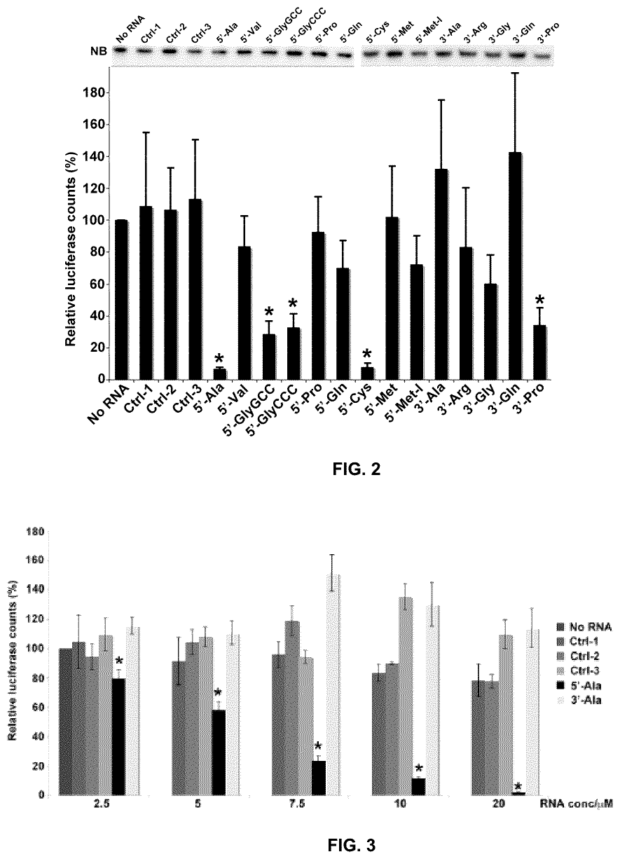

The data from these experiments show that although several tiRNAs significantly inhibit translation, 5′-tiRNA^{Ala} and 5′-tiRNA^{Cys} are particularly potent translational inhibitors (FIG. 2, bottom). Northern blotting analysis confirmed that luciferase transcripts were not degraded under these conditions (FIG. 2, top). Data from a dose response experiment comparing 5′- and 3′-tiRNA^{Ala} shows that 5′-tiRNA^{Ala} represses translation at low micromolar concentrations (FIG. 3). Since the abundance of tiRNAs in angiogenin- or ansenite-treated cells is ˜10-fold less than that of tRNA (intracellular tRNA concentration is estimated to be ˜10-200 μM depending on cell type), similar inhibitory concentrations of tiRNAs are likely found in stressed cells. These calculations indicate that the ratio of tiRNAs to mRNAs in stressed cells is ˜8, which is consistent with their ability to repress translation in vivo.

Additional experiments were performed to determine whether 5′-tiRNA^{Ala} and 3′-tiRNA^{Ala} inhibit the translation of capped luciferase transcripts. These experiments were performed using the RRL translation assay described above with 10 nm of capped Firefly mRNA. To prepare capped Firefly luciferase mRNA, 2 μg of commercial uncapped Firefly luciferase mRNA (Promega) was capped by Vaccinia Virus Capping enzyme using ScriptCap m⁷G Capping System (EPICENTRE Biotechnologies) according to the manufacture's recommendations. Capped mRNA was purified by standard ethanol precipitation and quantified by spectrophotometry (Beckman DU 640 instrument).

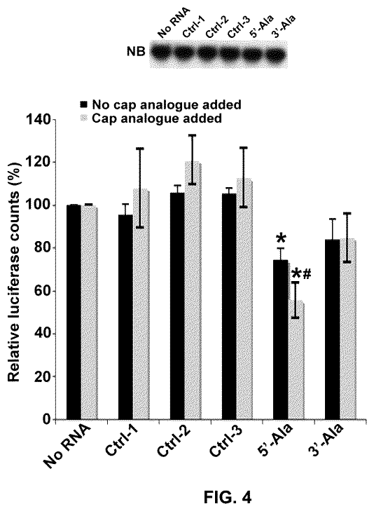

The data from these experiments show that 5′-tiRNA^{Ala} (but not 3′-tiRNA^{Ala}) also significantly inhibits the translation of capped luciferase transcripts (FIG. 4, bottom), and that the addition of a cap analogue that inhibits cap-dependent translation significantly increases the relative translational repression. The cap analogue reduces the basal level of capped mRNA translation by ˜2.5-fold. Northern blotting analysis (performed as described above) confirmed that luciferase transcripts are not degraded under these conditions (FIG. 4, top). Since translation of uncapped mRNAs or capped mRNAs in the presence of cap analogue depends on eIF4G binding to the 5′-terminus of mRNA (De Gregorio et al., RNA 4:828-836, 1998), these data suggest that 5′-tiRNA^{Ala} interferes with some functions of eIF4G (e.g., binding to mRNA and/or to eIF4E).

### Example 3. Encephalomyocarditis Virus Internal Ribosomal Entry Site (EMCV IRES)-Mediated Translation

Additional experiments were performed in order to determine the role of eIF4G in tiRNA-mediated translational repression. In these experiments, the translation of capped or uncapped bicistronic reporter transcripts encoding an upstream Firefly luciferase and a downstream EMCV IRES-driven Renilla luciferase in the absence or presence of tiRNAs was quantitated. These experiments were performed using the methods generally described above with the modifications described below.

RNA transcripts for use in the RRL assay were prepared by first linearizing the bicistronic reporter plasmid (pF/R) (Bochkov et al., BioTechniques 41:283-284, 286, 288, 2006) by digestion with HpaI (New England Biolabs), separating the cut plasmids on 1% agarose gel, and then purifying the plasmids from the gel with QIAquick Gel Extraction Kit (Qiagen). Riboprobe T7 in vitro transcription System (Promega) was used to synthesize pF/R RNA using T7 RNA Polymerase according to the manufacturer's recommendations. Subsequently, in vitro transcribed RNA was purified using Trizol (Invitrogen) extraction followed by isopropanol precipitation. Purified RNA was analyzed for purity on a formaldehyde gel and quantified by spectrophotometry (Beckman DU 640). One hundred nanograms of pF/R bicistronic mRNA were used per translation reaction and the activities of Firefly and Renilla luciferase were measured using ⅕ of translation reactions with the Dual-Luciferase Reporter Assay System (Promega).

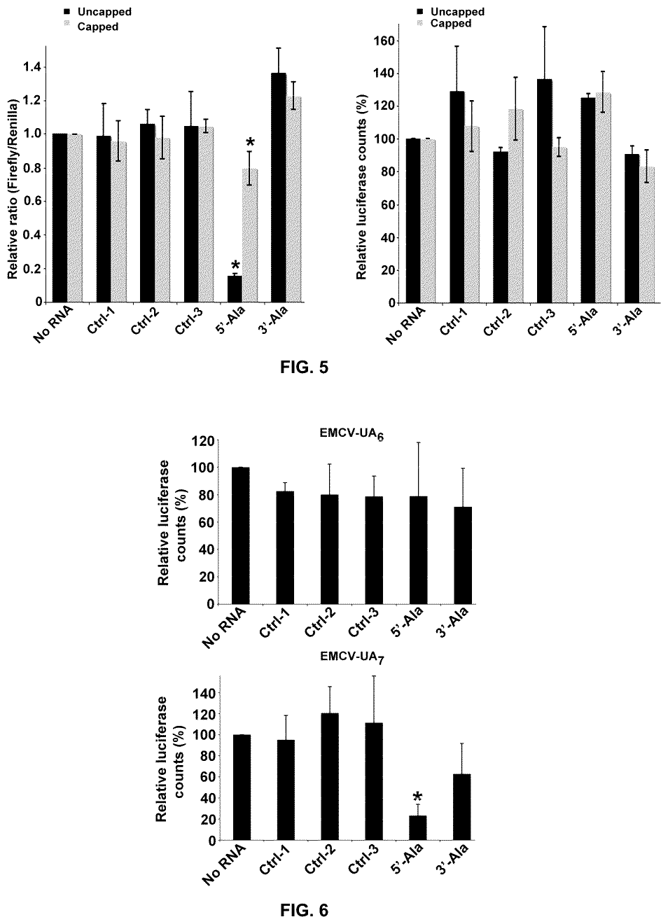

The data from these experiments show that 5′- (but not 3′-tiRNA^{Ala}) significantly reduces the Firefly/Renilla luciferase ratio (FIG. 5, left panel) without affecting the translation of Renilla luciferase (FIG. 5, right panel). This result indicates that 5′-tiRNA^{Ala} does not prevent the recruitment of eIF4G to the EMCV IRES. As this result was not consistent with the ability of tiRNAs to preferentially inhibit the eIF4G-dependent translation of uncapped RNA, additional experiments were performed using an EMCV IRES variant that has reduced affinity for eIF4G and less efficient translation initiation (Bockhov et al., supra; Kaminski et al., EMBO J. 13:1673-1681, 1998).

The recruitment of eIF4G to the EMCV IRES is critically dependent upon binding to the J-K domain which includes a UA₆ bifurcation loop located upstream from the AUG translation start site. A variant bifurcation loop (UA₇) reduces the binding of eIF4G to reduce translation efficiency and infectivity of encephalomyocarditis virus. Additional experiments were performed to compare the ability of control RNAs and tiRNAs to inhibit the translation of luciferase from monocistronic luciferase constructs expressing the wild type (UA₆) or mutant (UA₇) EMCV IRES. The experiments were performed using the methods generally described above with the modifications described below.

The pCDNA3-EMCV-R-luc (EMCV-UA₆) plasmid used in these experiments was prepared by amplifying a fragment of the pF/R plasmid encoding EMCV IRES by PCR and subcloning the fragment into pCDNA3 vector using BamHI and XhoI sites. The ORF of Renilla luciferase was amplified by PCR and inserted into pCDNA3-EMCV construct using XhoI and XbaI sites.

RNA transcripts for use in the RRL assay were prepared by first linearizing the plasmids pCDNA3-EMCV-R-luc (EMCV-UA₆), pRL-5boxB, and pEMCV-RL-5boxB by digestion with XbaI (New England Biolabs), and separating and purifying the linearized plasmids as described above. Riboprobe T7 in vitro transcription System (Promega) was used to synthesize corresponding RNA and the RNA was purified as described above. Luciferase expression was also measured as described above.

The resulting data show that 5′-tiRNA^{Ala} does not inhibit translation from the wild type (UA₆) IRES (FIG. 6, upper panel), but potently inhibits translation from the mutant (UA₇) IRES (FIG. 6, lower panel). These data suggest that 5′-tiRNA^{Ala}-mediated translational repression is a function of the strength of the eIF4G-RNA interaction. Recombinant eIF4E inhibits the translation of uncapped mRNAs by ˜50%. This is presumably due to the fact that eIF4E:eIF4G complexes do not efficiently bind to the 5′-end of uncapped mRNA. The ability of tiRNAs to inhibit EMCV-IRES (UA₇) makes it unlikely that eIF4E is the primary target for translational repression. To further support this conclusion, additional experiments were performed to determine whether recombinant eIF4E competitively inhibits tiRNA^{Ala}-induced translational repression in RRL. These experiments were performed as described above.

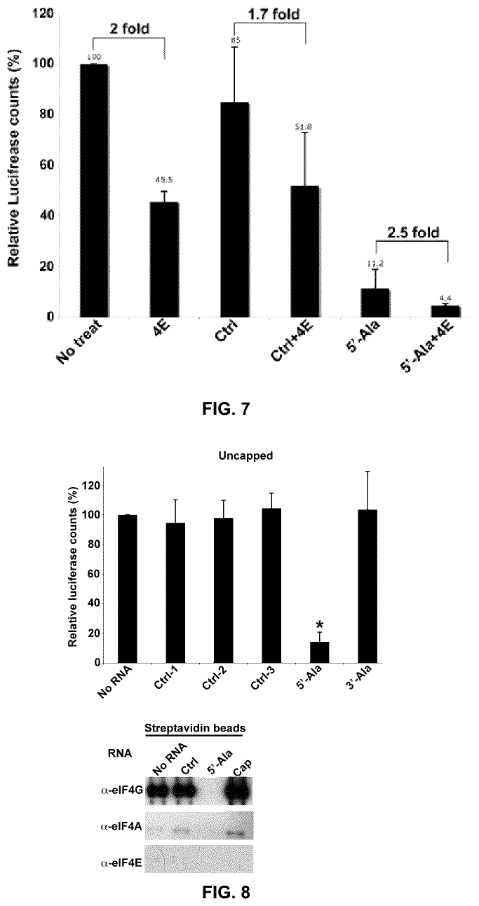

The resulting data show that eIF4E does not competitively inhibit tiRNA^{Ala}-induced translational repression (FIG. 7). In fact, the combination of tiRNA^{Ala} and recombinant eIF4E results in 2.5-fold more inhibition than tiRNA^{Ala} alone: a result suggesting that these two factors act independently to inhibit translation.

### Example 4. 5′-tiRNAs Displace eIF4G

Additional experiments were performed to determine whether 5′-tiRNAs displace eIF4G from RNA. In these experiments, biotin-tagged capped or uncapped luciferase transcripts were added to heterologous (80% RRL+20% U2OS) lysates containing control RNA or tiRNAs in the above described in vitro translation assays. After streptavidin pull down, the reporter RNA-bound proteins were quantitated by immunoblotting. These experiments were performed as described above with the additions or modifications described below.

Poly-A biotinylated mRNAs used for streptavidin pull-down assays were prepared by polyadenylating capped or uncapped pRL-5boxB RNAs using Poly(A) Polymerase Tailing Kit (EPICENTRE Biotechnologies) according to the manufacturer's recommendation, but in the presence of 10 nM biotin-ATP (PerkinElmer).

Streptavidin agarose beads (Invitrogen) (40 μL per sample) were washed twice with RNAse-free Biotin Binding Buffer (10 mM Tris-HCl, pH 7.2, 100 mM NaCl, 1 mM EDTA, 0.1% NP-40). Five hundred pmoles of biotinylated RNAs were added to streptavidin beads and incubated for 1 hour at room temperature with rotation in 0.75 mL of Biotin Binding Buffer. After incubation, immobilized biotinylated RNA-streptavidin complexes were washed twice with RNAse-free Wash Buffer II (15 mM Tris HCl, pH 7.2, 750 mM NaCl, 1 mM EDTA, 0.1% NP-40) and once with ice-cold RNAse-free Wash Buffer I (15 mM Tris HCl, pH 7.2, 150 mM NaCl, 1 mM EDTA, 0.1% NP-40) at room temperature to remove unbound RNA. Pre-cleared U20S lysates (200 μl of lysate per reaction, corresponding to 20% of lysate (in Lysis Buffer: 50 mM Tris-HCl, pH 7.2, 100 mM NaCl, 1 mM EDTA, 0.5% NP-40, with protease inhibitor cocktail “Complete.” Roche) prepared from one 15-cm dish of 80% confluent U2OS cells) were added to the biotinylated RNA-streptavidin bead complexes, incubated for 2 hours at 4° C. with rotation, and washed 3 times with Wash Buffers (15 mM Tris HCl, pH 7.2, 1 mM EDTA, 0.1% NP-40) containing different NaCl concentrations (0.1 M. 0.3 M or 0.5 M). Proteins were eluted using 60 μl of 1×SDS PAGE Loading Buffer.

For pull-down of biotinylated polyA mRNAs, 200 ng of capped or uncapped pRL-5boxB mRNAs were used for in vitro translation in rabbit reticulocyte lysate supplemented with 20% U2OS extract under the conditions described above. This heterologous in vitro translation system (RRL with cell extract) is capable of translating mRNAs with similar to Flexi Rabbit Reticulocyte Lysate System efficiency, but allows for the detection of eIF4G (human) by Western Blotting (the antibodies used do not detect eIF4G of rabbit origin). One hundred picomoles of control RNAs or tiRNAs were added to translation reactions. After completion of translation, streptavidin agarose beads (Invitrogen) (40 μL per sample) were added to reactions and incubated at 4° C. for 30 minutes. Streptavidin beads were precipitated by centrifugation (1000 rpm, 5 minutes), the supernatants were removed, and the beads were washed once with ice-cold Wash buffer (15 mM Tris HCl, pH 7.2, 150 mM NaCl, 1 mM EDTA, 0.1% NP-40). The resulting mRNA-protein complexes were eluted from the beads using 60 μl of 1×SDS PAGE Loading Buffer.

For Western blotting, proteins were separated on a 4-20% gradient SDS-PAGE (Invitrogen) and transferred to nitrocellulose filter membranes (0.45 μm pore size; Invitrogen). The membranes were blocked with 5% normal horse serum (NHS) in 1×TBS at room temperature for 1 hour and incubated with protein-specific antibodies in TBS containing 5% NHS overnight at 4° C. The membranes were washed three times with 1×TBS containing 0.1% Tween-20 and incubated with secondary antibodies conjugated with horseradish peroxidase (GE Healthcare). After washing, the specific proteins were detected using the Super Signal chemiluminescent detection system (Pierce) and autoradiography on X-Omat film (Kodak).

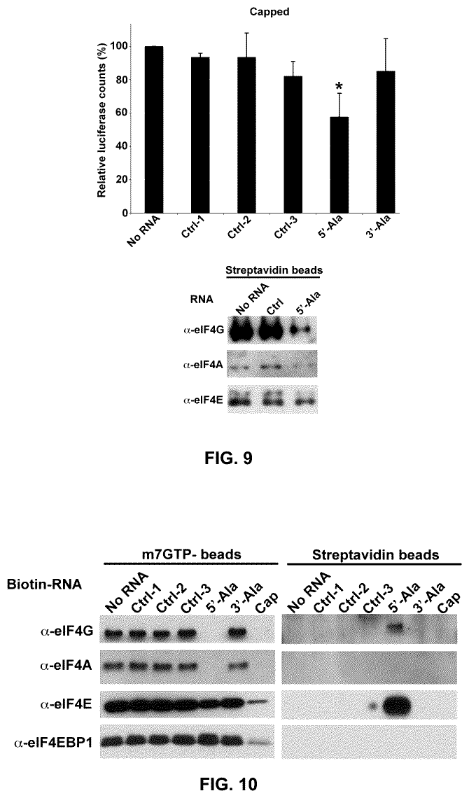

The data from these experiments show that uncapped RNA is bound to eIF4G, but not eIF4E (FIG. 8, lower panel): these results are consistent with the ability of eIF4G to bind to the 5′-end of uncapped transcripts. 5″-tiRNA^{Ala}, but not control RNAs or the cap analogue, quantitatively displaces eIF4G from these uncapped transcripts (FIG. 8, lower panel). In contrast, biotin-tagged capped mRNA is bound to eIF4G and eIF4E (FIG. 9, lower panel). 5′-tiRNA^{Ala} only partially displaces eIF4G (˜75% displacement) suggesting that stabilization of eIF4G conferred by cap-bound eIF4E may inhibit tiRNA-induced displacement (FIG. 9). 5′-tiRNAs also modestly displace eIF4E suggesting that its effect on eIF4G may reduce the affinity of eIF4E:cap interactions.

### Example 5. 5′-tiRNAs Displace eIF4F from the m′G Cap

Additional experiments were performed in order to determine whether 5′-tiRNAs affect eIF4F:cap interactions. For these experiments, eIF4E complexes were assembled (e.g., eIF4F (eIF4E:eIF4G) and eIF4E:4E-BP1) on m⁷GTP-Sepharose from U2OS cell lysates. The Sepharose-bound complexes were incubated with 3′-end biotinylated control or tiRNAs before analyzing retained components of the eIF4E-containing complexes using Western blotting. The methods used to perform these experiments are described in detail below.

### 7-Methyl GTP Sepharose Chromatography

A 7-methyl-GTP-Sepharose 4B (m⁷GTP-Sepharose, GE Healthcare) suspension was washed twice with ice-cold RNAse-free Buffer A (15 mM Tris HCl, pH 7.0, 100 mM NaCl, 1 mM EDTA) to remove sodium azide. U2OS cells were grown until 70-80% confluence in 15-cm dishes under standard conditions, and then collected by scraping with Lysis Buffer (50 mM Tris-HCl, pH 7.2, 100 mM NaCl, 1 mM EDTA, 0.5% NP-40) supplemented with protease inhibitors (Protease Inhibitor Cocktail “Complete,” Roche) into Eppendorf tubes followed by tumbling at 4ºC for 15 minutes. Cell debris and nuclei were removed by centrifugation (20 minutes, 13200 rpm, 4° C.), the cytoplasmic fraction (supernatant) was applied to pre-washed m⁷GTP-Sepharose, and incubated for 1 hour at 4° ° C. Typically, 10-20 μl of m⁷GTP-Sepharose suspension was used per sample of synthetic RNA (50 or 100 pmoles). After incubation, the m′GTP-Sepharose was washed three times with Lysis Buffer and m⁷GTP-bound protein complexes were divided into equal parts. Synthetic RNAs (50 or 100 pmoles, final concentrations 20-40 nM) were added to the complexes and incubated for 1 hour at 4° C. Unbound proteins were removed by washing once with Lysis Buffer and the proteins bound to m′GTP-Sepharose were eluted with 60 μl of 1×SDS PAGE Loading Buffer. Eluted proteins were analyzed by Western Blotting using protein-specific antibodies.

Goat polyclonal anti-eIF3b, goat polyclonal anti-eIF4A, rabbit polyclonal anti-eIF4G, mouse monoclonal anti-eIF4E were purchased from Santa Cruz Biotechnology. Rabbit polyclonal anti-eIF4EBP1 was purchased from Cell Signaling. Anti-mouse, anti-goat, and anti-rabbit secondary antibodies conjugated with horseradish peroxidase (HRP) were purchased from GE Healthcare.

The resulting data show that although control RNAs did not displace initiation factors from m⁷GTP-Sepharose, 5′-tiRNA^{Ala} (but not 3′-tiRNA^{Ala}) completely displaces eIF4G and eIF4A, and partially displaces (˜50%) eIF4E from the beads (FIG. 10, left panel). In contrast, 4E-BP1 is not displaced from the beads, suggesting that the retained eIF4E is complexed with 4E-BP1. Cap analogue displaces both eIF4F and eIF4E:4E-BP1 from m⁷GTP-Sepharose (FIG. 10, left panel; cap). The supernatants containing displaced initiation factors were collected and streptavidin beads were used to capture biotin-RNA oligonucleotides and their associated proteins. Western blot analysis reveals that biotin-5′-tiRNA^{Ala} is in a stable complex with eIF4G and eIF4E (FIG. 10, right panel). These data suggest that 5′-tiRNA^{Ala} binds, directly or indirectly, to an eIF4E:eIF4G complex and causes its disassociation from m⁷GTP-Sepharose in U2OS lysate much more efficiently than it displaces eIF4G from capped mRNA in RRL (FIG. 9). Similar results were obtained using heterologous lysate (80% RRL+20% U2OS extract, FIG. 11), suggesting that this difference is not conferred by the nature of the lysate.

### Example 6. Structure/Function Analyses of tiRNAs

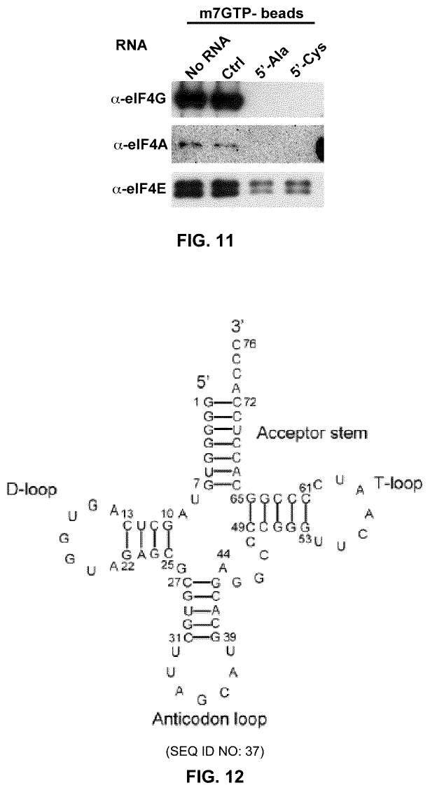

tRNA^{Ala} and tRNA^{Cys} are the only human tRNAs with terminal oligo-guanine (TOG) motifs (4-5 guanine residues) at their 5′ ends (up-to-date alignments for H. sapiens tRNAs can be found at the Lowe Lab website at the address: lowelab.ucsc.edu/GtRNAdb/Hsapi19/Hsapi19-align.html) (see, secondary structure of tRNA^{Ala}, FIG. 12). This structural feature in tRNA^{Ala} and tRNA^{Cys} is extremely well-conserved among all vertebrates, insects, and worms (>95% of all tRNA^{Ala} and tRNA^{Cys} genes), well conserved in plants (>95% of all tRNA^{Ala} genes), and less conserved in fungi and protozoa (<50% of all tRNA^{Ala} genes). In contrast, 5′-TOG motifs are extremely rare in all other tRNAs.

Additional experiments were performed to determine whether these 5′-TOG motifs are required for translation repression. In these experiments, the ability of truncation and substitution mutants to inhibit translation was determined using the RRL translation assays described herein using Firefly luciferase mRNAs. The specific tiRNAs and control oligonucleotides used in these experiments were synthesized and purified by Integrated DNA Technology (at least 95% homogenous), and are listed below.

Ctrl1:
(SEQ ID NO: 1)
5′-UGA AGG GUU UUU UGU GUC UCU AUU UCC UUC-3′
(piR006650)
Ctrl2:
(SEQ ID NO: 2)
5′-phospho-UGU GAG UCA CGU GAG GGC AGA AUC
UGC UC-3′
(piR58620)
Ctrl3:
(SEQ ID NO: 3)
5′-phospho-GCA UUC ACU UGG AUA GUA AAU CCA
AGC UGA A-3′
(random)
5′Ala:
(SEQ ID NO: 4)
5′-phospho-GGG GGU GUA GCU CAG UGG UAG AGC
GCG UGC-3′
U4G:
(SEQ ID NO: 18)
5′-phospho-UGG GGU GUA GCU CAG UGG UAG AGC
GCG UGC-3′
4G:
(SEQ ID NO: 19)
5′-phospho-GG GGU GUA GCU CAG UGG UAG AGC
GCG UGC-3′
UU3G:
(SEQ ID NO: 20)
5′-phospho-UUG GGU GUA GCU CAG UGG UAG AGC
GCG UGC-3′
3G:
(SEQ ID NO: 21)
5′-phospho-G GGU GUA GCU CAG UGG UAG AGC
GCG UGC-3′
GG-UU:
(SEQ ID NO: 22)
5′-phospho-GGG GGU GUA GCU CAG UUU UAG AGC
GCG UGC-3′
27 mer:
(SEQ ID NO: 23)
5′-phospho-GGG GGU GUA GCU CAG UGG UAG AGC
GCG-3′
24 mer:
(SEQ ID NO: 24)
5′-phospho-GGG GGU GUA GCU CAG UGG UAG
AGC-3′
21 mer:
(SEQ ID NO: 25)
5′-phospho-GGG GGU GUA GCU CAG UGG UAG-3′

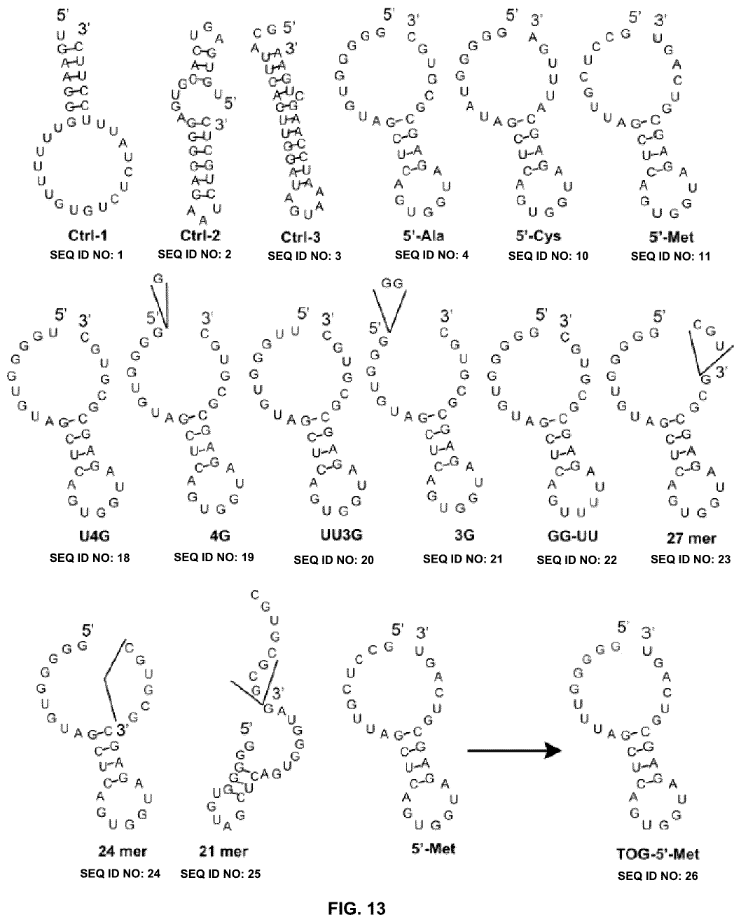

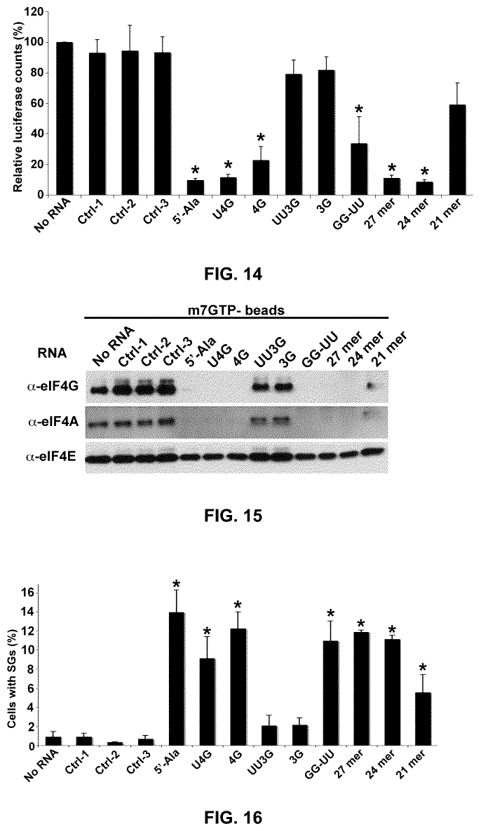

To determine whether the 5′-TOG structural feature is required for translational repression the ability of truncation and substitution mutants of tiRNAs (FIG. 13) to inhibit the translation of reporter mRNA in RRL was determined. These data show that while 5′-tiRNA^{Ala} mutants with a singly truncated (4G) or G to U substituted (U4G) guanine residue retain their activity, mutants with doubly truncated (3G) or GG to UU substituted (UU3G) guanine residues are completely inactive (FIG. 14). Thus, at least four guanines at or near to the 5′-end of tiRNAs are absolutely required for translational repression. In contrast, substitution of two invariant guanine residues within the D-loop region (GG to UU) does not prevent translational repression. RNAfold predicts that 5′-tiRNAs are composed of a single stranded 5′-TOG motif followed by a stem-loop structure resembling the D-loop of tRNA, followed by a single stranded 3′ region (FIGS. 12 and 13). Truncations from the 3′ end of 5′-tiRNA^{Ala} retain their activity until the region of the tRNA D-loop is encroached upon (i.e., 21-mer; see, FIG. 14) suggesting that the structure formed by the D-loop region may contribute to translational repression.

Additional experiments were performed to determine whether the ability of the 5′-tiRNA^{Ala} mutants to inhibit mRNA translation closely correlates with their ability to displace eIF4F from m⁷GTP-Sepharose, or their ability to induce stress granule assembly (when transfected into U2OS cells). The m⁷GTP-Sepharose experiments were performed as described above using the 5′-tiRNA^{Ala} mutants described herein. The transfection of U20S cells and the methods for determining stress granule assembly are described below.

### Cell Transfections

Cells were transfected with the RNA oligonucleotides using Lipofectamine 2000 (Invitrogen). Before transfection, RNA-complexes were pre-incubated in serum-free medium (Opti-MEM medium, Invitrogen) for 20 minutes at room temperature. U2OS cells (1×10⁵/well) were plated in 24-well plates for 24 hours, and then transfected with 750 nM synthetic tiRNAs using 2.5 μl Lipofectamine 2000.

### Immunofluorescence Microscopy and Quantification of Stress Granules

Cells (1×10⁵) were seeded onto coverslips (Fisher Scientific) and were transfected with synthetic tiRNAs using Lipofectamine 2000 24-hours later (Invitrogen). After 7 hours, the cells were fixed in 4% para-formaldehyde for 15 minutes and permeabilized using 100% chilled methanol for 10 minutes. The cells were rinsed several times with PBS and incubated overnight with blocking buffer (5% normal horse serum in PBS containing 0.02% sodium azide) at 4° C. An appropriate primary antibody diluted in blocking buffer (1:200 for anti-eIF3b, anti-eIF4G, and anti-G3BP antibody) was then added to the cells and incubated for 1 hour at room temperature or overnight at 4° C. (Kedersha et al., Methods Enzymol. 448:521-522, 2007). The cells were washed three times with PBS and incubated with the appropriate secondary antibodies (Jackson Immunoresearch, ML grade) diluted 1:200 in blocking buffer containing 0.5 μg/ml Hoechst 33258 dye (Molecular probes) for 1 hour at room temperature. After washing with PBS, the cover slips were mounted in polyvinyl mounting medium, and the cells were viewed and photographed with an Eclipse E800 (Nikon) microscope equipped with a digital camera (CCD-SPOT RT: Diagnostic Instrument) using 60× oil immersion objective lens. The images were merged and analyzed using Adobe Photoshop (v. 10).

### Quantification of Stress Granules

U20S cells (1×10⁵) were seeded onto coverslips (Fisher Scientific) and were transfected 24 hours later with the indicated RNA oligonucleotides (final concentration of 750 nM) using Lipofectamine 2000. After 7 hours, the cells were subjected to immunofluorescence microscopy as described above. The coverslips were coded and all quantifications were done blindly and repeated at least twice. The percentage of cells with stress granules was quantified by counting 200-350 cells/experiment.

The resulting data show that the ability of 5′-tiRNA^{Ala} and its mutants to inhibit mRNA translation closely correlates with their ability to displace eIF4F from m⁷GTP-Sepharose (FIG. 15). In addition, the ability of 5′-tiRNA^{Ala} and its mutants to inhibit mRNA translation closely correlates with their ability to induce the assembly of stress granules when transfected into U20S cells (FIG. 16).

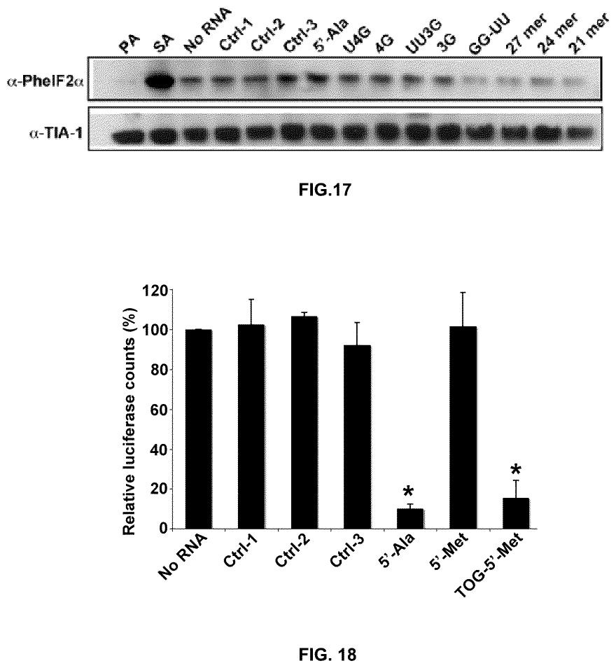

Additional immunoblotting experiments were performed to determine whether transfection with these oligonucleotides induced the phosphorylation of eIF2α, a classical trigger of stress granule assembly. These data show that transfection of wild type or mutant 5′-tiRNA^{Ala} does not induce phosphorylation of eIF2a (FIG. 17). The data show that 5′-tiRNA^{Ala} inhibits translation in live cells (in which most of the target transcripts are presumably capped). The close correlation between translational repression in RRL, displacement of eIF4F from m′GTP-Sepharose, and stress granule assembly suggests that eIF4F is a target for tiRNA-induced translational repression.

### Example 7. Addition of 5′-TOG Activates 5′-tiRNA^{Met}

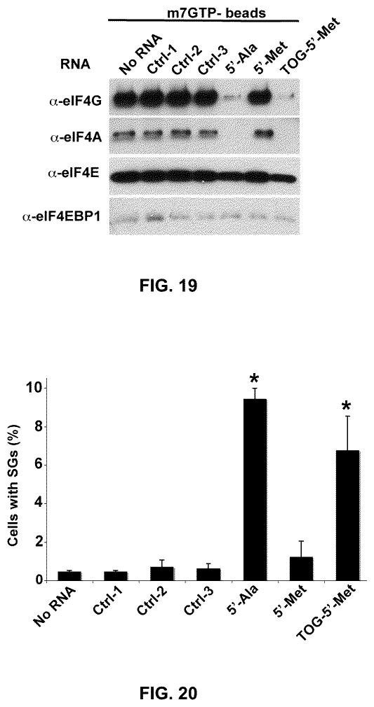

Additional experiments were performed to confirm the importance of the 5′-TOG motif for tiRNA activity. In these experiments, the first 5 nucleotides of non-TOG-containing 5′-tiRNA^{Met} was substituted with 5 guanine residues (FIG. 13) (5′-phospho-GGG GGU UUA GCG CAG UAG GUA GCG CGU CAG U-3′: SEQ ID NO: 26). This substituted 5′-tiRNA^{Met} was used in RRL translation assays, the eIF4F displacement assays using m⁷GTP-Sepharose assays, and stress granule formation assays (cellular assays) performed as described above. The resulting data show that although endogenous 5′-tiRNA^{Met} does not inhibit translation in RRL (FIG. 18), displace eIF4F from m⁷GTP-Sepharose (FIG. 19), or induce the assembly of stress granules (FIG. 20), the addition of a 5′-TOG motif to 5′-tiRNA^{Met} is sufficient to confer these activities on this tRNA fragment (FIGS. 18-20).

### Example 8. Identification of tiRNA-Interacting

Pull-down of RNA-protein complexes using biotinylated control RNA or 5′-tiRNA^{Ala} immobilized on streptavidin beads was used to purify proteins that interact with these RNAs. RNA-bound proteins were identified by mass spectrometry. Independent pull-down experiments were performed to confirm the ability of several of these proteins to interact with tiRNA^{Ala}. These experiments were performed using biotinylated control or 5′-tiRNA^{Ala} using streptavidin beads as described above. The individual 3′-biotinylated oligonucleotides used in these assays were synthesized by and purchased from Integrated DNA Technology (listed below). Silver staining and mass spectrometry identification of tiRNA-binding proteins was performed as described below.

Biotinylated Oligonucleotides
Ctrl1-bio:
(SEQ ID NO: 27)
5′-UGA AGG GUU UUU UGU GUC UCU AUU UCC UUC-
3′-biotin
Ctrl2-bio:
(SEQ ID NO: 28)
5′-phospho-UGU GAG UCA CGU GAG GGC AGA AUC
UGC UC-3′-biotin
Ctrl3-bio:
(SEQ ID NO: 29)
5′-Phospho-GCA UUC ACU UGG AUA GUA AAU CCA
AGC UGA A-3′-biotin
5′-Ala-bio:
(SEQ ID NO: 30)
5′-phospho-GGG GGU GUA GCU CAG UGG UAG AGC
GCG UGC-3′-biotin
3′-Ala-bio:
(SEQ ID NO: 31)
5′-CUU AGC AUG CAC GAG GCC CCG GGU UCA AUC
CCC GGC ACC UCCA-3′-biotin

### Silver Staining of SDS PAGE Gels

To detect proteins recovered from affinity purification with biotinylated RNAs, eluted protein samples were run on gradient 4-20% SDS PAGE gels (Invitrogen) and fixed with Protein Fixation Buffer (30% ethanol: 10% acetic acid) for 30 minutes. SilverSNAP Stain Kit II (Pierce) was used according to the manufacture's recommendation to detect proteins.

Mass Spectrometry Identification of tiRNA-Binding Proteins

For mass spectrometry, protein solutions from affinity purification using 3′-end biotinylated RNAs were precipitated with trichloroacetic acid (TCA). Briefly, eluted protein samples were adjusted to 20% final volume of TCA using 100% TCA (in water) solution. The resulting mixture was placed on ice for 20 minutes followed by centrifugation (20 minutes, 13200 rpm, 4° C.). Protein pellet was washed once with 1 ml of cold (−20° C.) acetone (HPLC grade, Sigma), followed by centrifugation (20 minutes, 13200 rpm, 4° C.). The supernatant was carefully removed and the pellet was air-dried for 10 minutes at room temperature. The identification of tiRNA-binding proteins was done by Taplin Mass Spectrometry Facility according to standard protocols (Harvard Medical School).

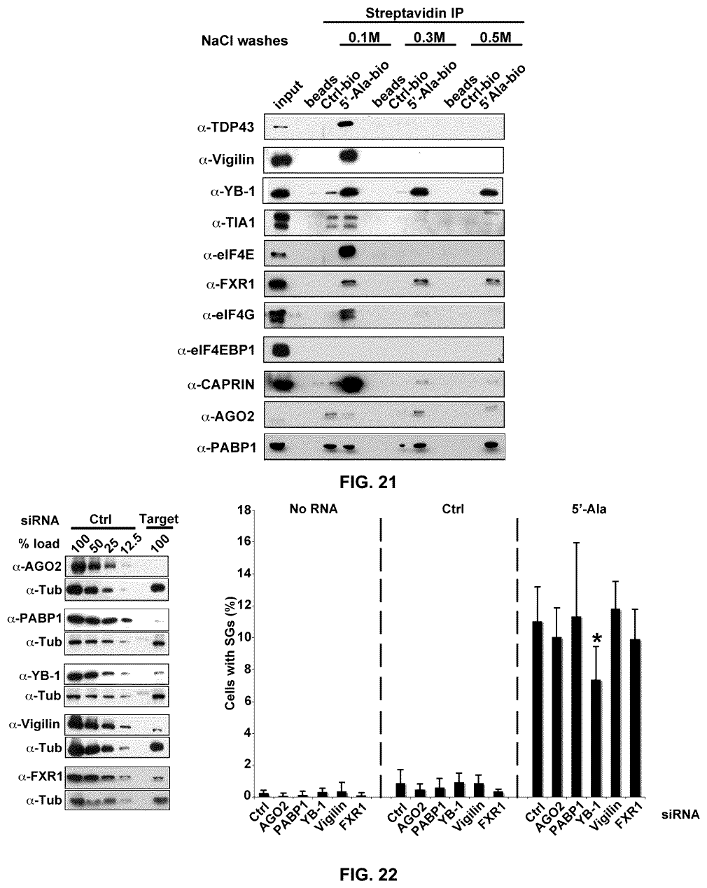

The resulting data confirmed that several proteins involved in the regulation of RNA metabolism, including TDP-43 (TARDBP), Vigilin (HDLBP), YB-1 (YBX1), eIF4E, FXR1, CIF4G, CAPRIN1 (CAPRIN), Argonaute-2 (EIF2C2), and PABP1 (PABPC1) bind to 5′-tiRNA^{Ala} more strongly than control RNA (FIG. 21). Some of these interactions (e.g., YB-1, FXR-1, and PABP1) are maintained under high salt wash conditions.

### Example 9. tiRNA-Binding Proteins are Required for 5′-tiRNA^{Ala}-Induced Stress Granule Assembly

Additional experiments were performed to determine whether the identified 5′-tiRNA^{Ala} binding proteins are required for translational repression. In these experiments, AGO2, PABP1, YB-1, Vigilin, and FXR1 were knocked down using siRNA prior to quantifying tiRNA-induced stress granule assembly in U2OS cells (assays performed as described above). These data show that, of the tested binding proteins, YB-1 was the only 5′-tiRNA^{Ala} binding protein required for the assembly of stress granules (FIG. 22). YB-1 is a protein that inhibits translation of both uncapped and capped mRNAs by displacing eIF4G from the eIF4F complex and by displacing eIF4E from the m⁷G cap.

### Example 10. Synthetic DNA Equivalents of 5′-tiRNA^{Ala} and Anti-Proliferative DNAs Inhibit Translation and Induce Stress Granule Formation

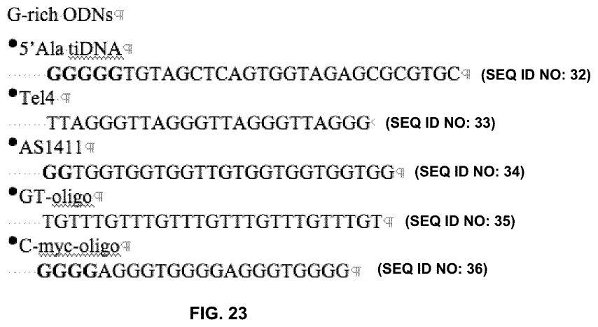

Experiments were performed to test the ability of synthetic DNA equivalents of 5′-tiRNA^{Ala} and anti-proliferative G-rich oligodeoxynucleotides (shown in FIG. 23) to inhibit translation of luciferase transcripts in RRL assays, induce stress granule formation in cells, and displace eIF4G, eIF4A, and eIF4E from m⁷GTP-Sepharose in vitro (performed generally as described above).

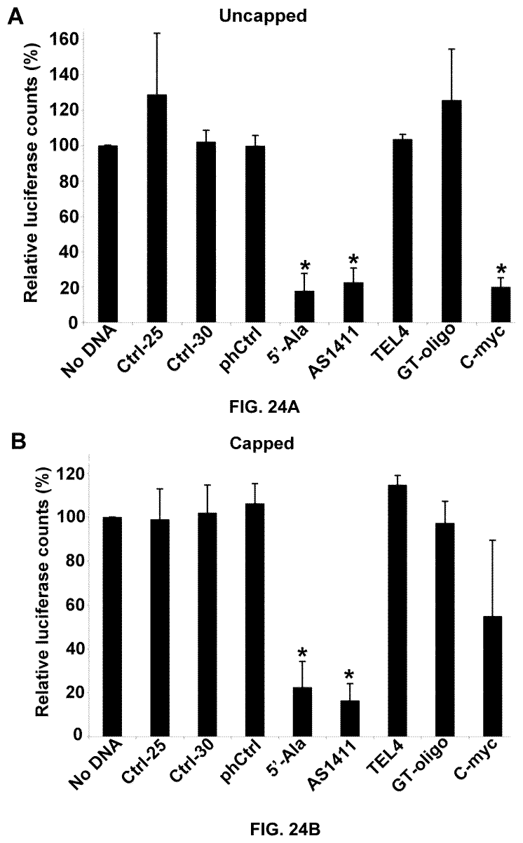

The resulting data show that 5′-tiDNA^{Ala}, AS1411, and c-myc oligodeoxynucleotides are potent inhibitors of translation in the RRL assay (none of these compounds alters the amount of luciferase RNA as assessed by Northern blotting analysis performed as described above) (FIG. 24). In contrast, neither TEL4, nor GT-oligo inhibit translation in this assay. Thus, some G-rich oligodeoxynucleotides inhibit translation, whereas others do not.

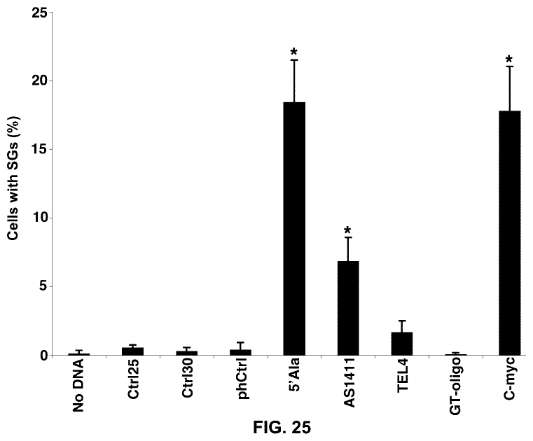

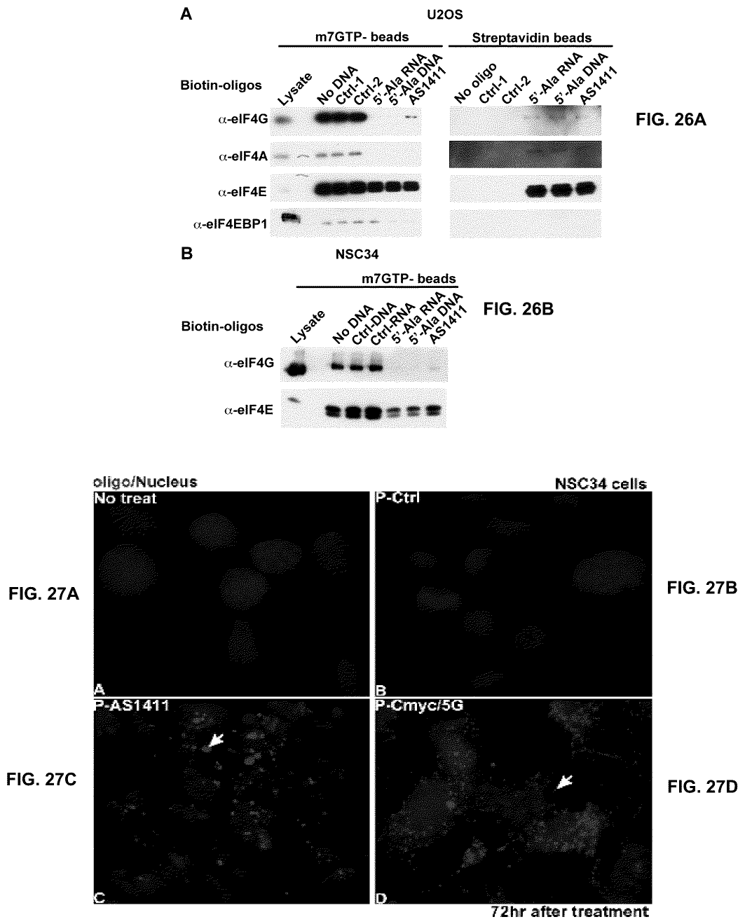

The data also show that the ability of the 5′-tiRNAs to inhibit translation correlates with their ability to trigger stress granule assembly in cells (following cellular transfection with these molecules) (FIG. 25). In U2OS cells, 5′-tiDNA^{Ala}, AS1411, and c-myc oligodeoxynucleotides induce stress granule assembly, whereas TEL4 and GT-oligo did not (FIG. 25). Similar results were observed in NSC34 cells, indicating that selected G-rich oligodeoxynucleotides can promote stress granule assembly in motor neurons. The molecular mechanism(s) by which these oligodeoxynucleotides mediate translation inhibition was further investigated by determining their ability to displace eIF4F from m⁷-GTP Sepharose. The data from these experiments show that, in lysates from both U2OS (FIG. 26A) and NSC34 cells (FIG. 26B), 5′-tiRNA^{Ala}, 5′-tiDNA^{Ala}, and AS1411 displace eIF4G, eIF4A, and eIF4E from m⁷G in vitro. These data suggest that these oligodeoxynucleotides mimic angiogenin-induced tiRNAs in their ability to trigger a stress response program.

### Example 11. Cellular Uptake of Synthetic 5′ G-rich Oligodeoxynucleotides

Additional experiments were performed in order to determine whether the 5′ G-rich oligodeoxynucleotides would be taken up by motor neurons in the absence of transfection agents. In these experiments NSC34 cells were treated with biotin-labeled C-myc, biotin-labeled AS1411, or biotin-labeled C-rich control oligodeoxynucleotides and cellular uptake was detected using Cy3-streptavidin. The resulting data show that AS1411 and c-myc, but not control oligodeoxynucleotides were found in cytoplasmic puncta within NSC34 cells: a result that is consistent with endosomal uptake (FIG. 27). In addition, some cells showed diffuse fluorescence consistent with cytoplasmic localization. Similar results were obtained with 5′-tiDNA^{Ala}. These results indicate the ability of these G-rich oligodeoxynucleotides to spontaneously enter motor neurons and gain access to the cytoplasm.

### Example 12. Functional Effect of G-rich Oligodeoxynucleotides

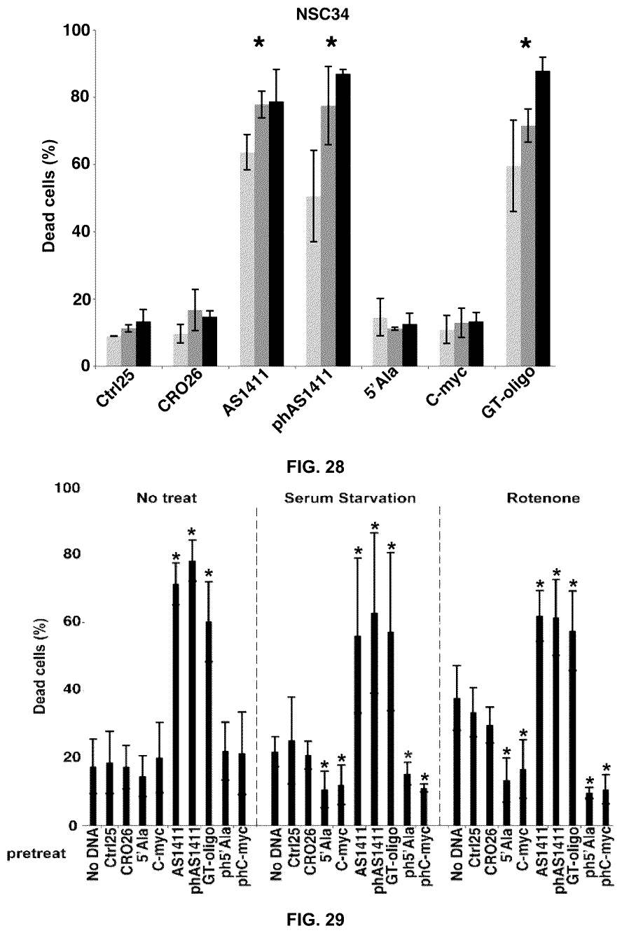

Additional experiments were performed to test the effect of the G-rich oligodeoxynucleotides on motor neurons. A first set of dose response experiments were performed in order to determine whether the tested G-rich oligodeoxynucleotides were toxic to motor neurons in vitro. These data show that the AS1411 and oligo-GT oligodeoxynucleotides markedly increased NSC34 cell death at the end of a 72-hour incubation (FIG. 28). In contrast, the control piDNA (ctrl 25, AAAAAACTCG AGATGGCGCACGCTG: SEQ ID NO: 38), C-rich control (CRO26, CCTCCTCCTCCTTCTCCTCCTCCTCC: SEQ ID NO: 39), 5′-tiDNA^{Ala}, and c-myc oligodeoxynucleotides do not reduce the numbers of viable NSC34 cells.

A second set of experiments was performed to determine whether the tested G-rich oligodeoxynucleotides are neuroprotective. In these experiments NSC34 cells were cultured in the presence of the individual oligodeoxynucleotides, then subjected to nutrient (serum starvation) or mitochondrial (rotenone) stress for 24 hours. NSC34 cells treated with either 5′-tiDNA^{Ala} or c-myc oligodeoxynucleotides, but not AS1411 or oligo-GT oligodeoxynucleotides were significantly protected from the adverse effects of stress (cell death: FIG. 29). These data suggest that there are three different functional classes of G-rich oligodeoxynucleotides: Class I oligodeoxynucleotides (e.g., oligo-GT) that reduce NSC34 cell survival: Class II oligodeoxynucleotides (e.g., 5′-tiDNA^{Ala} and c-myc) that promote NSC34 cell survival by activating a stress response program: and Class III oligodeoxynucleotides (e.g., AS1411) that reduce NSC34 survival, but also activate a stress response program. The modest therapeutic effects of AS1411 may be a consequence of this cytoprotective stress response program. These data identify 5′-tiDNA^{Ala} and c-myc oligodeoxynucleotides as promising compounds for the development of therapeutic agent for treating a neurological disorder (e.g., a neurological disorder that involves motor neuron death, such as amyotrophic lateral sclerosis).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Sequence Appendix

Additional tRNA sequences that can be used to generate any of the neuroprotective molecules described herein are listed below.

Homo_sapiens_chr6.trna95-AlaAGC (58249908-58249836)
Ala (AGC) 73 bp Sc: 42.26
(SEQ ID NO: 176)
GGGGAATTAGCTCAAGCGGTAGAGCGCTCCCTTAGCATGCGAGAGGTAGCGGGATCGACG
CCCCCATTCTCTA
Homo_sapiens_chr6.trna25-AlaAGC (26859897-26859969)
Ala (AGC) 73 bp Sc: 46.89
(SEQ ID NO: 177)
GGGGGATTAGCTCAAGCGGTAGGGTGCCTGCTTAGCATGCAAGAGGTAGCAGGATCGACG
CCTGCATTCTCCA
Homo_sapiens_chr1.trna87-AlaAGC (148284076-148284006)
Ala (AGC) 71 bp Sc: 48.21
(SEQ ID NO: 178)
GGGGGTGTAGATCAGTGGTAGGGCGCACGCTTAGCATGCATGAGGCCCTGGGTCAATCCC
CAGCACCTCCA
Homo_sapiens_chr6.trna94-AlaAGC (58250620-58250548)
Ala (AGC) 73 bp Sc: 54.62
(SEQ ID NO: 179)
GGGGGATTAGCTCAAGCGGTAGAGCGCCTGCTTAGCATGCAAGAGGTAGCAGGATCGATG
CCTGCATTCTCCA
Homo_sapiens_chr6.trna160-AlaAGC (26881822-26881750)
Ala (AGC) 73 bp Sc: 54.69
(SEQ ID NO: 180)
GGGGAATTGGCTCAAGCGGTAGAGCGCTTGCTTAGCATGCAAGAGGTAGCAGGATCGACG
CCTGCACTCTCCA
Homo_sapiens_chr6.trna23-AlaAGC (26836235-26836307)
Ala (AGC) 73 bp Sc: 54.69
(SEQ ID NO: 181)
GGGGAATTGGCTCAAGCGGTAGAGCGCTTGCTTAGCATGCAAGAGGTAGCAGGATCGACG
CCTGCACTCTCCA
Homo_sapiens_chr6.trna90-AlaAGC (58295475-58295403)
Ala (AGC) 73 bp Sc: 54.84
(SEQ ID NO: 182)
GGGGAATTAGCGCAAGTGGTAGAGTGCTTGCTTAGCATGCAAGAGGTAGTGGGATCGATG
CCCACATTCTCCA
Homo_sapiens_chr6.trna89-AlaAGC (58304654-58304582)
Ala (AGC) 73 bp Sc: 57.89
(SEQ ID NO: 183)
GGGGAATTAGCCCAAGTGGTAGAGCGCTTGCTTAGCATGCAAGAGGTAGTGGGATCGATG
CCCACATTCTCCA
Homo_sapiens_chr14.trna9-AlaAGC (88515195-88515267)
Ala (AGC) 73 bp Sc: 59.85
(SEQ ID NO: 184)
GGGGAATTAGCTCAAGTGGTAGAGCGCTCGCTTAGCATGCGAGAGGTAGTGGGATCGATG
CCCGCATTCTCCA
Homo_sapiens_chr6.trna18-AlaAGC (26781569-26781641)
Ala (AGC) 73 bp Sc: 60.72
(SEQ ID NO: 185)
GGGGAATTAGCTCAAGTGGTAGAGCGCTTGCTTAGCATGCAAGAGGTAGTGGGATCAATG
CCCACATTCTCCA
Homo_sapiens_chr6.trna22-AlaAGC (26813585-26813657)
Ala (AGC) 73 bp Sc: 61.81
(SEQ ID NO: 186)
GGGGAATTAGCTCAAGCGGTAGAGCGCTTGCTTAGCATGCAAGAGGTAGTGGGATCGATG
CCCACATTCTCCA
Homo_sapiens_chr6.trna93-AlaAGC (58272659-58272587)
Ala (AGC) 73 bp Sc: 61.81
(SEQ ID NO: 187)
GGGGAATTAGCTCAAGCGGTAGAGCGCTTGCTTAGCATGCAAGAGGTAGTGGGATCGATG
CCCACATTCTCCA
Homo_sapiens_chr6.trna159-AlaAGC (26904057-26903985)
Ala (AGC) 73 bp Sc: 62.45
(SEQ ID NO: 188)
GGGGAATTAGCTCAAGTGGTAGAGCGCTTGCTTAGCATGCAAGAGGTAGTGGGATCGATG
CCCACATTCTCCA
Homo_sapiens_chr6.trna19-AlaAGC (26790694-26790766)
Ala (AGC) 73 bp Sc: 62.45
(SEQ ID NO: 189)
GGGGAATTAGCTCAAGTGGTAGAGCGCTTGCTTAGCATGCAAGAGGTAGTGGGATCGATG
CCCACATTCTCCA
Homo_sapiens_chr6.trna91-AlaAGC (58290710-58290638)
Ala (AGC) 73 bp Sc: 62.45
(SEQ ID NO: 190)
GGGGAATTAGCTCAAGTGGTAGAGCGCTTGCTTAGCATGCAAGAGGTAGTGGGATCGATG
CCCACATTCTCCA
Homo_sapiens_chr6.trna166-AlaAGC (26680143-26680071)
Ala (AGC) 73 bp Sc: 63.06
(SEQ ID NO: 191)
GGGGAATTAGCTCAAATGGTAGAGCGCTCGCTTAGCATGCGAGAGGTAGCGGGATCGATG
CCCGCATTCTCCA
Homo_sapiens_chr6.trna20-AlaAGC (26795464-26795536)
Ala (AGC) 73 bp Sc: 63.16
(SEQ ID NO: 192)
GGGGAATTAGCTCAAGTGGTAGAGCGCTTGCTTAGCACGCAAGAGGTAGTGGGATCGATG
CCCACATTCTCCA
Homo_sapiens_chr6.trna161-AlaAGC (26879341-26879269)
Ala (AGC) 73 bp Sc: 63.36
(SEQ ID NO: 193)
GGGGAATTAGCTCAGGCGGTAGAGCGCTCGCTTAGCATGCGAGAGGTAGCGGGATCGACG
CCCGCATTCTCCA
Homo_sapiens_chr6.trna24-AlaAGC (26838716-26838788)
Ala (AGC) 73 bp Sc: 63.36
(SEQ ID NO: 194)
GGGGAATTAGCTCAGGCGGTAGAGCGCTCGCTTAGCATGCGAGAGGTAGCGGGATCGACG
CCCGCATTCTCCA
Homo_sapiens_chr2.trna3-AlaAGC (27127586-27127658)
Ala (AGC) 73 bp Sc: 63.68
(SEQ ID NO: 195)
GGGGGATTAGCTCAAATGGTAGAGCGCTCGCTTAGCATGCGAGAGGTAGCGGGATCGATG
CCCGCATCCTCCA
Homo_sapiens_chr8.trna6-AlaAGC (67188978-67189050)
Ala (AGC) 73 bp Sc: 63.68
(SEQ ID NO: 196)
GGGGGATTAGCTCAAATGGTAGAGCGCTCGCTTAGCATGCGAGAGGTAGCGGGATCGATG
CCCGCATCCTCCA
Homo_sapiens_chr6.trna68-AlaAGC (28795460-28795531)
Ala (AGC) 72 bp Sc: 70.19
(SEQ ID NO: 197)
GGGGGTGTAGCTCAGTGGTAGAGCGCGTGCTTAGCATGCACGAGGCCCCGGGTTCAATCC
CTGGCACCTCCA
Homo_sapiens_chr6.trna105-AlaAGC (28887899-28887828)
Ala (AGC) 72 bp Sc: 71.23
(SEQ ID NO: 198)
GGGGGTATAGCTCAGCGGTAGAGCGCGTGCTTAGCATGCACGAGGTCCTGGGTTCAATCC
CCAATACCTCCA
Homo_sapiens_chr6.trna67-AlaAGC (28786345-28786416)
Ala (AGC) 72 bp Sc: 75.68
(SEQ ID NO: 199)
GGGGGTGTAGCTCAGTGGTAGAGCGCGTGCTTAGCATGCACGAGGCCCTGGGTTCAATCC
CCAGCACCTCCA
Homo_sapiens_chr6.trna120-AlaAGC (28734064-28733993)
Ala (AGC) 72 bp Sc: 75.78
(SEQ ID NO: 200)
GGGGATGTAGCTCAGTGGTAGAGCGCATGCTTAGCATGCATGAGGTCCCGGGTTCGATCC
CCAGCATCTCCA
Homo_sapiens_chr6.trna65-AlaAGC (28682912-28682983)
Ala (AGC) 72 bp Sc: 76.69
(SEQ ID NO: 201)
GGGGGTGTAGCTCAGTGGTAGAGCGCGTGCTTAGCATGTACGAGGTCCCGGGTTCAATCC
CCGGCACCTCCA
Homo_sapiens_chr6.trna101-AlaAGC (28939512-28939441)
Ala (AGC) 72 bp Sc: 77.18
(SEQ ID NO: 202)
GGGGGTGTAGCTCAGTGGTAGAGCGCGTGCTTAGCATGCACGAGGCCCCGGGTTCAATCC
CCGGCACCTCCA
Homo_sapiens_chr6.trna102-AlaAGC (28914271-28914200)
Ala (AGC) 72 bp Sc: 77.18
(SEQ ID NO: 203)
GGGGGTGTAGCTCAGTGGTAGAGCGCGTGCTTAGCATGCACGAGGCCCCGGGTTCAATCC
CCGGCACCTCCA
Homo_sapiens_chr6.trna108-AlaAGC (28871791-28871720)
Ala (AGC) 72 bp Sc: 81.12
(SEQ ID NO: 204)
GGGGGTATAGCTCAGTGGTAGAGCGCGTGCTTAGCATGCACGAGGTCCTGGGTTCGATCC
CCAGTACCTCCA
Homo_sapiens_chr6.trna117-AlaCGC (28771759-28771688)
Ala (CGC) 72 bp Sc: 56.63
(SEQ ID NO: 205)
GGGGGTGTAGATCAGTGGTAGAGCGCATGCTTCGCATGTACGAGGTCCCTGGTTCAATCC
CTGGTACCTCCA
Homo_sapiens_chr6.trna70-AlaCGC (28805071-28805142)
Ala (CGC) 72 bp Sc: 70.09
(SEQ ID NO: 206)
GGGGGTGTAGCTCAGTGGTAGAGCGCGTGCTTCGCATGTACGAGGCCCCGGGTTCGACCC
CCGGCTCCTCCA
Homo_sapiens_chr2.trna13-AlaCGC (156965527-156965598)
Ala (CGC) 72 bp Sc: 73.12
(SEQ ID NO: 207)
GGGGATGTAGCTCAGTGGTAGAGCGCGCGCTTCGCATGTGTGAGGTCCCGGGTTCAATCC
CCGGCATCTCCA
Homo_sapiens_chr6.trna119-AlaCGC (28749663-28749592)
Ala (CGC) 72 bp Sc: 74.81
(SEQ ID NO: 208)
GGGGATGTAGCTCAGTGGTAGAGCGCATGCTTCGCATGTATGAGGCCCCGGGTTCGATCC
CCGGCATCTCCA
Homo_sapiens_chr6.trna10-AlaCGC (26661710-26661781)
Ala (CGC) 72 bp Sc: 78.91
(SEQ ID NO: 209)
GGGGATGTAGCTCAGTGGTAGAGCGCATGCTTCGCATGTATGAGGTCCCGGGTTCGATCC
CCGGCATCTCCA
Homo_sapiens_chr11.trna18-AlaTGC (50190526-50190455)
Ala (TGC) 72 bp Sc: 51.06
(SEQ ID NO: 210)
GGGGGTGTAGCTCAGTGGTAGAGCGGATGCTTTGCATGTATGAGACTTTGGGTTGGATCC
CCAGCACCTCCA
Homo_sapiens_chr6.trna107-AlaTGC (28878626-28878556)
Ala (TGC) 71 bp Sc: 59.75
(SEQ ID NO: 211)
GGGGGTGTAGCTCAGTGGTAGAGCGCATGCTTTGCATGTATGAGGCCTCGGTTCGATCCC
CGACACCTCCA
Homo_sapiens_chr6.trna113-AlaTGC (28834191-28834120)
Ala (TGC) 72 bp Sc: 67.60
(SEQ ID NO: 212)
GGGGGTGTAGCTCAGTGGTAGAGCACATGCTTTGCATGTGTGAGGCCCCGGGTTCGATCC
CCGGCACCTCCA
Homo_sapiens_chr6.trna104-AlaTGC (28893062-28892991)
Ala (TGC) 72 bp Sc: 70.95
(SEQ ID NO: 213)
GGGGGTGTAGCTCAGTGGTAGAGCGCATGCTTTGCATGTATGAGGCCTCGGGTTCGATCC
CCGACACCTCCA
Homo_sapiens_chr12.trna8-AlaTGC (123990465-123990536)
Ala (TGC) 72 bp Sc: 73.41
(SEQ ID NO: 214)
GGGGATGTAGCTCAGTGGTAGAGCGCATGCTTTGCACGTATGAGGCCCCGGGTTCAATCC
CCGGCATCTCCA
Homo_sapiens_chr12.trna13-AlaTGC (123972325-123972254)
Ala (TGC) 72 bp Sc: 74.45
(SEQ ID NO: 215)
GGGGATGTAGCTCAGTGGTAGAGCGCATGCTTTGCATGTATGAGGCCCCGGGTTCGATCC
CCGGCATCTCCA
Homo_sapiens_chr5.trna8-AlaTGC (180566474-180566545)
Ala (TGC) 72 bp Sc: 74.45
(SEQ ID NO: 216)
GGGGATGTAGCTCAGTGGTAGAGCGCATGCTTTGCATGTATGAGGCCCCGGGTTCGATCC
CCGGCATCTCCA
Homo_sapiens_chr6.trna66-AlaTGC (28719201-28719272)
Ala (TGC) 72 bp Sc: 78.55
(SEQ ID NO: 217)
GGGGATGTAGCTCAGTGGTAGAGCGCATGCTTTGCATGTATGAGGTCCCGGGTTCGATCC
CCGGCATCTCCA
Homo_sapiens_chr6.trna110-AlaTGC (28865597-28865526)
Ala (TGC) 72 bp Sc: 79.16
(SEQ ID NO: 218)
GGGGGTGTAGCTCAGTGGTAGAGCGCATGCTTTGCATGTATGAGGTCCCGGGTTCGATCC
CCGGCACCTCCA
Homo_sapiens_chr3.trna11-ArgACG (45705567-45705495)
Arg (ACG) 73 bp Sc: 68.07
(SEQ ID NO: 219)
GGGCCAGTGGCGCAATGGATAACGCGTCTGACTACGGATCAGAAGATTCTAGGTTCGACT
CCTGGCTGGCTCG
Homo_sapiens_chr6.trna138-ArgACG (27746395-27746323)
Arg (ACG) 73 bp Sc: 68.07
(SEQ ID NO: 220)
GGGCCAGTGGCGCAATGGATAACGCGTCTGACTACGGATCAGAAGATTCTAGGTTCGACT
CCTGGCTGGCTCG
Homo_sapiens_chr6.trna156-ArgACG (27289674-27289602)
Arg (ACG) 73 bp Sc: 68.07
(SEQ ID NO: 221)
GGGCCAGTGGCGCAATGGATAACGCGTCTGACTACGGATCAGAAGATTCTAGGTTCGACT
CCTGGCTGGCTCG
Homo_sapiens_chr6.trna36-ArgACG (27290931-27291003)
Arg (ACG) 73 bp Sc: 68.07
(SEQ ID NO: 222)
GGGCCAGTGGCGCAATGGATAACGCGTCTGACTACGGATCAGAAGATTCTAGGTTCGACT
CCTGGCTGGCTCG
Homo_sapiens_chr14.trna7-ArgACG (22468750-22468822)
Arg (ACG) 73 bp Sc: 72.37
(SEQ ID NO: 223)
GGGCCAGTGGCGCAATGGATAACGCGTCTGACTACGGATCAGAAGATTCCAGGTTCGACT
CCTGGCTGGCTCG
Homo_sapiens_chr6.trna6-ArgACG (26436347-26436419)
Arg (ACG) 73 bp Sc: 72.37
(SEQ ID NO: 224)
GGGCCAGTGGCGCAATGGATAACGCGTCTGACTACGGATCAGAAGATTCCAGGTTCGACT
CCTGGCTGGCTCG
Homo_sapiens_chr6.trna8-ArgACG (26645705-26645777)
Arg (ACG) 73 bp Sc: 72.37
(SEQ ID NO: 225)
GGGCCAGTGGCGCAATGGATAACGCGTCTGACTACGGATCAGAAGATTCCAGGTTCGACT
CCTGGCTGGCTCG
Homo_sapiens_chr17.trna23-ArgCCG (63446547-63446475)
Arg (CCG) 73 bp Sc: 65.49
(SEQ ID NO: 226)
GACCCAGTGGCCTAATGGATAAGGCATCAGCCTCCGGAGCTGGGGATTGTGGGTTCGAGT
CCCATCTGGGTCG
Homo_sapiens_chr17_random.trna1-ArgCCG (1739927-1739999)
Arg (CCG) 73 bp Sc: 65.49
(SEQ ID NO: 227)
GACCCAGTGGCCTAATGGATAAGGCATCAGCCTCCGGAGCTGGGGATTGTG
GGTTCGAGTCCCATCTGGGTCG
Homo_sapiens_chr16.trna1-ArgCCG (3140676-3140748)
Arg (CCG) 73 bp Sc: 69.88
(SEQ ID NO: 228)
GGCCGCGTGGCCTAATGGATAAGGCGTCTGATTCCGGATCAGAAGATTGAGGGTTCGAGT
CCCTTCGTGGTCG
Homo_sapiens_chr6.trna114-ArgCCG (28818780-28818708)
Arg (CCG) 73 bp Sc: 69.88
(SEQ ID NO: 229)
GGCCGCGTGGCCTAATGGATAAGGCGTCTGATTCCGGATCAGAAGATTGAGGGTTCGAGT
CCCTTCGTGGTCG
Homo_sapiens_chr6.trna73-ArgCCG (28957144-28957216)
Arg (CCG) 73 bp Sc: 69.88
(SEQ ID NO: 230)
GGCCGCGTGGCCTAATGGATAAGGCGTCTGATTCCGGATCAGAAGATTGAGGGTTCGAGT
CCCTTCGTGGTCG
Homo_sapiens_chr16.trna12-ArgCCT (3183919-3183991)
Arg (CCT) 73 bp Sc: 59.77
(SEQ ID NO: 231)
GCCCCAGTGGCCTGATGGATAAGGTACTGGCCTCCTAAGCCAGGGATTGTGGGTTCGAGT
TCCACCTGGGGTA
Homo_sapiens_chr7.trna3-ArgCCT (138675986-138676058)
Arg (CCT) 73 bp Sc: 67.28
(SEQ ID NO: 232)
GCCCCAGTGGCCTAATGGATAAGGCATTGGCCTCCTAAGCCAGGGATTGTGGGTTCGAGT
CCCATCTGGGGTG
Homo_sapiens_chr16.trna2-ArgCCT (3142902-3142974)
Arg (CCT) 73 bp Sc: 71.53
(SEQ ID NO: 233)
GCCCCGGTGGCCTAATGGATAAGGCATTGGCCTCCTAAGCCAGGGATTGTGGGTTCGAGT
CCCACCCGGGGTA
Homo_sapiens_chr17.trna21-ArgCCT (70542193-70542121)
Arg (CCT) 73 bp Sc: 73.41
(SEQ ID NO: 234)
GCCCCAGTGGCCTAATGGATAAGGCACTGGCCTCCTAAGCCAGGGATTGTGGGTTCGAGT
CCCACCTGGGGTG
Homo_sapiens_chr17.trna18-ArgCCT (70541596-70541668)
Arg (CCT) 73 bp Sc: 73.88
(SEQ ID NO: 235)
GCCCCAGTGGCCTAATGGATAAGGCACTGGCCTCCTAAGCCAGGGATTGTGGGTTCGAGT
CCCACCTGGGGTA
Homo_sapiens_chr9.trna2-ArgTCG (112000624-112000696)
Arg (TCG) 73 bp Sc: 56.18
(SEQ ID NO: 236)
GGCCGTGTGGCCTAATGGATAAGGCGTCTGACTTCGGATCAAAAGATTGCAGGTTTGAGT
TCTGCCACGGTCG
Homo_sapiens_chr6.trna124-ArgTCG (28618942-28618870)
Arg (TCG) 73 bp Sc: 69.08
(SEQ ID NO: 237)
GACCACGTGGCCTAATGGATAAGGCGTCTGACTTCGGATCAGAAGATTGAGGGTTCGAAT
CCCTTCGTGGTTG
Homo_sapiens_chr6.trna3-ArgTCG (26407884-26407956)
Arg (TCG) 73 bp Sc: 69.55
(SEQ ID NO: 238)
GACCACGTGGCCTAATGGATAAGGCGTCTGACTTCGGATCAGAAGATTGAGGGTTCGAAT
CCCTTCGTGGTTA
Homo_sapiens_chr17.trna19-ArgTCG (70542803-70542875)
Arg (TCG) 73 bp Sc: 70.52
(SEQ ID NO: 239)
GACCGCGTGGCCTAATGGATAAGGCGTCTGACTTCGGATCAGAAGATTGAGGGTTCGAGT
CCCTTCGTGGTCG
Homo_sapiens_chr6.trna4-ArgTCG (26431025-26431097)
Arg (TCG) 73 bp Sc: 72.33
(SEQ ID NO: 240)
GACCACGTGGCCTAATGGATAAGGCGTCTGACTTCGGATCAGAAGATTGAGGGTTCGAAT
CCCTCCGTGGTTA
Homo_sapiens_chr15.trna4-ArgTCG (87679308-87679380)
Arg (TCG) 73 bp Sc: 76.93
(SEQ ID NO: 241)
GGCCGCGTGGCCTAATGGATAAGGCGTCTGACTTCGGATCAGAAGATTGCAGGTTCGAGT
CCTGCCGCGGTCG
Homo_sapiens_chr1.trna86-ArgTCT (157378098-157378025)
Arg (TCT) 74 bp Sc: 76.29
(SEQ ID NO: 242)
GTCTCTGTGGCGCAATGGACGAGCGCGCTGGACTTCTAATCCAGAGGTTCCGGGTTCGAG
TCCCGGCAGAGATG
Homo_sapiens_chr9.trna4-ArgTCT (130142266-130142176)
Arg (TCT) 91 bp Sc: 67.17
(SEQ ID NO: 243)
GGCTCTGTGGCGCAATGGATAGCGCATTGGACTTCTAGCTGAGCCTAGTGTGGTCATTCA
AAGGTTGTGGGTTCGAGTCCCACCAGAGTCG
Homo_sapiens_chr6.trna52-ArgTCT (27637942-27638028)
Arg (TCT) 87 bp Sc: 65.24
(SEQ ID NO: 244)
GGCTCTGTGGCGCAATGGATAGCGCATTGGACTTCTAGCCTAAATCAAGAGATTCAAAGG
TTGCGGGTTCGAGTCCCTCCAGAGTCG
Homo_sapiens_chr11.trna3-ArgTCT (59075343-59075428)
Arg (TCT) 86 bp Sc: 71.13
(SEQ ID NO: 245)
GGCTCTGTGGCGCAATGGATAGCGCATTGGACTTCTAGATAGTTAGAGAAATTCAAAGGT
TGTGGGTTCGAGTCCCACCAGAGTCG
Homo_sapiens_chr17.trna4-ArgTCT (7964968-7965055)
Arg (TCT) 88 bp Sc: 71.27
(SEQ ID NO: 246)
GGCTCTGTGGCGCAATGGATAGCGCATTGGACTTCTAGTGACGAATAGAGCAATTCAAAG
GTTGTGGGTTCGAATCCCACCAGAGTCG
Homo_sapiens_chr1.trna9-ArgTCT (94085717-94085801)
Arg (TCT) 85 bp Sc: 71.18
(SEQ ID NO: 247)
GGCTCCGTGGCGCAATGGATAGCGCATTGGACTTCTAGAGGCTGAAGGCATTCAAAGGTT
CCGGGTTCGAGTCCCGGCGGAGTCG
Homo_sapiens_chr1.trna20-AsnATT (146185653-146185726)
Asn (ATT) 74 bp Sc: 52.07
(SEQ ID NO: 248)
GTCTCTGTGGCGCAATCGGTCAGAGCGTTCGGCTATTAACCGAACGGTGAGTAGTTCAAG
ACCACCCAGGGACG
Homo_sapiens_chr1.trna13-AsnGTT (144097086-144097161)
Asn (GTT) 76 bp Sc: 44.21
(SEQ ID NO: 249)
GTCTCTGTGGCGCCATCGGTTAGTGCCTTCGGCTGTTTGAACCGAAAGGCTGGTGGTTCA
AGCCCACCCAGAGATG
Homo_sapiens_chr1.trna93-AsnGTT (147936754-147936681)
Asn (GTT) 74 bp Sc: 46.92
(SEQ ID NO: 250)
ATCTCCGTGGAGCAATTGGTTAGCGCGTTCGGCCGTTAACCGGAAAGTTGGTGGTTCGAG
CCTACCCAGGGACG
Homo_sapiens_chr1.trna134-AsnGTT (16731553-16731480)
Asn (GTT) 74 bp Sc: 48.81
(SEQ ID NO: 251)
GTCTCTGTGGTGCAATCGGTTAGCGCGTTCGGCTGTTAACCATAAGGTTGGTGGTTACAG
ACCACCCAGGGACG
Homo_sapiens_chr1.trna50-AsnGTT (159858089-159858162)
Asn (GTT) 74 bp Sc: 49.69
(SEQ ID NO: 252)
GTTTCTGTAGCGCGATCGGTTAGCGCCTTCGGCTGTTAAACGAAAGGTTGGTGGTTCGTT
CCCACCCCGGGACA
Homo_sapiens_chr1.trna10-AsnGTT (142481551-142481624)
Asn (GTT) 74 bp Sc: 49.96
(SEQ ID NO: 253)
GTCTCTGTGGCGCAATCGGTTAGCGCGTTTGACTGTTAACTGAAAGGTTGGTGGTGCAAG
CCCATCCAGGGATG
Homo_sapiens_chr1.trna122-AsnGTT (143200273-143200200)
Asn (GTT) 74 bp Sc: 49.96
(SEQ ID NO: 254)
GTCTCTGTGGCGCAATCGGTTAGCGCGTTTGACTGTTAACTGAAAGGTTGGTGGTGCAAG
CCCATCCAGGGATG
Homo_sapiens_chr1.trna123-AsnGTT (143020044-143019971)
Asn (GTT) 74 bp Sc: 49.96
(SEQ ID NO: 255)
GTCTCTGTGGCGCAATCGGTTAGCGCGTTTGACTGTTAACTGAAAGGTTGGTGGTGCAAG
CCCATCCAGGGATG
Homo_sapiens_chr1.trna24-AsnGTT (146344161-146344234)
Asn (GTT) 74 bp Sc: 49.96
(SEQ ID NO: 256)
GTCTCTGTGGCGCAATCGGTTAGCGCGTTTGACTGTTAACTGAAAGGTTGGTGGTGCAAG
CCCATCCAGGGATG
Homo_sapiens_chr1.trna30-AsnGTT (147875233-147875306)
Asn (GTT) 74 bp Sc: 49.96
(SEQ ID NO: 257)
GTCTCTGTGGCGCAATCGGTTAGCGCGTTTGACTGTTAACTGAAAGGTTGGTGGTGCAAG
CCCATCCAGGGATG
Homo_sapiens_chr1.trna101-AsnGTT (147551200-147551127)
Asn (GTT) 74 bp Sc: 51.19
(SEQ ID NO: 258)
ATCTCCGTGGAGCAATTGGTTAGCGCGTTCGGCTGTTAACCGGAAAGTTGGTGGTTCGAG
CCTACCCAGGGACG
Homo_sapiens_chr1.trna12-AsnGTT (143193197-143193270)
Asn (GTT) 74 bp Sc: 53.66
(SEQ ID NO: 259)
GTCTCTGTGGTGCAATCGGTTAGCGCGTTCCGCTGTTAACCGAAAGCTTGGTGGTTTGAG
CCCACCCAGGGATG
Homo_sapiens_chr1.trna6-AsnGTT (17074545-17074618)
Asn (GTT) 74 bp Sc: 56.35
(SEQ ID NO: 260)
GTCTCTGTGGTGCAATCGGTTAGCGCGTTCGGCTGTTAACCATAAGGTTGGTGGTTAGAG
ACCACCCAGGGACG
Homo_sapiens_chr1.trna97-AsnGTT (147592969-147592896)
Asn (GTT) 74 bp Sc: 59.90
(SEQ ID NO: 261)
GTCTCTGTGGCGCAATCGGCTAGCGCGTTTGGCTGTTAACTAAAAAGTTGGTGGTTCGAA
CACACCCAGAGGCG
Homo_sapiens_chr1.trna11-AsnGTT (143012968-143013041)
Asn (GTT) 74 bp Sc: 60.57
(SEQ ID NO: 262)
GTCTCTGTGGTGCAATCGGTTAGCGCGTTCCGCTGTTAACCGAAAGCTTGGTGGTTCGAG
CCCACCCAGGGATG
Homo_sapiens_chr1.trna95-AsnGTT (147882314-147882241)
Asn (GTT) 74 bp Sc: 60.57
(SEQ ID NO: 263)
GTCTCTGTGGTGCAATCGGTTAGCGCGTTCCGCTGTTAACCGAAAGCTTGGTGGTTCGAG
CCCACCCAGGGATG
Homo_sapiens_chr1_random.trna2-AsnGTT (906435-906508)
Asn (GTT) 74 bp Sc: 66.79
(SEQ ID NO: 264)
GTCTCTGTGGCGCAATCGGCTAGCGCGTTTGGCTGTTAACTAAAAGGTTGGCGGTTCGAA
CCCACCCAGAGGCG
Homo_sapiens_chr1.trna113-AsnGTT (145987464-145987391)
Asn (GTT) 74 bp Sc: 70.44
(SEQ ID NO: 265)
GTCTCTGTGGCGCAATCGGTTAGCGCGTTCGGCTGTTAACTGAAAGGTTAGTGGTTCGAG
CCCACCCGGGGACG
Homo_sapiens_chr1.trna115-AsnGTT (144690464-144690391)
Asn (GTT) 74 bp Sc: 70.44
(SEQ ID NO: 266)
GTCTCTGTGGCGCAATCGGTTAGCGCGTTCGGCTGTTAACTGAAAGGTTAGTGGTTCGAG
CCCACCCGGGGACG
Homo_sapiens_chr1.trna89-AsnGTT (147978495-147978422)
Asn (GTT) 74 bp Sc: 71.43
(SEQ ID NO: 267)
GTCTCTGTGGCGCAATCGGCTAGCGCGTTTGGCTGTTAACTAAAAGGTTGGTGGTTCGAA
CCCACCCAGAGGCG
Homo_sapiens_chr1.trna25-AsnGTT (146467429-146467502)
Asn (GTT) 74 bp Sc: 72.61
(SEQ ID NO: 268)
GTCTCTGTGGCGTAGTCGGTTAGCGCGTTCGGCTGTTAACCGAAAAGTTGGTGGTTCGAG
CCCACCCAGGAACG
Homo_sapiens_chr1.trna103-AsnGTT (147497267-147497194)
Asn (GTT) 74 bp Sc: 77.16
(SEQ ID NO: 269)
GTCTCTGTGGCGCAATGGGTTAGCGCGTTCGGCTGTTAACCGAAAGGTTGGTGGTTCGAG
CCCATCCAGGGACG
Homo_sapiens_chr1.trna135-AsnGTT (16719740-16719667)
Asn (GTT) 74 bp Sc: 77.65
(SEQ ID NO: 270)
GTCTCTGTGGCGCAATCGGTTAGCGCGTTCGGCTGTTAACTGAAAGGTTGGTGGTTCGAG
CCCACCCAGGGACG
Homo_sapiens_chr1.trna7-AsnGTT (17088759-17088832)
Asn (GTT) 74 bp Sc: 80.35
(SEQ ID NO: 271)
GTCTCTGTGGCGCAATCGGTTAGCGCGTTCGGCTGTTAACCGAAAGATTGGTGGTTCGAG
CCCACCCAGGGACG
Homo_sapiens_chr1.trna107-AsnGTT (147027053-147026980)
Asn (GTT) 74 bp Sc: 80.52
(SEQ ID NO: 272)
GTCTCTGTGGCGCAATCGGTTAGCGCATTCGGCTGTTAACCGAAAGGTTGGTGGTTCGAG
CCCACCCAGGGACG
Homo_sapiens_chr1.trna108-AsnGTT (146865011-146864938)
Asn (GTT) 74 bp Sc: 80.52
(SEQ ID NO: 273)
GTCTCTGTGGCGCAATCGGTTAGCGCATTCGGCTGTTAACCGAAAGGTTGGTGGTTCGAG
CCCACCCAGGGACG
Homo_sapiens_chr1.trna26-AsnGTT (146614739-146614812)
Asn (GTT) 74 bp Sc: 82.24
(SEQ ID NO: 274)
GTCTCTGTGGCGCAATCGGTTAGCGCGTTCGGCTGTTAACCGAAAGGTTGGTGGTTCGAG
CCCACCCAGGGACG
Homo_sapiens_chr1.trna83-AsnGTT (159664564-159664491)
Asn (GTT) 74 bp Sc: 82.24
(SEQ ID NO: 275)
GTCTCTGTGGCGCAATCGGTTAGCGCGTTCGGCTGTTAACCGAAAGGTTGGTGGTTCGAG
CCCACCCAGGGACG
Homo_sapiens_chr10.trna4-AsnGTT (22558517-22558444)
Asn (GTT) 74 bp Sc: 82.24
(SEQ ID NO: 276)
GTCTCTGTGGCGCAATCGGTTAGCGCGTTCGGCTGTTAACCGAAAGGTTGGTGGTTCGAG
CCCACCCAGGGACG
Homo_sapiens_chr13.trna7-AsnGTT (30146174-30146101)
Asn (GTT) 74 bp Sc: 82.24
(SEQ ID NO: 277)
GTCTCTGTGGCGCAATCGGTTAGCGCGTTCGGCTGTTAACCGAAAGGTTGGTGGTTCGAG
CCCACCCAGGGACG
Homo_sapiens_chr17.trna31-AsnGTT (34161633-34161560)
Asn (GTT) 74 bp Sc: 82.24
(SEQ ID NO: 278)
GTCTCTGTGGCGCAATCGGTTAGCGCGTTCGGCTGTTAACCGAAAGGTTGGTGGTTCGAG
CCCACCCAGGGACG
Homo_sapiens_chr19.trna1-AsnGTT (1334562-1334635)
Asn (GTT) 74 bp Sc: 82.24
(SEQ ID NO: 279)
GTCTCTGTGGCGCAATCGGTTAGCGCGTTCGGCTGTTAACCGAAAGGTTGGTGGTTCGAG
CCCACCCAGGGACG
Homo_sapiens_chr1.trna47-AsnGTT (159776655-159776728)
Asn (GTT) 74 bp Sc: 83.66
(SEQ ID NO: 280)
GTCTCTGTGGCGCAATCGGTTAGCGCGTTCGGCTGTTAACCGAAAGGTTGGTGGTTCGAT
CCCACCCAGGGACG
Homo_sapiens_chr1.trna48-AspGTC (159841212-159841283)
Asp (GTC) 72 bp Sc: 30.22
(SEQ ID NO: 281)
TCCTTGTTACTATAGTGGTAAGTATCTCTGCCTGTCATGCATGAGAGAGGGGGTCGATTC
CCTGACGGGGAG
Homo_sapiens_chr12.trna7-AspGTC (121426877-121426947)
Asp (GTC) 71 bp Sc: 32.33
(SEQ ID NO: 282)
TCCTTGTTAGTATAGTGGTGAGTGTTTCTGCCTGTCATGTGGAGACTGGAGTTTGAGTCC
CCAACAGGGAG
Homo_sapiens_chr1.trna46-AspGTC (159768539-159768610)
Asp (GTC) 72 bp Sc: 34.08
(SEQ ID NO: 283)
TACTCGTTAGTATAGTGGTGCGTATCCCCGTCTGTCACGCGGGAGAGCGGGGTTCGCTCT
CCCGACGGGGAG
Homo_sapiens_chr3.trna3-AspGTC (185848859-185848789)
Asp (GTC) 71 bp Sc: 43.84
(SEQ ID NO: 284)
TTCTTGTTAATATAGTGGTGAGTATTCCCACCTGTCATGCGGGAGACGGGGTTCAATTCC
CTGATGGGGAG
Homo_sapiens_chr5.trna22-AspGTC (141754243-141754172)
Asp (GTC) 72 bp Sc: 54.87
(SEQ ID NO: 285)
TCCTCATCAGTATAGTGGTGAGTATCCCCGCCTGTCACGCGGGAGACTGGGGTTCGATTC
CCTGAGGAGGAG
Homo_sapiens_chr9.trna6-AspGTC (76707881-76707810)
Asp (GTC) 72 bp Sc: 54.95
(SEQ ID NO: 286)
TCCTCGTTAGTATGGTGGTGAGTATCCCTGCCTGTCACGCGGGAGACCGGGGTTCGATTC
CCCAACGGGGAG
Homo_sapiens_chr6.trna144-AspGTC (27659286-27659215)
Asp (GTC) 72 bp Sc: 64.62
(SEQ ID NO: 287)
TCCTCGTTAGTATAGTGGTGAGTGTCCCCGTCTGTCACGCGGGAGACCGGGGTTCGATTC
CCCGACGGGGAG
Homo_sapiens_chr1.trna69-AspGTC (159706900-159706829)
Asp (GTC) 72 bp Sc: 72.92
(SEQ ID NO: 288)
TCCTCGTTAGTATAGTGGTGAGTATCCCCGCCTGTCACGCGGGAGACCGGGGTTCGATTC
CCCGACGGGGAG
Homo_sapiens_chr1.trna72-AspGTC (159699519-159699448)
Asp (GTC) 72 bp Sc: 72.92
(SEQ ID NO: 289)
TCCTCGTTAGTATAGTGGTGAGTATCCCCGCCTGTCACGCGGGAGACCGGGGTTCGATTC
CCCGACGGGGAG
Homo_sapiens_chr1.trna75-AspGTC (159692109-159692038)
Asp (GTC) 72 bp Sc: 72.92
(SEQ ID NO: 290)
TCCTCGTTAGTATAGTGGTGAGTATCCCCGCCTGTCACGCGGGAGACCGGGGTTCGATTC
CCCGACGGGGAG
Homo_sapiens_chr1.trna78-AspGTC (159684728-159684657)
Asp (GTC) 72 bp Sc: 72.92
(SEQ ID NO: 291)
TCCTCGTTAGTATAGTGGTGAGTATCCCCGCCTGTCACGCGGGAGACCGGGGTTCGATTC
CCCGACGGGGAG
Homo_sapiens_chr1.trna81-AspGTC (159677310-159677239)
Asp (GTC) 72 bp Sc: 72.92
(SEQ ID NO: 292)
TCCTCGTTAGTATAGTGGTGAGTATCCCCGCCTGTCACGCGGGAGACCGGGGTTCGATTC
CCCGACGGGGAG
Homo_sapiens_chr12.trna10-AspGTC (123990217-123990146)
Asp (GTC) 72 bp Sc: 72.92
(SEQ ID NO: 293)
TCCTCGTTAGTATAGTGGTGAGTATCCCCGCCTGTCACGCGGGAGACCGGGGTTCGATTC
CCCGACGGGGAG
Homo_sapiens_chr12.trna12-AspGTC (123977915-123977844)
Asp (GTC) 72 bp Sc: 72.92
(SEQ ID NO: 294)
TCCTCGTTAGTATAGTGGTGAGTATCCCCGCCTGTCACGCGGGAGACCGGGGTTCGATTC
CCCGACGGGGAG
Homo_sapiens_chr12.trna4-AspGTC (94953930-94954001)
Asp (GTC) 72 bp Sc: 72.92
(SEQ ID NO: 295)
TCCTCGTTAGTATAGTGGTGAGTATCCCCGCCTGTCACGCGGGAGACCGGGGTTCGATTC
CCCGACGGGGAG
Homo_sapiens_chr17.trna38-AspGTC (8066352-8066281)
Asp (GTC) 72 bp Sc: 72.92
(SEQ ID NO: 296)
TCCTCGTTAGTATAGTGGTGAGTATCCCCGCCTGTCACGCGGGAGACCGGGGTTCGATTC
CCCGACGGGGAG
Homo_sapiens_chr6.trna45-AspGTC (27555432-27555503)
Asp (GTC) 72 bp Sc: 72.92
(SEQ ID NO: 297)
TCCTCGTTAGTATAGTGGTGAGTATCCCCGCCTGTCACGCGGGAGACCGGGGTTCGATTC
CCCGACGGGGAG
Homo_sapiens_chr6.trna48-AspGTC (27579502-27579573)
Asp (GTC) 72 bp Sc: 72.92
(SEQ ID NO: 298)
TCCTCGTTAGTATAGTGGTGAGTATCCCCGCCTGTCACGCGGGAGACCGGGGTTCGATTC
CCCGACGGGGAG
Homo_sapiens_chr12.trna5-AspGTC (97421412-97421483)
Asp (GTC) 72 bp Sc: 74.76
(SEQ ID NO: 299)
TCCTCGTTAGTATAGTGGTTAGTATCCCCGCCTGTCACGCGGGAGACCGGGGTTCAATTC
CCCGACGGGGAG
Homo_sapiens_chr17.trna30-CysGCA (34243572-34243501)
Cys (GCA) 72 bp Sc: 51.58
(SEQ ID NO: 300)
GGGGGTAGGGCTCAGGGATAGAGCATTTGACTGCAGATCAAGAGGTCCCCGGTTCGAATC
TAGGTGCCCCCT
Homo_sapiens_chr7.trna19-CysGCA (148923309-148923238)
Cys (GCA) 72 bp Sc: 57.52
(SEQ ID NO: 301)
GGGGGTATAGCTCACAGGTAGAGCATTTGACTGCAGATCAAGAGGTCCCCGGTTCAAATC
CGGTTACTCCCT
Homo_sapiens_chr7.trna8-CysGCA (148884735-148884806)
Cys (GCA) 72 bp Sc: 59.93
(SEQ ID NO: 302)
GGGCGTATAGCTCAGGGGTAGAGCATTTGACTGCAGATCAAGAGGTCCCCAGTTCAAATC
TGGGTGCCCACT
Homo_sapiens_chr7.trna14-CysGCA (148992848-148992919)
Cys (GCA) 72 bp Sc: 60.15
(SEQ ID NO: 303)
GGGGGTATAGCTCACAGGTAGAGCATTTGACTGCAGATCAAGAGGTCCCCGGTTCAAATC
TGGGTGCCCCCT
Cys (GCA) 72 bp Sc: 65.84
(SEQ ID NO: 304)
GGGCGTATAGCTCAGGGGTAGAGCATTTGACTGCAGATCAAGAGGTCCCCAGTTCAAATC
TGGGTGCCCCCT
Homo_sapiens_chr7.trna18-CysGCA (148941160-148941089)
Cys (GCA) 72 bp Sc: 67.69
(SEQ ID NO: 305)
GGGGGTATAGCTCAGGGGTAGAGCATTTGACTGCAAATCAAGAGGTCCCTGATTCAAATC
CAGGTGCCCCCT
Homo_sapiens_chr7.trna23-CysGCA (148703854-148703783)
Cys (GCA) 72 bp Sc: 68.91
(SEQ ID NO: 306)
GGGGGTATAGTTCAGGGGTAGAGCATTTGACTGCAGATCAAGAGGTCCCTGGTTCAAATC
CAGGTGCCCCCT
Homo_sapiens_chr7.trna16-CysGCA (149019276-149019205)
Cys (GCA) 72 bp Sc: 69.30
(SEQ ID NO: 307)
GGGGATATAGCTCAGGGGTAGAGCATTTGACTGCAGATCAAGAGGTCCCCGGTTCAAATC
CGGGTGCCCCCC
Homo_sapiens_chr7.trna7-CysGCA (148874564-148874635)
Cys (GCA) 72 bp Sc: 69.62
(SEQ ID NO: 308)
GGGGGTATAGCTCAGGGGTAGAGCACTTGACTGCAGATCAAGAAGTCCTTGGTTCAAATC
CAGGTGCCCCCT
Homo_sapiens_chr7.trna10-CysGCA (148912749-148912820)
Cys (GCA) 72 bp Sc: 69.82
(SEQ ID NO: 309)
GGGGGTATAGCTCAGGGGTAGAGCATTTGACTGCAGATCAAGAGGTCTCTGGTTCAAATC
CAGGTGCCCCCT
Homo_sapiens_chr17.trna29-CysGCA (34271534-34271463)
Cys (GCA) 72 bp Sc: 70.63
(SEQ ID NO: 310)
GGGGGTATAGCTCAGGGGTAGAGCATTTGACTGCAGATCAAGAAGTCCCCGGTTCAAATC
CGGGTGCCCCCT
Homo_sapiens_chr7.trna25-CysGCA (148683770-148683699)
Cys (GCA) 72 bp Sc: 71.19
(SEQ ID NO: 311)
GGGGGTATAGCTCAGGGGTAGAGCATTTGACTGCAGATCAAGAGGTCCCCAGTTCAAATC
TGGGTGCCCCCT
Homo_sapiens_chr7.trna17-CysGCA (148975050-148974979)
Cys (GCA) 72 bp Sc: 71.81
(SEQ ID NO: 312)
GGGGGTATAGCTTAGGGGTAGAGCATTTGACTGCAGATCAAAAGGTCCCTGGTTCAAATC
CAGGTGCCCCTT
Homo_sapiens_chr7.trna21-CysGCA (148743233-148743162)
Cys (GCA) 72 bp Sc: 73.76
(SEQ ID NO: 313)
GGGGGTATAGCTTAGCGGTAGAGCATTTGACTGCAGATCAAGAGGTCCCCGGTTCAAATC
CGGGTGCCCCCT
Homo_sapiens_chr7.trna22-CysGCA (148705605-148705534)
Cys (GCA) 72 bp Sc: 73.86
(SEQ ID NO: 314)
GGGGGTATAGCTCAGGGGTAGAGCATTTGACTGCAGATCAAGAGGTCCCTGGTTCAAATC
CAGGTGCCCCCC
Homo_sapiens_chr3.trna6-CysGCA (133433403-133433332)
Cys (GCA) 72 bp Sc: 74.20
(SEQ ID NO: 315)
GGGGGTATAGCTCAGGGGTAGAGCATTTGACTGCAGATCAAGAGGTCCCTGGTTCAAATC
CAGGTGCCCCCT
Homo_sapiens_chr7.trna13-CysGCA (148963711-148963782)
Cys (GCA) 72 bp Sc: 74.20
(SEQ ID NO: 316)
GGGGGTATAGCTCAGGGGTAGAGCATTTGACTGCAGATCAAGAGGTCCCTGGTTCAAATC
CAGGTGCCCCCT
Homo_sapiens_chr7.trna15-CysGCA (149035693-149035764)
Cys (GCA) 72 bp Sc: 74.20
(SEQ ID NO: 317)
GGGGGTATAGCTCAGGGGTAGAGCATTTGACTGCAGATCAAGAGGTCCCTGGTTCAAATC
CAGGTGCCCCCT
Homo_sapiens_chr7.trna6-CysGCA (148659153-148659224)
Cys (GCA) 72 bp Sc: 74.20
(SEQ ID NO: 318)
GGGGGTATAGCTCAGGGGTAGAGCATTTGACTGCAGATCAAGAGGTCCCTGGTTCAAATC
CAGGTGCCCCCT
Homo_sapiens_chr14.trna8-CysGCA (72499432-72499503)
Cys (GCA) 72 bp Sc: 74.26
(SEQ ID NO: 319)
GGGGGTATAGCTCAGGGGTAGAGCATTTGACTGCAGATCAAGAGGTCCCCGGTTCAAATC
CGGGTGCCCCCT
Homo_sapiens_chr1.trna126-CysGCA (93754494-93754422)
Cys (GCA) 73 bp Sc: 74.38
(SEQ ID NO: 320)
GGGGGTATAGCTCAGGTGGTAGAGCATTTGACTGCAGATCAAGAGGTCCCCGGTTCAAAT
CCGGGTGCCCCCT
Homo_sapiens_chr3.trna7-CysGCA (133430705-133430634)
Cys (GCA) 72 bp Sc: 74.42
(SEQ ID NO: 321)
GGGGGTGTAGCTCAGTGGTAGAGCATTTGACTGCAGATCAAGAGGTCCCTGGTTCAAATC
CAGGTGCCCCCT
Homo_sapiens_chr15.trna3-CysGCA (77824052-77824124)
Cys (GCA) 73 bp Sc: 74.95
(SEQ ID NO: 322)
GGGGGTATAGCTCAGTGGGTAGAGCATTTGACTGCAGATCAAGAGGTCCCCGGTTCAAAT
CCGGGTGCCCCCT
Homo_sapiens_chr17.trna28-CysGCA (34279142-34279071)
Cys (GCA) 72 bp Sc: 77.18
(SEQ ID NO: 323)
GGGGGTATAGCTCAGTGGTAGAGCATTTGACTGCAGATCAAGAGGTCCCTGGTTCAAATC
CGGGTGCCCCCT
Homo_sapiens_chr7.trna20-CysGCA (148917168-148917097)
Cys (GCA) 72 bp Sc: 77.55
(SEQ ID NO: 324)
GGGGGTATAGCTCAGGGGTAGAGCACTTGACTGCAGATCAAGAGGTCCCTGGTTCAAATC
CAGGTGCCCCCT
Homo_sapiens_chr17.trna15-CysGCA (34277424-34277495)
Cys (GCA) 72 bp Sc: 77.71
(SEQ ID NO: 325)
GGGGGTATAGCTCAGTGGTAGAGCATTTGACTGCAGATCAAGAGGTCCCCGGTTCAAATC
CGGGTGCCCCCT
Homo_sapiens_chr17.trna26-CysGCA (34564341-34564270)
Cys (GCA) 72 bp Sc: 77.71
(SEQ ID NO: 326)
GGGGGTATAGCTCAGTGGTAGAGCATTTGACTGCAGATCAAGAGGTCCCCGGTTCAAATC
CGGGTGCCCCCT
Homo_sapiens_chr17.trna27-CysGCA (34563584-34563513)
Cys (GCA) 72 bp Sc: 77.71
(SEQ ID NO: 327)
GGGGGTATAGCTCAGTGGTAGAGCATTTGACTGCAGATCAAGAGGTCCCCGGTTCAAATC
CGGGTGCCCCCT
Homo_sapiens_chr4.trna3-CysGCA (124649526-124649455)
Cys (GCA) 72 bp Sc: 77.71
(SEQ ID NO: 328)
GGGGGTATAGCTCAGTGGTAGAGCATTTGACTGCAGATCAAGAGGTCCCCGGTTCAAATC
CGGGTGCCCCCT
Homo_sapiens_chr7.trna5-CysGCA (148638214-148638285)
Cys (GCA) 72 bp Sc: 79.54
(SEQ ID NO: 329)
GGGGGCATAGCTCAGTGGTAGAGCATTTGACTGCAGATCAAGAGGTCCCTGGTTCAAATC
CAGGTGCCCCCT
Homo_sapiens_chr16.trna21-GlnCTG (70282391-70282464)
Gln (CTG) 74 bp Sc: 20.83
(SEQ ID NO: 330)
GGCAGTATGGTAGAGTGGTTAAGATCATGAACTCTGAAGTCAGAGATACTTGAATTTGAA
TGCTGGTTCTGTCA
Homo_sapiens_chr20.trna1-GlnCTG (17803142-17803219)
Gln (CTG) 78 bp Sc: 21.25
(SEQ ID NO: 331)
GGCAGTGTAGCCCAGAGGTTCAAGGGCATTCGCTCTGGTATCAGAAGGGTCTGGGTTCAA
ATCCCTTGTGCACTGCTT
Homo_sapiens_chr9.trna5-GlnCTG (125695415-125695343)
Gln (CTG) 73 bp Sc: 22.97
(SEQ ID NO: 332)
GAGCTGTAGCATAGTGATTAGGGACATGGACTCTGGAGCCAAATCTGCCTGGGTTCTAGT
CCCAGCTGTCTCA
Homo_sapiens_chr12.trna3-GlnCTG (73137449-73137521)
Gln (CTG) 73 bp Sc: 34.73
(SEQ ID NO: 333)
GGTTCCATGGTGTAATGGTAAGCACCCTGGACTCTGAATCCAGCAACCAGAGTTCCAGTC
TCAGCGTGGACCT
Homo_sapiens_chr1.trna114-GlnCTG (144943455-144943384)
Gln (CTG) 72 bp Sc: 40.01
(SEQ ID NO: 334)
GGTTCCATGGTGTAATGGTGACCACTTTGGACTCTGAATACAGTGATCAGAGTTCAAGTC
TCACTGGAACCT
Homo_sapiens_chr1.trna120-GlnCTG (144090748-144090677)
Gln (CTG) 72 bp Sc: 44.40
(SEQ ID NO: 335)
GGTTCCATGGTGTAATGGTGAGCACTTTGGACTCTGAATACAGTGATCAGAGTTCAAGTC
TCACTGGGACCT
Homo_sapiens_chr1.trna121-GlnCTG (143550864-143550793)
Gln (CTG) 72 bp Sc: 44.40
(SEQ ID NO: 336)
GGTTCCATGGTGTAATGGTGAGCACTTTGGACTCTGAATACAGTGATCAGAGTTCAAGTC
TCACTGGGACCT
Homo_sapiens_chr1_random.trna1-GlnCTG (553277-553348)
Gln (CTG) 72 bp Sc: 44.40
(SEQ ID NO: 337)
GGTTCCATGGTGTAATGGTGAGCACTTTGGACTCTGAATACAGTGATCAGAGTTCAAGTC
TCACTGGGACCT
Homo_sapiens_chr1.trna22-GlnCTG (146267561-146267632)
Gln (CTG) 72 bp Sc: 56.30
(SEQ ID NO: 338)
GGTTCCATGGTGTAATGGTAAGCACTCTGGACTCTGAATCCAGCCATCTGAGTTCGAGTC
TCTGTGGAACCT
Homo_sapiens_chr6.trna131-GlnCTG (27867185-27867114)
Gln (CTG) 72 bp Sc: 66.25
(SEQ ID NO: 339)
GGCCCCATGGTGTAATGGTCAGCACTCTGGACTCTGAATCCAGCGATCCGAGTTCAAATC
TCGGTGGGACCC
Homo_sapiens_chr6.trna42-GlnCTG (27371191-27371262)
Gln (CTG) 72 bp Sc: 68.41
(SEQ ID NO: 340)
GGTTCCATGGTGTAATGGTTAGCACTCTGGACTCTGAATCCGGTAATCCGAGTTCAAATC
TCGGTGGAACCT
Homo_sapiens_chr1.trna112-GlnCTG (146204077-146204006)
Gln (CTG) 72 bp Sc: 68.84
(SEQ ID NO: 341)
GGTTCCATGGTGTAATGGTAAGCACTCTGGACTCTGAATCCAGCGATCCGAGTTCGAGTC
TCGGTGGAACCT
Homo_sapiens_chr1.trna28-GlnCTG (147452749-147452820)
Gln (CTG) 72 bp Sc: 68.84
(SEQ ID NO: 342)
GGTTCCATGGTGTAATGGTAAGCACTCTGGACTCTGAATCCAGCGATCCGAGTTCGAGTC
TCGGTGGAACCT
Homo_sapiens_chr1.trna15-GlnCTG (144674661-144674732)
Gln (CTG) 72 bp Sc: 70.16
(SEQ ID NO: 343)
GGTTCCATGGTGTAATGGTGAGCACTCTGGACTCTGAATCCAGCGATCCGAGTTCGAGTC
TCGGTGGAACCT
Homo_sapiens_chr1.trna19-GlnCTG (145971662-145971733)
Gln (CTG) 72 bp Sc: 70.16
(SEQ ID NO: 344)
GGTTCCATGGTGTAATGGTGAGCACTCTGGACTCTGAATCCAGCGATCCGAGTTCGAGTC
TCGGTGGAACCT
Homo_sapiens_chr6.trna146-GlnCTG (27623581-27623510)
Gln (CTG) 72 bp Sc: 72.00
(SEQ ID NO: 345)
GGTTCCATGGTGTAATGGTTAGCACTCTGGACTCTGAATCCAGCGATCCGAGTTCAAGTC
TCGGTGGAACCT
Homo_sapiens_chr15.trna7-GlnCTG (63948525-63948454)
Gln (CTG) 72 bp Sc: 73.65
(SEQ ID NO: 346)
GGTTCCATGGTGTAATGGTTAGCACTCTGGACTCTGAATCCAGCGATCCGAGTTCAAATC
TCGGTGGAACCT
Homo_sapiens_chr17.trna3-GlnCTG (7963795-7963866)
Gln (CTG) 72 bp Sc: 73.65
(SEQ ID NO: 347)
GGTTCCATGGTGTAATGGTTAGCACTCTGGACTCTGAATCCAGCGATCCGAGTTCAAATC
TCGGTGGAACCT
Homo_sapiens_chr6.trna1-GlnCTG (18944381-18944452)
Gln (CTG) 72 bp Sc: 73.65
(SEQ ID NO: 348)
GGTTCCATGGTGTAATGGTTAGCACTCTGGACTCTGAATCCAGCGATCCGAGTTCAAATC
TCGGTGGAACCT
Homo_sapiens_chr6.trna49-GlnCTG (27595287-27595358)
Gln (CTG) 72 bp Sc: 73.65
(SEQ ID NO: 349)
GGTTCCATGGTGTAATGGTTAGCACTCTGGACTCTGAATCCAGCGATCCGAGTTCAAATC
TCGGTGGAACCT
Homo_sapiens_chr6.trna99-GlnCTG (29017428-29017357)
Gln (CTG) 72 bp Sc: 73.65
(SEQ ID NO: 350)
GGTTCCATGGTGTAATGGTTAGCACTCTGGACTCTGAATCCAGCGATCCGAGTTCAAATC
TCGGTGGAACCT
Homo_sapiens_chr6.trna79-GlnTTG (37395973-37396045)
Gln (TTG) 73 bp Sc: 21.80
(SEQ ID NO: 351)
GTGCAGAGTAGTACAGTGGTTAAACCATGGTCTTTGGAGCCAGACTGCCTGGGGTCGGAT
CCCAGCTCTCACA
Homo_sapiens_chr16.trna14-GlnTTG (3359814-3359885)
Gln (TTG) 72 bp Sc: 30.33
(SEQ ID NO: 352)
TAGGACTTGGTGTAATGGGTAGCACAGAGAATTTTGGATTCTCAGGGGTGGGTTCAATTC
CTTTCGTCCTAG
Homo_sapiens_chr4.trna4-GlnTTG (40603572-40603500)
Gln (TTG) 73 bp Sc: 31.57
(SEQ ID NO: 353)
GACCATGTGGCCTAAGGGAAAAGACATCTCACTTTGGGTCAGAAGATTGAGGGTTCAAGT
CCTTTCATGGTCA
Homo_sapiens_chr2.trna23-GlnTTG (117499050-117498979)
Gln (TTG) 72 bp Sc: 33.55
(SEQ ID NO: 354)
TAGGACATGGTGTAATAGGTAGAATGGAGAATTTTGAATTCTCAGGGGTAGGTTCAATTC
CTATAGTTCTAG
Homo_sapiens_chr6.trna84-GlnTTG (145545552-145545623)
Gln (TTG) 72 bp Sc: 59.31
(SEQ ID NO: 355)
GGTCCCATGGTGTAATGGTTAGCACTCTGGGCTTTGAATCCAGCAATCCGAGTTCGAATC
TTGGTGGGACCT
Homo_sapiens_chr6.trna130-GlnTTG (27871690-27871619)
Gln (TTG) 72 bp Sc: 68.31
(SEQ ID NO: 356)
GGCCCCATGGTGTAATGGTTAGCACTCTGGACTTTGAATCCAGCGATCCGAGTTCAAATC
TCGGTGGGACCT
Homo_sapiens_chr6.trna173-GlnTTG (26420025-26419954)
Gln (TTG) 72 bp Sc: 68.31
(SEQ ID NO: 357)
GGCCCCATGGTGTAATGGTTAGCACTCTGGACTTTGAATCCAGCGATCCGAGTTCAAATC
TCGGTGGGACCT
Homo_sapiens_chr6.trna174-GlnTTG (26419474-26419403)
Gln (TTG) 72 bp Sc: 68.31
(SEQ ID NO: 358)
GGCCCCATGGTGTAATGGTTAGCACTCTGGACTTTGAATCCAGCGATCCGAGTTCAAATC
TCGGTGGGACCT
Homo_sapiens_chr17.trna16-GlnTTG (44624889-44624960)
Gln (TTG) 72 bp Sc: 73.77
(SEQ ID NO: 359)
GGTCCCATGGTGTAATGGTTAGCACTCTGGACTTTGAATCCAGCGATCCGAGTTCAAATC
TCGGTGGGACCT
Homo_sapiens_chr1.trna29-GluCTC (147600896-147600964)
Glu (CTC) 69 bp Sc: 21.64
(SEQ ID NO: 360)
TCCCTGGTAGTCTAGTGGCTAAAGTTTGGCGCTCTCACCGCCGGGACTGGTTGATTCCAG
ATCAGGGGA
Homo_sapiens_chr3.trna8-GluCTC (126895938-126895867)
Glu (CTC) 72 bp Sc: 29.87
(SEQ ID NO: 361)
CCCCTGGTGGTCTATCGGTTAGGATTCAGACCTCTCACCACTGCTACCCATGCTCGATTC
CTGGTCAGGGAA
Homo_sapiens_chr18.trna1-GluCTC (41553749-41553820)
Glu (CTC) 72 bp Sc: 32.13
(SEQ ID NO: 362)
CCCCGGGTGGTGTAGTGGATGGGATTTGGCGCTCTCACCACCATGGCCCGGATTTGATTC
CCGGTCAGGGAA
Homo_sapiens_chr8.trna3-GluCTC (59667352-59667422)
Glu (CTC) 71 bp Sc: 33.87
(SEQ ID NO: 363)
TCCTTGATGTCTAGTGGTTAGGATTTGGTGCTCTCACTGCAGCAGCCTGGGTTCATTTCT
CAGTCAGGGAA
Homo_sapiens_chr13.trna4-GluCTC (40928132-40928061)
Glu (CTC) 72 bp Sc: 41.44
(SEQ ID NO: 364)
CCCCTGGTGGTCTAGTGCTTAGGATTCGGTGCTCTCACCGCTGCTGCCTGCGTTCGATTC
CCGGTCAGGGAA
Homo_sapiens_chr1.trna59-GluCTC (247135070-247135141)
Glu (CTC) 72 bp Sc: 71.50
(SEQ ID NO: 365)
TCCCTGGTGGTCTAGTGGTTAGGATTCGGCGCTCTCACCGCCGCGGCCCGGGTTCGATTC
CCGGTCAGGAAA
Homo_sapiens_chr1.trna116-GluCTC (144110661-144110590)
Glu (CTC) 72 bp Sc: 76.61
(SEQ ID NO: 366)
TCCCTGGTGGTCTAGTGGTTAGGATTCGGCGCTCTCACCGCCGCGGCCCGGGTTCGATTC
CCGGTCAGGGAA
Homo_sapiens_chr1.trna71-GluCTC (159705884-159705813)
Glu (CTC) 72 bp Sc: 76.61
(SEQ ID NO: 367)
TCCCTGGTGGTCTAGTGGTTAGGATTCGGCGCTCTCACCGCCGCGGCCCGGGTTCGATTC
CCGGTCAGGGAA
Homo_sapiens_chr1.trna74-GluCTC (159698504-159698433)
Glu (CTC) 72 bp Sc: 76.61
(SEQ ID NO: 368)
TCCCTGGTGGTCTAGTGGTTAGGATTCGGCGCTCTCACCGCCGCGGCCCGGGTTCGATTC
CCGGTCAGGGAA
Homo_sapiens_chr1.trna77-GluCTC (159691093-159691022)
Glu (CTC) 72 bp Sc: 76.61
(SEQ ID NO: 369)
TCCCTGGTGGTCTAGTGGTTAGGATTCGGCGCTCTCACCGCCGCGGCCCGGGTTCGATTC
CCGGTCAGGGAA
Homo_sapiens_chr1.trna80-GluCTC (159683713-159683642)
Glu (CTC) 72 bp Sc: 76.61
(SEQ ID NO: 370)
TCCCTGGTGGTCTAGTGGTTAGGATTCGGCGCTCTCACCGCCGCGGCCCGGGTTCGATTC
CCGGTCAGGGAA
Homo_sapiens_chr6.trna77-GluCTC (29057955-29058026)
Glu (CTC) 72 bp Sc: 76.61
(SEQ ID NO: 371)
TCCCTGGTGGTCTAGTGGTTAGGATTCGGCGCTCTCACCGCCGCGGCCCGGGTTCGATTC
CCGGTCAGGGAA
Homo_sapiens_chr6.trna87-GluCTC (126143157-126143086)
Glu (CTC) 72 bp Sc: 76.61
(SEQ ID NO: 372)
TCCCTGGTGGTCTAGTGGTTAGGATTCGGCGCTCTCACCGCCGCGGCCCGGGTTCGATTC
CCGGTCAGGGAA
Homo_sapiens_chr1.trna31-GluTTC (147986426-147986494)
Glu (TTC) 69 bp Sc: 21.27
(SEQ ID NO: 373)
TCCCTGGTAGTCTAGTGGCTAAAGTTTGGCGCTTTCACCGCCGGGACTGGTTGATTCCAG
ATCAGGGGA
Homo_sapiens_chr2.trna6-GluTTC (74977554-74977622)
Glu (TTC) 69 bp Sc: 23.54
(SEQ ID NO: 374)
GCCTGTGGTCTAGTGGTTAGAATTCAGTGTTTTCAGTGCTCTAGTCCAGGTTCAATTCCT
GGTCAGGGA
Homo_sapiens_chr1.trna64-GluTTC (170424230-170424162)
Glu (TTC) 69 bp Sc: 26.19
(SEQ ID NO: 375)
CCCTGTGGTCTAGTGGTTAGGAGTTGGTGCTTTCGTCATGACAGCCCAGGTTCAATTCCT
GGTTAGAGA
Homo_sapiens_chr14.trna14-GluTTC (31306637-31306567)
Glu (TTC) 71 bp Sc: 28.80
(SEQ ID NO: 376)
ACCCTGTGGTCTAGTGGCTAAGACTTTGTGCTTTCATTGCTGCATCCTAGGTTCAATTCC
CAGTCAGGGAA
Homo_sapiens_chr1.trna94-GluTTC (147931051-147930979)
Glu (TTC) 73 bp Sc: 40.45
(SEQ ID NO: 377)
TCCCTGGTGGTCTAGTGGCTAGGATTCGGCGCTTTCACCGCCTGCAGCTCGAGTTCGATT
CCTGGTCAGGGAA
Homo_sapiens_chr1.trna49-GluTTC (159849132-159849203)
Glu (TTC) 72 bp Sc: 54.07
(SEQ ID NO: 378)
GCGTTGGTGGTGTAGTGGTGAGCACAGCTGCCTTTCAAGCAGTTAACGCGGGTTCGATTC
CCGGGTAACGAA
Homo_sapiens_chr1.trna133-GluTTC (16734432-16734361)
Glu (TTC) 72 bp Sc: 72.33
(SEQ ID NO: 379)
TCCCTGGTGGTCTAGTGGCTAGGATTCGGCGCTTTCACCGCCGCGGCCCGGGTTCGATTC
CCGGTCAGGGAA
Homo_sapiens_chr1.trna84-GluTTC (159658578-159658507)
Glu (TTC) 72 bp Sc: 72.33
(SEQ ID NO: 380)
TCCCTGGTGGTCTAGTGGCTAGGATTCGGCGCTTTCACCGCCGCGGCCCGGGTTCGATTC
CCGGTCAGGGAA
Homo_sapiens_chr1.trna5-GluTTC (17071665-17071736)
Glu (TTC) 72 bp Sc: 75.96
(SEQ ID NO: 381)
TCCCTGGTGGTCTAGTGGCTAGGATTCGGCGCTTTCACCGCCGCGGCCCGGGTTCGATTC
CCGGCCAGGGAA
Homo_sapiens_chr13.trna3-GluTTC (44390133-44390062)
Glu (TTC) 72 bp Sc: 76.26
(SEQ ID NO: 382)
TCCCACATGGTCTAGCGGTTAGGATTCCTGGTTTTCACCCAGGCGGCCCGGGTTCGACTC
CCGGTGTGGGAA
Homo_sapiens_chr15.trna11-GluTTC (23878545-23878474)
Glu (TTC) 72 bp Sc: 76.26
(SEQ ID NO: 383)
TCCCACATGGTCTAGCGGTTAGGATTCCTGGTTTTCACCCAGGCGGCCCGGGTTCGACTC
CCGGTGTGGGAA
Homo_sapiens_chr13.trna5-GluTTC (40532945-40532874)
Glu (TTC) 72 bp Sc: 76.46
(SEQ ID NO: 384)
TCCCATATGGTCTAGCGGTTAGGATTCCTGGTTTTCACCCAGGTGGCCCGGGTTCGACTC
CCGGTATGGGAA
Homo_sapiens_chr2.trna20-GluTTC (130811242-130811171)
Glu (TTC) 72 bp Sc: 76.46
(SEQ ID NO: 385)
TCCCATATGGTCTAGCGGTTAGGATTCCTGGTTTTCACCCAGGTGGCCCGGGTTCGACTC
CCGGTATGGGAA
Homo_sapiens_chr1.trna2-GlyCCC (16926367-16926437)
Gly (CCC) 71 bp Sc: 63.06
(SEQ ID NO: 386)
GCCTTGGTGGTGCAGTGGTAGAATTCTCGCCTCCCACGTGGGAGACCCGGGTTCAATTCC
CGGCCAATGCA
Homo_sapiens_chr1.trna130-GlyCCC (16877423-16877353)
Gly (CCC) 71 bp Sc: 69.75
(SEQ ID NO: 387)
GCGTTGGTGGTTTAGTGGTAGAATTCTCGCCTCCCATGCGGGAGACCCGGGTTCAATTCC
CGGCCACTGCA
Homo_sapiens_chr17.trna13-GlyCCC (19704767-19704837)
Gly (CCC) 71 bp Sc: 70.13
(SEQ ID NO: 388)
GCATTGGTGGTTCAATGGTAGAATTCTCGCCTCCCACGCAGGAGACCCAGGTTCGATTCC
TGGCCAATGCA
Homo_sapiens_chr16.trna34-GlyCCC (626807-626737)
Gly (CCC) 71 bp Sc: 76.98
(SEQ ID NO: 389)
GCGCCGCTGGTGTAGTGGTATCATGCAAGATTCCCATTCTTGCGACCCGGGTTCGATTCC
CGGGCGGCGCA
Homo_sapiens_chr2.trna27-GlyCCC (70329697-70329627)
Gly (CCC) 71 bp Sc: 76.98
(SEQ ID NO: 390)
GCGCCGCTGGTGTAGTGGTATCATGCAAGATTCCCATTCTTGCGACCCGGGTTCGATTCC
CGGGCGGCGCA
Homo_sapiens_chr1.trna132-GlyCCC (16745091-16745021)
Gly (CCC) 71 bp Sc: 78.31
(SEQ ID NO: 391)
GCATTGGTGGTTCAGTGGTAGAATTCTCGCCTCCCACGCGGGAGACCCGGGTTCAATTCC
CGGCCAATGCA
Homo_sapiens_chr1.trna4-GlyCCC (17061003-17061073)
Gly (CCC) 71 bp Sc: 78.31
(SEQ ID NO: 392)
GCATTGGTGGTTCAGTGGTAGAATTCTCGCCTCCCACGCGGGAGACCCGGGTTCAATTCC
CGGCCAATGCA
Homo_sapiens_chr6.trna82-GlyGCC (142620469-142620539)
Gly (GCC) 71 bp Sc: 46.73
(SEQ ID NO: 393)
GCATGGGTGATTCAGTGGTAGAATTTTCACCTGCCATGCAGGAGGTCCAGGTTCATTTCC
TGGCCTATGCA
Homo_sapiens_chr16.trna18-GlyGCC (69380098-69380168)
Gly (GCC) 71 bp Sc: 56.35
(SEQ ID NO: 394)
GCATTGGTGGTTCAGTGGTAGAATTCTCGCCTGCCATGCGGGCGGCCGGGCTTCGATTCC
TGGCCAATGCA
Homo_sapiens_chr1.trna43-GlyGCC (159716980-159717050)
Gly (GCC) 71 bp Sc: 69.02
(SEQ ID NO: 395)
GCATAGGTGGTTCAGTGGTAGAATTCTTGCCTGCCACGCAGGAGGCCCAGGTTTGATTCC
TGGCCCATGCA
Homo_sapiens_chr16.trna25-GlyGCC (69369685-69369615)
Gly (GCC) 71 bp Sc: 73.97
(SEQ ID NO: 396)
GCATTGGTGGTTCAGTGGTAGAATTCTCGCCTGCCACGCGGGAGGCCCGGGTTTGATTCC
CGGCCAGTGCA
Homo_sapiens_chr1.trna68-GlyGCC (159760331-159760261)
Gly (GCC) 71 bp Sc: 81.62
(SEQ ID NO: 397)
GCATTGGTGGTTCAGTGGTAGAATTCTCGCCTGCCACGCGGGAGGCCCGGGTTCGATTCC
CGGCCAATGCA
Homo_sapiens_chr16.trna19-GlyGCC (69380911-69380981)
Gly (GCC) 71 bp Sc: 81.62
(SEQ ID NO: 398)
GCATTGGTGGTTCAGTGGTAGAATTCTCGCCTGCCACGCGGGAGGCCCGGGTTCGATTCC
CGGCCAATGCA
Homo_sapiens_chr16.trna24-GlyGCC (69370513-69370443)
Gly (GCC) 71 bp Sc: 81.62
(SEQ ID NO: 399)
GCATTGGTGGTTCAGTGGTAGAATTCTCGCCTGCCACGCGGGAGGCCCGGGTTCGATTCC
CGGCCAATGCA
Homo_sapiens_chr17.trna5-GlyGCC (7969789-7969859)
Gly (GCC) 71 bp Sc: 81.62
(SEQ ID NO: 400)
GCATTGGTGGTTCAGTGGTAGAATTCTCGCCTGCCACGCGGGAGGCCCGGGTTCGATTCC
CGGCCAATGCA
Homo_sapiens_chr2.trna19-GlyGCC (156965975-156965905)
Gly (GCC) 71 bp Sc: 81.62
(SEQ ID NO: 401)
GCATTGGTGGTTCAGTGGTAGAATTCTCGCCTGCCACGCGGGAGGCCCGGGTTCGATTCC
CGGCCAATGCA
Homo_sapiens_chr6.trna128-GlyGCC (27978735-27978665)
Gly (GCC) 71 bp Sc: 81.62
(SEQ ID NO: 402)
GCATTGGTGGTTCAGTGGTAGAATTCTCGCCTGCCACGCGGGAGGCCCGGGTTCGATTCC
CGGCCAATGCA
Homo_sapiens_chr1.trna35-GlyGCC (159679718-159679788)
Gly (GCC) 71 bp Sc: 82.15
(SEQ ID NO: 403)
GCATGGGTGGTTCAGTGGTAGAATTCTCGCCTGCCACGCGGGAGGCCCGGGTTCGATTCC
CGGCCCATGCA
Homo_sapiens_chr1.trna37-GlyGCC (159687091-159687161)
Gly (GCC) 71 bp Sc: 82.15
(SEQ ID NO: 404)
GCATGGGTGGTTCAGTGGTAGAATTCTCGCCTGCCACGCGGGAGGCCCGGGTTCGATTCC
CGGCCCATGCA
Homo_sapiens_chr1.trna39-GlyGCC (159694522-159694592)
Gly (GCC) 71 bp Sc: 82.15
(SEQ ID NO: 405)
GCATGGGTGGTTCAGTGGTAGAATTCTCGCCTGCCACGCGGGAGGCCCGGGTTCGATTCC
CGGCCCATGCA
Homo_sapiens_chr1.trna41-GlyGCC (159701882-159701952)
Gly (GCC) 71 bp Sc: 82.15
(SEQ ID NO: 406)
GCATGGGTGGTTCAGTGGTAGAATTCTCGCCTGCCACGCGGGAGGCCCGGGTTCGATTCC
CGGCCCATGCA
Homo_sapiens_chr21.trna2-GlyGCC (17749048-17748978)
Gly (GCC) 71 bp Sc: 82.15
(SEQ ID NO: 407)
GCATGGGTGGTTCAGTGGTAGAATTCTCGCCTGCCACGCGGGAGGCCCGGGTTCGATTCC
CGGCCCATGCA
Homo_sapiens_chr1.trna82-GlyTCC (159676656-159676585)
Gly (TCC) 72 bp Sc: 55.96
(SEQ ID NO: 408)
GCGTTGGTGGTATAGTGGTGAGCATAGTTGCCTTCCAAGCAGTTGACCCGGGCTCGATTC
CCGCCCAACGCA
Homo_sapiens_chr17.trna10-GlyTCC (8065591-8065662)
Gly (TCC) 72 bp Sc: 71.94
(SEQ ID NO: 409)
GCGTTGGTGGTATAGTGGTAAGCATAGCTGCCTTCCAAGCAGTTGACCCGGGTTCGATTC
CCGGCCAACGCA
Homo_sapiens_chr1.trna117-GlyTCC (144109292-144109221)
Gly (TCC) 72 bp Sc: 73.26
(SEQ ID NO: 410)
GCGTTGGTGGTATAGTGGTGAGCATAGCTGCCTTCCAAGCAGTTGACCCGGGTTCGATTC
CCGGCCAACGCA
Homo_sapiens_chr1.trna45-GlyTCC (159767527-159767598)
Gly (TCC) 72 bp Sc: 73.26
(SEQ ID NO: 411)
GCGTTGGTGGTATAGTGGTGAGCATAGCTGCCTTCCAAGCAGTTGACCCGGGTTCGATTC
CCGGCCAACGCA
Homo_sapiens_chr1.trna70-GlyTCC (159706242-159706171)
Gly (TCC) 72 bp Sc: 73.26
(SEQ ID NO: 412)
GCGTTGGTGGTATAGTGGTGAGCATAGCTGCCTTCCAAGCAGTTGACCCGGGTTCGATTC
CCGGCCAACGCA
Homo_sapiens_chr1.trna73-GlyTCC (159698861-159698790)
Gly (TCC) 72 bp Sc: 73.26
(SEQ ID NO: 413)
GCGTTGGTGGTATAGTGGTGAGCATAGCTGCCTTCCAAGCAGTTGACCCGGGTTCGATTC
CCGGCCAACGCA
Homo_sapiens_chr1.trna76-GlyTCC (159691451-159691380)
Gly (TCC) 72 bp Sc: 73.26
(SEQ ID NO: 414)
GCGTTGGTGGTATAGTGGTGAGCATAGCTGCCTTCCAAGCAGTTGACCCGGGTTCGATTC
CCGGCCAACGCA
Homo_sapiens_chr1.trna79-GlyTCC (159684070-159683999)
Gly (TCC) 72 bp Sc: 73.26
(SEQ ID NO: 415)
GCGTTGGTGGTATAGTGGTGAGCATAGCTGCCTTCCAAGCAGTTGACCCGGGTTCGATTC
CCGGCCAACGCA
Homo_sapiens_chr19.trna2-GlyTCC (4675082-4675153)
Gly (TCC) 72 bp Sc: 76.83
(SEQ ID NO: 416)
GCGTTGGTGGTATAGTGGTTAGCATAGCTGCCTTCCAAGCAGTTGACCCGGGTTCGATTC
CCGGCCAACGCA
Homo_sapiens_chr3.trna4-HisGTG (149799324-149799253)
His (GTG) 72 bp Sc: 22.51
(SEQ ID NO: 417)
GCAGTGACTGTATAGTGGTTAGCACTCTGTGTTGTGGCCACAGCAACCATGGTTCAAATC
TGAGTCATGACA
Homo_sapiens_chr1.trna106-HisGTG (147422523-147422452)
His (GTG) 72 bp Sc: 61.09
(SEQ ID NO: 418)
GCCATGATCGTATAGTGGTTAGTACTCTGCGCTGTGGCCGCAGCAACCTCGGTTCGAATC
CGAGTCACGGCA
Homo_sapiens_chr1.trna111-HisGTG (146241540-146241469)
His (GTG) 72 bp Sc: 64.63
(SEQ ID NO: 419)
GCCGTGATCGTATAGTGGTTAGTACTCTGCGTTGTGGCCGCAGCAACCTCGGTTCGAATC
CGAGTCACGGCA
Homo_sapiens_chr1.trna118-HisGTG (144108309-144108238)
His (GTG) 72 bp Sc: 64.63
(SEQ ID NO: 420)
GCCGTGATCGTATAGTGGTTAGTACTCTGCGTTGTGGCCGCAGCAACCTCGGTTCGAATC
CGAGTCACGGCA
Homo_sapiens_chr1.trna16-HisGTG (145011397-145011468)
His (GTG) 72 bp Sc: 64.63
(SEQ ID NO: 421)
GCCGTGATCGTATAGTGGTTAGTACTCTGCGTTGTGGCCGCAGCAACCTCGGTTCGAATC
CGAGTCACGGCA
Homo_sapiens_chr1.trna21-HisGTG (146220095-146220166)
His (GTG) 72 bp Sc: 64.63
(SEQ ID NO: 422)
GCCGTGATCGTATAGTGGTTAGTACTCTGCGTTGTGGCCGCAGCAACCTCGGTTCGAATC
CGAGTCACGGCA
Homo_sapiens_chr15.trna1-HisGTG (43280641-43280712)
His (GTG) 72 bp Sc: 64.63
(SEQ ID NO: 423)
GCCGTGATCGTATAGTGGTTAGTACTCTGCGTTGTGGCCGCAGCAACCTCGGTTCGAATC
CGAGTCACGGCA
Homo_sapiens_chr15.trna8-HisGTG (43279974-43279903)
His (GTG) 72 bp Sc: 64.63
(SEQ ID NO: 424)
GCCGTGATCGTATAGTGGTTAGTACTCTGCGTTGTGGCCGCAGCAACCTCGGTTCGAATC
CGAGTCACGGCA
Homo_sapiens_chr15.trna9-HisGTG (43278167-43278096)
His (GTG) 72 bp Sc: 64.63
(SEQ ID NO: 425)
GCCGTGATCGTATAGTGGTTAGTACTCTGCGTTGTGGCCGCAGCAACCTCGGTTCGAATC
CGAGTCACGGCA
Homo_sapiens_chr6.trna33-HisGTG (27233885-27233956)
His (GTG) 72 bp Sc: 64.63
(SEQ ID NO: 426)
GCCGTGATCGTATAGTGGTTAGTACTCTGCGTTGTGGCCGCAGCAACCTCGGTTCGAATC
CGAGTCACGGCA
Homo_sapiens_chr9.trna7-HisGTG (14424009-14423938)
His (GTG) 72 bp Sc: 64.63
(SEQ ID NO: 427)
GCCGTGATCGTATAGTGGTTAGTACTCTGCGTTGTGGCCGCAGCAACCTCGGTTCGAATC
CGAGTCACGGCA
Homo_sapiens_chr6.trna38-IleAAT (27349718-27349791)
Ile (AAT) 74 bp Sc: 75.35
(SEQ ID NO: 428)
GGCTGGTTAGTTCAGTTGGTTAGAGCGTGGTGCTAATAACGCCAAGGTCGTGGGTTCGAT
CCCCATATCGGCCA
Homo_sapiens_chr6.trna57-IleAAT (27744341-27744414)
Ile (AAT) 74 bp Sc: 76.09
(SEQ ID NO: 429)
GGCCGGTTAGCTCAGTCGGCTAGAGCGTGGTGCTAATAACGCCAAGGTCGCGGGTTCGAT
CCCCGTACGGGCCA
Homo_sapiens_chr6.trna165-IleAAT (26829273-26829200)
Ile (AAT) 74 bp Sc: 78.57
(SEQ ID NO: 430)
GGCCGGTTAGCTCAGTTGGTCAGAGCGTGGTGCTAATAACGCCAAGGTCGCGGGTTCGAT
CCCCGTACGGGCCA
Homo_sapiens_chr6.trna28-IleAAT (26888811-26888884)
Ile (AAT) 74 bp Sc: 78.57
(SEQ ID NO: 431)
GGCCGGTTAGCTCAGTTGGTCAGAGCGTGGTGCTAATAACGCCAAGGTCGCGGGTTCGAT
CCCCGTACGGGCCA
Homo_sapiens_chr6.trna163-IleAAT (26853307-26853234)
Ile (AAT) 74 bp Sc: 79.57
(SEQ ID NO: 432)
GGCCGGTTAGCTCAGTTGGTTAGAGCGTGGTGCTAATAACGCTAAGGTCGCGGGTTCGAT
CCCCGTACGGGCCA
Homo_sapiens_chr14.trna10-IleAAT (101853182-101853255)
Ile (AAT) 74 bp Sc: 80.66
(SEQ ID NO: 433)
GGCCGGTTAGCTCAGTTGGTTAGAGCGTGGTGCTAATAACGCCAAGGTCGCGGGTTCGAT
CCCCGTACGGGCCA
Homo_sapiens_chr17.trna9-IleAAT (8031636-8031709)
Ile (AAT) 74 bp Sc: 80.66
(SEQ ID NO: 434)
GGCCGGTTAGCTCAGTTGGTTAGAGCGTGGTGCTAATAACGCCAAGGTCGCGGGTTCGAT
CCCCGTACGGGCCA
Homo_sapiens_chr6.trna11-IleAAT (26662329-26662402)
Ile (AAT) 74 bp Sc: 80.66
(SEQ ID NO: 435)
GGCCGGTTAGCTCAGTTGGTTAGAGCGTGGTGCTAATAACGCCAAGGTCGCGGGTTCGAT
CCCCGTACGGGCCA
Homo_sapiens_chr6.trna154-IleAAT (27313402-27313329)
Ile (AAT) 74 bp Sc: 80.66
(SEQ ID NO: 436)
GGCCGGTTAGCTCAGTTGGTTAGAGCGTGGTGCTAATAACGCCAAGGTCGCGGGTTCGAT
CCCCGTACGGGCCA
Homo_sapiens_chr6.trna158-IleAAT (27253046-27252973)
Ile (AAT) 74 bp Sc: 80.66
(SEQ ID NO: 437)
GGCCGGTTAGCTCAGTTGGTTAGAGCGTGGTGCTAATAACGCCAAGGTCGCGGGTTCGAT
CCCCGTACGGGCCA
Homo_sapiens_chr17.trna34-IleAAT (8071107-8071034)
Ile (AAT) 74 bp Sc: 80.89
(SEQ ID NO: 438)
GGCCGGTTAGCTCAGTTGGTTAGAGCGTGGTGCTAATAACGCCAAGGTCGCGGGTTCGAA
CCCCGTACGGGCCA
Homo_sapiens_chr6.trna153-IleAAT (27351042-27350969)
Ile (AAT) 74 bp Sc: 82.10
(SEQ ID NO: 439)
GGCTGGTTAGCTCAGTTGGTTAGAGCGTGGTGCTAATAACGCCAAGGTCGCGGGTTCGAT
CCCCGTACTGGCCA
Homo_sapiens_chr6.trna59-IleAAT (27763946-27764019)
Ile (AAT) 74 bp Sc: 83.32
(SEQ ID NO: 440)
GGCCGGTTAGCTCAGTTGGTTAGAGCGTGGTGCTAATAACGCCAAGGTCGCGGGTTCGAT
CCCCGTACTGGCCA
Homo_sapiens_chr6.trna80-IleAAT (58257213-58257286)
Ile (AAT) 74 bp Sc: 83.55
(SEQ ID NO: 441)
GGCCGGTTAGCTCAGTTGGTTAGAGCGTGGCGCTAATAACGCCAAGGTCGCGGGTTCGAT
CCCCGTACGGGCCA
Homo_sapiens_chrX.trna5-IleGAT (3843344-3843271)
Ile (GAT) 74 bp Sc: 72.42
(SEQ ID NO: 442)
GGCCGGTTAGCTCAGTTGGTAAGAGCGTGGTGCTGATAACACCAAGGTCGCGGGCTCGAC
TCCCGCACCGGCCA
Homo_sapiens_chrX.trna6-IleGAT (3804915-3804842)
Ile (GAT) 74 bp Sc: 72.42
(SEQ ID NO: 443)
GGCCGGTTAGCTCAGTTGGTAAGAGCGTGGTGCTGATAACACCAAGGTCGCGGGCTCGAC
TCCCGCACCGGCCA
Homo_sapiens_chrX.trna7-IleGAT (3766491-3766418)
Ile (GAT) 74 bp Sc: 72.42
(SEQ ID NO: 444)
GGCCGGTTAGCTCAGTTGGTAAGAGCGTGGTGCTGATAACACCAAGGTCGCGGGCTCGAC
TCCCGCACCGGCCA
Homo_sapiens_chrX_random.trna1-IleGAT (118398-118471)
Ile (GAT) 74 bp Sc: 72.42
(SEQ ID NO: 445)
GGCCGGTTAGCTCAGTTGGTAAGAGCGTGGTGCTGATAACACCAAGGTCGCGGGCTCGAC
TCCCGCACCGGCCA
Homo_sapiens_chrX random.trna2-IleGAT (406943-407016)
Ile (GAT) 74 bp Sc: 72.42
(SEQ ID NO: 446)
GGCCGGTTAGCTCAGTTGGTAAGAGCGTGGTGCTGATAACACCAAGGTCGCGGGCTCGAC
TCCCGCACCGGCCA
Homo_sapiens_chrX random.trna3-IleGAT (465544-465617)
Ile (GAT) 74 bp Sc: 72.42
(SEQ ID NO: 447)
GGCCGGTTAGCTCAGTTGGTAAGAGCGTGGTGCTGATAACACCAAGGTCGCGGGCTCGAC
TCCCGCACCGGCCA
Homo_sapiens_chrX random.trna4-IleGAT (399021-398948)
Ile (GAT) 74 bp Sc: 72.42
(SEQ ID NO: 448)
GGCCGGTTAGCTCAGTTGGTAAGAGCGTGGTGCTGATAACACCAAGGTCGCGGGCTCGAC
TCCCGCACCGGCCA
Homo_sapiens_chrX random.trna5-IleGAT (86496-86423)
Ile (GAT) 74 bp Sc: 72.42
(SEQ ID NO: 449)
GGCCGGTTAGCTCAGTTGGTAAGAGCGTGGTGCTGATAACACCAAGGTCGCGGGCTCGAC
TCCCGCACCGGCCA
Homo_sapiens_chr6.trna29-IleTAT (27096104-27096197)
Ile (TAT) 94 bp Sc: 67.58
(SEQ ID NO: 450)
GCTCCAGTGGCGCAATCGGTTAGCGCGCGGTACTTATATGGCAGTATGTGTGCGAGTGAT
GCCGAGGTTGTGAGTTCGAGCCTCACCTGGAGCA
Homo_sapiens_chr6.trna55-IleTAT (27707179-27707272)
Ile (TAT) 94 bp Sc: 68.23
(SEQ ID NO: 451)
GCTCCAGTGGCGCAATCGGTTAGCGCGCGGTACTTATACAACAGTATATGTGCGGGTGAT
GCCGAGGTTGTGAGTTCGAGCCTCACCTGGAGCA
Homo_sapiens_chr6.trna63-IleTAT (28613346-28613439)
Ile (TAT) 94 bp Sc: 65.86
(SEQ ID NO: 452)
GCTCCAGTGGCGCAATCGGTTAGCGCGCGGTACTTATAAGACAGTGCACCTGTGAGCAAT
GCCGAGGTTGTGAGTTCAAGCCTCACCTGGAGCA
Homo_sapiens_chr2.trna5-IleTAT (42891180-42891272)
Ile (TAT) 93 bp Sc: 68.11
(SEQ ID NO: 453)
GCTCCAGTGGCGCAATCGGTTAGCGCGCGGTACTTATACAGCAGTACATGCAGAGCAATG
CCGAGGTTGTGAGTTCGAGCCTCACCTGGAGCA
Homo_sapiens_chr19.trna10-IleTAT (44594740-44594648)
Ile (TAT) 93 bp Sc: 68.39
(SEQ ID NO: 454)
GCTCCAGTGGCGCAATCGGTTAGCGCGCGGTACTTATATGACAGTGCGAGCGGAGCAATG
CCGAGGTTGTGAGTTCGATCCTCACCTGGAGCA
Homo_sapiens_chr3.trna5-LeuAAG (149703999-149703918)
Leu (AAG) 82 bp Sc: 20.79
(SEQ ID NO: 455)
GGTAGCATGGCTGAGTGGTCTAAGATTCTGAATTAAGTCTCCAGTCTCTTTGGGGGCGTG
GTTTTCAATCCCACCGCTGCTA
Homo_sapiens_chr20.trna6-LeuAAG (48385830-48385749)
Leu (AAG) 82 bp Sc: 40.72
(SEQ ID NO: 456)
GGTAGGGTGGCCGAGCGGTCTAAGGCACTGTATTAAGACTCCAGTCTCTTCAGAGGCATG
GGTTTGAATCCCACTGCTGCCA
Homo_sapiens_chr2.trna4-LeuAAG (30131072-30131144)
Leu (AAG) 73 bp Sc: 46.12
(SEQ ID NO: 457)
GGGCCAGTGGCTCAATGGATAATGCGTCTGACTAAGAATCAGAAGATTCCAGCCTTGACT
CCTGGCTGGCTCA
Homo_sapiens_chr6.trna126-LeuAAG (28554460-28554379)
Leu (AAG) 82 bp Sc: 59.09
(SEQ ID NO: 458)
GGTAGCGTGGCCGAGTGGTCTAAGACGCTGGATTAAGGCTCCAGTCTCTTCGGGGGCGTG
GGTTTGAATCCCACCGCTGCCA
Homo_sapiens_chr6.trna78-LeuAAG (29064758-29064839)
Leu (AAG) 82 bp Sc: 68.10
(SEQ ID NO: 459)
GGTAGCGTGGCCGAGCGGTCTAAGGCGCTGGATTAAGGCTCCAGTCTCTTCGGGGGCGTG
GGTTCAAATCCCACCGCTGCCA
Homo_sapiens_chr14.trna1-LeuAAG (20148131-20148212)
Leu (AAG) 82 bp Sc: 69.84
(SEQ ID NO: 460)
GGTAGCGTGGCCGAGCGGTCTAAGGCGCTGGATTAAGGCTCCAGTCTCTTCGGGGGCGTG
GGTTCGAATCCCACCGCTGCCA
Homo_sapiens_chr16.trna16-LeuAAG (22215962-22216043)
Leu (AAG) 82 bp Sc: 69.84
(SEQ ID NO: 461)
GGTAGCGTGGCCGAGCGGTCTAAGGCGCTGGATTAAGGCTCCAGTCTCTTCGGGGGCGTG
GGTTCGAATCCCACCGCTGCCA
Homo_sapiens_chr5.trna7-LeuAAG (180547307-180547388)
Leu (AAG) 82 bp Sc: 69.84
(SEQ ID NO: 462)
GGTAGCGTGGCCGAGCGGTCTAAGGCGCTGGATTAAGGCTCCAGTCTCTTCGGGGGCGTG
GGTTCGAATCCCACCGCTGCCA
Homo_sapiens_chr6.trna98-LeuAAG (29019459-29019378)
Leu (AAG) 82 bp Sc: 69.84
(SEQ ID NO: 463)
GGTAGCGTGGCCGAGCGGTCTAAGGCGCTGGATTAAGGCTCCAGTCTCTTCGGGGGCGTG
GGTTCGAATCCCACCGCTGCCA
Homo_sapiens_chr5.trna16-LeuAAG (180533731-180533650)
Leu (AAG) 82 bp Sc: 71.96
(SEQ ID NO: 464)
GGTAGCGTGGCCGAGCGGTCTAAGGCGCTGGATTAAGGCTCCAGTCTCTTCGGAGGCGTG
GGTTCGAATCCCACCGCTGCCA
Homo_sapiens_chr5.trna19-LeuAAG (180457161-180457080)
Leu (AAG) 82 bp Sc: 71.96
(SEQ ID NO: 465)
GGTAGCGTGGCCGAGCGGTCTAAGGCGCTGGATTAAGGCTCCAGTCTCTTCGGAGGCGTG
GGTTCGAATCCCACCGCTGCCA
Homo_sapiens_chr5.trna3-LeuAAG (180461446-180461527)
Leu (AAG) 82 bp Sc: 71.96
(SEQ ID NO: 466)
GGTAGCGTGGCCGAGCGGTCTAAGGCGCTGGATTAAGGCTCCAGTCTCTTCGGAGGCGTG
GGTTCGAATCCCACCGCTGCCA
Homo_sapiens_chr1.trna66-LeuCAA (159848443-159848360)
Leu (CAA) 84 bp Sc: 59.01
(SEQ ID NO: 467)
GTCAGGATGGCCGAGCAGTCTTAAGGCGCTGCGTTCAAATCGCACCCTCCGCTGGAGGCG
TGGGTTCGAATCCCACTTTTGACA
Homo_sapiens_chr11.trna1-LeuCAA (9253366-9253439)
Leu (CAA) 74 bp Sc: 60.99
(SEQ ID NO: 468)
GCCTCCTTAGTGCAGTAGGTAGCGCATCAGTCTCAAAATCTGAATGGTCCTGAGTTCAAG
CCTCAGAGGGGGCA
Homo_sapiens_chr1.trna58-LeuCAA (247134677-247134782)
Leu (CAA) 106 bp Sc: 65.69
(SEQ ID NO: 469)
GTCAGGATGGCCGAGTGGTCTAAGGCGCCAGACTCAAGGTAAGCACCTTGCCTGCGGGCT
TTCTGGTCTCCGGATGGAGGCGTGGGTTCGAATCCCACTTCTGACA
Homo_sapiens_chr6.trna141-LeuCAA (27678433-27678327)
Leu (CAA) 107 bp Sc: 68.19
(SEQ ID NO: 470)
GTCAGGATGGCCGAGTGGTCTAAGGCGCCAGACTCAAGTTGCTACTTCCCAGGTTTGGGG
CTTCTGGTCTCCGCATGGAGGCGTGGGTTCGAATCCCACTTCTGACA
Homo_sapiens_chr6.trna140-LeuCAA (27681503-27681396)
Leu (CAA) 108 bp Sc: 68.53
(SEQ ID NO: 471)
GTCAGGATGGCCGAGTGGTCTAAGGCGCCAGACTCAAGCTTACTGCTTCCTGTGTTCGGG
TCTTCTGGTCTCCGTATGGAGGCGTGGGTTCGAATCCCACTTCTGACA
Homo_sapiens_chr6.trna100-LeuCAA (28972084-28971979)
Leu (CAA) 106 bp Sc: 68.23
(SEQ ID NO: 472)
GTCAGGATGGCCGAGTGGTCTAAGGCGCCAGACTCAAGCTAAGCTTCCTCCGCGGTGGGG
ATTCTGGTCTCCAATGGAGGCGTGGGTTCGAATCCCACTTCTGACA
Homo_sapiens_chr6.trna74-LeuCAA (29016809-29016913)
Leu (CAA) 105 bp Sc: 69.73
(SEQ ID NO: 473)
GTCAGGATGGCCGAGTGGTCTAAGGCGCCAGACTCAAGCTTGGCTTCCTCGTGTTGAGGA
TTCTGGTCTCCAATGGAGGCGTGGGTTCGAATCCCACTTCTGACA
Homo_sapiens_chr5.trna20-LeuCAG (159324696-159324619)
Leu (CAG) 78 bp Sc: 20.45
(SEQ ID NO: 474)
GGCAGTGGAGTTTAGTGGTTAAGGACCTGCTCAGACATCACAGGTAGGTAGATCTGGGTT
CAAACCCTAGCCCTGGCA
Homo_sapiens_chr16.trna17-LeuCAG (55891364-55891446)
Leu (CAG) 83 bp Sc: 75.90
(SEQ ID NO: 475)
GTCAGGATGGCCGAGCGGTCTAAGGCGCTGCGTTCAGGTCGCAGTCTCCCCTGGAGGCGT
GGGTTCGAATCCCACTTCTGACA
Homo_sapiens_chr16.trna26-LeuCAG (55891975-55891893)
Leu (CAG) 83 bp Sc: 75.90
(SEQ ID NO: 476)
GTCAGGATGGCCGAGCGGTCTAAGGCGCTGCGTTCAGGTCGCAGTCTCCCCTGGAGGCGT
GGGTTCGAATCCCACTTCTGACA
Homo_sapiens_chr1.trna34-LeuCAG (159677947-159678029)
Leu (CAG) 83 bp Sc: 77.22
(SEQ ID NO: 477)
GTCAGGATGGCCGAGCGGTCTAAGGCGCTGCGTTCAGGTCGCAGTCTCCCCTGGAGGCGT
GGGTTCGAATCCCACTCCTGACA
Homo_sapiens_chr1.trna36-LeuCAG (159685365-159685447)
Leu (CAG) 83 bp Sc: 77.22
(SEQ ID NO: 478)
GTCAGGATGGCCGAGCGGTCTAAGGCGCTGCGTTCAGGTCGCAGTCTCCCCTGGAGGCGT
GGGTTCGAATCCCACTCCTGACA
Homo_sapiens_chr1.trna38-LeuCAG (159692746-159692828)
Leu (CAG) 83 bp Sc: 77.22
(SEQ ID NO: 479)
GTCAGGATGGCCGAGCGGTCTAAGGCGCTGCGTTCAGGTCGCAGTCTCCCCTGGAGGCGT
GGGTTCGAATCCCACTCCTGACA
Homo_sapiens_chr1.trna40-LeuCAG (159700156-159700238)
Leu (CAG) 83 bp Sc: 77.22
(SEQ ID NO: 480)
GTCAGGATGGCCGAGCGGTCTAAGGCGCTGCGTTCAGGTCGCAGTCTCCCCTGGAGGCGT
GGGTTCGAATCCCACTCCTGACA
Homo_sapiens_chr1.trna42-LeuCAG (159707537-159707619)
Leu (CAG) 83 bp Sc: 77.22
(SEQ ID NO: 481)
GTCAGGATGGCCGAGCGGTCTAAGGCGCTGCGTTCAGGTCGCAGTCTCCCCTGGAGGCGT
GGGTTCGAATCCCACTCCTGACA
Homo_sapiens_chr1.trna67-LeuCAG (159766838-159766756)
Leu (CAG) 83 bp Sc: 77.22
(SEQ ID NO: 482)
GTCAGGATGGCCGAGCGGTCTAAGGCGCTGCGTTCAGGTCGCAGTCTCCCCTGGAGGCGT
GGGTTCGAATCCCACTCCTGACA
Homo_sapiens_chr6.trna7-LeuCAG (26629415-26629497)
Leu (CAG) 83 bp Sc: 77.22
(SEQ ID NO: 483)
GTCAGGATGGCCGAGCGGTCTAAGGCGCTGCGTTCAGGTCGCAGTCTCCCCTGGAGGCGT
GGGTTCGAATCCCACTCCTGACA
Homo_sapiens_chrX.trna2-LeuTAA (55224554-55224480)
Leu (TAA) 75 bp Sc: 28.07
(SEQ ID NO: 484)
GTTAAGATGGCAGAGCCCGGCAATTGCATAAGACTTAAAACTTTATAATCAGAGGTTCAA
CTCCTCTCATTAACA
Homo_sapiens_chr6.trna81-LeuTAA (69971099-69971181)
Leu (TAA) 83 bp Sc: 34.00
(SEQ ID NO: 485)
ACTCATTTGGCTGAGTGGTTAAGGCATTGGACTTAAGATCCAATGGAGTAGTGGCTGTGT
GGGTTTAAACCCCACTACTGGTA
Homo_sapiens_chr4.trna2-LeuTAA (156604502-156604428)
Leu (TAA) 75 bp Sc: 48.70
(SEQ ID NO: 486)
GTTAAGATGGCAGAGCCTGGTAATTGCATAAAACTTAAAATTTTATAATCAGAGGTTCAA
CTCCTCTTCTTAACA
Homo_sapiens_chr6.trna155-LeuTAA (27306395-27306313)
Leu (TAA) 83 bp Sc: 74.34
(SEQ ID NO: 487)
ACCGGGATGGCTGAGTGGTTAAGGCGTTGGACTTAAGATCCAATGGACAGGTGTCCGCGT
GGGTTCGAGCCCCACTCCCGGTA
Homo_sapiens_chr11.trna4-LeuTAA (59075804-59075886)
Leu (TAA) 83 bp Sc: 79.52
(SEQ ID NO: 488)
ACCAGAATGGCCGAGTGGTTAAGGCGTTGGACTTAAGATCCAATGGATTCATATCCGCGT
GGGTTCGAACCCCACTTCTGGTA
Homo_sapiens_chr6.trna134-LeuTAA (27796959-27796877)
Leu (TAA) 83 bp Sc: 80.48
(SEQ ID NO: 489)
ACCGGGATGGCCGAGTGGTTAAGGCGTTGGACTTAAGATCCAATGGGCTGGTGCCCGCGT
GGGTTCGAACCCCACTCTCGGTA
Homo_sapiens_chr6.trna83-LeuTAA (144579377-144579459)
Leu (TAA) 83 bp Sc: 80.77
(SEQ ID NO: 490)
ACCAGGATGGCCGAGTGGTTAAGGCGTTGGACTTAAGATCCAATGGACATATGTCCGCGT
GGGTTCGAACCCCACTCCTGGTA
Homo_sapiens_chr16.trna27-LeuTAG (22114614-22114533)
Leu (TAG) 82 bp Sc: 68.14
(SEQ ID NO: 491)
GGTAGCGTGGCCGAGTGGTCTAAGGCGCTGGATTTAGGCTCCAGTCATTTCGATGGCGTG
GGTTCGAATCCCACCGCTGCCA
Homo_sapiens_chr14.trna2-LeuTAG (20163369-20163450)
Leu (TAG) 82 bp Sc: 68.82
(SEQ ID NO: 492)
GGTAGTGTGGCCGAGCGGTCTAAGGCGCTGGATTTAGGCTCCAGTCTCTTCGGGGGCGTG
GGTTCGAATCCCACCACTGCCA
Homo_sapiens_chr17.trna42-LeuTAG (7964438-7964357)
Leu (TAG) 82 bp Sc: 72.19
(SEQ ID NO: 493)
GGTAGCGTGGCCGAGCGGTCTAAGGCGCTGGATTTAGGCTCCAGTCTCTTCGGAGGCGTG
GGTTCGAATCCCACCGCTGCCA
Homo_sapiens_chr11.trna2-LysCTT (51216476-51216548)
Lys (CTT) 73 bp Sc: 40.00
(SEQ ID NO: 494)
AACCGAATAGCTTAGTTGATGAAGCGTGAGACTCTTAATCTCAGGGTAGTGGGTTCAAGC
CCCACATTGGACA
Homo_sapiens_chr19.trna6-LysCTT (57117280-57117208)
Lys (CTT) 73 bp Sc: 51.97
(SEQ ID NO: 495)
CTGCAGCTAGCTCAGTCGGTAGAGCATGAGACTCTTAATCTCAGGGTCATGGGTTCGTGC
CCCATGTTGGGTG
Homo_sapiens_chr19.trna5-LysCTT (40758590-40758662)
Lys (CTT) 73 bp Sc: 52.67
(SEQ ID NO: 496)
GCCCAGCTAGCTCAGTCGGTAGAGCATAAGACTCTTAATCTCAGGGTTGTGGATTCGTGC
CCCATGCTGGGTG
Homo_sapiens_chr5.trna24-LysCTT (26234368-26234296)
Lys (CTT) 73 bp Sc: 53.34
(SEQ ID NO: 497)
GCCCGACTACCTCAGTCGGTGGAGCATGGGACTCTTCATCCCAGGGTTGTGGGTTCGAGC
CCCACATTGGGCA
Homo_sapiens_chr16.trna5-LysCTT (3154940-3155012)
Lys (CTT) 73 bp Sc: 54.80
(SEQ ID NO: 498)
GCCTGGCTAGCTCAGTCGGCAAAGCATGAGACTCTTAATCTCAGGGTCGTGGGCTCGAGC
TCCATGTTGGGCG
Homo_sapiens_chr1.trna127-LysCTT (55196202-55196130)
Lys (CTT) 73 bp Sc: 58.75
(SEQ ID NO: 499)
GCCCAGCTAGCTCAGTCGGTAGAGCATGAGACTCTTAATCTCAGGGTCATGGGTTTGAGC
CCCACGTTTGGTG
Homo_sapiens_chr18.trna4-LysCTT (41923341-41923269)
Lys (CTT) 73 bp Sc: 60.42
(SEQ ID NO: 500)
GACGAGCTAGCTCAGTCGGTAGAGCATGGGACTCTTAATCCCAGGGTCGTGGGTTTGAGC
CCCATGTTGGGCA
Homo_sapiens_chr16.trna30-LysCTT (3170628-3170556)
Lys (CTT) 73 bp Sc: 64.91
(SEQ ID NO: 501)
GCCCGGCTAGCTCAGTCGATAGAGCATGAGACTCTTAATCTCAGGGTCGTGGGTTCGAGC
CGCACGTTGGGCG
Homo_sapiens_chr16.trna10-LysCTT (3181502-3181574)
Lys (CTT) 73 bp Sc: 74.61
(SEQ ID NO: 502)
GCCCGGCTAGCTCAGTCGGTAGAGCATGGGACTCTTAATCTCAGGGTCGTGGGTTCGAGC
CCCACGTTGGGCG
Homo_sapiens_chr16.trna32-LysCTT (3147479-3147407)
Lys (CTT) 73 bp Sc: 76.19
(SEQ ID NO: 503)
GCCCGGCTAGCTCAGTCGGTAGAGCATGAGACCCTTAATCTCAGGGTCGTGGGTTCGAGC
CCCACGTTGGGCG
Homo_sapiens_chr1.trna119-LysCTT (144106951-144106879)
Lys (CTT) 73 bp Sc: 80.47
(SEQ ID NO: 504)
GCCCGGCTAGCTCAGTCGGTAGAGCATGAGACTCTTAATCTCAGGGTCGTGGGTTCGAGC
CCCACGTTGGGCG
Homo_sapiens_chr16.trna7-LysCTT (3165693-3165765)
Lys (CPT) 73 bp Sc: 80.47
(SEQ ID NO: 505)
GCCCGGCTAGCTCAGTCGGTAGAGCATGAGACTCTTAATCTCAGGGTCGTGGGTTCGAGC
CCCACGTTGGGCG
Homo_sapiens_chr5.trna11-LysCTT (180581657-180581585)
Lys (CTT) 73 bp Sc: 80.47
(SEQ ID NO: 506)
GCCCGGCTAGCTCAGTCGGTAGAGCATGAGACTCTTAATCTCAGGGTCGTGGGTTCGAGC
CCCACGTTGGGCG
Homo_sapiens_chr5.trna9-LysCTT (180567361-180567433)
Lys (CTT) 73 bp Sc: 80.47
(SEQ ID NO: 507)
GCCCGGCTAGCTCAGTCGGTAGAGCATGAGACTCTTAATCTCAGGGTCGTGGGTTCGAGC
CCCACGTTGGGCG
Homo_sapiens_chr6.trna13-LysCTT (26664753-26664825)
Lys (CTT) 73 bp Sc: 80.47
(SEQ ID NO: 508)
GCCCGGCTAGCTCAGTCGGTAGAGCATGAGACTCTTAATCTCAGGGTCGTGGGTTCGAGC
CCCACGTTGGGCG
Homo_sapiens_chr14.trna13-LysCTT (57776438-57776366)
Lys (CPT) 73 bp Sc: 80.72
(SEQ ID NO: 509)
GCCCGGCTAGCTCAGTCGGTAGAGCATGGGACTCTTAATCCCAGGGTCGTGGGTTCGAGC
CCCACGTTGGGCG
Homo_sapiens_chr15.trna2-LysCTT (76939959-76940031)
Lys (CTT) 73 bp Sc: 80.72
(SEQ ID NO: 510)
GCCCGGCTAGCTCAGTCGGTAGAGCATGGGACTCTTAATCCCAGGGTCGTGGGTTCGAGC
CCCACGTTGGGCG
Homo_sapiens_chr19.trna3-LysTTT (19713207-19713277)
Lys (TTT) 71 bp Sc: 28.27
(SEQ ID NO: 511)
ACCCTGTGGTACAGGGGCTAATATGCTGGGCCTTTACCACTTCAGCCCAGGTTCGATTCC
TGGTCAGGGAA
Homo_sapiens_chr19.trna7-LysTTT (54729817-54729745)
Lys (TTT) 73 bp Sc: 48.15
(SEQ ID NO: 512)
ACCTGGGTAGCTTAGTTGGTAGAGCATTGGACTTTTAATTTGAGGGCCCAGGTTTCAAGT
CCCTGTTTGGGTG
Homo_sapiens_chr12.trna1-LysTTT (27734573-27734645)
Lys (TTT) 73 bp Sc: 49.92
(SEQ ID NO: 513)
ACCCAGATAGCTCAGTCAGTAGAGCATCAGACTTTTAATCTGAGGGTCCAAGGTTCATGT
CCCTTTTTGGGTG
Homo_sapiens_chr6.trna53-LysTTT (27651825-27651897)
Lys (TTT) 73 bp Sc: 59.40
(SEQ ID NO: 514)
ACCTGGGTAGCTCAGTAGGTAGAACATCAGACTTTTAATCTGAGGGTCTAGGGTTCAAGT
CCCTGTCCAGGCG
Homo_sapiens_chr1.trna55-LysTTT (203709894-203709966)
Lys (TTT) 73 bp Sc: 62.89
(SEQ ID NO: 515)
GCCCGGAGAGCTCAGTGGGTAGAGCATCAGACTTTTAATCTGAGGGTCCAGGGTTCAAGT
CCTCGTTCGGGCA
Homo_sapiens_chr7_random.trna1-LysTTT (397621-397697)
Lys (TTT) 77 bp Sc: 68.40
(SEQ ID NO: 516)
GCCCACGTAGCTCAATGGTCAGAGCGTGCGGCTTTTAACCGCAAGGAAGGCTGCGAGTTC
GACCCTCGCCGTGGGCT
Homo_sapiens_chr6.trna71-LysTTT (28823500-28823572)
Lys (TTT) 73 bp Sc: 72.00
(SEQ ID NO: 517)
GCCTGGATAGCTCAGTTGGTAGAACATCAGACTTTTAATCTGACGGTGCAGGGTTCAAGT
CCCTGTCCAGGCG
Homo_sapiens_chr6.trna149-LysTTT (27410820-27410748)
Lys (TTT) 73 bp Sc: 81.41
(SEQ ID NO: 518)
GCCTGGGTAGCTCAGTCGGTAGAGCATCAGACTTTTAATCTGAGGGTCCAGGGTTCAAGT
CCCTGTCCAGGCG
Homo_sapiens_chr11.trna5-LysTTT (59080478-59080550)
Lys (TTT) 73 bp Sc: 82.14
(SEQ ID NO: 519)
GCCCGGATAGCTCAGTCGGTAGAGCATCAGACTTTTAATCTGAGGGTCCGGGGTTCAAGT
CCCTGTTCGGGCG
Homo_sapiens_chr6.trna143-LysTTT (27667644-27667572)
Lys (TTT) 73 bp Sc: 83.31
(SEQ ID NO: 520)
GCCTGGATAGCTCAGTCGGTAGAGCATCAGACTTTTAATCTGAGGGTCCAGGGTTCAAGT
CCCTGTTCAGGCG
Homo_sapiens_chr1.trna54-LysTTT (202742278-202742350)
Lys (TTT) 73 bp Sc: 83.80
(SEQ ID NO: 521)
GCCCGGATAGCTCAGTCGGTAGAGCATCAGACTTTTAATCTGAGGGTCCAGGGTTCAAGT
CCCTGTTCGGGCG
Homo_sapiens_chr1.trna62-LysTTT (202742853-202742781)
Lys (TTT) 73 bp Sc: 83.80
(SEQ ID NO: 522)
GCCCGGATAGCTCAGTCGGTAGAGCATCAGACTTTTAATCTGAGGGTCCAGGGTTCAAGT
CCCTGTTCGGGCG
Homo_sapiens_chr11.trna14-LysTTT (59084456-59084384)
Lys (TTT) 73 bp Sc: 83.80
(SEQ ID NO: 523)
GCCCGGATAGCTCAGTCGGTAGAGCATCAGACTTTTAATCTGAGGGTCCAGGGTTCAAGT
CCCTGTTCGGGCG
Homo_sapiens_chr17.trna2-LysTTT (7963198-7963270)
Lys (TTT) 73 bp Sc: 83.80
(SEQ ID NO: 524)
GCCCGGATAGCTCAGTCGGTAGAGCATCAGACTTTTAATCTGAGGGTCCAGGGTTCAAGT
CCCTGTTCGGGCG
Homo_sapiens_chr6.trna76-LysTTT (29026785-29026857)
Lys (TTT) 73 bp Sc: 83.80
(SEQ ID NO: 525)
GCCCGGATAGCTCAGTCGGTAGAGCATCAGACTTTTAATCTGAGGGTCCAGGGTTCAAGT
CCCTGTTCGGGCG
Homo_sapiens_chr11.trna11-LysTTT (121935865-121935937)
Lys (TTT) 73 bp Sc: 83.96
(SEQ ID NO: 526)
GCCTGGATAGCTCAGTTGGTAGAGCATCAGACTTTTAATCTGAGGGTCCAGGGTTCAAGT
CCCTGTTCAGGCG
Homo_sapiens_chr16.trna23-LysTTT (72069789-72069717)
Lys (TTT) 73 bp Sc: 84.43
(SEQ ID NO: 527)
GCCTGGATAGCTCAGTTGGTAGAGCATCAGACTTTTAATCTGAGGGTCCAGGGTTCAAGT
CCCTGTTCAGGCA
Homo_sapiens_chr9.trna1-MetCAT (19393996-19394070)
Met (CAT) 75 bp Sc: 43.49
(SEQ ID NO: 528)
AGCAGAGTGGTGCAGTGGAAGCATACCTATGGGCCCATAACCCAGAGGTTGATGGATGGA
AACCATCCTCTGCTA
Homo_sapiens_chr6.trna61-MetCAT (27853643-27853714)
Met (CAT) 72 bp Sc: 62.43
(SEQ ID NO: 529)
AGCAGAGTGGCGCAGCGGAAGCGTGCTGGGCCCATAACCCAGAGGTCGATGGATCTAAAC
CATCCTCTGCTA
Homo_sapiens_chr6.trna92-MetCAT (58276523-58276451)
Met (CAT) 73 bp Sc: 64.33
(SEQ ID NO: 530)
GCCCTCTTAGTGCAGCTGGCAGCGCGTCAGTTTCATAATCTGAAAGTCCTGAGTTCAAGC
CTCAGAGAGGGCA
Homo_sapiens_chr1.trna32-MetCAT (151910350-151910421)
Met (CAT) 72 bp Sc: 68.98
(SEQ ID NO: 531)
AGCAGAGTGGCGCAGCGGAAGCGTGCTGGGCCCATAACCCAGAGGTCGATGGATCGAAAC
CATCCTCTGCTA
Homo_sapiens_chr17.trna20-MetCAT (78045957-78045886)
Met (CAT) 72 bp Sc: 68.98
(SEQ ID NO: 532)
AGCAGAGTGGCGCAGCGGAAGCGTGCTGGGCCCATAACCCAGAGGTCGATGGATCGAAAC
CATCCTCTGCTA
Homo_sapiens_chr6.trna129-MetCAT (27978321-27978250)
Met (CAT) 72 bp Sc: 68.98
(SEQ ID NO: 533)
AGCAGAGTGGCGCAGCGGAAGCGTGCTGGGCCCATAACCCAGAGGTCGATGGATCGAAAC
CATCCTCTGCTA
Homo_sapiens_chr6.trna142-MetCAT (27668650-27668579)
Met (CAT) 72 bp Sc: 68.98
(SEQ ID NO: 534)
AGCAGAGTGGCGCAGCGGAAGCGTGCTGGGCCCATAACCCAGAGGTCGATGGATCGAAAC
CATCCTCTGCTA
Homo_sapiens_chr6.trna150-MetCAT (27408814-27408743)
Met (CAT) 72 bp Sc: 68.98
(SEQ ID NO: 535)
AGCAGAGTGGCGCAGCGGAAGCGTGCTGGGCCCATAACCCAGAGGTCGATGGATCGAAAC
CATCCTCTGCTA
Homo_sapiens_chr6.trna169-MetCAT (26438579-26438508)
Met (CAT) 72 bp Sc: 68.98
(SEQ ID NO: 536)
AGCAGAGTGGCGCAGCGGAAGCGTGCTGGGCCCATAACCCAGAGGTCGATGGATCGAAAC
CATCCTCTGCTA
Homo_sapiens_chr6.trna171-MetCAT (26421402-26421331)
Met (CAT) 72 bp Sc: 68.98
(SEQ ID NO: 537)
AGCAGAGTGGCGCAGCGGAAGCGTGCTGGGCCCATAACCCAGAGGTCGATGGATCGAAAC
CATCCTCTGCTA
Homo_sapiens_chr6.trna2-MetCAT (26394733-26394804)
Met (CAT) 72 bp Sc: 68.98
(SEQ ID NO: 538)
AGCAGAGTGGCGCAGCGGAAGCGTGCTGGGCCCATAACCCAGAGGTCGATGGATCGAAAC
CATCCTCTGCTA
Homo_sapiens_chr16.trna22-MetCAT (85975201-85975129)
Met (CAT) 73 bp Sc: 74.51
(SEQ ID NO: 539)
GCCTCGTTAGCGCAGTAGGCAGCGCGTCAGTCTCATAATCTGAAGGTCGTGAGTTCGAGC
CTCACACGGGGCA
Homo_sapiens_chr6.trna21-MetCAT (26809691-26809763)
Met (CAT) 73 bp Sc: 75.06
(SEQ ID NO: 540)
GCCCTCTTAGCGCAGCTGGCAGCGCGTCAGTCTCATAATCTGAAGGTCCTGAGTTCAAGC
CTCAGAGAGGGCA
Homo_sapiens_chr6.trna162-MetCAT (26866601-26866529)
Met (CAT) 73 bp Sc: 75.76
(SEQ ID NO: 541)
GCCCTCTTAGCGCAGCGGGCAGCGCGTCAGTCTCATAATCTGAAGGTCCTGAGTTCGAGC
CTCAGAGAGGGCA
Homo_sapiens_chr6.trna164-MetCAT (26843625-26843553)
Met (CAT) 73 bp Sc: 75.76
(SEQ ID NO: 542)
GCCCTCTTAGCGCAGCGGGCAGCGCGTCAGTCTCATAATCTGAAGGTCCTGAGTTCGAGC
CTCAGAGAGGGCA
Homo_sapiens_chr6.trna27-MetCAT (26874423-26874495)
Met (CAT) 73 bp Sc: 75.76
(SEQ ID NO: 543)
GCCCTCTTAGCGCAGCGGGCAGCGCGTCAGTCTCATAATCTGAAGGTCCTGAGTTCGAGC
CTCAGAGAGGGCA
Homo_sapiens_chr6.trna75-MetCAT (29020331-29020403)
Met (CAT) 73 bp Sc: 76.58
(SEQ ID NO: 544)
GCCTCCTTAGCGCAGTAGGCAGCGCGTCAGTCTCATAATCTGAAGGTCCTGAGTTCGAAC
CTCAGAGGGGGCA
Homo_sapiens_chr6.trna97-MetCAT (29029093-29029021)
Met (CAT) 73 bp Sc: 76.58
(SEQ ID NO: 545)
GCCTCCTTAGCGCAGTAGGCAGCGCGTCAGTCTCATAATCTGAAGGTCCTGAGTTCGAAC
CTCAGAGGGGGCA
Homo_sapiens_chr16.trna20-MetCAT (70017897-70017969)
Met (CAT) 73 bp Sc: 78.21
(SEQ ID NO: 546)
GCCCTCTTAGCGCAGTGGGCAGCGCGTCAGTCTCATAATCTGAAGGTCCTGAGTTCGAGC
CTCAGAGAGGGCA
Homo_sapiens_chr8.trna10-MetCAT (124238723-124238651)
Met (CAT) 73 bp Sc: 79.85
(SEQ ID NO: 547)
GCCTCGTTAGCGCAGTAGGTAGCGCGTCAGTCTCATAATCTGAAGGTCGTGAGTTCGATC
CTCACACGGGGCA
Homo_sapiens_chr6.trna56-PheGAA (27740524-27740599)
Phe (GAA) 76 bp Sc: 53.54
(SEQ ID NO: 548)
GCCGAAATAGCTCAATTGGGAGAGTGTTAGACTGAAGATCTTCTGCAGGTCTCTGGTTCA
ATTCCGGGTTTCGACA
Homo_sapiens_chr6.trna112-PheGAA (28839426-28839353)
Phe (GAA) 74 bp Sc: 56.32
(SEQ ID NO: 549)
GCTGAAATAGCTCAGTTGGGAGAGCGTTAGACTGAAGATCTTAAAGTTCCCTGGTTCAAC
CCTGGGTTTCAGCC
Homo_sapiens_chr6.trna72-PheGAA (28840143-28840215)
Phe (GAA) 73 bp Sc: 59.98
(SEQ ID NO: 550)
GCCAAAATTGCTCAGTTGGGAGAGCGTTAGACTGAAGATCTAAAGGTCCCTGGTTCGATC
CCGGGTTTCACCA
Homo_sapiens_chr6.trna103-PheGAA (28899145-28899072)
Phe (GAA) 74 bp Sc: 67.94
(SEQ ID NO: 551)
GCCGAAATAGCTCAGTTGGGAGAGCGTTAGACCGAAGATCTTAAAGGTCCCTGGTTCAAT
CCCGGGTTTCGGCA
Homo_sapiens_chr6.trna106-PheGAA (28883661-28883589)
Phe (GAA) 73 bp Sc: 82.13
(SEQ ID NO: 552)
GCCGAGATAGCTCAGTTGGGAGAGCGTTAGACTGAAGATCTAAAGGTCCCTGGTTCAATC
CCGGGTTTCGGCA
Homo_sapiens_chr11.trna13-PheGAA (59090501-59090429)
Phe (GAA) 73 bp Sc: 82.45
(SEQ ID NO: 553)
GCCGAAATAGCTCAGTTGGGAGAGCGTTAGACTGAAGATCTAAAGGTCCCTGGTTCAATC
CCGGGTTTCGGCA
Homo_sapiens_chr11.trna15-PheGAA (59081618-59081546)
Phe (GAA) 73 bp Sc: 84.19
(SEQ ID NO: 554)
GCCGAAATAGCTCAGTTGGGAGAGCGTTAGACTGAAGATCTAAAGGTCCCTGGTTCGATC
CCGGGTTTCGGCA
Homo_sapiens_chr12.trna11-PheGAA (123978414-123978342)
Phe (GAA) 73 bp Sc: 84.19
(SEQ ID NO: 555)
GCCGAAATAGCTCAGTTGGGAGAGCGTTAGACTGAAGATCTAAAGGTCCCTGGTTCGATC
CCGGGTTTCGGCA
Homo_sapiens_chr13.trna1-PheGAA (93999977-93999905)
Phe (GAA) 73 bp Sc: 84.19
(SEQ ID NO: 556)
GCCGAAATAGCTCAGTTGGGAGAGCGTTAGACTGAAGATCTAAAGGTCCCTGGTTCGATC
CCGGGTTTCGGCA
Homo_sapiens_chr19.trna14-PheGAA (1334433-1334361)
Phe (GAA) 73 bp Sc: 84.19
(SEQ ID NO: 557)
GCCGAAATAGCTCAGTTGGGAGAGCGTTAGACTGAAGATCTAAAGGTCCCTGGTTCGATC
CCGGGTTTCGGCA
Homo_sapiens_chr6.trna109-PheGAA (28866550-28866478)
Phe (GAA) 73 bp Sc: 84.19
(SEQ ID NO: 558)
GCCGAAATAGCTCAGTTGGGAGAGCGTTAGACTGAAGATCTAAAGGTCCCTGGTTCGATC
CCGGGTTTCGGCA
Homo_sapiens_chr6.trna96-PheGAA (29057500-29057428)
Phe (GAA) 73 bp Sc: 84.19
(SEQ ID NO: 559)
GCCGAAATAGCTCAGTTGGGAGAGCGTTAGACTGAAGATCTAAAGGTCCCTGGTTCGATC
CCGGGTTTCGGCA
Homo_sapiens_chr1.trna65-ProAGG (165951420-165951349)
Pro (AGG) 72 bp Sc: 75.92
(SEQ ID NO: 560)
GGCTCGTTGGTCTAGGGGTATGATTCTCGCTTAGGGTGCGAGAGGTCCCGGGTTCAAATC
CCGGACGAGCCC
Homo_sapiens_chr11.trna9-ProAGG (75624205-75624276)
Pro (AGG) 72 bp Sc: 75.92
(SEQ ID NO: 561)
GGCTCGTTGGTCTAGGGGTATGATTCTCGCTTAGGGTGCGAGAGGTCCCGGGTTCAAATC
CCGGACGAGCCC
Homo_sapiens_chr14.trna22-ProAGG (20151471-20151400)
Pro (AGG) 72 bp Sc: 75.92
(SEQ ID NO: 562)
GGCTCGTTGGTCTAGGGGTATGATTCTCGCTTAGGGTGCGAGAGGTCCCGGGTTCAAATC
CCGGACGAGCCC
Homo_sapiens_chr14.trna23-ProAGG (20147406-20147335)
Pro (AGG) 72 bp Sc: 75.92
(SEQ ID NO: 563)
GGCTCGTTGGTCTAGGGGTATGATTCTCGCTTAGGGTGCGAGAGGTCCCGGGTTCAAATC
CCGGACGAGCCC
Homo_sapiens_chr16.trna29-ProAGG (3172707-3172636)
Pro (AGG) 72 bp Sc: 75.92
(SEQ ID NO: 564)
GGCTCGTTGGTCTAGGGGTATGATTCTCGCTTAGGGTGCGAGAGGTCCCGGGTTCAAATC
CCGGACGAGCCC
Homo_sapiens_chr16.trna9-ProAGG (3179635-3179706)
Pro (AGG) 72 bp Sc: 75.92
(SEQ ID NO: 565)
GGCTCGTTGGTCTAGGGGTATGATTCTCGCTTAGGGTGCGAGAGGTCCCGGGTTCAAATC
CCGGACGAGCCC
Homo_sapiens_chr6.trna12-ProAGG (26663477-26663548)
Pro (AGG) 72 bp Sc: 75.92
(SEQ ID NO: 566)
GGCTCGTTGGTCTAGGGGTATGATTCTCGCTTAGGGTGCGAGAGGTCCCGGGTTCAAATC
CCGGACGAGCCC
Homo_sapiens_chr7.trna2-ProAGG (128210740-128210811)
Pro (AGG) 72 bp Sc: 75.92
(SEQ ID NO: 567)
GGCTCGTTGGTCTAGGGGTATGATTCTCGCTTAGGGTGCGAGAGGTCCCGGGTTCAAATC
CCGGACGAGCCC
Homo_sapiens_chr16.trna11-ProAGG (3181990-3182061)
Pro (AGG) 72 bp Sc: 77.31
(SEQ ID NO: 568)
GGCTCGTTGGTCTAGGGGTATGATTCTCGCTTAGGATGCGAGAGGTCCCGGGTTCAAATC
CCGGACGAGCCC
Homo_sapiens_chr16.trna4-ProAGG (3150387-3150481)
Pro (AGG) 95 bp Sc: 43.10
(SEQ ID NO: 569)
GGCTCGTTGGTCTAGGGGTGTGGTTCTCGCTTAGGGACCACAGGGACAAGCCCGGGAGAC
CCAAGAGGTCCCGGGTTCAAATCCCGGACGAGCCC
Homo_sapiens_chr6.trna30-ProCGG (27167500-27167571)
Pro (CGG) 72 bp Sc: 71.93
(SEQ ID NO: 570)
GGCTCGTTGGTCTAGGGGTATGATTCTCGCTTCGGGTGTGAGAGGTCCCGGGTTCAAATC
CCGGACGAGCCC
Homo_sapiens_chr1.trna52-ProCGG (165950586-165950657)
Pro (CGG) 72 bp Sc: 76.52
(SEQ ID NO: 571)
GGCTCGTTGGTCTAGGGGTATGATTCTCGCTTCGGGTGCGAGAGGTCCCGGGTTCAAATC
CCGGACGAGCCC
Homo_sapiens_chr16.trna6-ProCGG (3162050-3162121)
Pro (CGG) 72 bp Sc: 76.52
(SEQ ID NO: 572)
GGCTCGTTGGTCTAGGGGTATGATTCTCGCTTCGGGTGCGAGAGGTCCCGGGTTCAAATC
CCGGACGAGCCC
Homo_sapiens_chr17.trna37-ProCGG (8066947-8066876)
Pro (CGG) 72 bp Sc: 76.52
(SEQ ID NO: 573)
GGCTCGTTGGTCTAGGGGTATGATTCTCGCTTCGGGTGCGAGAGGTCCCGGGTTCAAATC
CCGGACGAGCCC
Homo_sapiens_chr14.trna6-ProTGG (20222015-20222086)
Pro (TGG) 72 bp Sc: 76.15
(SEQ ID NO: 574)
GGCTCGTTGGTCTAGGGGTATGATTCTCGCTTTGGGTGCGAGAGGTCCCGGGTTCAAATC
CCGGACGAGCCC
Homo_sapiens_chr16.trna28-ProTGG (3174205-3174134)
Pro (TGG) 72 bp Sc: 76.15
(SEQ ID NO: 575)
GGCTCGTTGGTCTAGGGGTATGATTCTCGCTTTGGGTGCGAGAGGTCCCGGGTTCAAATC
CCGGACGAGCCC
Homo_sapiens_chr16.trna3-ProTGG (3148924-3148995)
Pro (TGG) 72 bp Sc: 76.15
(SEQ ID NO: 576)
GGCTCGTTGGTCTAGGGGTATGATTCTCGCTTTGGGTGCGAGAGGTCCCGGGTTCAAATC
CCGGACGAGCCC
Homo_sapiens_chr16.trna8-ProTGG (3178095-3178166)
Pro (TGG) 72 bp Sc: 76.15
(SEQ ID NO: 577)
GGCTCGTTGGTCTAGGGGTATGATTCTCGCTTTGGGTGCGAGAGGTCCCGGGTTCAAATC
CCGGACGAGCCC
Homo_sapiens_chr5.trna14-ProTGG (180548531-180548460)
Pro (TGG) 72 bp Sc: 76.15
(SEQ ID NO: 578)
GGCTCGTTGGTCTAGGGGTATGATTCTCGCTTTGGGTGCGAGAGGTCCCGGGTTCAAATC
CCGGACGAGCCC
Homo_sapiens_chr11.trna12-ProTGG (75624588-75624517)
Pro (TGG) 72 bp Sc: 76.24
(SEQ ID NO: 579)
GGCTCGTTGGTCTAGGGGTATGATTCTCGGTTTGGGTCCGAGAGGTCCCGGGTTCAAATC
CCGGACGAGCCC
Homo_sapiens_chr14.trna3-ProTGG (20171005-20171076)
Pro (TGG) 72 bp Sc: 79.61
(SEQ ID NO: 580)
GGCTCGTTGGTCTAGTGGTATGATTCTCGCTTTGGGTGCGAGAGGTCCCGGGTTCAAATC
CCGGACGAGCCC
Homo_sapiens_chr5.trna17-LeuAAG (180524251-180524170)
Leu (AAG) 82 bp Sc: 25.68
(SEQ ID NO: 581)
GATAGCAAGGCCGAGCGGTCTAAGGCTCCGGATTAAGGCGCCGGTGTCTTCGGAGGCATG
GGTTCGAATTCCACCTCTGCCA
Homo_sapiens_chr20.trna5-IleAAT (50651822-50651745)
Ile (AAT) 78 bp Sc: 23.04
(SEQ ID NO: 582)
GACCAATTAGCAAGCACAGTTGGCTAGAACATGGTGCTAATAAGGCCACGGTCAGGGGTT
CAATTCCCTTATGGGCTG
Homo_sapiens_chr12.trna9-IleAAT (128282151-128282225)
Ile (AAT) 75 bp Sc: 27.18
(SEQ ID NO: 583)
TGGCTGATGAGCTCAGCTGGTGGGAGCATGGTGTTAATGAGGCTGAGGTCGTGGGTTCAA
TCCCCACCGGGCTAT
Homo_sapiens_chr6.trna39-IleAAT (27359843-27359916)
Ile (AAT) 74 bp Sc: 44.67
(SEQ ID NO: 584)
GGCCGGTTAACTTAATTGGTTAGAGCGTGGTGCTAATAATGTCAAGGTTGCGGGTTGGAT
CCCCGAACGGGCCA
Homo_sapiens_chr5.trna1-CysACA (151968789-151968964)
Cys (ACA) 176 bp Sc: 29.48
(SEQ ID NO: 585)
GGCTGTATAGCTCAGTGGTAGAGCATTTGACTACAGAATCCTATACTCAGGGGAAGGAGA
ACTGGGGGTTTCTCAGTGGGTCAAAGGACTTGTAGTGGTAAATCAAAAGCAACTCTATAA
GCTATGTAACAAACTTTAAAGTCATATGTAGCTGGGTTCAAATCCTGTTTCTGCCA
Homo_sapiens_chr6.trna41-SerACT (27369650-27369723)
Ser (ACT) 74 bp Sc: 49.68
(SEQ ID NO: 586)
GGCCGGTTAGCTCAGTTGGTTAGAGCGTGCTGCTACTAATGCCAGGGTCGAGGTTTCGAT
CCCCGTACGGGCCT
Homo_sapiens_chr20.trna2-SerAGA (29552364-29552435)
Ser (AGA) 72 bp Sc: 20.36
(SEQ ID NO: 587)
GGTAATGTAGCCTCGTGGTTAGGGGCTGCATTCTAGAGCTATGCTGCCCAGGTTCAAATT
CTGGTGCCACTC
Homo_sapiens_chr6.trna111-AlaAGC (28854594-28854523)
Ala (AGC) 72 bp Sc: 32.22
(SEQ ID NO: 588)
GGGGGTATAGCTCAGGGACAGAGCACGTGGTTAGCATGCGTGAGGTCCTGCGTTCAACTT
CCAGTATTTCCA
Homo_sapiens_chr16.trna33-ProAGG (3142681-3142610)
Pro (AGG) 72 bp Sc: 23.16
(SEQ ID NO: 589)
CGCTCTTTGGTCTAGGGGTATGATCTTCGCTTAGGGTGCGAGAGGTGCCTGGATCAACTC
CTTCACAAGCCG
Homo_sapiens_chr2.trna7-ProAGG (77749101-77749171)
Pro (AGG) 71 bp Sc: 30.29
(SEQ ID NO: 590)
GGCTGGTTGGTCTAGGGCTATGATTCTCACTTAGGGTGCAAGAGGTCCTGGTTCAAATCC
CAGAGGAGCCC
Homo_sapiens_chr2.trna8-ProAGG (87193084-87193155)
Pro (AGG) 72 bp Sc: 37.07
(SEQ ID NO: 591)
GGCTGGTTGGTCTAGGGCTATGATTCTCACTTAGGGTGCAAAAGGTCCTGGGTTCAAATC
CCAGAGGAGCCC
Homo_sapiens_chr10.trna3-LeuCAA (34631489-34631422)
Leu (CAA) 68 bp Sc: 20.90
(SEQ ID NO: 592)
CATAGGGTAGTGGCTAAGAACCTAAACTCTAAATTTAGATGTCCTGAGTTCAAATCCCAG
CTGTATGC
Homo_sapiens_chr20.trna3-ValCAC (43384415-43384487)
Val (CAC) 73 bp Sc: 29.18
(SEQ ID NO: 593)
GCTTCTGTAATGTAGTGGTTATCACATTCGCCTCACACATGAAAGGTCACCAGTTTGAGA
CCGGGCCAAAACA
Homo_sapiens_chr1.trna1-ValCAC (16924648-16924720)
Val (CAC) 73 bp Sc: 30.83
(SEQ ID NO: 594)
GTTTCTGTAGTATAGTGGTTATCATGTTTGCCTCACATGTGAAAGACCCTTGGCTCGAGA
CTGGAGGGAAACA
Homo_sapiens_chr1.trna131-ValCAC (16746819-16746747)
Val (CAC) 73 bp Sc: 41.96
(SEQ ID NO: 595)
GTTTCTGTGGTGTAGTGGTTATTATGTTCGCTTCACATATGAAAGGTCTCTGGTTCGAGA
CTGCGTGGGAACA
Homo_sapiens_chr1.trna3-ValCAC (17059280-17059352)
Val (CAC) 73 bp Sc: 41.96
(SEQ ID NO: 596)
GTTTCTGTGGTGTAGTGGTTATTATGTTCGCTTCACATATGAAAGGTCTCTGGTTCGAGA
CTGCGTGGGAACA
Homo_sapiens_chr2.trna1-GlyCCC (11609278-11609349)
Gly (CCC) 72 bp Sc: 43.42
(SEQ ID NO: 597)
TACTCAGTGGTCTAGTGGTTAGGATTCAGCGCTCCCACCGCCGCAGCCCGGGTTCGATTC
CCGGTCATGGAA
Homo_sapiens_chr1.trna91-GlyCCC (147946904-147946834)
Gly (CCC) 71 bp Sc: 49.64
(SEQ ID NO: 598)
GCACTGGTGGTTCAGTGGTAGAATTCTCGCCTCCCACGCGGGAGACCCGGGTTTAATTCC
CGGTCAAGATA
Homo_sapiens_chr11.trna7-ArgCCT (63418343-63418411)
Arg (CCT) 69 bp Sc: 21.47
(SEQ ID NO: 599)
GCATTCGTAGTTCAGCGGCAGAAATTTCGTCTCCTACGCGGGAGACTCGGGTTCGACTTC
GGCCATGCA
Homo_sapiens_chr1.trna88-ArgCCT (147994965-147994895)
Arg (CCT) 71 bp Sc: 21.62
(SEQ ID NO: 600)
GGATTTGTGGTCCAGTGGTAGAATTCTCACCGCCTGCATAGGAGACCCTGGTTTAATTCC
TGGCCAATGCA
Homo_sapiens_chr1.trna96-ArgCCT (147609775-147609705)
Arg (CCT) 71 bp Sc: 27.53
(SEQ ID NO: 601)
GGATTGGTGGTCCAGTGGTAGAATTCTCACCGCCTGCATAGGAGACCCTGGTTTAATTCC
TGGCCAATGCA
Homo_sapiens_chr1.trna14-ArgCCT (144652936-144653006)
Arg (CCT) 71 bp Sc: 34.62
(SEQ ID NO: 602)
GCATTGGTAGTTCAGCGGTGGCATTCTCCCCACCTACGCGGGAGACCTGGGTTCAACTCC
CGGCCAAAGCA
Homo_sapiens_chr1.trna18-ArgCCT (145949915-145949985)
Arg (CCT) 71 bp Sc: 34.62
(SEQ ID NO: 603)
GCATTGGTAGTTCAGCGGTGGCATTCTCCCCACCTACGCGGGAGACCTGGGTTCAACTCC
CGGCCAAAGCA
Homo_sapiens_chr14.trna11-SupCTA (77770663-77770591)
Sup (CTA) 73 bp Sc: 26.26
(SEQ ID NO: 604)
GGCGGGATAATGTAGTGGTTAAAGGCATGGGCTCTAGAGCCAGACTTCCTGGGTTCAAAT
CTCAGACCTGCTA
Homo_sapiens_chr3.trna1-SupCTA (13808887-13808954)
Sup (CTA) 68 bp Sc: 26.42
(SEQ ID NO: 605)
GGCAGGGTAGGGTAGAGGTTAAAACCATGGATTCTAGAGCCAGATGGGTTCAAATCCCGG
CTCTGCCG
Homo_sapiens_chrX.trna3-GluCTC (51322923-51322852)
Glu (CTC) 72 bp Sc: 20.35
(SEQ ID NO: 606)
TCCCTGGTGTTCCGGTGGTTAGGATTTGGCATTCTCACTGTTGTGGTGCGGATTCAATCC
TGGCTTAGGGTA
Homo_sapiens_chr12.trna14-GluCTC (112871000-112870928)
Glu (CTC) 73 bp Sc: 22.05
(SEQ ID NO: 607)
CCGTGGATAGCCCAGCGGCTATGGGAGCCGGGCTCTCACTCTGACGTCCTGGGTTCAAGT
CCCAGTGTGCACA
Homo_sapiens_chr2.trna25-GluCTC (71127068-71126996)
Glu (CTC) 73 bp Sc: 22.62
(SEQ ID NO: 608)
CCCCTGGCGGTCTAGTGGTTAGGATTCGGCGCTCTCATCCACCGCGGCCTGGGTTCGACT
CGTGGTCAGAGTG
Homo_sapiens_chr8.trna7-GluCTC (70234894-70234968)
Glu (CTC) 75 bp Sc: 22.64
(SEQ ID NO: 609)
CTCTGGTGGTTTAGTGGCTAGGATTCACCTCTCTCACTGCTGCAGCCCAGGGTTCCATTC
CCTGGGAGTCAGATG
Homo_sapiens_chr2.trna24-GluCTC (95580992-95580921)
Glu (CTC) 72 bp Sc: 23.39
(SEQ ID NO: 610)
CCCCTTGTAGTCTAGTGGTTAGAATTCTGCGGTCTCACAGCCGCGGCCCGGGTTCGATTC
CCATTCCGGGAA
Homo_sapiens_chr3.trna10-GluCTC (105362285-105362214)
Glu (CTC) 72 bp Sc: 25.40
(SEQ ID NO: 611)
TTATTATTATACCTGTGGTTAGGATTCGGCGCTCTCACCGCCACGACCCGGGTTCAATTC
CCGGTCAGGGAA
Homo_sapiens_chr8.trna1-GluCTC (11830789-11830860)
Glu (CTC) 72 bp Sc: 26.22
(SEQ ID NO: 612)
CCCCTGGTAGTCTAGTGGTTAGGCTTTGCCGCTCTCAGTGCCGCTGCCTGGGTTGGATTC
CCAGTCATGTGA
Homo_sapiens_chr20.trna7-GluCTC (13918541-13918470)
Glu (CTC) 72 bp Sc: 27.08
(SEQ ID NO: 613)
TCCCTGCTTGTCTAGTGGTTAGAATTCAGCACTCTCACTGCCACAGCCCAGGTTCAATTC
CCTGTCAGAGAA
Homo_sapiens_chr2.trna12-GluCTC (150927523-150927595)
Glu (CTC) 73 bp Sc: 29.78
(SEQ ID NO: 614)
TTCCCCTTGGTCTAGTGGTTAGGATTCAACACTCTCACCGCCGCAGCCCGGGTTTGATTC
CCAGGCAGGGAAG
Homo_sapiens_chr2.trna18-GluCTC (159446488-159446417)
Glu (CTC) 72 bp Sc: 31.33
(SEQ ID NO: 615)
CCCCTGGTGGTCTAGTGCTTAGGATTTGGCACTCTCGCCACCGCAGCCTGCGTTCAATTC
CCGGTCAGGGAA
Homo_sapiens_chr13.trna2-GluCTC (57356622-57356551)
Glu (CTC) 72 bp Sc: 34.44
(SEQ ID NO: 616)
TCCCTGGTGGTCTAATGGTTAGGAGTCGGCACTCTCACCGCCGCGGCTGGGGTTTGATTC
CCAGTCATGTAA
Homo_sapiens_chr2.trna15-GlnCTG (219199376-219199451)
Gln (CTG) 76 bp Sc: 22.40
(SEQ ID NO: 617)
GTGAGACTGCACAGCCCAGTGGTGCAGGGCATGGCTCTGACACCTGGCGGCCTGGGTTCA
AATCCCAGCTTCTACA
Homo_sapiens_chr1.trna128-GlnCTG (17053558-17053487)
Gln (CTG) 72 bp Sc: 25.51
(SEQ ID NO: 618)
GGTTCCATGATGTAATGGTGAGCGCTTTGGACTCTGAGTACGGTGATCAGCGTTCAAGTC
TCAGTGGGACCT
Homo_sapiens_chr5.trna21-GlnCTG (151228338-151228267)
Gln (CTG) 72 bp Sc: 25.76
(SEQ ID NO: 619)
GGTAGTGTAGTCTACTGGTTAAACGCTTGGGCTCTGACATTAACGTCCTGGGTTCAAATC
CCAGCTTTGTCA
Homo_sapiens_chr10.trna5-GlnCTG (20076688-20076614)
Gln (CTG) 75 bp Sc: 26.31
(SEQ ID NO: 620)
CTAGGACGTGGTGTAATAGGTAGCACAGAGAATTCTGGATTCTCAGGGGTAGGTTCAATT
CCTATAGAACCTAGG
Homo_sapiens_chr1.trna27-GlnCTG (147345988-147346059)
Gln (CTG) 72 bp Sc: 39.31
(SEQ ID NO: 621)
GGTTCCATGGTGTAATGGTGAGGGCTTTGGACTCTGACTACAGTGATCAGAGTTCAAGTC
TCAGTGGGACCT
Homo_sapiens_chr1.trna23-GlnCTG (146292313-146292384)
Gln (CTG) 72 bp Sc: 43.68
(SEQ ID NO: 622)
GGTTCCATGGGTTAATGGTGAGCACCCTGGACTCTGAATCAAGCGATCCGAGTTCAAATC
TCGGTGGTACCT
Homo_sapiens_chr15.trna6-LysCTT (74461893-74461820)
Lys (CTT) 74 bp Sc: 37.12
(SEQ ID NO: 623)
GCCTGGCTACCTCAGTTGGTAGAGCATGGGACTCTTAATCCCAGAGTCAGTGGGTTCAAG
CCTCACATTGAGTG
Homo_sapiens_chr16.trna13-LysCTT (3186154-3186226)
Lys (CPT) 73 bp Sc: 41.66
(SEQ ID NO: 624)
GCCCAGCTAGCTCAGCCGGTAGAGCACAAGACTCTTAATCTCAGGGTCGTGGGTTTGAGC
CCTGTGTTGAGCA
Homo_sapiens_chr1.trna51-LysCTT (163832774-163832846)
Lys (CTT) 73 bp Sc: 42.42
(SEQ ID NO: 625)
GTCTAGCTAGATCAGTTGGTAGAGCATAAGACTCTTAATCTCAGGGTCATGGGTTTGAGC
CCTACGTTGGGCG
Homo_sapiens_chr1.trna8-LysCTT (39742782-39742854)
Lys (CTT) 73 bp Sc: 46.90
(SEQ ID NO: 626)
ACCAGCATGTCTCAGTCGGTATAGTGTGAGACTCTTAATCTCAGGGTCGTGGGTTCAAGC
CCCACATTGGGCG
Homo_sapiens_chr8.trna9-PheGAA (124339978-124339906)
Phe (GAA) 73 bp Sc: 20.84
(SEQ ID NO: 627)
GCCAAAATAGCTCAGCTGGGAGAGTATTAGGTTGAAGATACAAAGTTCCTTGGCTCAATC
CAGAGTTTGGGGG
Homo_sapiens_chr6.trna116-PheGAA (28802906-28802834)
Phe (GAA) 73 bp Sc: 27.39
(SEQ ID NO: 628)
GCTGAGATAGCTCGGTTGGGAGGGCATCAGCCTGAAGATCTAAAGGAGACTGGTTCAATT
CTGGGTTTTGGCA
Homo_sapiens_chr1.trna92-PheGAA (147939600-147939529)
Phe (GAA) 72 bp Sc: 32.31
(SEQ ID NO: 629)
TGCATGGTTGTCTAGTGGCTAGGATTCGGTGCTGAAAGCGTCACGGCCCGGGTTCGATTC
CCGGTCAGGGAA
Homo_sapiens_chr1.trna100-PheGAA (147554054-147553983)
Phe (GAA) 72 bp Sc: 32.35
(SEQ ID NO: 630)
TGCATGGTTGTCTAGTGGCTAGGATTCGGTGCTGAAAGAGCCACGGCCCGGGTTCGATTC
CCGGTCAGGGAA
Homo_sapiens_chr6.trna88-PheGAA (79724801-79724729)
Phe (GAA) 73 bp Sc: 43.56
(SEQ ID NO: 631)
GCCAAAATAGCTCAGCTGGGAGAGCATTAGACTGAAGATCTAAAGGTCTCTGGTTTGATC
CTGGGTTTCAGAA
Homo_sapiens_chr3.trna12-CysGCA (17716468-17716396)
Cys (GCA) 73 bp Sc: 36.14
(SEQ ID NO: 632)
GGGGGTATATCTCAGGGGGCAGAGCATTTGACTGCAGATCAAGAGGTCCCCGGTTGAAAT
CCGGGTGCTGGAT
Homo_sapiens_chr10.trna1-ProGGG (22892585-22892657)
Pro (GGG) 73 bp Sc: 34.81
(SEQ ID NO: 633)
GGCAGTGTGGCATAGTGGTTAGAAATGTGCGCTCTGGGGCTGCTGATCCCAGGCTCAAAC
CCTGGCGCTGTCA
Homo_sapiens_chr15.trna5-TyrGTA (90055378-90055306)
Tyr (GTA) 73 bp Sc: 24.21
(SEQ ID NO: 634)
GTCAGTGTTGCACAACGGTTAAGTGAAGAGGCTGTAAACCCAGACTGGATGGGTTCAATT
CCCATCTCTGCCG
Homo_sapiens_chr7.trna24-TyrGTA (148684753-148684678)
Tyr (GTA) 76 bp Sc: 33.50
(SEQ ID NO: 635)
TCAATTATAGCTCAGTGGTAGAGCATTTAACTGTAGATCAAGAGGTCCCTGGATCAACTC
TGGGTGCCCCCTTTAA
Homo_sapiens_chr1.trna44-AspGTC (159759559-159759630)
Asp (GTC) 72 bp Sc: 27.96
(SEQ ID NO: 636)
TCCTTGTTACTATAGTGGTGAGTATCTCTGCCTGTCATGCGTGAGAGAGGGGGTCGATTC
CCCGACGGGGAG
Homo_sapiens_chr1.trna109-AsnGTT (146317757-146317684)
Asn (GTT) 74 bp Sc: 34.29
(SEQ ID NO: 637)
GTCTCTGTGGCACAATCGGTTAGCTTGTTCGGCTGTTAATCTAGAGGTTGGTGGTTAGAG
CCCACTGAGGGATG
Homo_sapiens_chr1.trna104-AsnGTT (147478645-147478572)
Asn (GTT) 74 bp Sc: 41.72
(SEQ ID NO: 638)
GTCTCTGTGGCACAATCGGTTAGAGCGTTCGGCTGTTAATCTAAAGGTTGGTGGCTAGAA
CCCACTGAGGGACG
Homo_sapiens_chr1.trna124-AsnGTT (142455154-142455081)
Asn (GTT) 74 bp Sc: 43.05
(SEQ ID NO: 639)
GTCTCTGTGGCACAATCGGTTAGCGCGTTCGGCTGTTAATCTAGAGGTTGGTGGTTAGAG
Homo_sapiens_chr8.trna13-LeuTAA (47859277-47859203)
Leu (TAA) 75 bp Sc: 20.57
(SEQ ID NO: 640)
GTTAAGATGGCATAGCCCAGCAATTGCATAAAACTTAAGACTTTATAATTAGAAGTTCAA
CACCTCTTCTTAACA
Homo_sapiens_chr20.trna4-LeuTAA (55366909-55366835)
Leu (TAA) 75 bp Sc: 25.92
(SEQ ID NO: 641)
GTTAAGATGGCAGAGCCCAGCGATTGCATAAAACTTAACACTTTATAATCAGAGGTTCAA
CTCCTCTTCTTAACA
Homo_sapiens_chr1.trna60-LeuTAA (236173653-236173579)
Leu (TAA) 75 bp Sc: 29.87
(SEQ ID NO: 642)
GTTAAGATGGCAGAGCCCAGCAATTGCATAAAACTTAAAACTTTACAATCAGAGGTTCAA
CTCCTCTTCTTAACA
Homo_sapiens_chr2.trna9-LeuTAA (117497887-117497961)
Leu (TAA) 75 bp Sc: 34.06
(SEQ ID NO: 643)
GTTAAGATGGCAGAGCCCAGCAATTGCATAAATCTTAAAACTTTATAATCAGAGGTTCGA
CTCCTCTTCTTAACA
Homo_sapiens_chr6.trna86-ValTAC (156910812-156910738)
Val (TAC) 75 bp Sc: 31.66
(SEQ ID NO: 644)
GTTAAGACGGCAGAGCCCGGCAATTGCGTAAAATTTACAACTTTATGGGCAGAGGTTCAA
TTCCTCTTCTTAACA
Homo_sapiens_chr14.trna15-LeuTAG (20215099-20215018)
Leu (TAG) 82 bp Sc: 32.18
(SEQ ID NO: 645)
GGTAGTGTGGTTGAATGGTCTAAGGCACTGAATTTAGGCTCCAGTCTCTTTGGGGACGTG
GGTTTAAATCCCACTGCTGCAA
Homo_sapiens_chr18.trna3-GlyTCC (53497175-53497246)
Gly (TCC) 72 bp Sc: 32.27
(SEQ ID NO: 646)
GTGTTGATGGTATAGTGGTGAGCATAGCTGCCTTCCAAGCAATTGACCCGACTTCAATTC
CCAGCCAACGCA
Homo_sapiens_chr18.trna2-GlyTCC (53496852-53496923)
Gly (TCC) 72 bp Sc: 33.06
(SEQ ID NO: 647)
GTGTTGATGGTATAGTGGTGAGCATAGCTGCCTTCCAAGCAATTGACCCGACTTCGATTC
CCAGCCAATGCA
Homo_sapiens_chr17.trna32-SerTGA (21952374-21952306)
Ser (TGA) 69 bp Sc: 23.94
(SEQ ID NO: 648)
GAAAAAGTCATAGGGGTTATGAGGCTGGCTTGAAACCAGCCTTAGGAGGTTCAATTCCTT
CCTTTTTTG
Homo_sapiens_chr2.trna10-SerTGA (131856612-131856681)
Ser (TGA) 70 bp Sc: 27.03
(SEQ ID NO: 649)
GAGAAGGTCATAGAGGTTATGGGATTGGCTTGAAACCAGTCTCTGGGGGGTTCGATTCCC
TCCTTTTTCA
Homo_sapiens_chr11.trna6-AlaTGC (60520084-60520153)
Ala (TGC) 70 bp Sc: 20.74
(SEQ ID NO: 650)
GGGGGAGTGGTGTGGTTACGAATGTGGCCTCTGCAAGCAGACAGCCTGGGTTCAATTCCC
AGCTTGGCCA
Homo_sapiens_chr6.trna122-AlaTGC (28709909-28709838)
Ala (TGC) 72 bp Sc: 42.75
(SEQ ID NO: 651)
GTGGATGTAGTTTAGTGGTAGAACGCGCGCTTTGCATGTATGAGGTCCCGGTTTCGATCC
CTGGCGTTTCCA
Homo_sapiens_chr1.trna61-ProTGG (205244853-205244777)
Pro (TGG) 77 bp Sc: 23.61
(SEQ ID NO: 652)
GCCAGGAGAGCTCAGTGGTGATGGGATGAGATCTGGACTCACACCTCTAGGCCTGGGTTC
AAATCCCAGGTCTAGCG
Homo_sapiens_chr1.trna57-ProTGG (236172485-236172556)
Pro (TGG) 72 bp Sc: 33.27
(SEQ ID NO: 653)
TAGGACTTGGTGTAATAGGTAGCACGAAGAGATTTGGATTCTCAGGGGTAGGTTCAATTC
CTATAGTTCTGG
Homo_sapiens_chr16.trna31-ProTGG (3161032-3160962)
Pro (TGG) 71 bp Sc: 37.53
(SEQ ID NO: 654)
GGCCTGTTGGTCTAGAGGTATGATTCTCGCTTTGGGTGCGAGAGGCCCCGGTGCGAGTCC
CAGAGGAGCCC
Homo_sapiens_chr4.trna5-SupTTA (7376810-7376739)
Sup (TTA) 72 bp Sc: 21.73
(SEQ ID NO: 655)
GGCAGCCTGGCTTAGTGGAAAGGGAATAGGCTTTAGAGCCAGACTGCCTGGGTTTGAATC
CCAGCCCCGCCA
Homo_sapiens_chr6.trna85-GluTTC (163130072-163130004)
Glu (TTC) 69 bp Sc: 22.25
(SEQ ID NO: 656)
TCCCTGGTCTAGTGGTTAGGATTTATTATTTTCATGGCTGTGGCCTGAGTTCAATTTCCA
ATCAGGGAA
Homo_sapiens_chr1.trna17-GluTTC (145016802-145016873)
Glu (TTC) 72 bp Sc: 23.38
(SEQ ID NO: 657)
TCCCTGGTGGTCTGGTGGCTAGAATTTAGCGCTTTCACCGCCGCAGCTCGGGTTGGATTA
CCAGTCAGGGAA
Homo_sapiens_chr1.trna102-GluTTC (147545481-147545409)
Glu (TTC) 73 bp Sc: 35.32
(SEQ ID NO: 658)
TCCGTGGTGGTCTAGTGGCTAGGATTCGGCGCTTTCACCGCCTGCAGCTCGAGTTCGATT
CCTGGTCAGGGAA
Homo_sapiens_chr2.trna17-GluTTC (203937446-203937376)
Glu (TTC) 71 bp Sc: 39.76
(SEQ ID NO: 659)
GCAATGGTGGTTCAGTGGTAGAATTCTCGCCTTTCACACAGGAGACCCGGGTTCAATTCC
TGACCCATGTA
Homo_sapiens_chr1.trna105-GluTTC (147428323-147428252)
Glu (TTC) 72 bp Sc: 41.70
(SEQ ID NO: 660)
TGTCTGGTGGTCAAGTGGCTAGGATTTGGCGCTTTCACTGCCGCGGCCCGCGTTCGATTC
CCGGTCAGGGAA
Homo_sapiens_chr1.trna110-GluTTC (146247725-146247654)
Glu (TTC) 72 bp Sc: 43.43
(SEQ ID NO: 661)
TCCTTGGTGGTCTAGTGGCTAGGATTCGGTGCTTTCACCTGTGCGGCCCGGGTTCAATTC
CCGATGAAGGAA
Homo_sapiens_chr4.trna1-GlnTTG (156603338-156603409)
Gln (TTG) 72 bp Sc: 20.26
(SEQ ID NO: 662)
TAGGACGTGGTGTGATAGGTAGCACAGAGAATTTTGGATTCTCAGGGGTAGGTTAAATTC
CTATAGTACTAG
Homo_sapiens_chr7.trna26-GlnTTG (57257993-57257922)
Gln (TTG) 72 bp Sc: 22.05
(SEQ ID NO: 663)
TAGGACGTGGTGTGATAGGTAGCATGGGGAATTTTGGATTCTCAGGGGTGGGTTCAATTC
CTATAGTTCTAG
Homo_sapiens_chr12.trna15-GlnTTG (96014895-96014822)
Gln (TTG) 74 bp Sc: 22.16
(SEQ ID NO: 664)
GGCAATGTAGCATTGTGGCTAAGTGCACAGGCTTTGGAAACTGGCAGGCCTGGGTTCAAA
TCCCAGCTTATTCA
Homo_sapiens_chr17.trna33-GlnTTG (19447250-19447179)
Gln (TTG) 72 bp Sc: 22.33
(SEQ ID NO: 665)
TAGGATGTGGTGTAATAGGTGGCATGGAGAATTTTGGATTATCAGGGGTAGGTTCAATTC
CTATAGTTCTAG
Homo_sapiens_chr8.trna14-GlnTTG (32992259-32992188)
Gln (TTG) 72 bp Sc: 22.66
(SEQ ID NO: 666)
TAGTACATAGTGTAATAGGTAGCACAGATAATTTTGGATTCTCAGGGGTAGGTTCAATTC
TTATAGTTCTAG
Homo_sapiens_chr19.trna12-GlnTTG (9011428-9011356)
Gln (TTG) 73 bp Sc: 23.15
(SEQ ID NO: 667)
AGCAGTGTAGCCTAGTGGCTAGGTCCTCTGACTTTGAAACCACGTGGTCTGGGTTTAAGT
CTCAGCTGTGCTA
Homo_sapiens_chr13.trna6-GlnTTG (35537818-35537747)
Gln (TTG) 72 bp Sc: 23.74
(SEQ ID NO: 668)
TAGGATGTGGTGTGACAGGTAGCATGGAGAATTTTGGATTCTCAGGGTTAGGTTCAATTC
CTATAGTTCTAG
Homo_sapiens_chr5.trna23-GlnTTG (77354604-77354532)
Gln (TTG) 73 bp Sc: 24.04
(SEQ ID NO: 669)
AGCTGTATATTATAGTGGAATAAATGTGGACTTTGAAGTTAGATACACCTGGGTTCAAAT
CCCAGTGCTGTCA
Homo_sapiens_chr2.trna11-GlnTTG (131859603-131859674)
Gln (TTG) 72 bp Sc: 25.61
(SEQ ID NO: 670)
TAGGACGTGGTGTGATAGGTAGCACGGAGAATTTTGGATTCTCAGGGATGGGTTCAATTC
CTGTAGTTCTAG
Homo_sapiens_chrY.trna1-GlnTTG (8300214-8300140)
Gln (TTG) 75 bp Sc: 27.14
(SEQ ID NO: 671)
TTTAGGACGTGGTGTAATAGGTAGCACAGAGAATTTTGGATTCTCAGGTGCAGGTTCAAT
TCCTATATTCTAGAG
Homo_sapiens_chr2.trna22-GlnTTG (130746452-130746381)
Gln (TTG) 72 bp Sc: 28.85
(SEQ ID NO: 672)
TAGGACGTGGTGTGATAGGTAGCATGGAGAATTTTGGATTCTCAGGGATGGGTTCAATTC
CTATAGTCCTAG
Homo_sapiens_chr12.trna16-GlnTTG (48497531-48497457)
Gln (TTG) 75 bp Sc: 29.85
(SEQ ID NO: 673)
TCTAGGATGTGGTGTGATAGGTAGCATGGAGAATTTTGGATTCTCAGGGGTAGGTTCAAT
TCCTATATTCTAGAA
Homo_sapiens_chr3.trna9-GlnTTG (108103639-108103568)
Gln (TTG) 72 bp Sc: 32.34
(SEQ ID NO: 674)
TAGGATGTGGTGTATTAGGTAGCACAGAGAATTTTGGATTCTCAGGGGTAGGTTCGATTC
CTATAATTCTAC
Homo_sapiens_chr7.trna4-GlnTTG (141149826-141149897)
Gln (TTG) 72 bp Sc: 34.30
(SEQ ID NO: 675)
TAGGACGTGGTGTAGTAGGTAGCATGGAGAATGTTGAATTCTCAGGGGTAGGTTCAATTC
CTATAGTTCTAG
Homo_sapiens_chrX.trna1-GlnTTG (55223391-55223462)
Gln (TTG) 72 bp Sc: 34.66
(SEQ ID NO: 676)
TAGGACATGGTGTGATAGGTAGCATGGAGAATTTTGGATTCTCAGGGGTAGGTTCAATTC
CTACAGTTCTAG
Homo_sapiens_chr2.trna28-GlnTTG (45791017-45790945)
Gln (TTG) 73 bp Sc: 35.83
(SEQ ID NO: 677)
GGCTGTGTACCTCAGTGGGCAAGGGTATGGACTTTGAAGCCAGACTATTTGGGTTCAAAT
CCCAGCTTGGCCT
Homo_sapiens_chr8.trna2-LysTTT (18164483-18164552)
Lys (TTT) 70 bp Sc: 20.79
(SEQ ID NO: 678)
TCCTATAGCCCAGTGATTAGGATTCTTTGCTTTTACTACCATGACCTGGGTTCAATACCC
AGTCAGGGAA
Homo_sapiens_chr2.trna16-LysTTT (223894631-223894559)
Lys (TTT) 73 bp Sc: 34.01
(SEQ ID NO: 679)
GTTGGGGTAACTCAGTTGGTAGAGTAGCAGACTTTTCATCTGAGGGTCCAGGGTTTAAGT
CCATGTCCAGGCA
Homo_sapiens_chr14.trna12-LysTTT (73125354-73125282)
Lys (TTT) 73 bp Sc: 38.73
(SEQ ID NO: 680)
ACCCAGATAGCTCAGTTGATAGAGCATCAGACTTTTAATCTGAGGGTCCAGGGTTCATGT
CCCTGTTCCTTAA
Homo_sapiens_chr6.trna118-LysTTT (28769039-28768966)
Lys (TTT) 74 bp Sc: 44.65
(SEQ ID NO: 681)
GCCTGGGTAGCTCAGTCGGTAGAGCTATCAGACTTTTAGCCTGAGGATTCAGGGTTCAAT
CCCTTGCTGGGGCG
Homo_sapiens_chr19.trna9-LysTTT (46440054-46439982)
Lys (TTT) 73 bp Sc: 53.63
(SEQ ID NO: 682)
GCCAGGATAGTTCAGGTGGTAGAGCATCAGACTTTTAACCTGAGGGTTCAGGGTTCAAGT
CTCTGTTTGGGCG
Homo_sapiens_chr17.trna25-SeCTCA (35527152-35527079)
SeC (TCA) 74 bp Sc: 47.88
(SEQ ID NO: 683)
GACCGTGTGGCCTTAATGGATAAGGTGTCTGACTTCAGATCAGAAGATTGAGGGTTTGAG
TCCCTTTGTGGTCA
Homo_sapiens_chr22.trna1-SeC(e)TCA (42877870-42877955)
SeC(e) (TCA) 86 bp Sc: 62.60
(SEQ ID NO: 684)
GCTCGGATGATCCTCAGTGGTCTGGGGTGCAGGCTTCAAACCTGTAGCT
GTCTAGTGACA GAGTGGTTCAATTCCACCTTTGTAGG
Homo_sapiens_chr19.trna8-SeC(e)TCA (50673785-50673700)
SeC(e) (TCA) 86 bp Sc: 75.99
(SEQ ID NO: 685)
GCCCGGATGATCCTCAGTGGTCTGGGGTGCAGGCTTCAAACCTGTAGCTGTCTAG
CGACAGAGTGGTTCAATTCCACCTTTCGGGC
Homo_sapiens_chr11.trna10-SerAGA (108541249-108541330)
Ser (AGA) 82 bp Sc: 29.13
(SEQ ID NO: 686)
TGAGTTGTAGCTGAGTGGTTAAGGCAACGAGCTAGAAATTCGTTGGTTTCTCTCTGTGCA
GGTTTGAATCCTGCTAATTATG
Homo_sapiens_chr7.trna12-SerAGA (148936400-148936471)
Ser (AGA) 72 bp Sc: 62.08
(SEQ ID NO: 687)
GGGTGTATGGCTCAGGGGTAGAGAATTTGACTAGAGATCAAGAGGTCCCTGGTTCAAATC
CAGGTGCCCCCT
Homo_sapiens_chr6.trna145-SerAGA (27629252-27629171)
Ser (AGA) 82 bp Sc: 79.96
(SEQ ID NO: 688)
GTAGTCGTGGCCGAGTGGTTAAGGTGATGGACTAGAAACCCATTGGGGTCTCCCCGCGCA
GGTTCGAATCCTGCCGACTACG
Homo_sapiens_chr6.trna50-SerAGA (27607966-27608047)
Ser (AGA) 82 bp Sc: 86.61
(SEQ ID NO: 689)
GTAGTCGTGGCCGAGTGGTTAAGGCGATGGACTAGAAATCCATTGGGGTTTCCCCACGCA
GGTTCGAATCCTGCCGACTACG
Homo_sapiens_chr17.trna35-SerAGA (8070734-8070653)
Ser (AGA) 82 bp Sc: 88.01
(SEQ ID NO: 690)
GTAGTCGTGGCCGAGTGGTTAAGGCGATGGACTAGAAATCCATTGGGGTCTCCCCGCGCA
GGTTCGAATCCTGCCGACTACG
Homo_sapiens_chr6.trna44-SerAGA (27554570-27554651)
Ser (AGA) 82 bp Sc: 88.01
(SEQ ID NO: 691)
GTAGTCGTGGCCGAGTGGTTAAGGCGATGGACTAGAAATCCATTGGGGTCTCCCCGCGCA
GGTTCGAATCCTGCCGACTACG
Homo_sapiens_chr6.trna46-SerAGA (27571572-27571653)
Ser (AGA) 82 bp Sc: 88.01
(SEQ ID NO: 692)
GTAGTCGTGGCCGAGTGGTTAAGGCGATGGACTAGAAATCCATTGGGGTCTCCCCGCGCA
GGTTCGAATCCTGCCGACTACG
Homo_sapiens_chr6.trna47-SerAGA (27578797-27578878)
Ser (AGA) 82 bp Sc: 88.01
(SEQ ID NO: 693)
GTAGTCGTGGCCGAGTGGTTAAGGCGATGGACTAGAAATCCATTGGGGTCTCCCCGCGCA
GGTTCGAATCCTGCCGACTACG
Homo_sapiens_chr6.trna5-SerAGA (26435796-26435877)
Ser (AGA) 82 bp Sc: 88.01
(SEQ ID NO: 694)
GTAGTCGTGGCCGAGTGGTTAAGGCGATGGACTAGAAATCCATTGGGGTCTCCCCGCGCA
GGTTCGAATCCTGCCGACTACG
Homo_sapiens_chr8.trna11-SerAGA (96351142-96351061)
Ser (AGA) 82 bp Sc: 88.01
(SEQ ID NO: 695)
GTAGTCGTGGCCGAGTGGTTAAGGCGATGGACTAGAAATCCATTGGGGTCTCCCCGCGCA
GGTTCGAATCCTGCCGACTACG
Homo_sapiens_chr6.trna147-SerAGA (27617614-27617533)
Ser (AGA) 82 bp Sc: 88.50
(SEQ ID NO: 696)
GTAGTCGTGGCCGAGTGGTTAAGGCGATGGACTAGAAATCCATTGGGGTTTCCCCGCGCA
GGTTCGAATCCTGCCGACTACG
Homo_sapiens_chr12.trna2-SerCGA (54870415-54870496)
Ser (CGA) 82 bp Sc: 89.14
(SEQ ID NO: 697)
GTCACGGTGGCCGAGTGGTTAAGGCGTTGGACTCGAAATCCAATGGGGTTTCCCCGCACA
GGTTCGAATCCTGTTCGTGACG
Homo_sapiens_chr6.trna137-SerCGA (27748289-27748208)
Ser (CGA) 82 bp Sc: 89.16
(SEQ ID NO: 698)
GCTGTGATGGCCGAGTGGTTAAGGTGTTGGACTCGAAATCCAATGGGGGTTCCCCGCGCA
GGTTCAAATCCTGCTCACAGCG
Homo_sapiens_chr6.trna35-SerCGA (27285607-27285688)
Ser (CGA) 82 bp Sc: 90.35
(SEQ ID NO: 699)
GCTGTGATGGCCGAGTGGTTAAGGCGTTGGACTCGAAATCCAATGGGGTCTCCCCGCGCA
GGTTCAAATCCTGCTCACAGCG
Homo_sapiens_chr17.trna41-SerCGA (7983005-7982924)
Ser (CGA) 82 bp Sc: 92.09
(SEQ ID NO: 700)
GCTGTGATGGCCGAGTGGTTAAGGCGTTGGACTCGAAATCCAATGGGGTCTCCCCGCGCA
GGTTCGAATCCTGCTCACAGCG
Homo_sapiens_chr6.trna175-SerGCT (26413780-26413697)
Ser (GCT) 84 bp Sc: 67.16
(SEQ ID NO: 701)
GGAGAGGCCTGGCCGAGTGGTTAAGGCGATGGACTGCTAATCCATTGTGCTCTGCACGCG
TGGGTTCGAATCCCATCCTCGTCG
Homo_sapiens_chr6.trna62-SerGCT (28288794-28288875)
Ser (GCT) 82 bp Sc: 83.46
(SEQ ID NO: 702)
GACGAGGTGGCCGAGTGGTTAAGGCGATGGACTGCTAATCCATTGTGCTCTGCACACGTG
GGTTCGAATCCCATCCTCGTCG
Homo_sapiens_chr15.trna10-SerGCT (38673396-38673315)
Ser (GCT) 82 bp Sc: 85.34
(SEQ ID NO: 703)
GACGAGGTGGCCGAGTGGTTAAGGCGATGGACTGCTAATCCATTGTGCTCTGCACGCGTG
GGTTCGAATCCCATCCTCGTCG
Homo_sapiens_chr17.trna7-SerGCT (8030909-8030990)
Ser (GCT) 82 bp Sc: 85.34
(SEQ ID NO: 704)
GACGAGGTGGCCGAGTGGTTAAGGCGATGGACTGCTAATCCATTGTGCTCTGCACGCGTG
GGTTCGAATCCCATCCTCGTCG
Homo_sapiens_chr6.trna123-SerGCT (28673177-28673096)
Ser (GCT) 82 bp Sc: 85.34
(SEQ ID NO: 705)
GACGAGGTGGCCGAGTGGTTAAGGCGATGGACTGCTAATCCATTGTGCTCTGCACGCGTG
GGTTCGAATCCCATCCTCGTCG
Homo_sapiens_chr11.trna8-SerGCT (65872167-65872248)
Ser (GCT) 82 bp Sc: 85.83
(SEQ ID NO: 706)
GACGAGGTGGCCGAGTGGTTAAGGCGATGGACTGCTAATCCATTGTGCTTTGCACGCGTG
GGTTCGAATCCCATCCTCGTCG
Homo_sapiens_chr6.trna43-SerGCT (27373754-27373835)
Ser (GCT) 82 bp Sc: 86.80
(SEQ ID NO: 707)
GACGAGGTGGCCGAGTGGTTAAGGCGATGGACTGCTAATCCATTGTGCTCTGCACGCGTG
GGTTCGAATCCCACCTTCGTCG
Homo_sapiens_chr6.trna31-SerGCT (27173064-27173145)
Ser (GCT) 82 bp Sc: 88.12
(SEQ ID NO: 708)
GACGAGGTGGCCGAGTGGTTAAGGCGATGGACTGCTAATCCATTGTGCTCTGCACGCGTG
GGTTCGAATCCCACCCTCGTCG
Homo_sapiens_chr2.trna21-SerTGA (130749563-130749494)
Ser (TGA) 70 bp Sc: 31.73
(SEQ ID NO: 709)
GAGAAGGTCACAGAGGTTATGGGATTGGCTTGAAACCAGTCTGTGGGGGGTTCGATTCCC
TCCTTTTTCA
Homo_sapiens_chr6.trna148-SerTGA (27581667-27581586)
Ser (TGA) 82 bp Sc: 84.78
(SEQ ID NO: 710)
GTAGTCGTGGCCGAGTGGTTAAGGCGATGGACTTGAAATCCATTGGGGTTTCCCCGCGCA
GGTTCGAATCCTGTCGGCTACG
Homo_sapiens_chr6.trna172-SerTGA (26420884-26420803)
Ser (TGA) 82 bp Sc: 88.25
(SEQ ID NO: 711)
GTAGTCGTGGCCGAGTGGTTAAGGCGATGGACTTGAAATCCATTGGGGTCTCCCCGCGCA
GGTTCGAATCCTGCCGACTACG
Homo_sapiens_chr6.trna51-SerTGA (27621447-27621528)
Ser (TGA) 82 bp Sc: 88.73
(SEQ ID NO: 712)
GTAGTCGTGGCCGAGTGGTTAAGGCGATGGACTTGAAATCCATTGGGGTTTCCCCGCGCA
GGTTCGAATCCTGCCGACTACG
Homo_sapiens_chr10.trna2-SerTGA (69194267-69194348)
Ser (TGA) 82 bp Sc: 90.86
(SEQ ID NO: 713)
GCAGCGATGGCCGAGTGGTTAAGGCGTTGGACTTGAAATCCAATGGGGTCTCCCCGCGCA
GGTTCGAACCCTGCTCGCTGCG
Homo_sapiens_chr17.trna11-SupCTA (15349410-15349483)
Sup (CTA) 74 bp Sc: 28.00
(SEQ ID NO: 714)
GGCAGTGACGCCTACTGGTTTAGAGCACAGATTCTAGATCGAGACATTCCTGGGTTCAAA
TCCCAGCACTGTTG
Homo_sapiens_chr21.trna1-SupTTA (14848387-14848457)
Sup (TTA) 71 bp Sc: 22.35
(SEQ ID NO: 715)
GGCAGTGACATGTAATGGTTATGAGGGTGGACTTTAACCACACTGCCTAGGTTCAAATCC
TGACTCTGTCA
Homo_sapiens_chr17.trna17-SupTTA (56218375-56218445)
Sup (TTA) 71 bp Sc: 57.62
(SEQ ID NO: 716)
GCCCGGATAGTTCAGTTGGTAGAGCATCAGACTTAATCAGAGGGTCCAGGGTTCAAGTCC
CTGTTTGGGTG
Homo_sapiens_chr17.trna24-ThrAGT (59957453-59957380)
Thr (AGT) 74 bp Sc: 43.90
(SEQ ID NO: 717)
AGCACCATGGCTTAGCTGGTTAAAGCACCTGTCTAGTAAACAGGAGATCCTGAGTTTCAA
TTCCAATGGTGCCT
Homo_sapiens_chr6.trna34-ThrAGT (27238029-27238102)
Thr (AGT) 74 bp Sc: 80.30
(SEQ ID NO: 718)
GGCCCTGTGGCTTAGCTGGTCAAAGCGCCTGTCTAGTAAACAGGAGATCCTGGGTTCGAA
TCCCAGCGGGGCCT
Homo_sapiens_chr6.trna60-ThrAGT (27802452-27802525)
Thr (AGT) 74 bp Sc: 81.60
(SEQ ID NO: 719
GGCTTCGTGGCTTAGCTGGTTAAAGCGCCTGTCTAGTAAACAGGAGATCCTGGGTTCGAA
TCCCAGCGAGGCCT
Homo_sapiens_chr17.trna40-ThrAGT (7983568-7983495)
Thr (AGT) 74 bp Sc: 81.60
(SEQ ID NO: 720)
GGCGCCGTGGCTTAGCTGGTTAAAGCGCCTGTCTAGTAAACAGGAGATCCTGGGTTCGAA
TCCCAGCGGTGCCT
Homo_sapiens_chr6.trna69-ThrAGT (28801774-28801847)
Thr (AGT) 74 bp Sc: 82.17
(SEQ ID NO: 721)
GGCTCCGTAGCTTAGTTGGTTAAAGCGCCTGTCTAGTAAACAGGAGATCCTGGGTTCGAC
TCCCAGCGGGGCCT
Homo_sapiens_chr6.trna135-ThrAGT (27760526-27760453)
Thr (AGT) 74 bp Sc: 83.05
(SEQ ID NO: 722)
GGCTCCGTGGCTTAGCTGGTTAAAGCGCCTGTCTAGTAAACAGGAGATCCTGGGTTCGAA
TCCCAGCGGGGCCT
Homo_sapiens_chr6.trna167-ThrAGT (26641197-26641124)
Thr (AGT) 74 bp Sc: 83.05
(SEQ ID NO: 723)
GGCTCCGTGGCTTAGCTGGTTAAAGCGCCTGTCTAGTAAACAGGAGATCCTGGGTTCGAA
TCCCAGCGGGGCCT
Homo_sapiens_chr17.trna36-ThrAGT (8070351-8070278)
Thr (AGT) 74 bp Sc: 84.06
(SEQ ID NO: 724)
GGCGCCGTGGCTTAGTTGGTTAAAGCGCCTGTCTAGTAAACAGGAGATCCTGGGTTCGAA
TCCCAGCGGTGCCT
Homo_sapiens_chr17.trna8-ThrAGT (8031203-8031276)
Thr (AGT) 74 bp Sc: 84.06
(SEQ ID NO: 725)
GGCGCCGTGGCTTAGTTGGTTAAAGCGCCTGTCTAGTAAACAGGAGATCCTGGGTTCGAA
TCCCAGCGGTGCCT
Homo_sapiens_chr19.trna4-ThrAGT (38359803-38359876)
Thr (AGT) 74 bp Sc: 84.06
(SEQ ID NO: 726)
GGCGCCGTGGCTTAGTTGGTTAAAGCGCCTGTCTAGTAAACAGGAGATCCTGGGTTCGAA
TCCCAGCGGTGCCT
Homo_sapiens_chr6.trna151-ThrCGT (27379618-27379547)
Thr (CGT) 72 bp Sc: 46.44
(SEQ ID NO: 727)
GGCAGAGTGGTGCAGCGGAAGCGTGCTGGGCCCGTAACCCAGAGGTCAATGGATCGAAGC
CATCCTTGGCTA
Homo_sapiens_chr6.trna54-ThrCGT (27694114-27694187)
Thr (CGT) 74 bp Sc: 63.29
(SEQ ID NO: 728)
GGCCCTGTAGCTCAGCGGTTGGAGCGCTGGTCTCGTAAACCTAGGGGTCGTGAGTTCAAA
TCTCACCAGGGCCT
Homo_sapiens_chr17.trna14-ThrCGT (26901213-26901284)
Thr (CGT) 72 bp Sc: 79.77
(SEQ ID NO: 729)
GGCGCGGTGGCCAAGTGGTAAGGCGTCGGTCTCGTAAACCGAAGATCGCGGGTTCGAACC
CCGTCCGTGCCT
Homo_sapiens_chr6.trna121-ThrCGT (28724036-28723963)
Thr (CGT) 74 bp Sc: 80.30
(SEQ ID NO: 730)
GGCTCTGTGGCTTAGTTGGCTAAAGCGCCTGTCTCGTAAACAGGAGATCCTGGGTTCGAA
TCCCAGCGGGGCCT
Homo_sapiens_chr16.trna15-ThrCGT (14287251-14287322)
Thr (CGT) 72 bp Sc: 80.42
(SEQ ID NO: 731)
GGCGCGGTGGCCAAGTGGTAAGGCGTCGGTCTCGTAAACCGAAGATCACGGGTTCGAACC
CCGTCCGTGCCT
Homo_sapiens_chr6.trna125-ThrCGT (28564822-28564749)
Thr (CGT) 74 bp Sc: 80.88
(SEQ ID NO: 732)
GGCTCTATGGCTTAGTTGGTTAAAGCGCCTGTCTCGTAAACAGGAGATCCTGGGTTCGAC
TCCCAGTGGGGCCT
Homo_sapiens_chr5.trna13-ThrTGT (180551364-180551293)
Thr (TGT) 72 bp Sc: 75.64
(SEQ ID NO: 733)
GGCTCCATAGCTCAGGGGTTAGAGCACTGGTCTTGTAAACCAGGGTCGCGAGTTCAAATC
TCGCTGGGGCCT
Homo_sapiens_chr14.trna4-ThrTGT (20219689-20219761)
Thr (TGT) 73 bp Sc: 78.79
(SEQ ID NO: 734)
GGCCCTATAGCTCAGGGGTTAGAGCACTGGTCTTGTAAACCAGGGGTCGCGAGTTCAAAT
CTCGCTGGGGCCT
Homo_sapiens_chr14.trna20-ThrTGT (20169231-20169159)
Thr (TGT) 73 bp Sc: 79.46
(SEQ ID NO: 735)
GGCTCCATAGCTCAGGGGTTAGAGCACTGGTCTTGTAAACCAGGGGTCGCGAGTTCAAAT
CTCGCTGGGGCCT
Homo_sapiens_chr14.trna21-ThrTGT (20151861-20151789)
Thr (TGT) 73 bp Sc: 80.94
(SEQ ID NO: 736)
GGCTCCATAGCTCAGGGGTTAGAGCGCTGGTCTTGTAAACCAGGGGTCGCGAGTTCAATT
CTCGCTGGGGCCT
Homo_sapiens_chr1.trna56-ThrTGT (220704970-220705042)
Thr (TGT) 73 bp Sc: 83.03
(SEQ ID NO: 737)
GGCTCCATAGCTCAGTGGTTAGAGCACTGGTCTTGTAAACCAGGGGTCGCGAGTTCGATC
CTCGCTGGGGCCT
Homo_sapiens_chr6.trna127-ThrTGT (28550381-28550308)
Thr (TGT) 74 bp Sc: 84.82
(SEQ ID NO: 738)
GGCTCTATGGCTTAGTTGGTTAAAGCGCCTGTCTTGTAAACAGGAGATCCTGGGTTCGAA
TCCCAGTAGAGCCT
Homo_sapiens_chr11.trna19-TrpCCA (45246849-45246776)
Trp (CCA) 74 bp Sc: 21.17
(SEQ ID NO: 739)
GGAAGGATGGGGCCAAGCTGGAAAGCCTGTGGGCTCCACAGTCATGTGCCTGGGTTCAAT
TCCCAGTTCTGCAT
Homo_sapiens_chr7.trna1-TrpCCA (98905243-98905314)
Trp (CCA) 72 bp Sc: 70.08
(SEQ ID NO: 740)
GACCTCGTGGCGCAACGGCAGCGCGTCTGACTCCAGATCAGAAGGTTGCGTGTTCAAATC
ACGTCGGGGTCA
Homo_sapiens_chr12.trna6-TrpCCA (97422161-97422232)
Trp (CCA) 72 bp Sc: 71.65
(SEQ ID NO: 741)
GACCTCGTGGCGCAACGGTAGCGCGTCTGACTCCAGATCAGAAGGCTGCGTGTTCGAATC
ACGTCGGGGTCA
Homo_sapiens_chr17.trna6-TrpCCA (8030401-8030472)
Trp (CCA) 72 bp Sc: 74.00
(SEQ ID NO: 742)
GACCTCGTGGCGCAACGGTAGCGCGTCTGACTCCAGATCAGAAGGTTGCGTGTTCAAATC
ACGTCGGGGTCA
Homo_sapiens_chr6.trna168-TrpCCA (26439722-26439651)
Trp (CCA) 72 bp Sc: 74.00
(SEQ ID NO: 743)
GACCTCGTGGCGCAACGGTAGCGCGTCTGACTCCAGATCAGAAGGTTGCGTGTTCAAATC
ACGTCGGGGTCA
Homo_sapiens_chr6.trna170-TrpCCA (26427380-26427309)
Trp (CCA) 72 bp Sc: 74.00
(SEQ ID NO: 744)
GACCTCGTGGCGCAACGGTAGCGCGTCTGACTCCAGATCAGAAGGTTGCGTGTTCAAATC
ACGTCGGGGTCA
Homo_sapiens_chr17.trna12-TrpCCA (19352086-19352157)
Trp (CCA) 72 bp Sc: 74.80
(SEQ ID NO: 745)
GACCTCGTGGCGCAATGGTAGCGCGTCTGACTCCAGATCAGAAGGTTGCGTGTTCAAGTC
ACGTCGGGGTCA
Homo_sapiens_chr17.trna39-TrpCCA (8064983-8064912)
Trp (CCA) 72 bp Sc: 74.81
(SEQ ID NO: 746)
GGCCTCGTGGCGCAACGGTAGCGCGTCTGACTCCAGATCAGAAGGTTGCGTGTTCAAATC
ACGTCGGGGTCA
Homo_sapiens_chr9.trna3-TrpCCA (114656810-114656908)
Trp (CCA) 99 bp Sc: 23.38
(SEQ ID NO: 747)
GGCAGAGGAGGGTGCAGTTGGCAGCCTGTCCAAGTCCAGCACGGTTGGAGCACAGGATTT
AGAATGGGATGGTCCTGGGTTCAAACCCCAGCTGCGCCC
Homo_sapiens_chr2.trna14-TyrATA (218818794-218818886)
Tyr (ATA) 93 bp Sc: 55.93
(SEQ ID NO: 748)
CCTTCAATAGTTCAGCTGGTAGAGCAGAGGACTATAGCTACTTCCTCAGTAGGAGACGTC
CTTAGGTTGCTGGTTCGATTCCAGCTTGAAGGA
Homo_sapiens_chr7.trna9-TyrGTA (148886066-148886138)
Tyr (GTA) 73 bp Sc: 49.38
(SEQ ID NO: 749)
GGGGGTATAGCTCAGGGCTAGAGCTTTTTGACTGTAGAGCAAGAGGTCCCTGGTTCAAAT
CCAGGTTCTCCCT
Homo_sapiens_chr14.trna19-TyrGTA (20191191-20191098)
Tyr (GTA) 94 bp Sc: 74.71
(SEQ ID NO: 750)
CCTTCGATAGCTCAGCTGGTAGAGCGGAGGACTGTAGCCTGTAGAAACATTTGTGGACAT
CCTTAGGTCGCTGGTTCGATTCCGGCTCGAAGGA
Homo_sapiens_chr14.trna18-TyrGTA (20195556-20195463)
Tyr (GTA) 94 bp Sc: 75.82
(SEQ ID NO: 751)
CCTTCGATAGCTCAGCTGGTAGAGCGGAGGACTGTAGATTGTATAGACATTTGCGGACAT
CCTTAGGTCGCTGGTTCGATTCCAGCTCGAAGGA
Homo_sapiens_chr14.trna17-TyrGTA (20198050-20197957)
Tyr (GTA) 94 bp Sc: 71.83
(SEQ ID NO: 752)
CCTTCGATAGCTCAGCTGGTAGAGCGGAGGACTGTAGACTGCGGAAACGTTTGTGGACAT
CCTTAGGTCGCTGGTTCAATTCCGGCTCGAAGGA
Homo_sapiens_chr14.trna16-TyrGTA (20201284-20201191)
Tyr (GTA) 94 bp Sc: 74.71
(SEQ ID NO: 753)
CCTTCGATAGCTCAGCTGGTAGAGCGGAGGACTGTAGATTGTACAGACATTTGCGGACAT
CCTTAGGTCGCTGGTTCGATTCCGGCTCGAAGGA
Homo_sapiens_chr14.trna5-TyrGTA (20221272-20221360)
Tyr (GTA) 89 bp Sc: 75.42
(SEQ ID NO: 754)
CCTTCGATAGCTCAGCTGGTAGAGCGGAGGACTGTAGTACTTAATGTGTGGTCATCCTTA
GGTCGCTGGTTCGATTCCGGCTCGAAGGA
Homo_sapiens_chr6.trna14-TyrGTA (26677065-26677155)
Tyr (GTA) 91 bp Sc: 76.45
(SEQ ID NO: 755)
CCTTCGATAGCTCAGTTGGTAGAGCGGAGGACTGTAGTTGGCTGTGTCCTTAGACATCCT
TAGGTCGCTGGTTCGAATCCGGCTCGAAGGA
Homo_sapiens_chr6.trna15-TyrGTA (26683777-26683866)
Tyr (GTA) 90 bp Sc: 72.82
(SEQ ID NO: 756)
CTTTCGATAGCTCAGTTGGTAGAGCGGAGGACTGTAGGTTCATTAAACTAAGGCATCCTT
AGGTCGCTGGTTCGAATCCGGCTCGAAGGA
Homo_sapiens_chr6.trna16-TyrGTA (26685311-26685399)
Tyr (GTA) 89 bp Sc: 77.01
(SEQ ID NO: 757)
CCTTCGATAGCTCAGTTGGTAGAGCGGAGGACTGTAGGCTCATTAAGCAAGGTATCCTTA
GGTCGCTGGTTCGAATCCGGCTCGGAGGA
Homo_sapiens_chr6.trna17-TyrGTA (26703081-26703169)
Tyr (GTA) 89 bp Sc: 73.91
(SEQ ID NO: 758)
CCTTCGATAGCTCAGCTGGTAGAGCGGAGGACTGTAGGGGTTTGAATGTGGTCATCCTTA
GGTCGCTGGTTCGAATCCGGCTCGGAGGA
Homo_sapiens_chr2.trna2-TyrGTA (27127154-27127242)
Tyr (GTA) 89 bp Sc: 75.87
(SEQ ID NO: 759)
CCTTCGATAGCTCAGTTGGTAGAGCGGAGGACTGTAGTGGATAGGGCGTGGCAATCCTTA
GGTCGCTGGTTCGATTCCGGCTCGAAGGA
Homo_sapiens_chr8.trna12-TyrGTA (66772173-66772086)
Tyr (GTA) 88 bp Sc: 46.11
(SEQ ID NO: 760)
TCTTCAATAGCTCAGCTGGTAGAGCGGAGGACTGTAGGTGCACGCCCGTGGCCATTCTTA
GGTGCTGGTTTGATTCCGACTTGGAGAG
Homo_sapiens_chr8.trna4-TyrGTA (67188156-67188248)
Tyr (GTA) 93 bp Sc: 73.04
(SEQ ID NO: 761)
CCTTCGATAGCTCAGCTGGTAGAGCGGAGGACTGTAGCTACTTCCTCAGCAGGAGACATC
CTTAGGTCGCTGGTTCGATTCCGGCTCGAAGGA
Homo_sapiens_chr8.trna5-TyrGTA (67188777-67188865)
Tyr (GTA) 89 bp Sc: 69.69
(SEQ ID NO: 762)
CCTTCGATAGCTCAGCTGGTAGAGCGGAGGACTGTAGGCGCGCGCCCGTGGCCATCCTTA
GGTCGCTGGTTCGATTCCGGCTCGAAGGA
Homo_sapiens_chr1.trna63-ValAAC (178450971-178450899)
Val (AAC) 73 bp Sc: 48.28
(SEQ ID NO: 763)
GTTTCCATAGTGTACTGGTTATCACATTCACCTAACACGCGAAAGGTCCTTGGTTTGAAA
CCAGGCAGAAACA
Homo_sapiens_chr6.trna115-ValAAC (28811256-28811185)
Val (AAC) 72 bp Sc: 64.08
(SEQ ID NO: 764)
GGGGGTGTAGCTCAGTGGTAGAGCGTATGCTTAACATTCATGAGGCTCTGGGTTCGATCC
CCAGCACTTCCA
Homo_sapiens_chr6.trna37-ValAAC (27311267-27311339)
Val (AAC) 73 bp Sc: 74.63
(SEQ ID NO: 765)
GTTTCCGTAGTGTAGTGGTTATCACGTTTGCCTAACACGCGAAAGGTCCCCGGTTCGAAA
CCGGGCAGAAACA
Homo_sapiens_chr6.trna136-ValAAC (27756936-27756864)
Val (AAC) 73 bp Sc: 79.68
(SEQ ID NO: 766)
GTTTCCGTAGTGTAGTGGTTATCACGTTCGCCTAACACGCGAAAGGTCCGCGGTTCGAAA
CCGGGCGGAAACA
Homo_sapiens_chr6.trna139-ValAAC (27726758-27726686)
Val (AAC) 73 bp Sc: 81.58
(SEQ ID NO: 767)
GTTTCCGTAGTGTAGTGGTTATCACGTTCGCCTAACACGCGAAAGGTCCCTGGATCAAAA
CCAGGCGGAAACA
Homo_sapiens_chr5.trna15-ValAAC (180548094-180548022)
Val (AAC) 73 bp Sc: 84.70
(SEQ ID NO: 768)
GTTTCCGTAGTGTAGTGGTCATCACGTTCGCCTAACACGCGAAAGGTCCCCGGTTCGAAA
CCGGGCGGAAACA
Homo_sapiens_chr3.trna2-ValAAC (170972712-170972784)
Val (AAC) 73 bp Sc: 86.79
(SEQ ID NO: 769)
GTTTCCGTAGTGTAGTGGTTATCACGTTCGCCTAACACGCGAAAGGTCCCCGGTTCGAAA
CCGGGCGGAAACA
Homo_sapiens_chr5.trna12-ValAAC (180577948-180577876)
Val (AAC) 73 bp Sc: 86.79
(SEQ ID NO: 770)
GTTTCCGTAGTGTAGTGGTTATCACGTTCGCCTAACACGCGAAAGGTCCCCGGTTCGAAA
CCGGGCGGAAACA
Homo_sapiens_chr5.trna4-ValAAC (180523760-180523832)
Val (AAC) 73 bp Sc: 86.79
(SEQ ID NO: 771)
GTTTCCGTAGTGTAGTGGTTATCACGTTCGCCTAACACGCGAAAGGTCCCCGGTTCGAAA
CCGGGCGGAAACA
Homo_sapiens_chr5.trna5-ValAAC (180529216-180529288)
Val (AAC) 73 bp Sc: 86.79
(SEQ ID NO: 772)
GTTTCCGTAGTGTAGTGGTTATCACGTTCGCCTAACACGCGAAAGGTCCCCGGTTCGAAA
CCGGGCGGAAACA
Homo_sapiens_chr6.trna132-ValAAC (27829230-27829158)
Val (AAC) 73 bp Sc: 86.79
(SEQ ID NO: 773)
GTTTCCGTAGTGTAGTGGTTATCACGTTCGCCTAACACGCGAAAGGTCCCCGGTTCGAAA
CCGGGCGGAAACA
Homo_sapiens_chr6.trna58-ValCAC (27758467-27758540)
Val (CAC) 74 bp Sc: 33.44
(SEQ ID NO: 774)
TTTTCTGTAGTGTAGTTGTTAACACGTTCGCCTCACACGCTTAAAGTTCTCTGGTTGGAT
ACCAGATGGAAATG
Homo_sapiens_chr1.trna129-ValCAC (16879160-16879088)
Val (CAC) 73 bp Sc: 52.98
(SEQ ID NO: 775)
GTTTCTGTGGTGTAGTGGTTATCATGTTCGCCTCACACGAGAAAAGTCCCTGATTCGAGA
CTGGGTGGGAACG
Homo_sapiens_chr6.trna32-ValCAC (27226001-27226073)
Val (CAC) 73 bp Sc: 56.94
(SEQ ID NO: 776)
GTTTCTGTAGTATGGTGGTTATCACGTTAGTCTCACACGTGAAAGGTCCCTGGTTCGAAA
CCAGGTGGAAACA
Homo_sapiens_chr1.trna99-ValCAC (147561360-147561290)
Val (CAC) 71 bp Sc: 60.62
(SEQ ID NO: 777)
GCACTGGTGGTTCAGTGGTAGAATTCTCGCCTCACACGCGGGACACCCGGGTTCAATTCC
CGGTCAAGGCA
Homo_sapiens_chr6.trna133-ValCAC (27804378-27804306)
Val (CAC) 73 bp Sc: 64.50
(SEQ ID NO: 778)
GTTTCCGTAGTGTAGTGGTTATTATGTTCGCCTCACACGCGAAAAGTCCCCGGTTCGAAA
TCAGGCGGGAACA
Homo_sapiens_chr6.trna157-ValCAC (27281918-27281846)
Val (CAC) 73 bp Sc: 73.23
(SEQ ID NO: 779)
GTTTCCGTAGTGGAGTGGTTATCACGTTCGCCTCACACGCGAAAGGTCCCCGGTTTGAAA
CCAGGCGGAAACA
Homo_sapiens_chr1.trna90-ValCAC (147950785-147950712)
Val (CAC) 74 bp Sc: 77.15
(SEQ ID NO: 780)
GTTTCCGTAGTGTAGTGGTTATCACGTTCGCCTCACACGCGTAAAGGTCCCCGGTTCGAA
ACCGGGCGGAAACA
Homo_sapiens_chr1.trna98-ValCAC (147565251-147565179)
Val (CAC) 73 bp Sc: 82.06
(SEQ ID NO: 781)
GTTTCCGTAGTGTAGTGGTTATCACGTTCGCCTCACACGCGAAAGGTCCCCGGTTCGAAA
CTGGGCGGAAACA
Homo_sapiens_chr19.trna13-ValCAC (4675719-4675647)
Val (CAC) 73 bp Sc: 84.20
(SEQ ID NO: 782)
GTTTCCGTAGTGTAGCGGTTATCACATTCGCCTCACACGCGAAAGGTCCCCGGTTCGATC
CCGGGCGGAAACA
Homo_sapiens_chr6.trna152-ValCAC (27356100-27356028)
Val (CAC) 73 bp Sc: 87.34
(SEQ ID NO: 783)
GCTTCTGTAGTGTAGTGGTTATCACGTTCGCCTCACACGCGAAAGGTCCCCGGTTCGAAA
CCGGGCAGAAGCA
Homo_sapiens_chr1.trna85-ValCAC (159636186-159636114)
Val (CAC) 73 bp Sc: 87.39
(SEQ ID NO: 784)
GTTTCCGTAGTGTAGTGGTTATCACGTTCGCCTCACACGCGAAAGGTCCCCGGTTCGAAA
CCGGGCGGAAACA
Homo_sapiens_chr5.trna10-ValCAC (180582073-180582001)
Val (CAC) 73 bp Sc: 87.39
(SEQ ID NO: 785)
GTTTCCGTAGTGTAGTGGTTATCACGTTCGCCTCACACGCGAAAGGTCCCCGGTTCGAAA
CCGGGCGGAAACA
Homo_sapiens_chr5.trna18-ValCAC (180461931-180461859)
Val (CAC) 73 bp Sc: 87.39
(SEQ ID NO: 786)
GTTTCCGTAGTGTAGTGGTTATCACGTTCGCCTCACACGCGAAAGGTCCCCGGTTCGAAA
CCGGGCGGAAACA
Homo_sapiens_chr5.trna2-ValCAC (180456676-180456748)
Val (CAC) 73 bp Sc: 87.39
(SEQ ID NO: 787)
GTTTCCGTAGTGTAGTGGTTATCACGTTCGCCTCACACGCGAAAGGTCCCCGGTTCGAAA
CCGGGCGGAAACA
Homo_sapiens_chr5.trna6-ValCAC (180533256-180533328)
Val (CAC) 73 bp Sc: 87.39
(SEQ ID NO: 788)
GTTTCCGTAGTGTAGTGGTTATCACGTTCGCCTCACACGCGAAAGGTCCCCGGTTCGAAA
CCGGGCGGAAACA
Homo_sapiens_chr6.trna9-ValCAC (26646261-26646333)
Val (CAC) 73 bp Sc: 87.39
(SEQ ID NO: 789)
GTTTCCGTAGTGTAGTGGTTATCACGTTCGCCTCACACGCGAAAGGTCCCCGGTTCGAAA
CCGGGCGGAAACA
Homo_sapiens_chr6.trna40-ValTAC (27366384-27366456)
Val (TAC) 73 bp Sc: 76.42
(SEQ ID NO: 790)
GTTTCCGTGGTGTAGTGGTTATCACATTCGCCTTACACGCGAAAGGTCCTCGGGTCGAAA
CCGAGCGGAAACA
Homo_sapiens_chr10.trna6-ValTAC (5935752-5935680)
Val (TAC) 73 bp Sc: 81.67
(SEQ ID NO: 791)
GGTTCCATAGTGTAGTGGTTATCACATCTGCTTTACACGCAGAAGGTCCTGGGTTCAAGC
CCCAGTGGAACCA
Homo_sapiens_chr11.trna16-ValTAC (59075108-59075036)
Val (TAC) 73 bp Sc: 82.67
(SEQ ID NO: 792)
GGTTCCATAGTGTAGCGGTTATCACGTCTGCTTTACACGCAGAAGGTCCTGGGTTCGAGC
CCCAGTGGAACCA
Homo_sapiens_chr11.trna17-ValTAC (59074750-59074678)
Val (TAC) 73 bp Sc: 85.12
(SEQ ID NO: 793)
GGTTCCATAGTGTAGTGGTTATCACGTCTGCTTTACACGCAGAAGGTCCTGGGTTCGAGC
CCCAGTGGAACCA
Homo_sapiens_chrX.trna4-ValTAC (18603022-18602950)
Val (TAC) 73 bp Sc: 85.12
(SEQ ID NO: 794)
GGTTCCATAGTGTAGTGGTTATCACGTCTGCTTTACACGCAGAAGGTCCTGGGTTCGAGC
CCCAGTGGAACCA
Homo_sapiens_chr9.trna8-GlnTTG (5085156-5085085)
Gln (TTG) 72 bp Sc: 21.75
(SEQ ID NO: 795)
TAGGATATGGTTTAATAGGTAGCATGGAGAATTTTGGAGTCTCAGGGATAGGTTCAATTC
CTATAGTTCCAG
Homo_sapiens_chr6.trna64-GlnTTG (28665135-28665206)
Gln (TTG) 72 bp Sc: 71.88
(SEQ ID NO: 796)
GGTCCCATGGTGTAATGGTTAGCACTCTGGACTTTGAATCCAGCAATCCGAGTTCGAATC
TCGGTGGGACCT

What is claimed is:

1. A neuroprotective molecule comprising:
a sequence of 25-35 contiguous nucleotides that is at least 80% identical to a contiguous sequence between nucleotide 1 and nucleotide 50 of a mature human tRNA; and
at least four contiguous guanosine-containing nucleotides;
wherein the sequence of 25-35 contiguous nucleotides comprises a D-loop stem structure, the at least four contiguous guanosine-containing nucleotides are located at the 5′ end of the neuroprotective molecule, and the neuroprotective molecule comprises at least one deoxyribonucleotide.

2. The neuroprotective molecule of claim 1, wherein the sequence of 25-35 contiguous nucleotides is at least 80% identical to a contiguous sequence between nucleotide 1 and nucleotide 50 of a mature human tRNA having a sequence selected from the group consisting of SEQ ID NOS: 4, 5, 8-11, 13-17, 32, 37, and 40-173.

3. A neuroprotective molecule comprising:
a sequence of 25-35 contiguous nucleotides that is at least 80% identical to a contiguous sequence between nucleotide 1 and nucleotide 50 of a mature human tRNA selected from the group consisting of: tRNA^{Arg}, tRNA^{Asp}, tRNA^{Glu}, tRNA^{Gln}, tRNA^{Gly}, tRNA^{His}, tRNA^{Ile}, tRNA^{Leu}, tRNA^{Lys}, tRNA^{Met}, tRNA^{Pro}, tRNA^{SeC}, tRNA^{Ser}, tRNA^{Sup}, tRNA^{Thr}, tRNA^{Trp}, tRNA^{Tyr}, tRNA^{Val} tRNA^{Asn}, and tRNA^{Phe}, and
at least four contiguous guanosine-containing nucleotides;
wherein the sequence of 25-35 contiguous nucleotides comprises a D-loop stem structure and the at least four contiguous guanosine-containing nucleotides are located at the 5′ end of the neuroprotective molecule.

4. A neuroprotective molecule of claim 3, wherein the sequence of 25-35 contiguous nucleotides is at least 80% identical to a contiguous sequence between nucleotide 1 and nucleotide 50 of a mature human tRNA having a sequence selected from the group consisting of SEQ ID NOS: 5, 8, 9, 11, 14-17, 32, 37, 56, 57, and 63-173.

5. The neuroprotective molecule of claim 4, wherein the sequence of 25-35 contiguous nucleotides is at least 80% identical to a contiguous sequence between nucleotide 1 and nucleotide 50 of a mature human tRNA having a sequence selected from the group of SEQ ID NOS: 11 and 107-116.

6. The neuroprotective molecule of claim 1, wherein the neuroprotective molecule further comprises a 5′-monophosphate.

7. The neuroprotective molecule of claim 1, wherein the neuroprotective molecule comprises at least one modified nucleotide.

8. The neuroprotective molecule of claim 7, wherein the modified nucleotide contains a modified base or a modified sugar.

9. The neuroprotective molecule of claim 1, wherein the neuroprotective molecule contains at least one modification in the phosphate backbone.

10. The neuroprotective molecule of claim 1, wherein the neuroprotective molecule contains a 5′- or a 3′-protective group.

11. The neuroprotective molecule of claim 1, wherein the neuroprotective molecule has a total length of between 39 to 60 nucleotides.

12. A pharmaceutical composition comprising at least one neuroprotective molecule of claim 1.

13. A method of inducing or increasing stress granule formation in a cell, the method comprising administering to a cell a neuroprotective molecule of claim 1, or an isolated C-myc oligonucleotide comprising the sequence of GGGGAGGGTGGGGAGGGT GGGG (SEQ ID NO: 174), wherein the neuroprotective molecule or the isolated C-myc oligonucleotide is administered in an amount sufficient to induce or increase stress granule formation in the cell.

14. A method of decreasing protein translation in a cell, the method comprising administering to a cell a neuroprotective molecule of claim 1, or an isolated C-myc oligonucleotide comprising the sequence of GGGGAGGGTGGGGAGGGTGGGG (SEQ ID NO: 174), where the neuroprotective molecule or the isolated C-myc oligonucleotide is administered in an amount sufficient to decrease protein translation in the cell.

15. A method of decreasing stress-induced cell death, the method comprising administering to a cell a neuroprotective molecule of claim 1, or an isolated C-myc oligonucleotide comprising the sequence of GGGGAGGGTGGGGAGGGTGGGG (SEQ ID NO: 174), where the neuroprotective molecule or the isolated C-myc oligonucleotide is administered in an amount sufficient to decrease cell death.

16. The method of claim 13, wherein the cell is in vitro.

17. The method of claim 13, wherein the cell is in vivo.

18. The method of claim 16, wherein the cell is a neuron.

19. The method of claim 18, wherein the cell is a motor neuron.

20. A method of treating a neurological disorder associated with neuron death in a subject, the method comprising administering a neuroprotective molecule of claim 1, or an isolated C-myc oligonucleotide comprising the sequence of GGGGAGGGTGGGGAGGGTGGGG (SEQ ID NO: 174), wherein the neuroprotective molecule or the isolated C-myc oligonucleotide is administered in an amount sufficient to treat the neurological disorder associated with neuron death in the subject.

21. The method of claim 20, wherein the neurological disorder associated with neuron death is selected from the group consisting of: amyotrophic lateral sclerosis, Alzheimer's disease, Parkinson's disease, Huntington's disease, muscular dystrophy, multiple sclerosis, and stroke.

22. The method of claim 20, wherein the neuroprotective molecule or the isolated C-myc oligonucleotide is administered intravenously, intraarterially, intracranially, ocularly, intraperitoneally, subcutaneously, or intramuscularly.
